(19) United States
(12) Plant Patent Application Publication
(10) Pub. No.: US 2026/0107858 P1
(43) Pub. Date: Apr. 16, 2026

(54) CACAO VARIETIES

(50) ;

(71) Applicant: Mars, Incorporated, McLean, VA (US)

(72) Inventor: Stefan Emiel ROYAERT, Mogi Mirim, Sao Paulo (BR)

(73) Assignee: Mars, Incorporated, McLean, VA (US)

(21) Appl. No.: 19/358,199

(22) Filed: Oct. 14, 2025

Related U.S. Application Data
(60) Provisional application No. 63/707,090, filed on Oct. 14, 2024.

(51) Int. Cl. A01H 6/00; A01H 5/08

(52) U.S. Cl. CPC A01H 6/00; A01H 5/08
USPC PLT/152

(57) ABSTRACT

A new and distinct variety of cacao plant selected from the group consisting of ‘CMC-001’, ‘CMC-002’, ‘CMC-003’, ‘CMC-004’, ‘CMC-005’, ‘CMC-006’, ‘CMC-007’, ‘CMC-008’, ‘CMC-009’, ‘CMC-010’, ‘CMC-011’, ‘CMC-012’, ‘CMC-013’, ‘CMC-014’, ‘CMC-015’, ‘CMC-016’, ‘CMC-017’, ‘CMC-018’, ‘CMC-019’, ‘CMC-021’, ‘CMC-021’, ‘CMC-023’, ‘CMC-024’, ‘CMC-025’, ‘CMC-026’, ‘CMC-027’, ‘CMC-028’, ‘CMC-029’, ‘CMC-030’, ‘CMC-031’, ‘CMC-032’, ‘CMC-033’, ‘CMC-034’, ‘CMC-035’, ‘CMC-036’, and ‘CMC-022’, is disclosed.

### CROSS-REFERENCE TO RELATED APPLICATION

This application claims the benefit of U.S. Provisional Application No. 63/707,090, filed Oct. 14, 2024, the disclosure of which is herein incorporated by reference in its entirety.

Latin name: Botanical classification: Theobroma cacao L.

Varietal denomination: Variety denomination: the varietal denominations of the cacao varieties disclosed herein are ‘CMC-001’, ‘CMC-002’, ‘CMC-003’, ‘CMC-004’, ‘CMC-005’, ‘CMC-006’, ‘CMC-007’, ‘CMC-008’, ‘CMC-009’, ‘CMC-010’, ‘CMC-011’, ‘CMC-012’, ‘CMC-013’, ‘CMC-014’, ‘CMC-015’, ‘CMC-016’, ‘CMC-017’, ‘CMC-018’, ‘CMC-019’, ‘CMC-020’, ‘CMC-021’, ‘CMC-023’, ‘CMC-024’, ‘CMC-025’, ‘CMC-026’, ‘CMC-027’, ‘CMC-028’, ‘CMC-029’, ‘CMC-030’, ‘CMC-031’, ‘CMC-032’, ‘CMC-033’, ‘CMC-034’, ‘CMC-035’, ‘CMC-036’, and ‘CMC-022’.

### BACKGROUND OF THE INVENTION

Cacao beans are produced by cacao trees which are found in warm, moist climates in areas about 20 degrees latitude north and south of the Equator. The cacao tree produces leaves, flowers and fruit throughout the year, and the ripe fruit or pod resembles a long cantaloupe, typically containing from about 20 to 40 almond-shaped cacao beans. Cacao butter and cacao solids are extracted from commercial cacao beans and processed to produce various cacao and chocolate products.

Different varieties of cacao trees have varying cacao bean yield, resistance to disease, and levels of compounds that impact the flavor and nutritional value of cacao products. Cacao is an important crop that has been appreciated by humans for thousands of years. There is a need for new cacao varieties, in particular improved varieties that are stable, high yielding, and agronomically sound.

### SUMMARY OF THE INVENTION

In order to meet these needs, the present invention is directed to improved varieties of Cacao plant.

Each new cacao plant of the present invention is stable and reproduces true to type in successive generations of asexual reproduction.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates a whole tree of variety ‘CMC-001’.

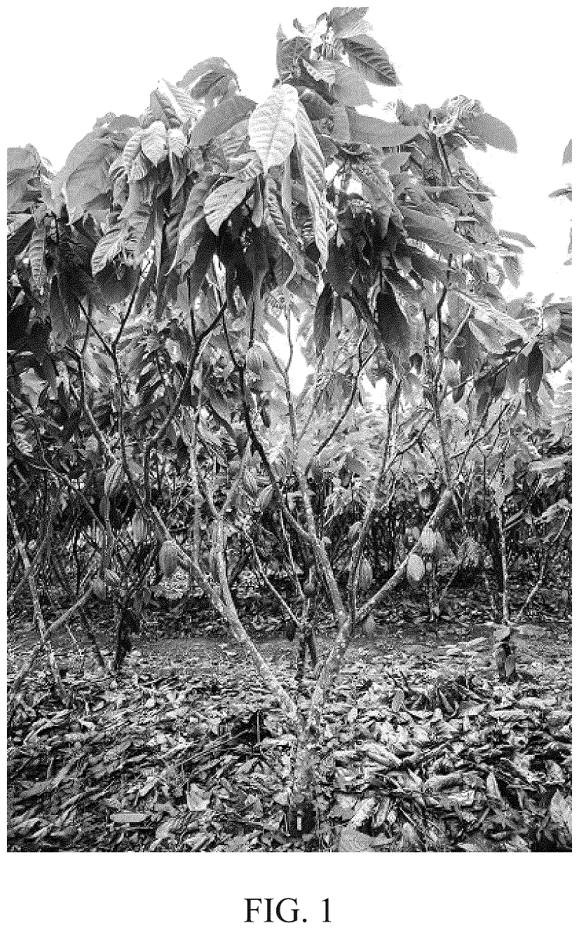

FIG. 2 illustrates a pod of variety ‘CMC-001’.

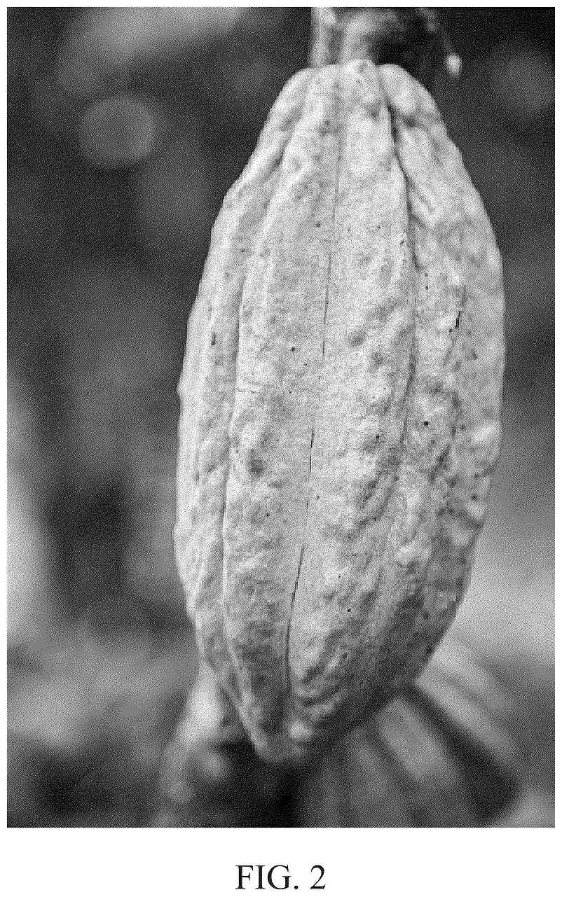

FIG. 3 illustrates a flower of variety ‘CMC-001’.

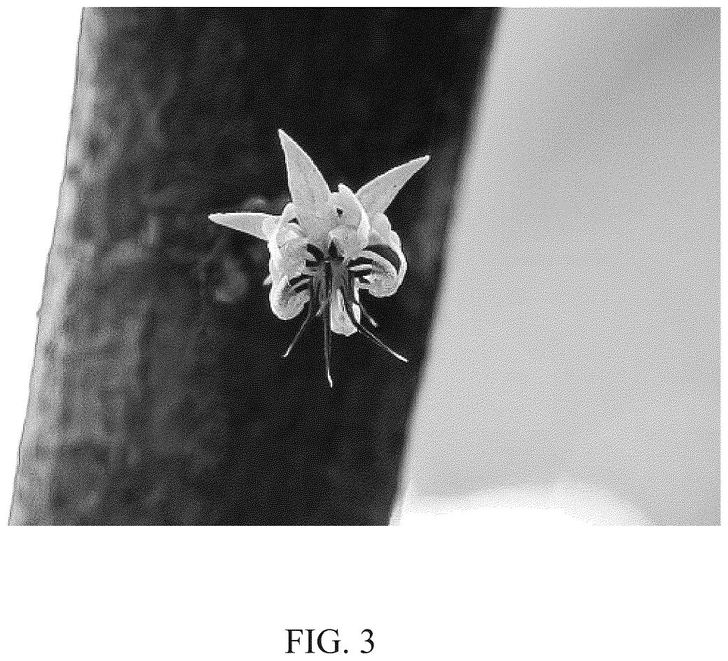

FIG. 4 illustrates flowers and flower buds of variety ‘CMC-001’.

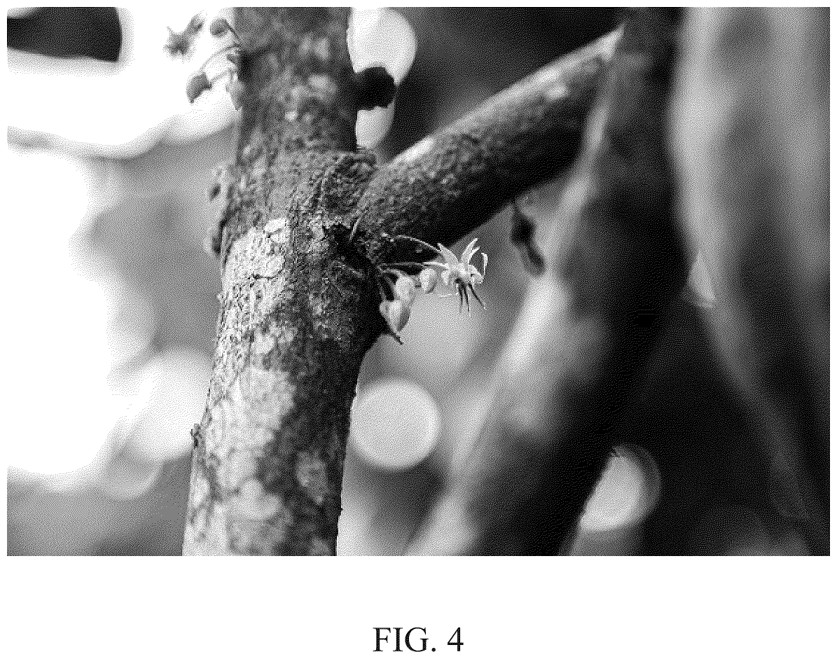

FIG. 5 illustrates leaves of variety ‘CMC-001’.

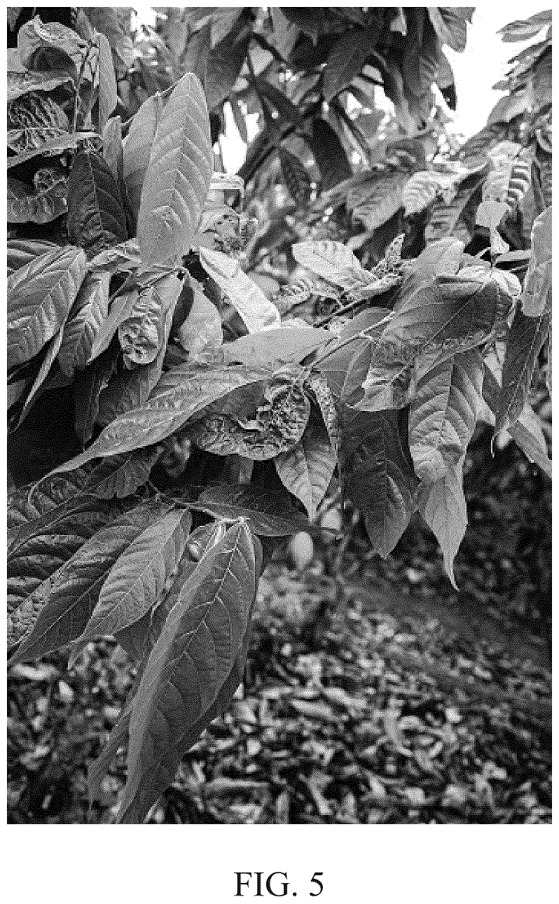

FIG. 6 illustrates a whole tree of variety ‘CMC-002’.

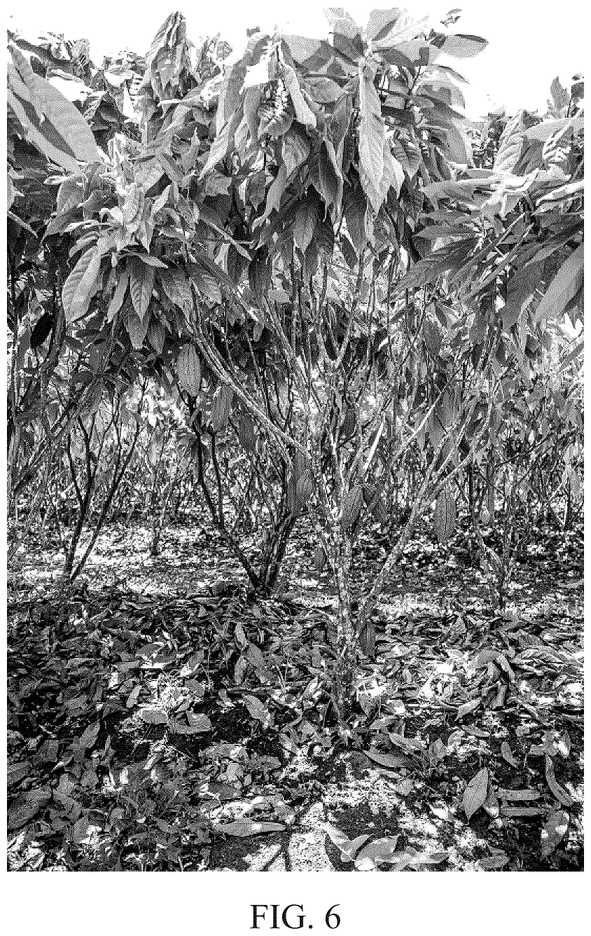

FIG. 7 illustrates a pod of variety ‘CMC-002’.

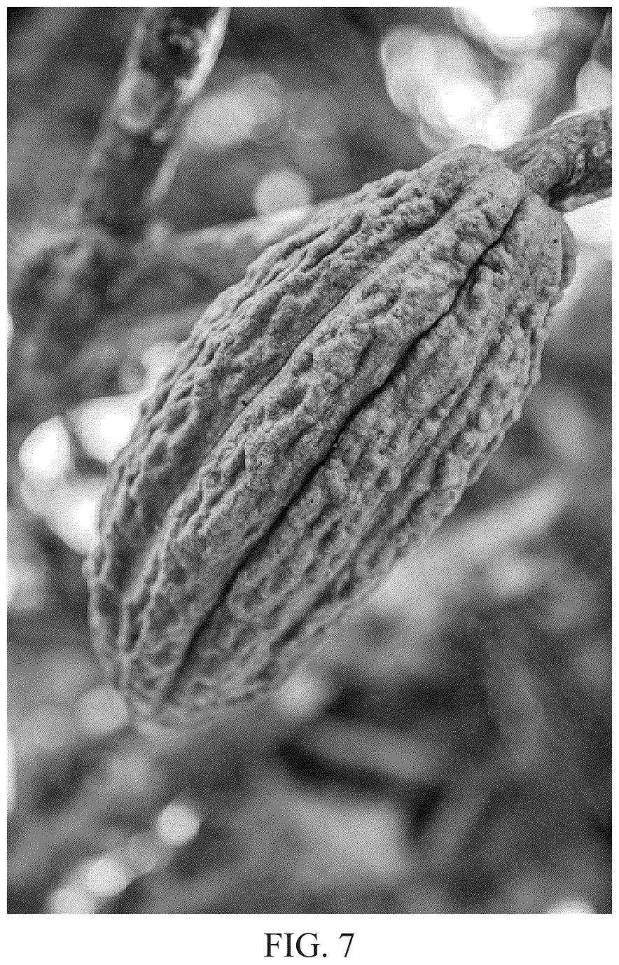

FIG. 8 illustrates a pod of variety ‘CMC-002’.

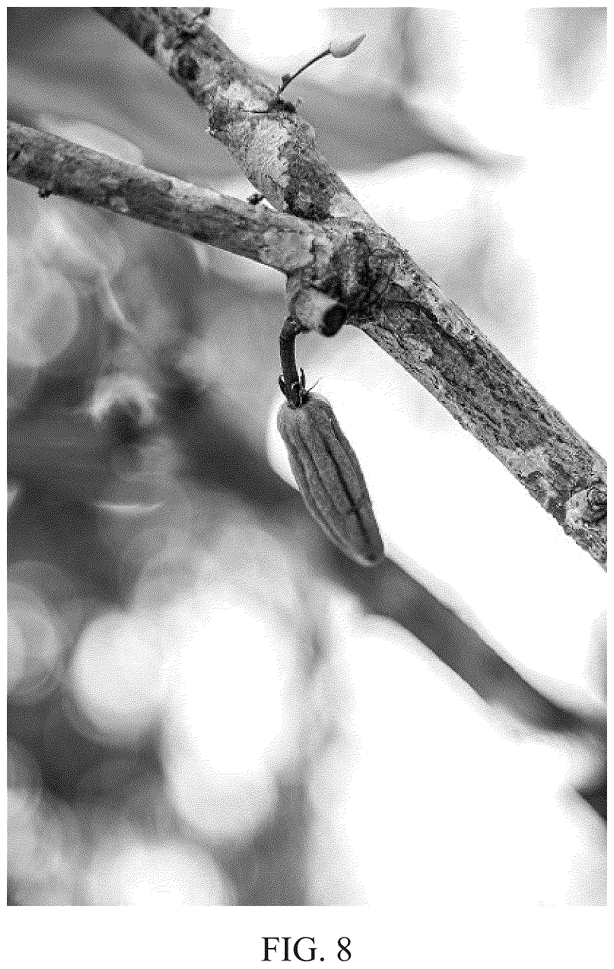

FIG. 9 illustrates a flower of variety ‘CMC-002’.

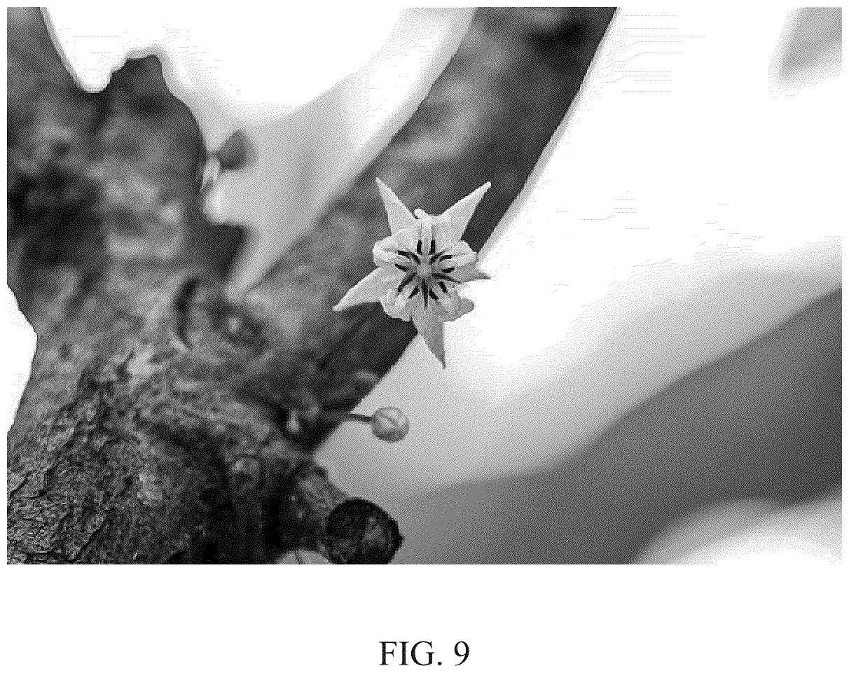

FIG. 10 illustrates flowers and flower buds of variety ‘CMC-002’.

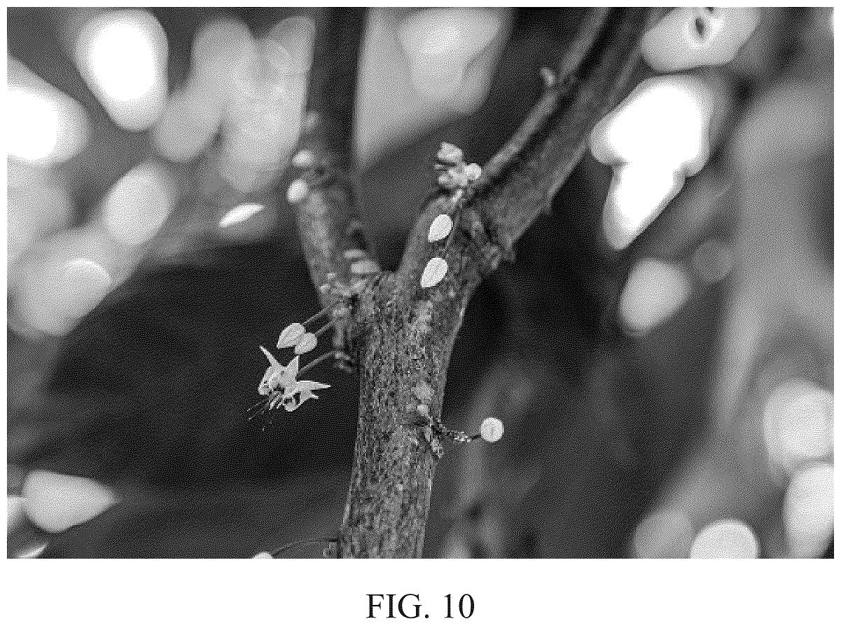

FIG. 11 illustrates leaves of variety ‘CMC-002’.

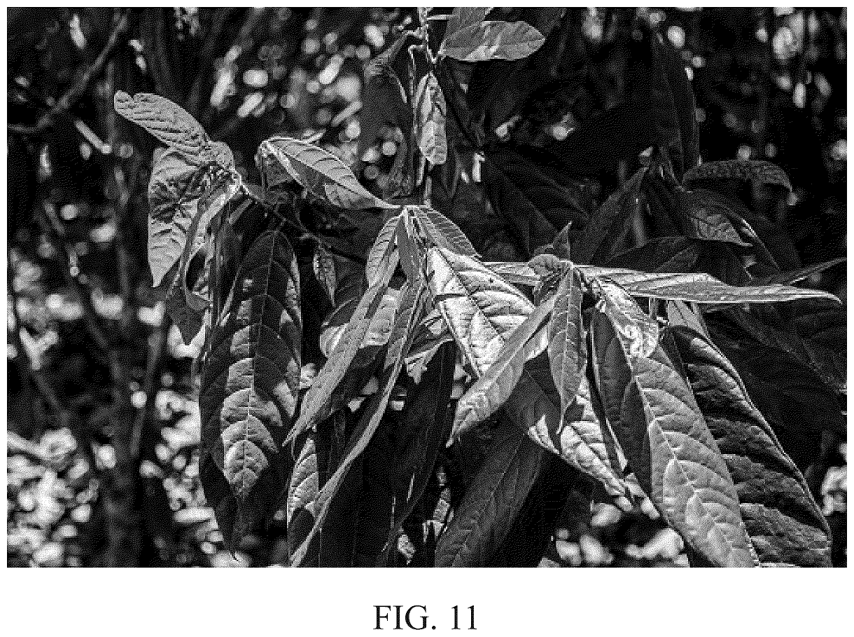

FIG. 12 illustrates a whole tree of variety ‘CMC-003’.

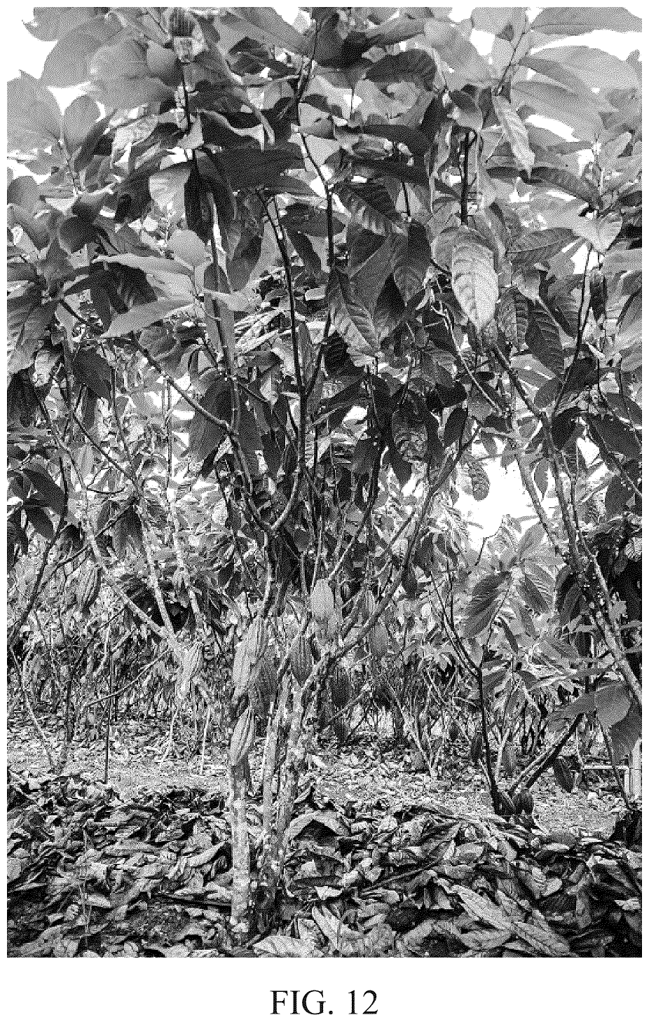

FIG. 13 illustrates a pod of variety ‘CMC-003’.

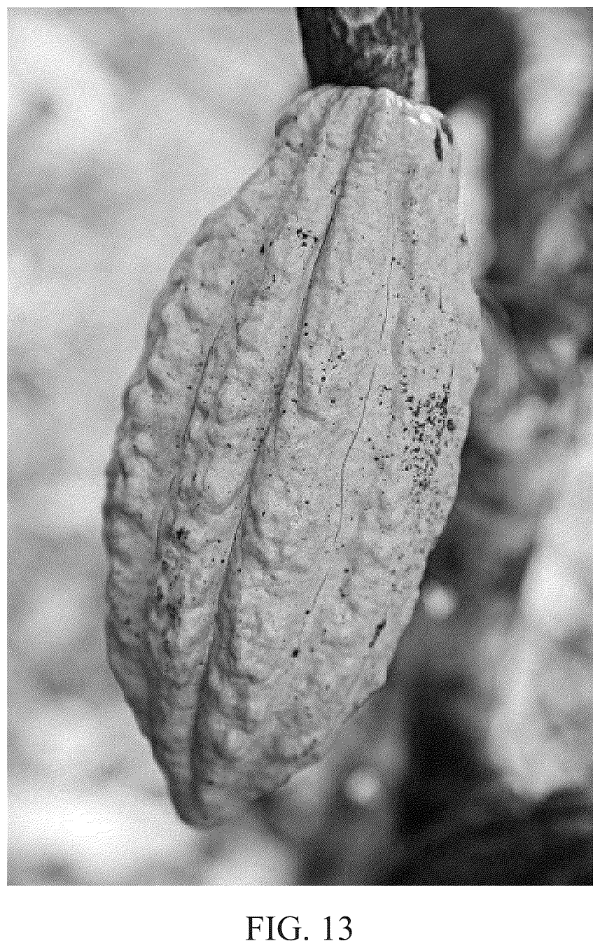

FIG. 14 illustrates a flower of variety ‘CMC-003’.

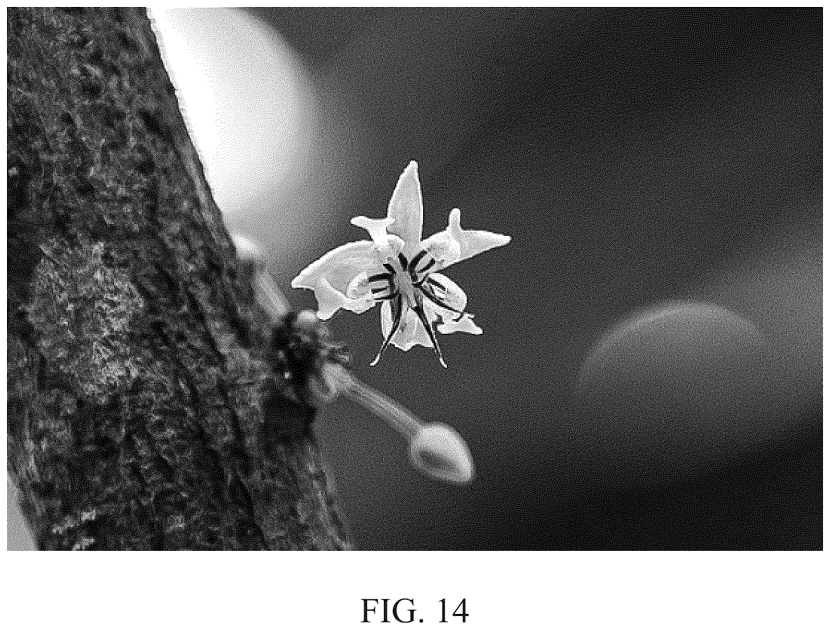

FIG. 15 illustrates flowers and flower buds of variety ‘CMC-003’.

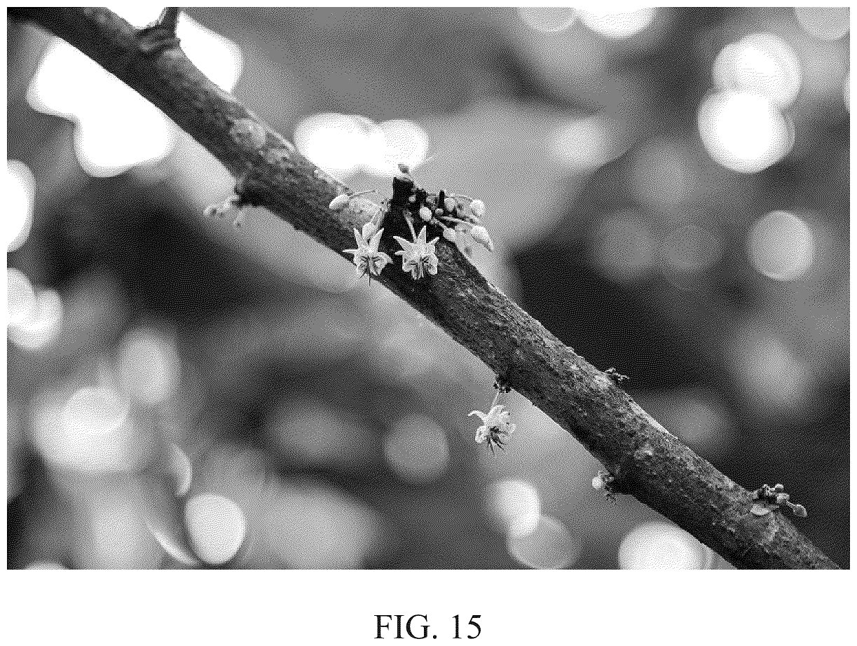

FIG. 16 illustrates leaves of variety ‘CMC-003’.

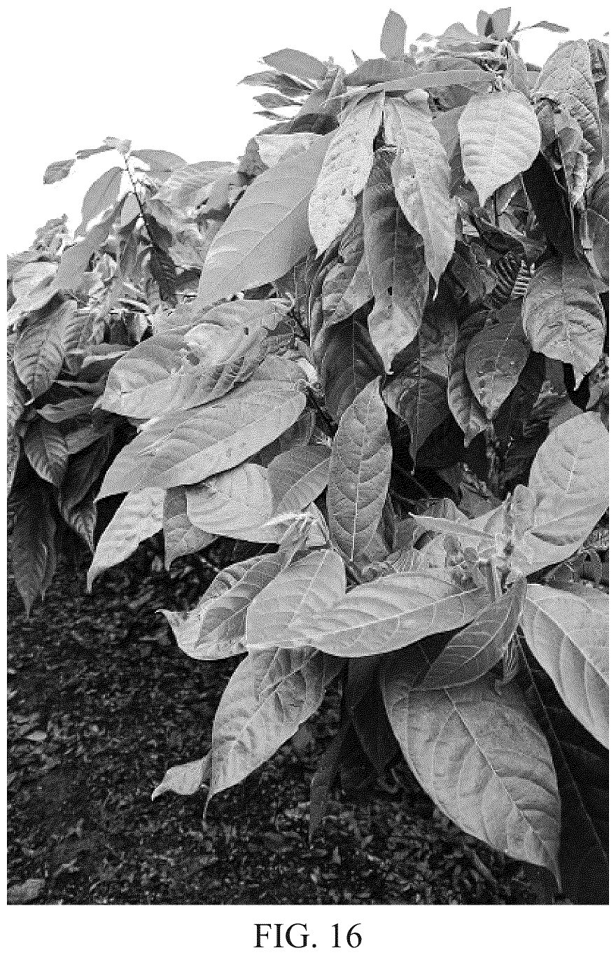

FIG. 17 illustrates a whole tree of variety ‘CMC-004’.

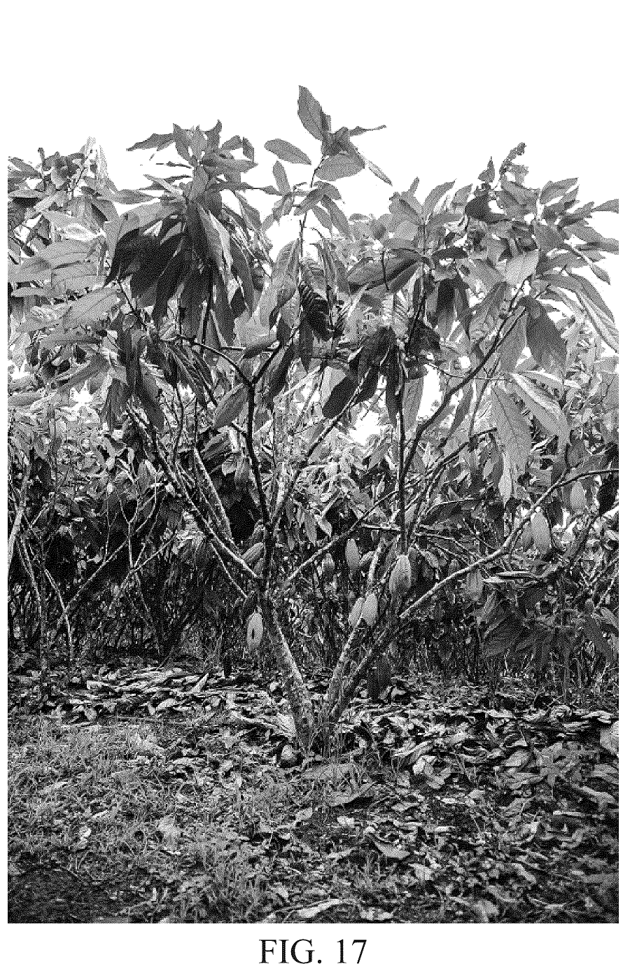

FIG. 18 illustrates a pod of variety ‘CMC-004’.

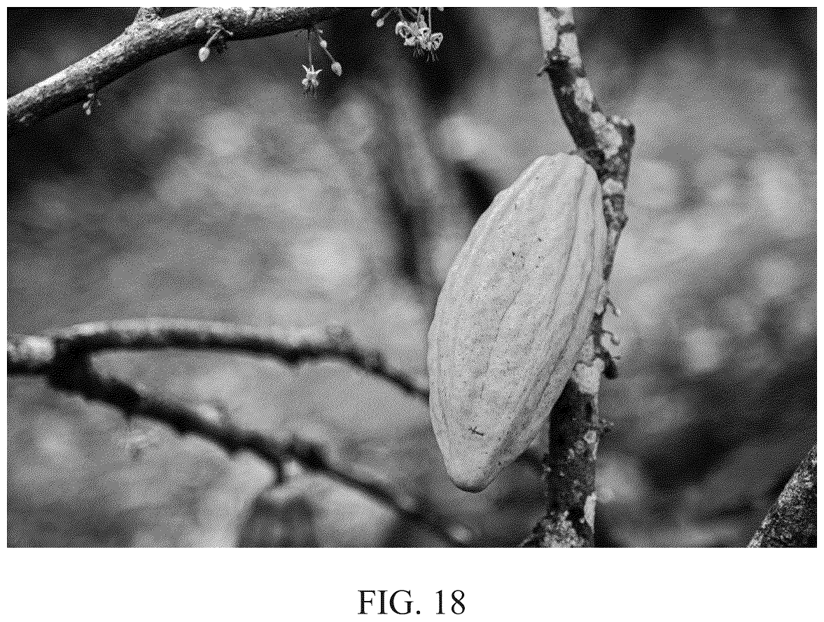

FIG. 19 illustrates a pod of variety ‘CMC-004’.

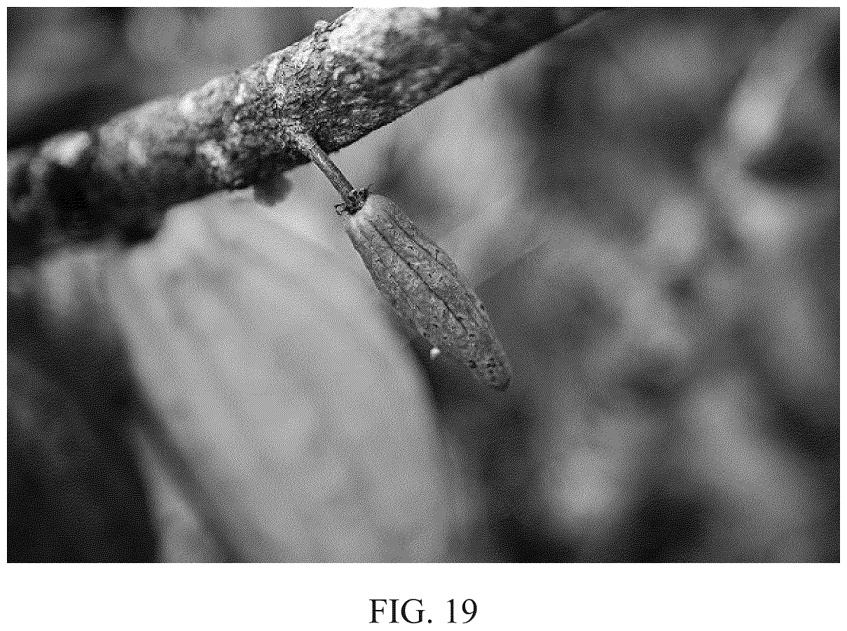

FIG. 20 illustrates a pod of variety ‘CMC-004’.

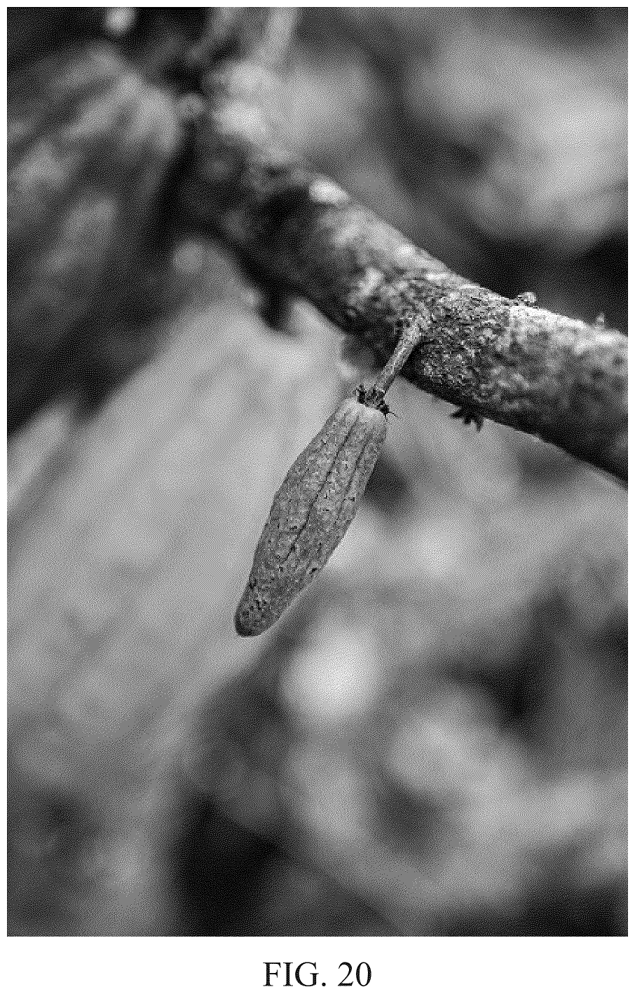

FIG. 21 illustrates a flower of variety ‘CMC-004’.

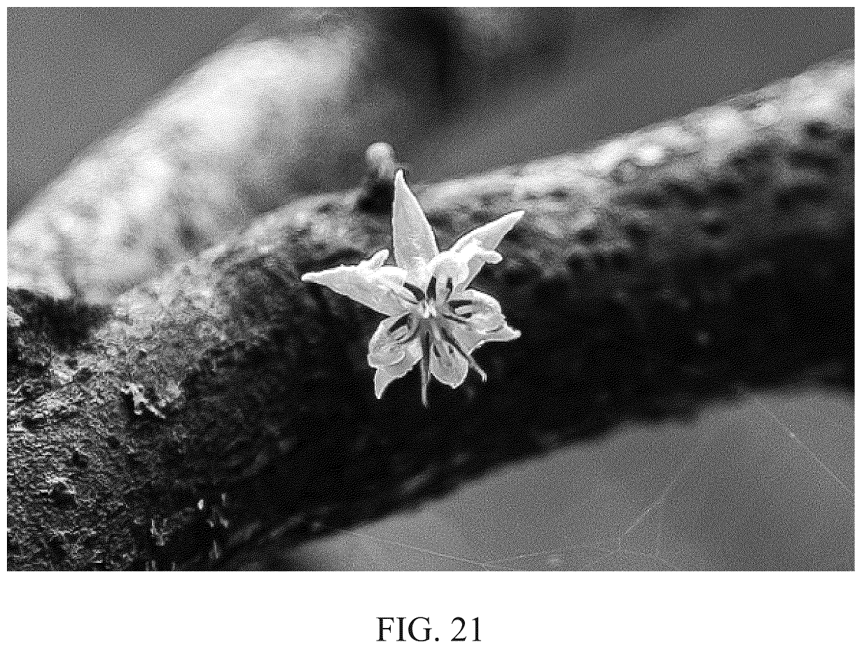

FIG. 22 illustrates a flower of variety ‘CMC-004’.

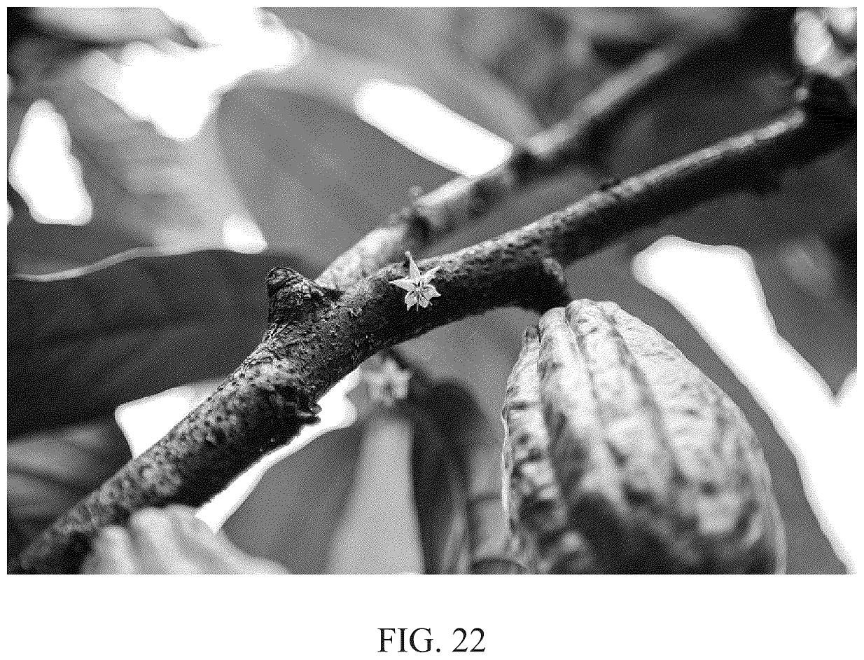

FIG. 23 illustrates flowers of variety ‘CMC-004’.

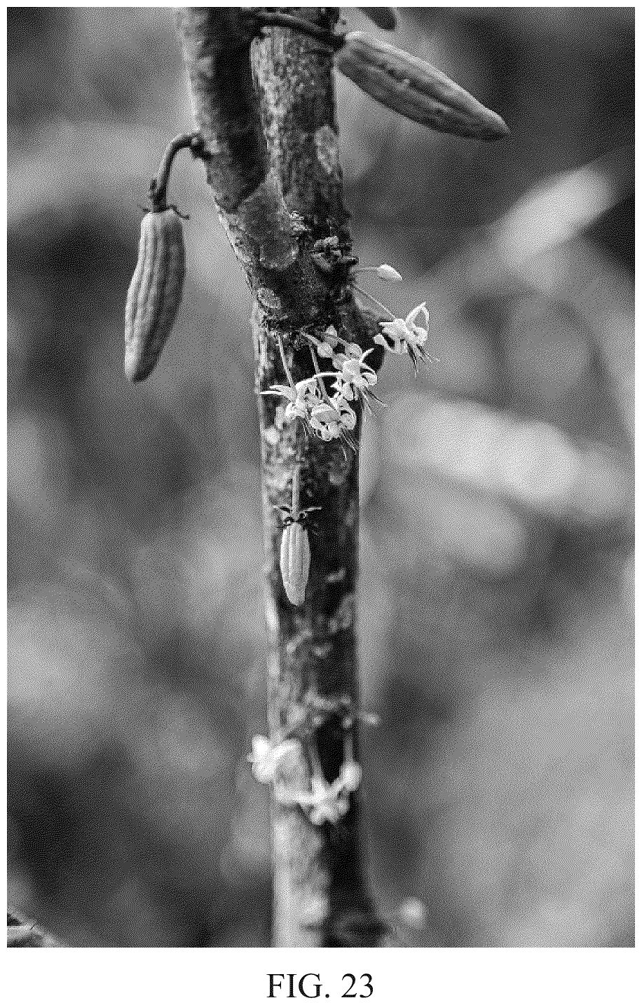

FIG. 24 illustrates leaves of variety ‘CMC-004’.

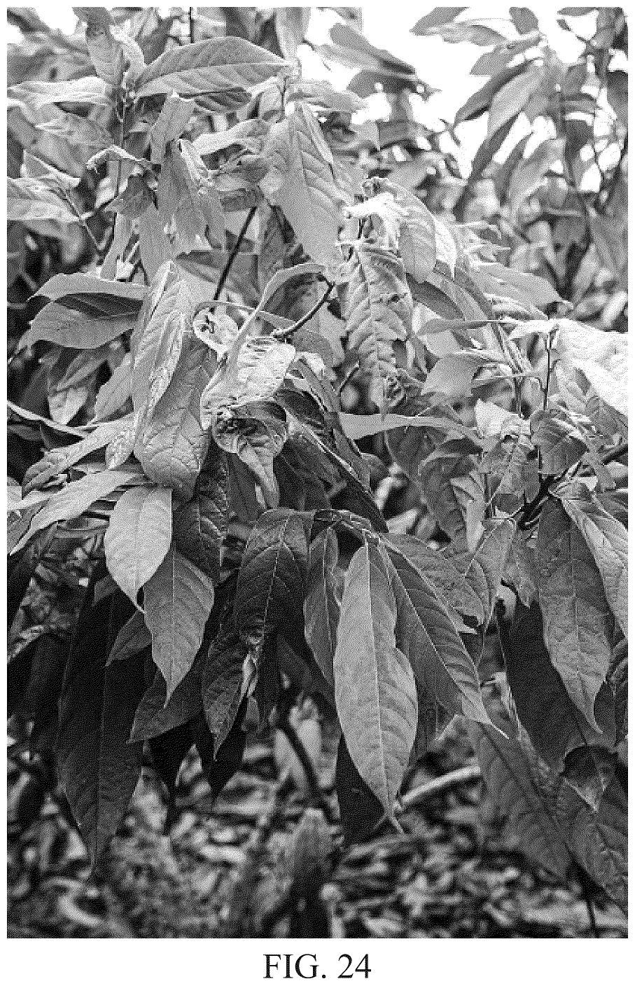

FIG. 25 illustrates a whole tree of variety ‘CMC-005’.

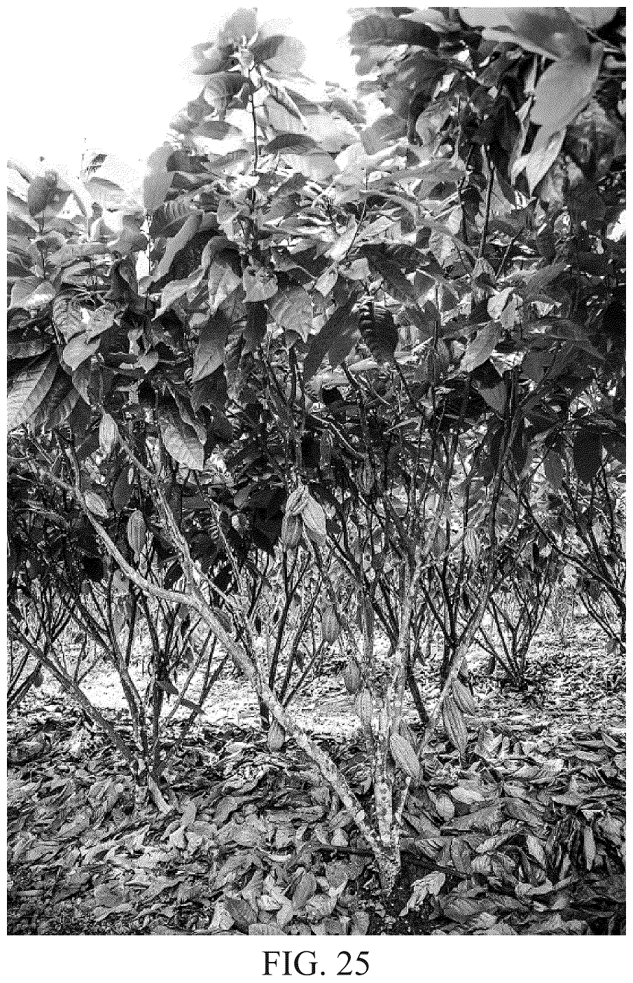

FIG. 26 illustrates a pod of variety ‘CMC-005’.

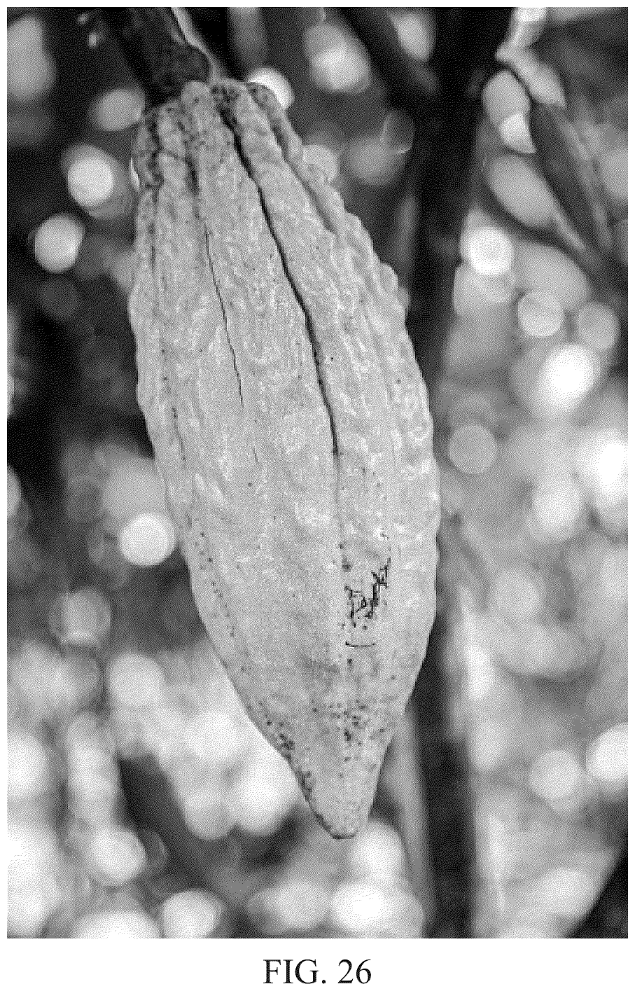

FIG. 27 illustrates a pod of variety ‘CMC-005’.

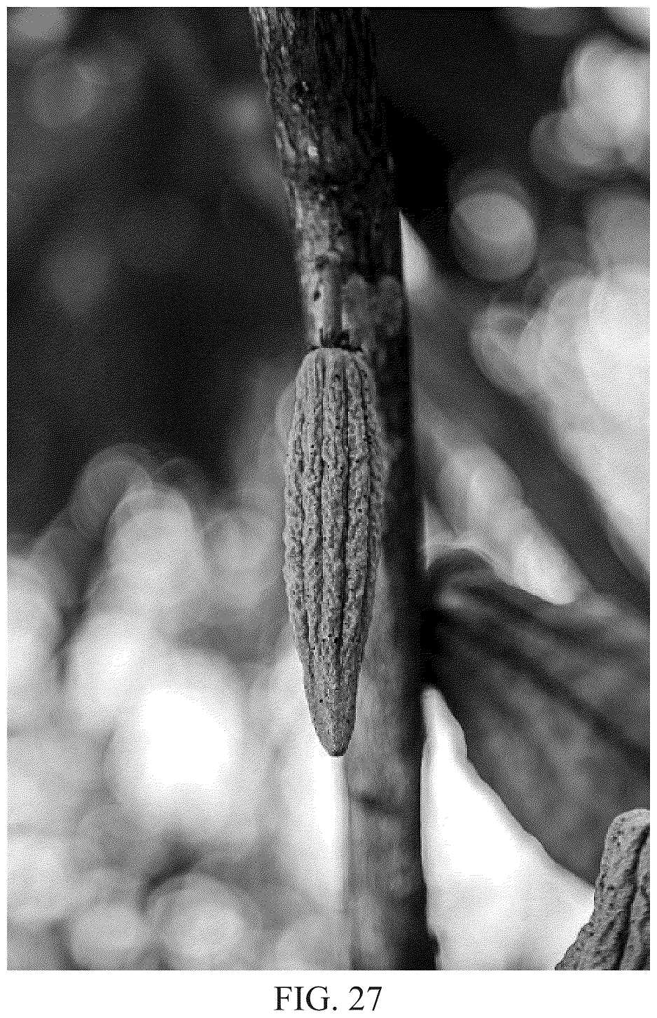

FIG. 28 illustrates a flower of variety ‘CMC-005’.

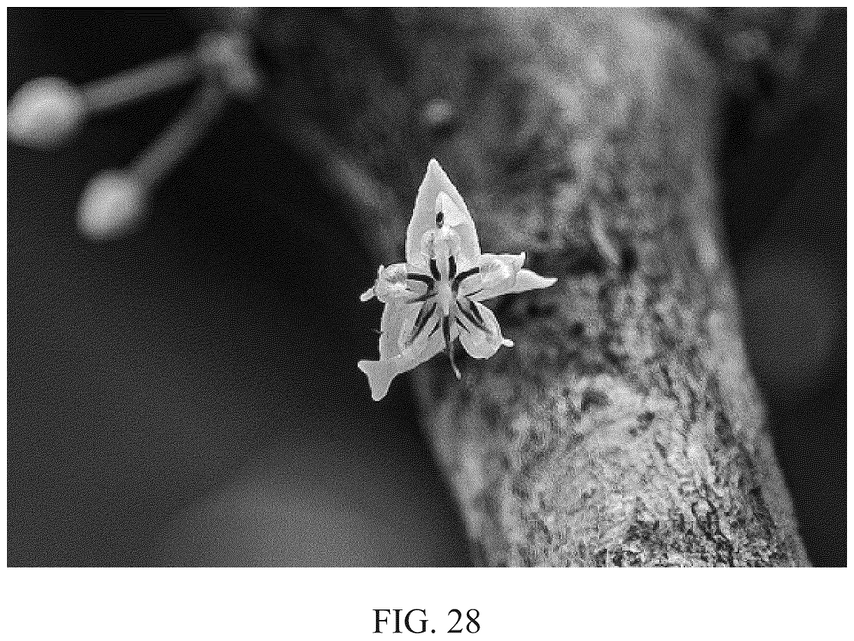

FIG. 29 illustrates flowers and flower buds of variety ‘CMC-005’.

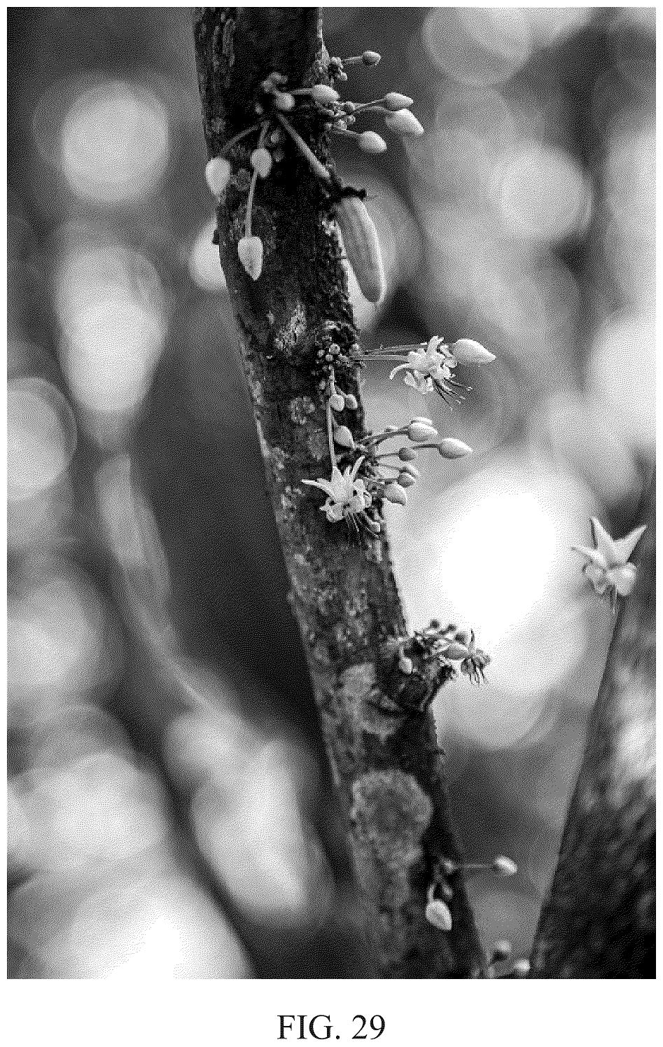

FIG. 30 illustrates leaves of variety ‘CMC-005’.

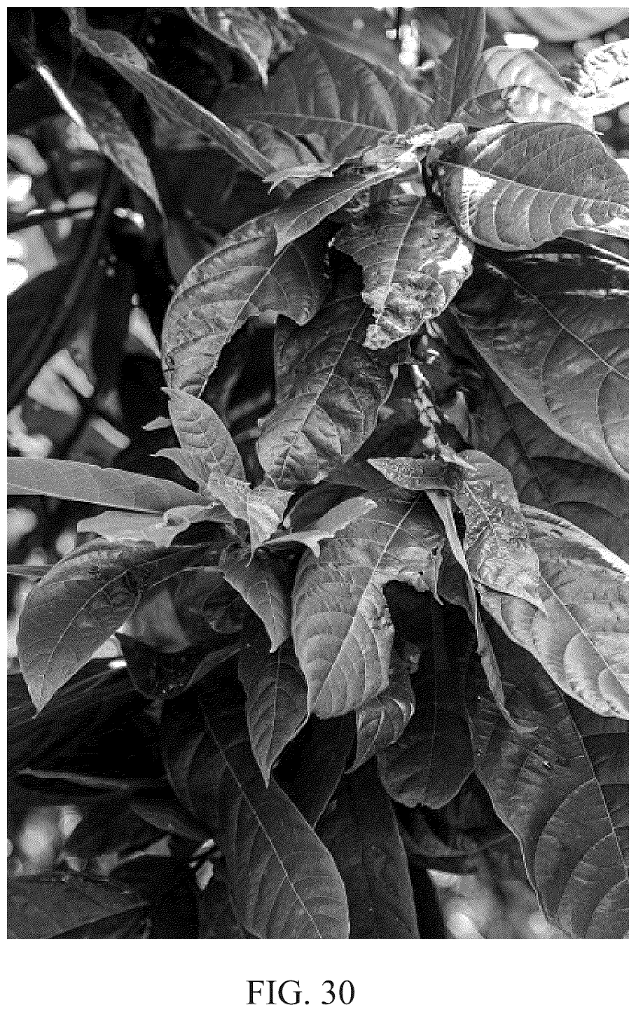

FIG. 31 illustrates a whole tree of variety ‘CMC-006’.

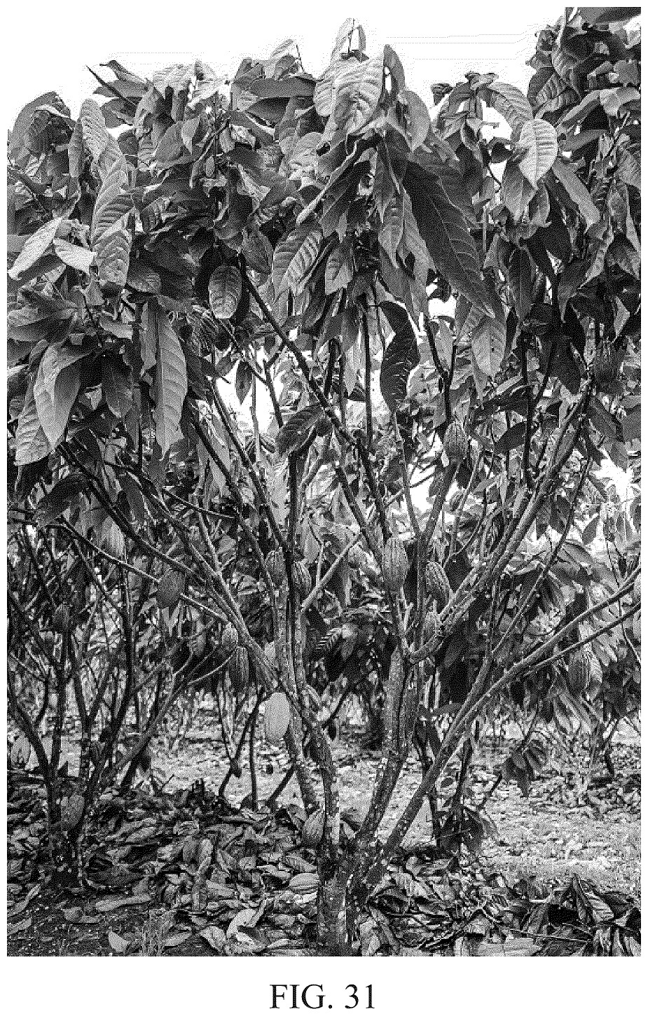

FIG. 32 illustrates a pod of variety ‘CMC-006’.

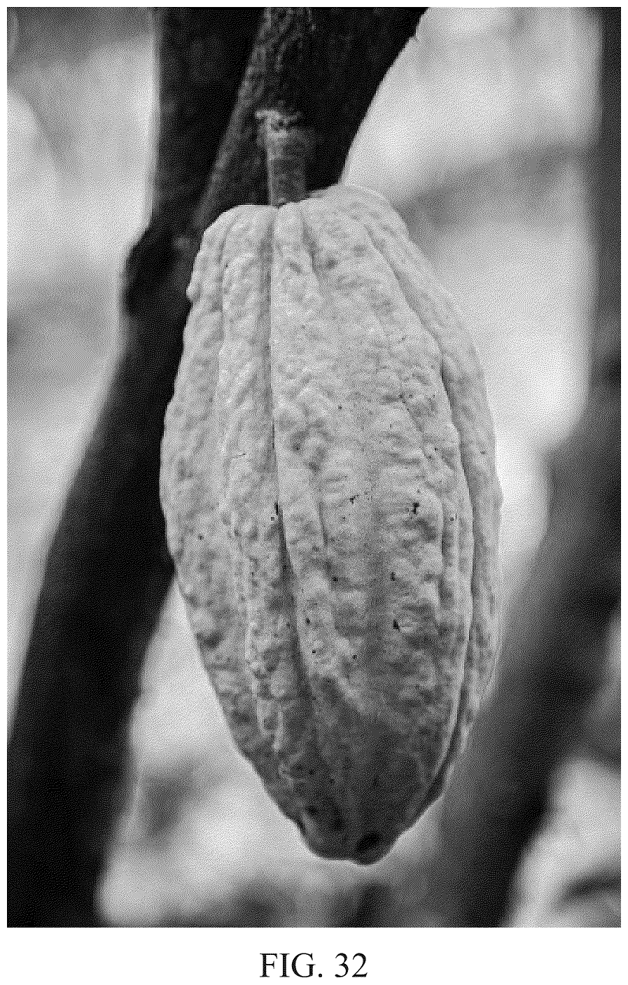

FIG. 33 illustrates a flower of variety ‘CMC-006’.

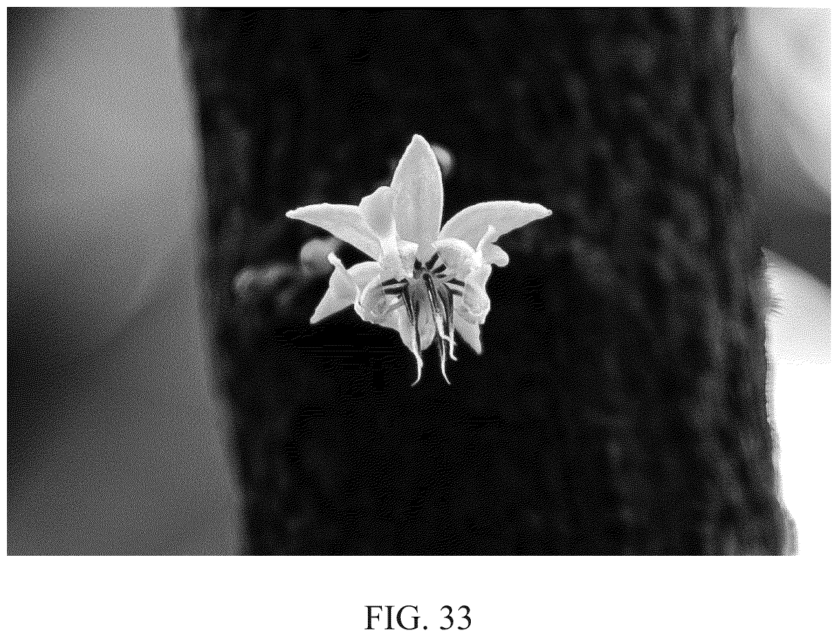

FIG. 34 illustrates a flower of variety ‘CMC-006’.

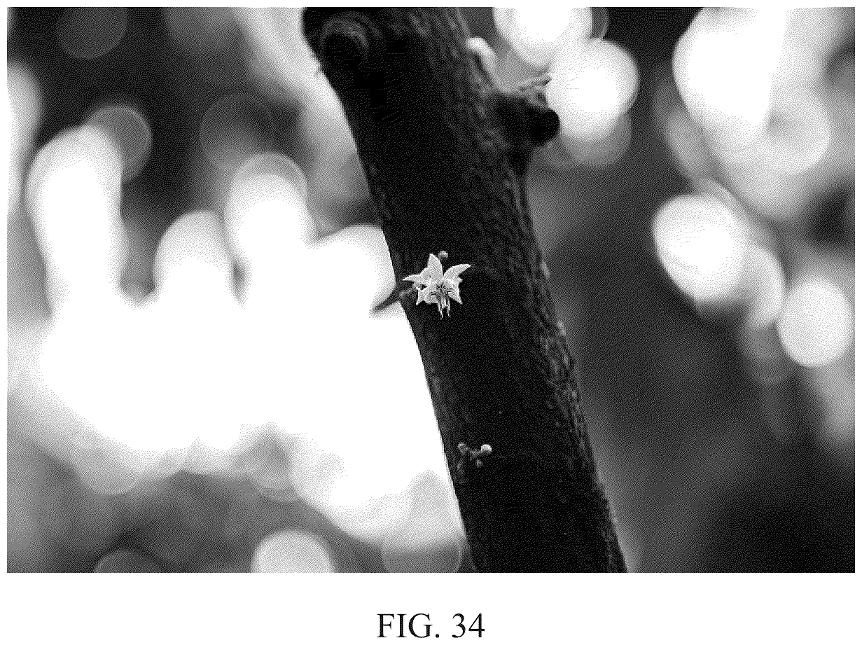

FIG. 35 illustrates flowers and flower buds of variety ‘CMC-006’.

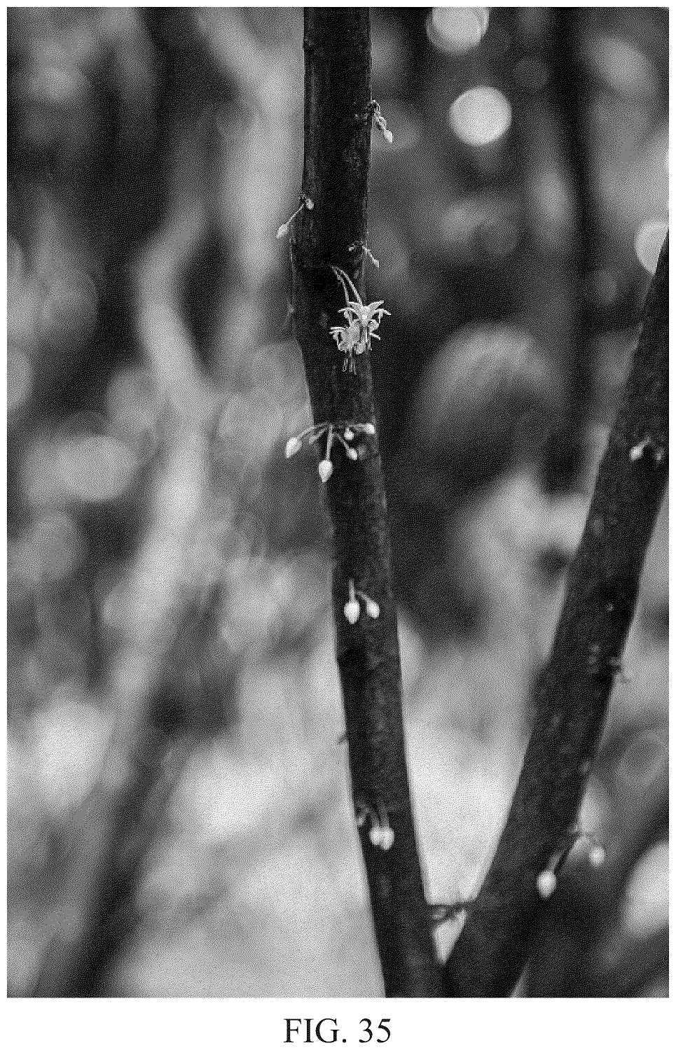

FIG. 36 illustrates leaves of variety ‘CMC-006’.

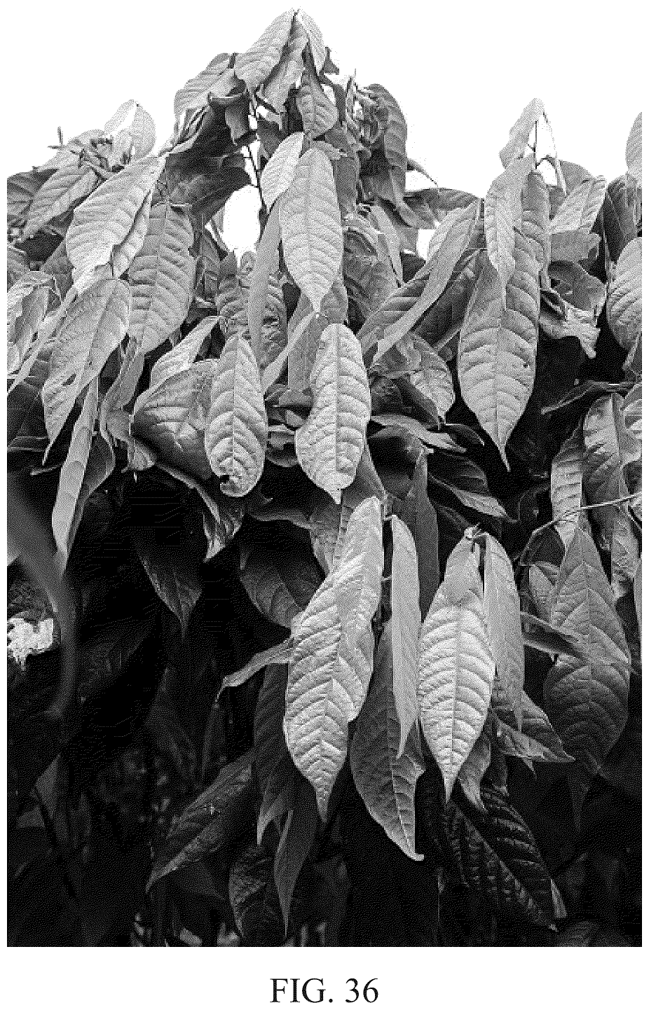

FIG. 37 illustrates a whole tree of variety ‘CMC-007’.

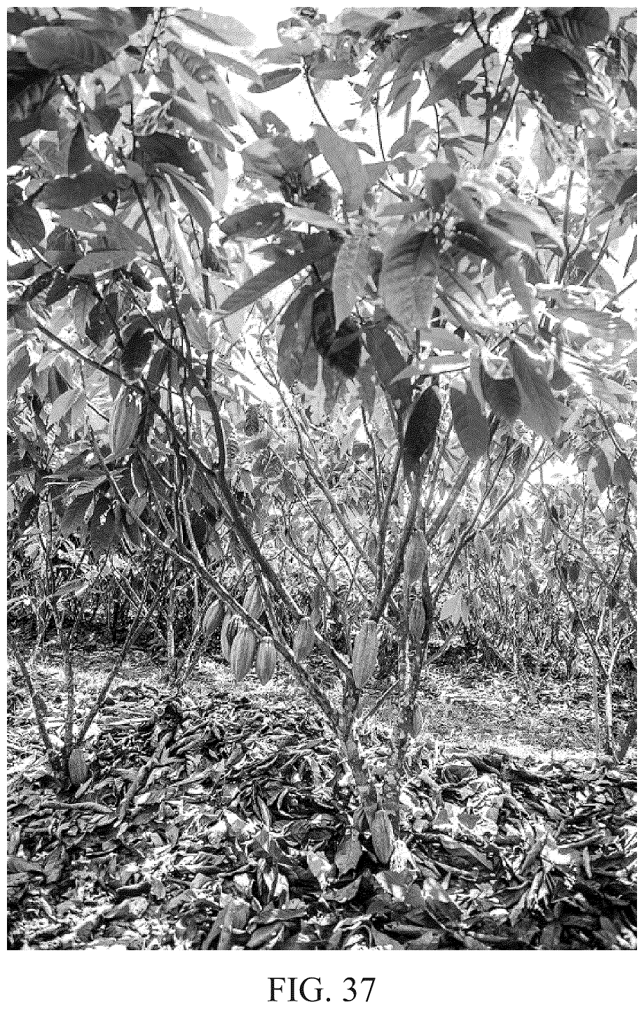

FIG. 38 illustrates a pod of variety ‘CMC-007’.

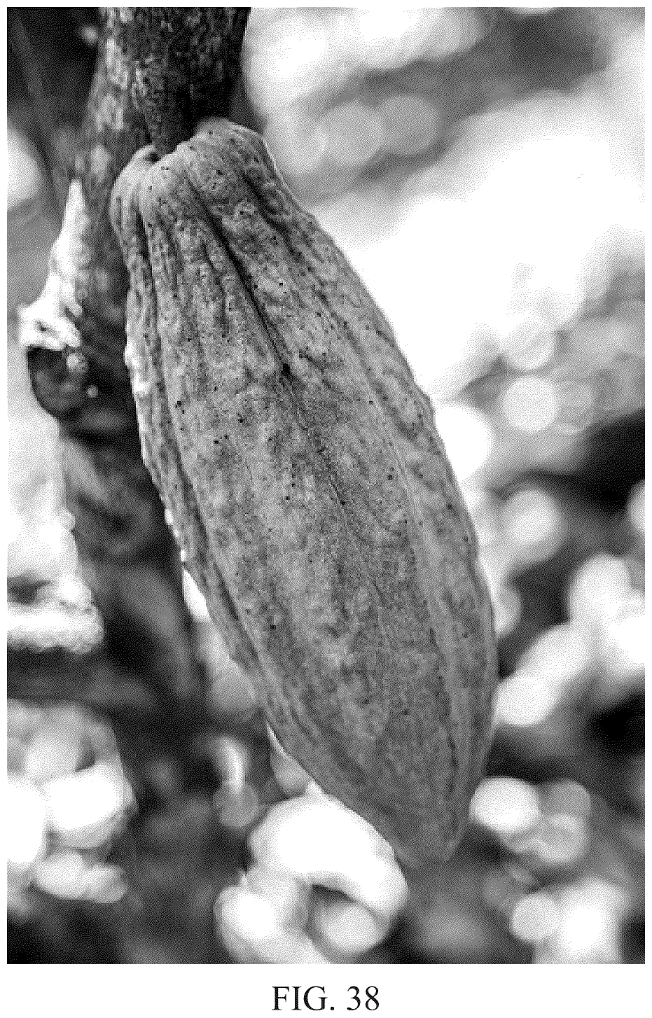

FIG. 39 illustrates a pod of variety ‘CMC-007’.

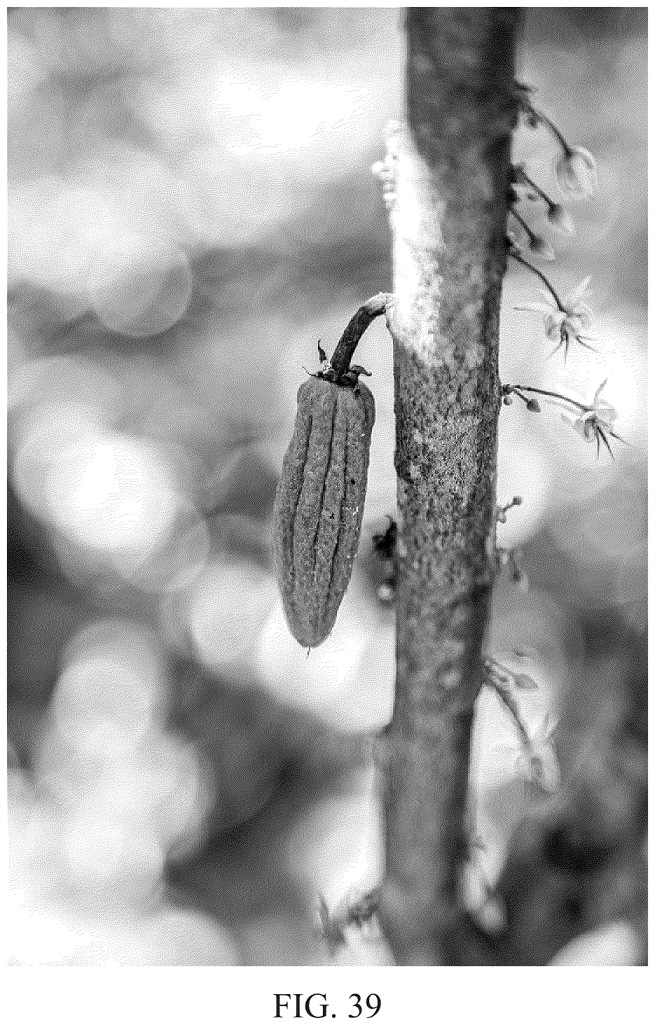

FIG. 40 illustrates a flower of variety ‘CMC-007’.

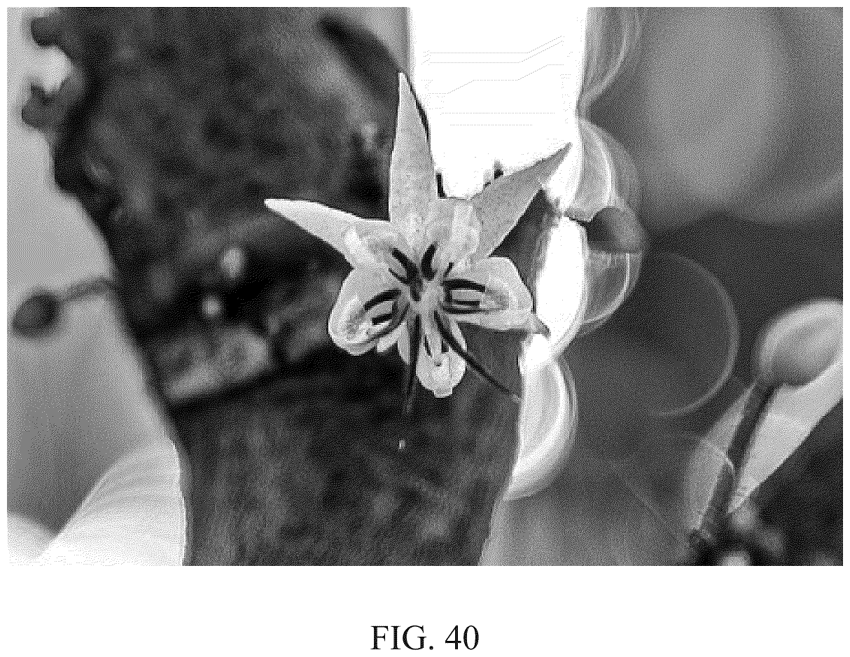

FIG. 41 illustrates flowers and flower buds of variety ‘CMC-007’.

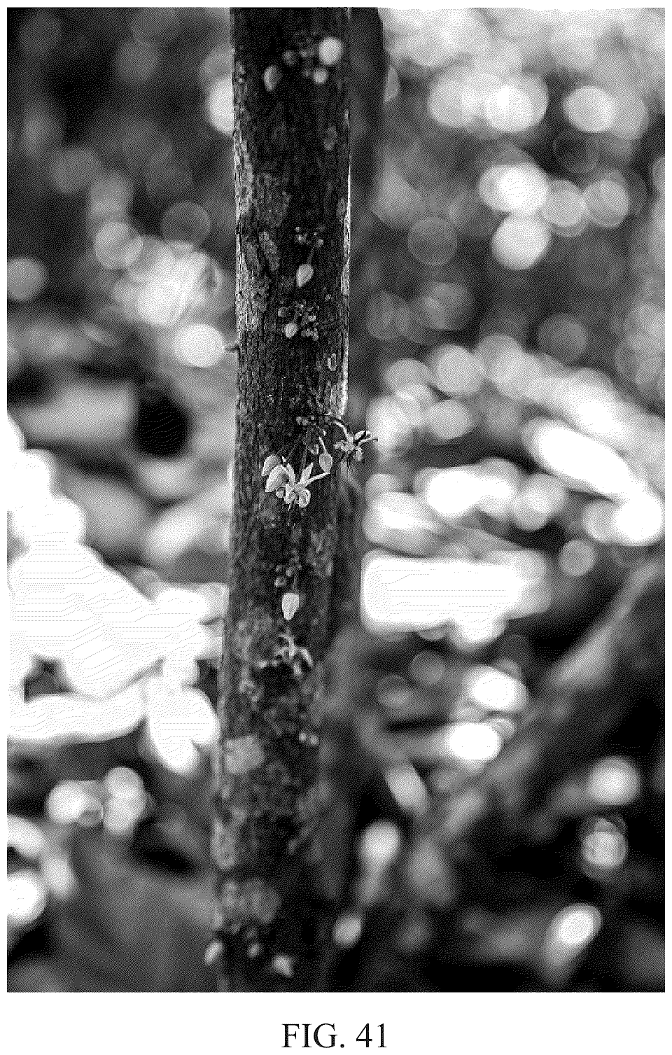

FIG. 42 illustrates leaves of variety ‘CMC-007’.

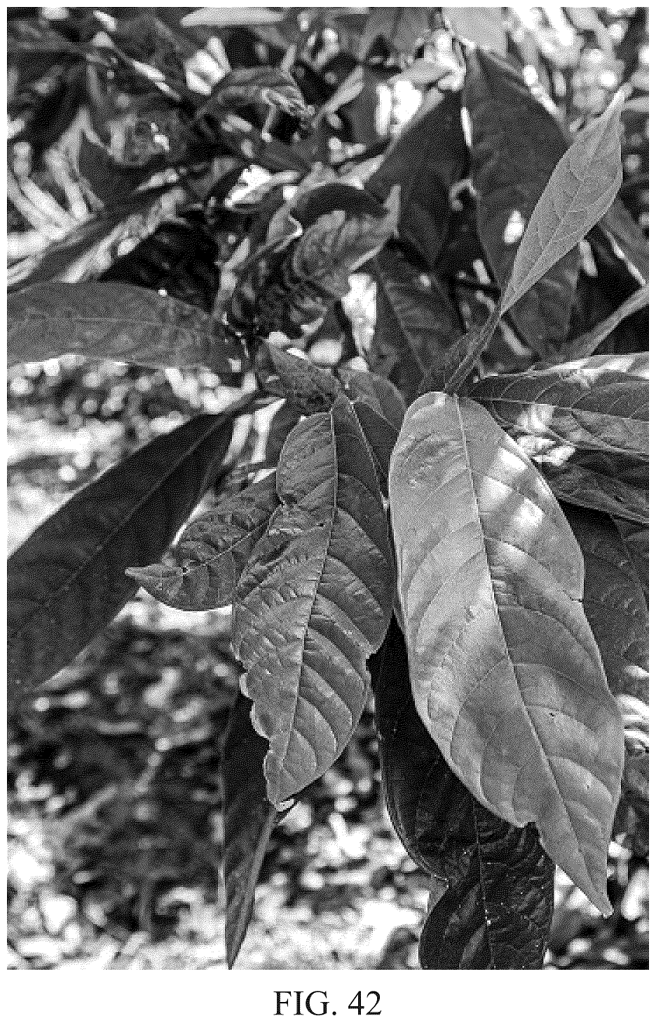

FIG. 43 illustrates a whole tree of variety ‘CMC-008’.

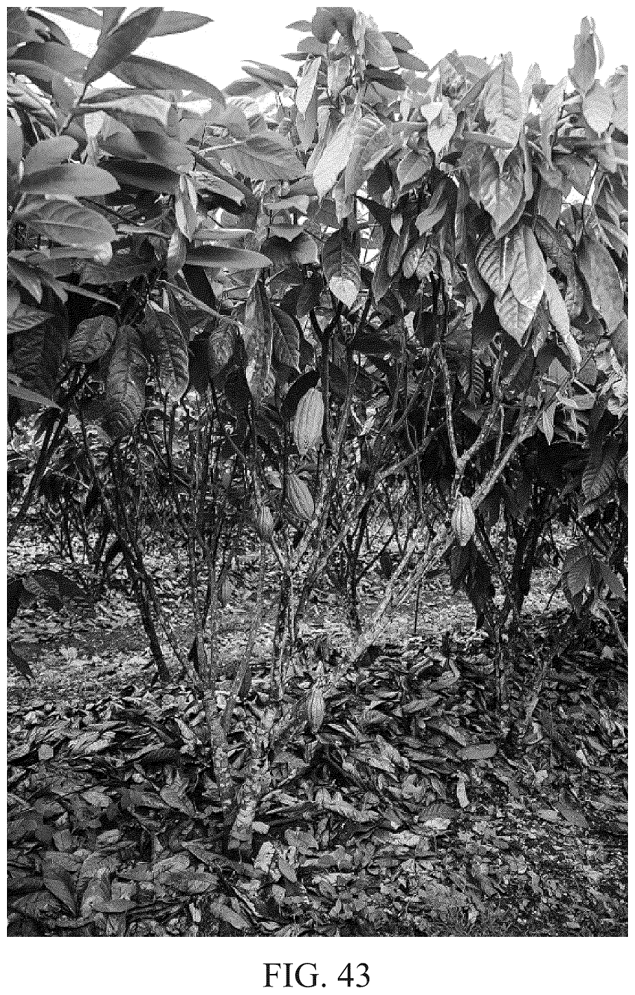

FIG. 44 illustrates a pod of variety ‘CMC-008’.

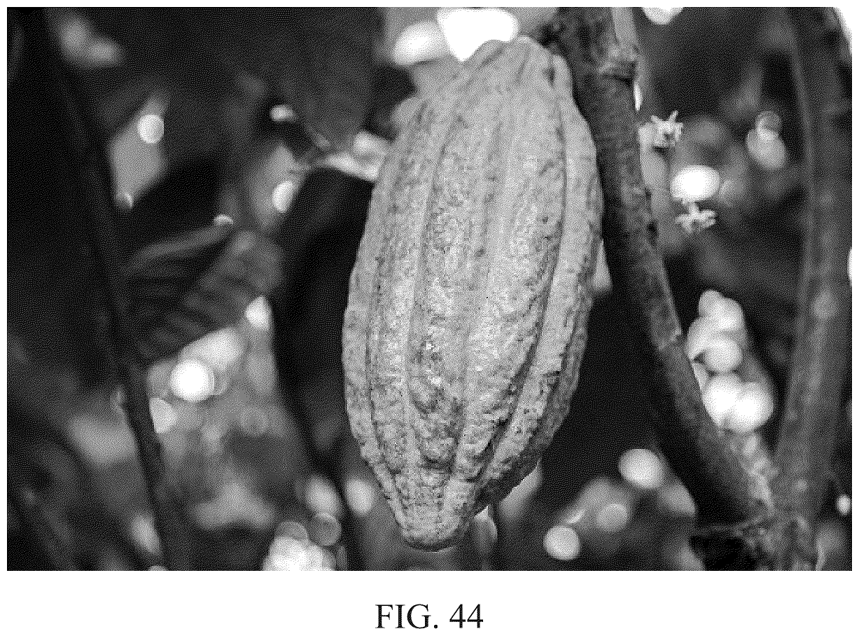

FIG. 45 illustrates a pod of variety ‘CMC-008’.

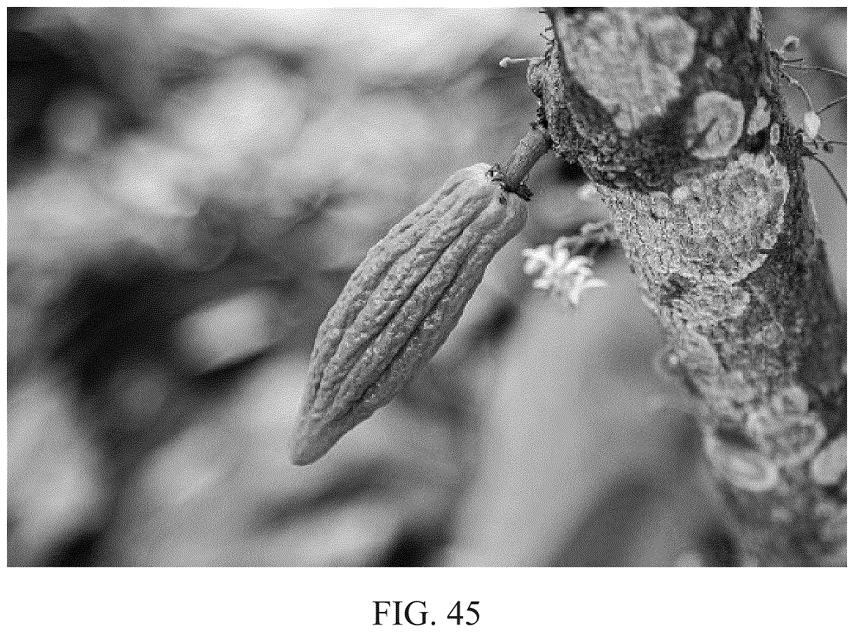

FIG. 46 illustrates a flower of variety ‘CMC-008’.

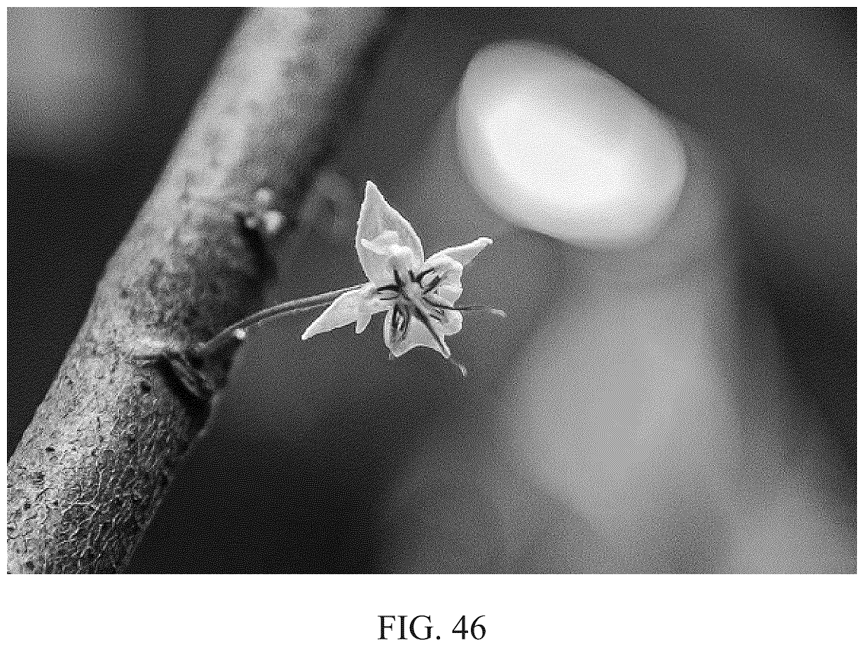

FIG. 47 illustrates flowers and flower buds of variety ‘CMC-008’.

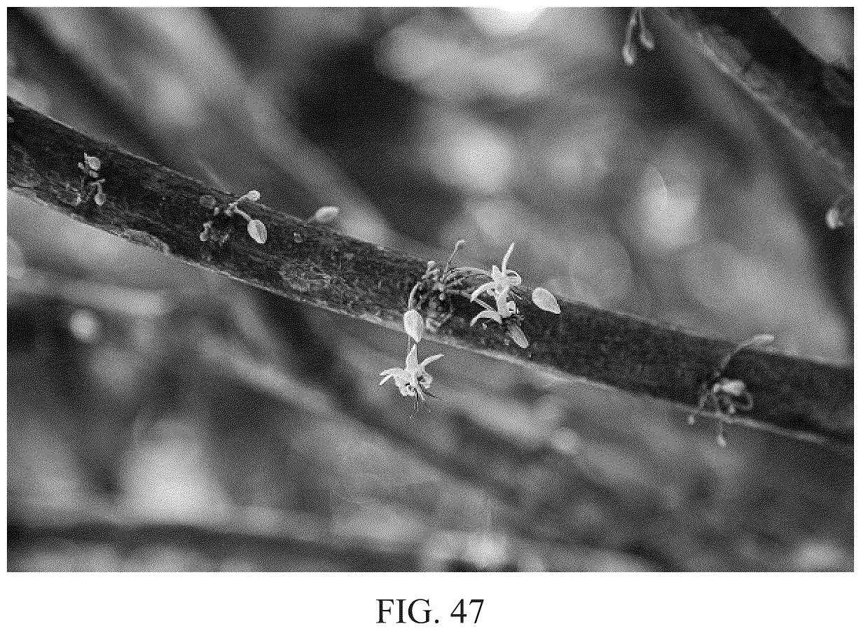

FIG. 48 illustrates leaves of variety ‘CMC-008’.

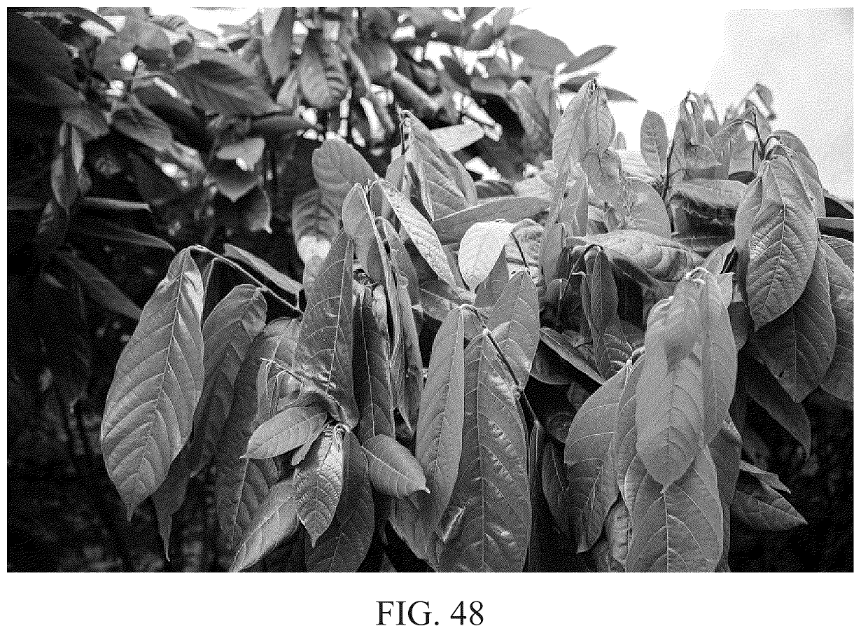

FIG. 49 illustrates a whole tree of variety ‘CMC-009’.

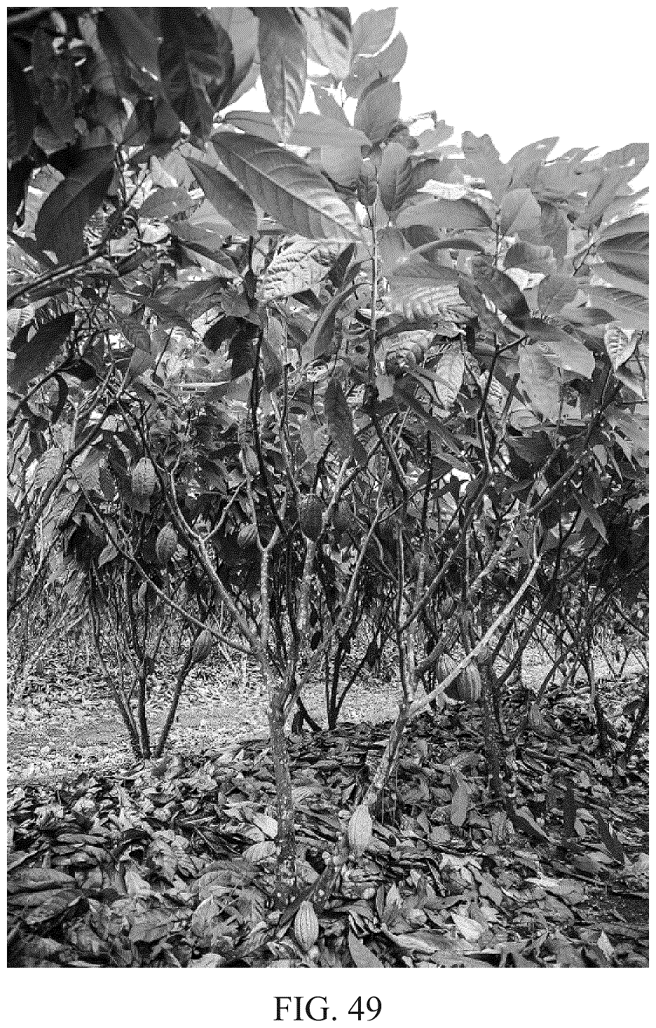

FIG. 50 illustrates a pod of variety ‘CMC-009’.

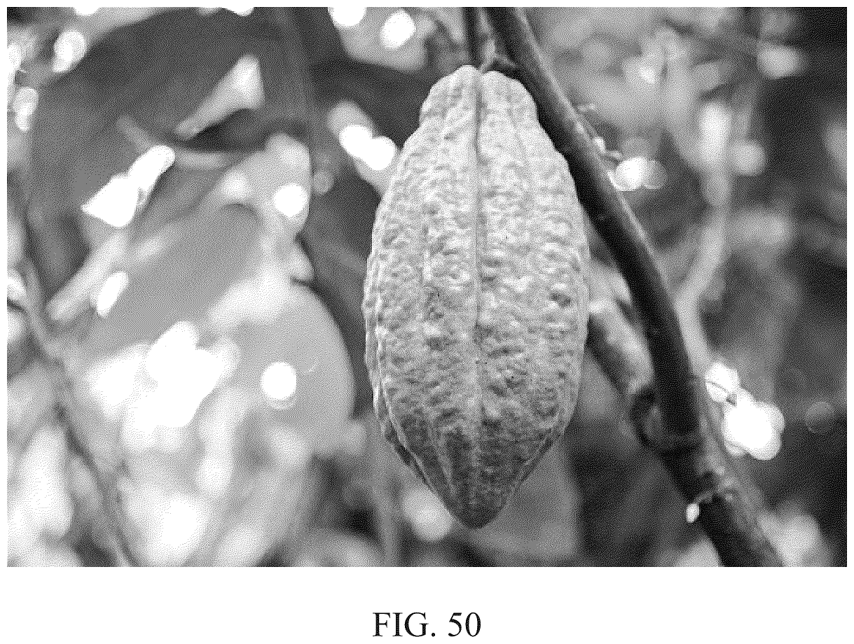

FIG. 51 illustrates a pod of variety ‘CMC-009’.

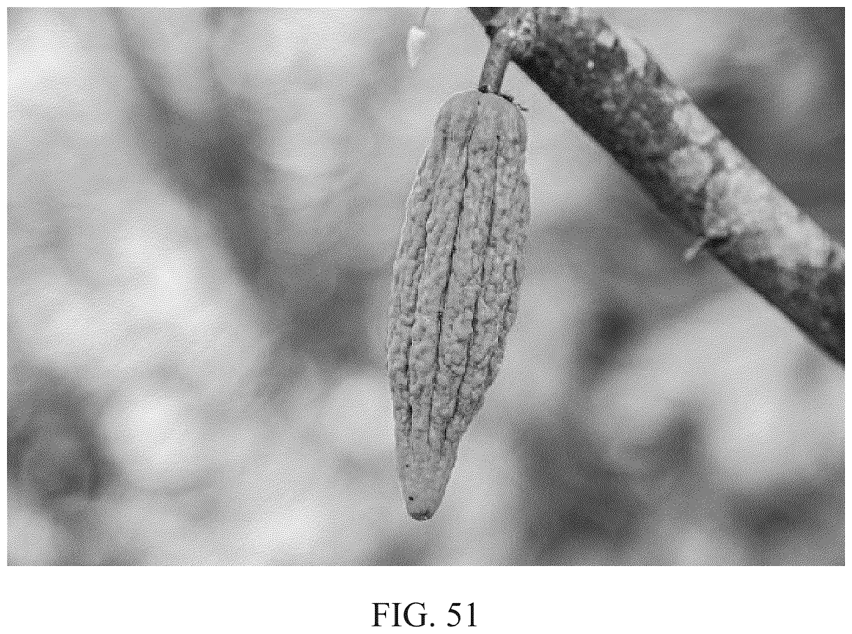

FIG. 52 illustrates a flower of variety ‘CMC-009’.

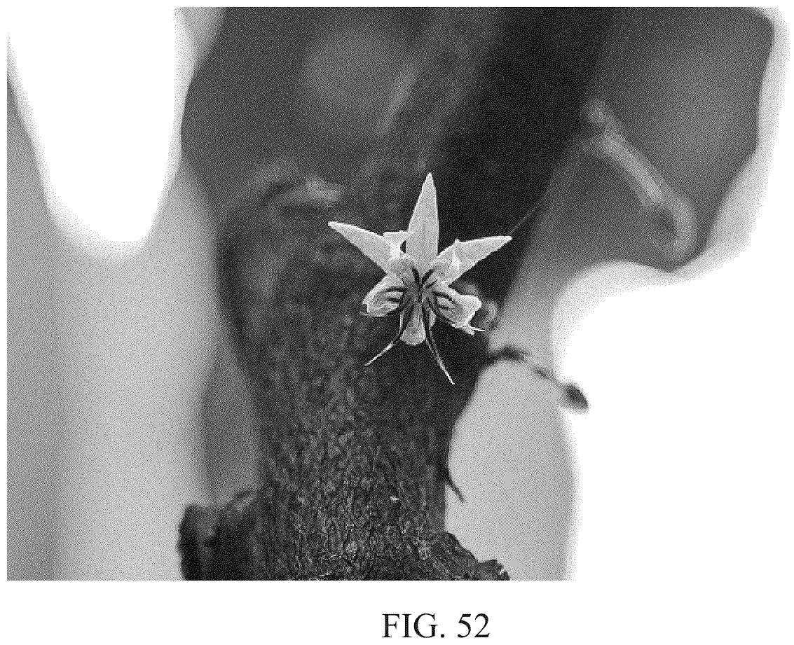

FIG. 53 illustrates flowers and flower buds of variety ‘CMC-009’.

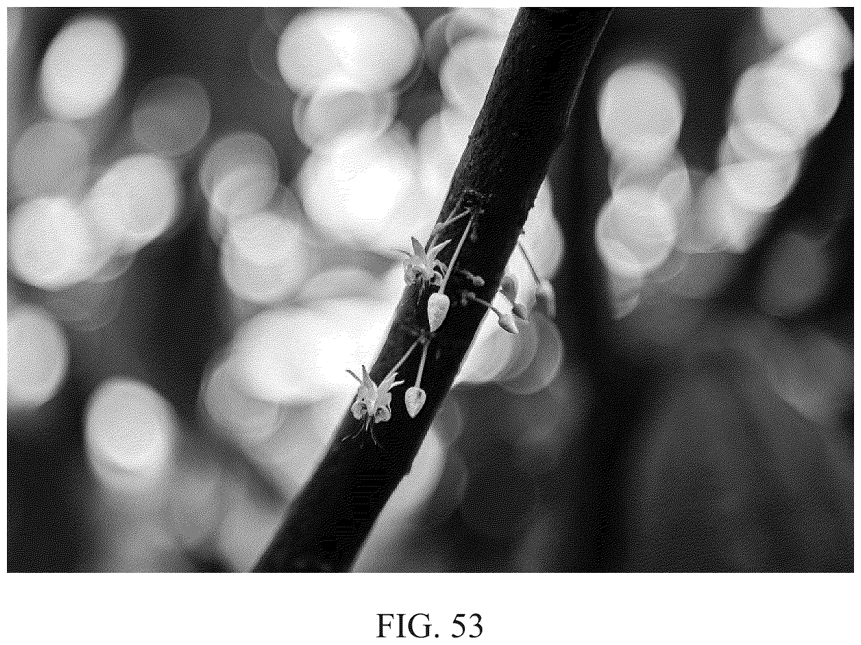

FIG. 54 illustrates leaves of variety ‘CMC-009’.

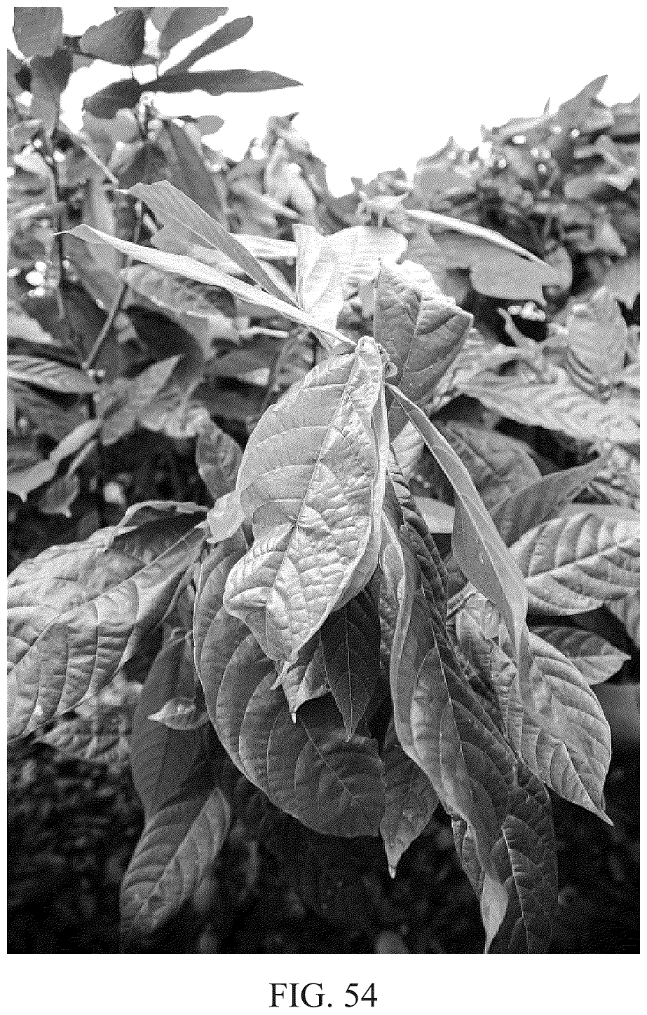

FIG. 55 illustrates a whole tree of variety ‘CMC-010’.

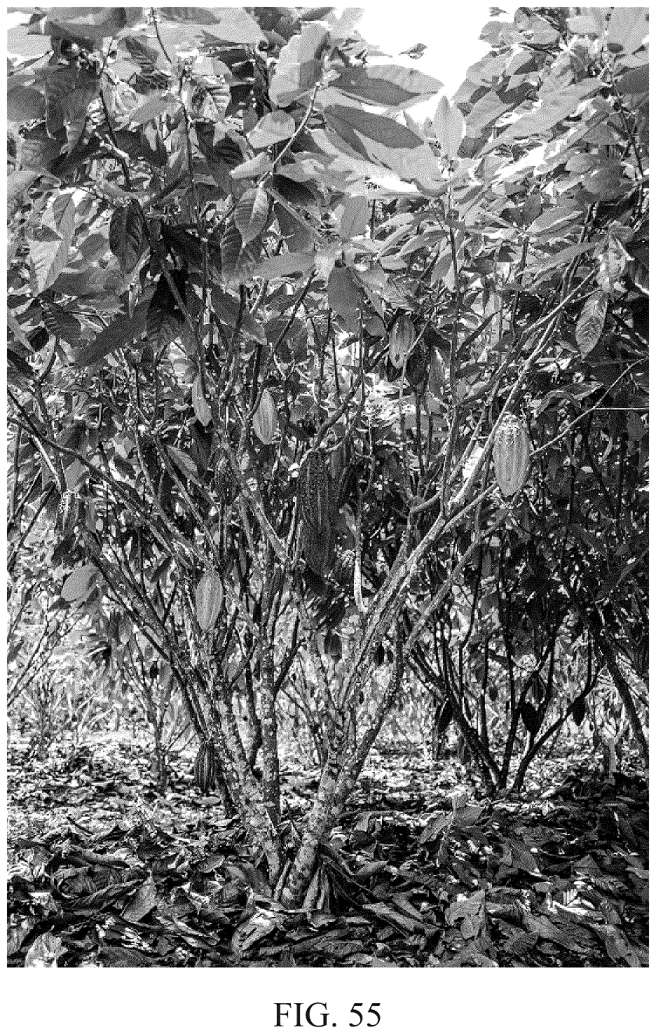

FIG. 56 illustrates a pod of variety ‘CMC-010’.

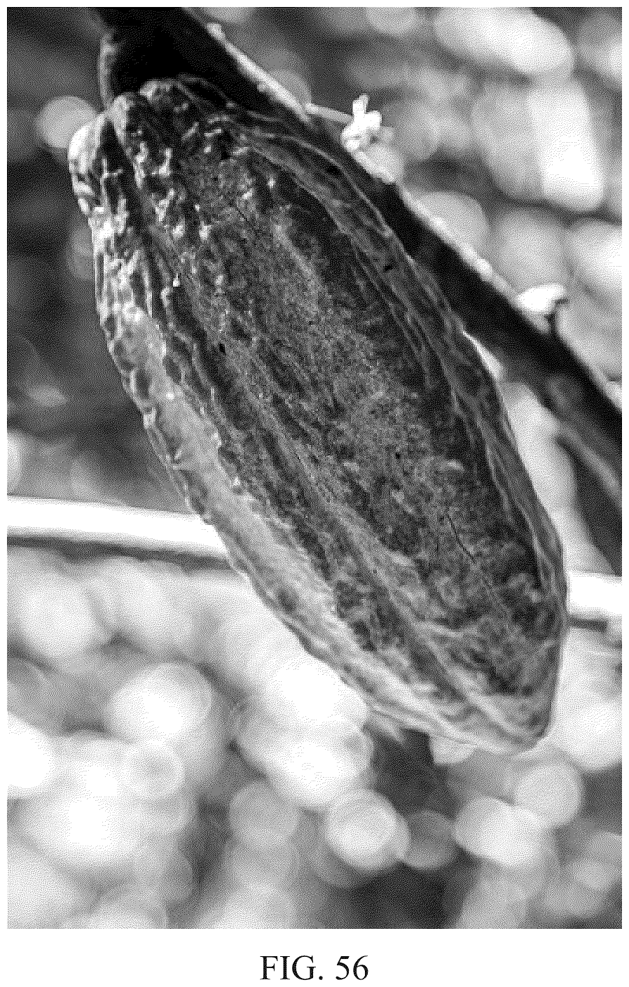

FIG. 57 illustrates a pod of variety ‘CMC-010’.

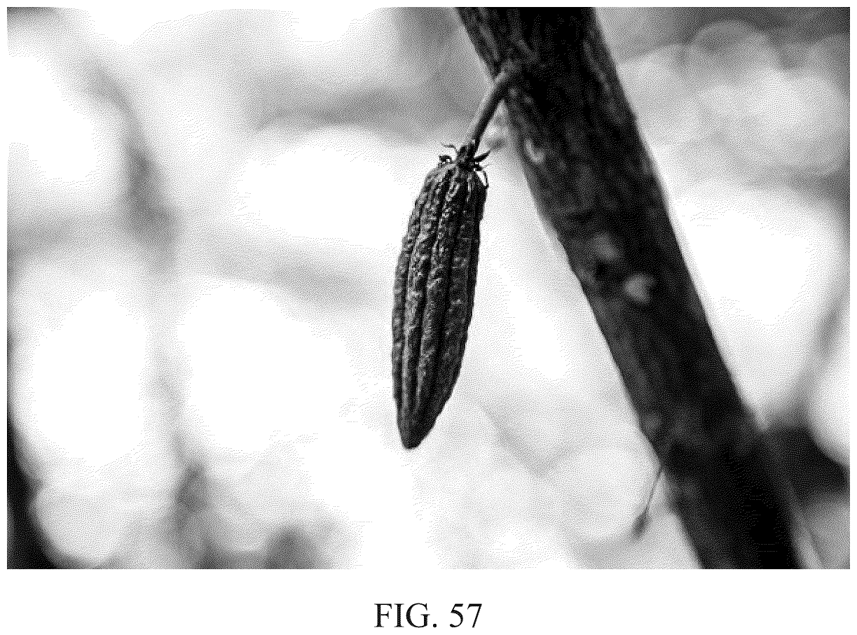

FIG. 58 illustrates a flower of variety ‘CMC-010’.

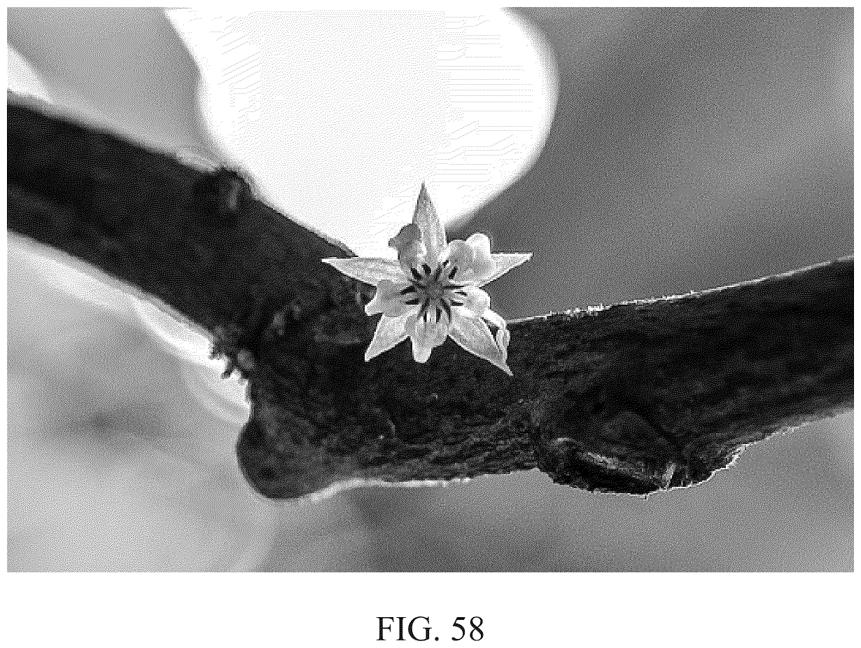

FIG. 59 illustrates flowers and flower buds of variety ‘CMC-010’.

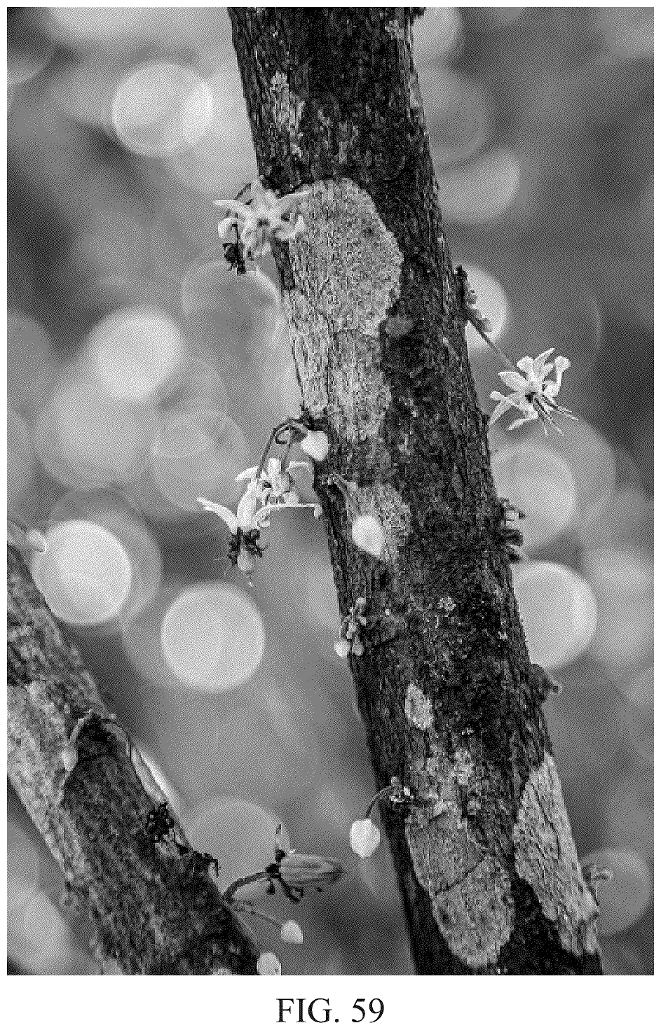

FIG. 60 illustrates leaves of variety ‘CMC-010’.

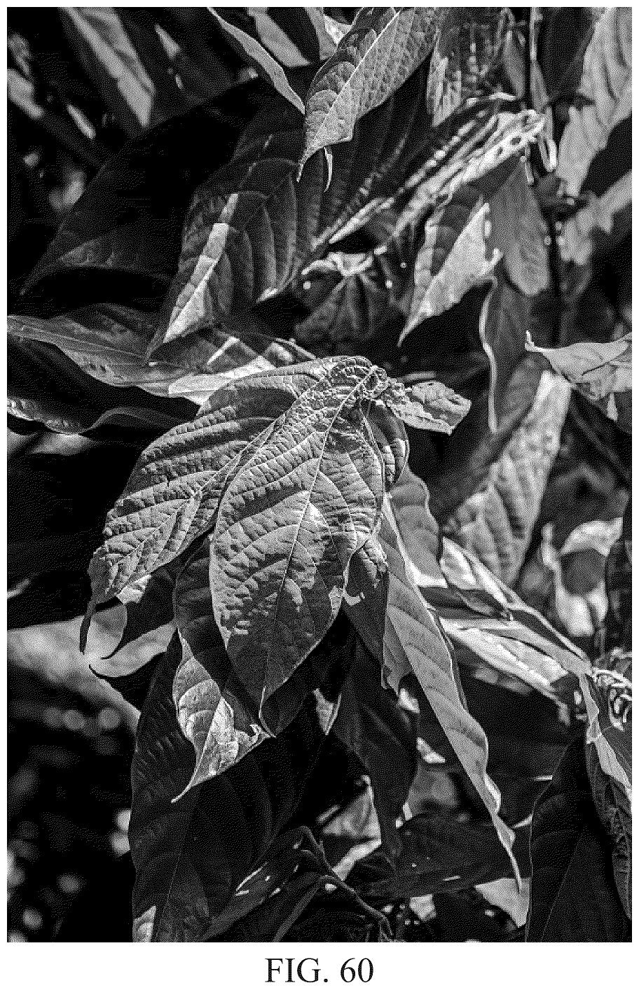

FIG. 61 illustrates a whole tree of variety ‘CMC-011’.

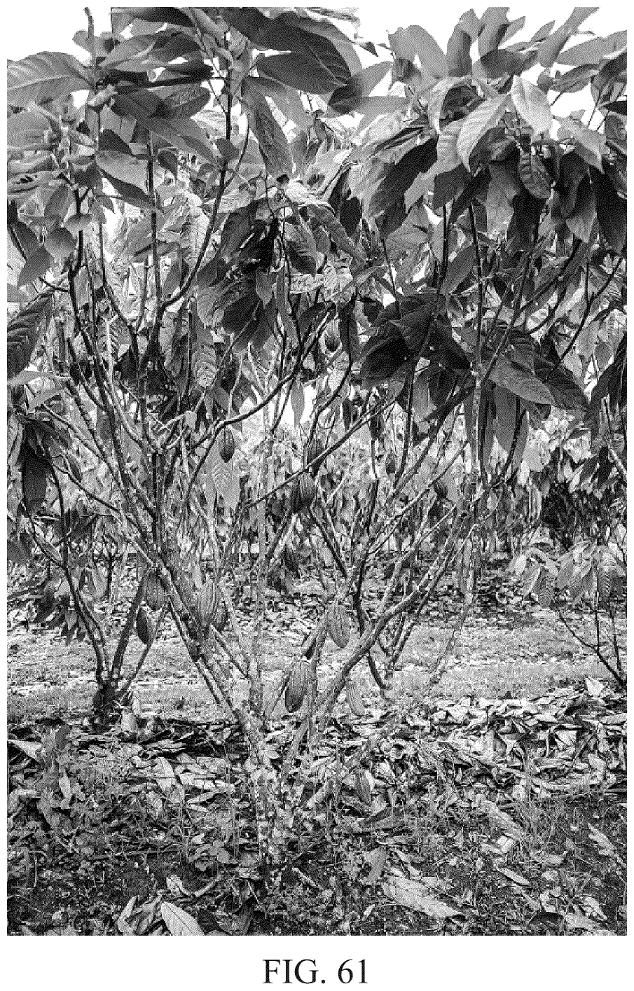

FIG. 62 illustrates a pod of variety ‘CMC-011’.

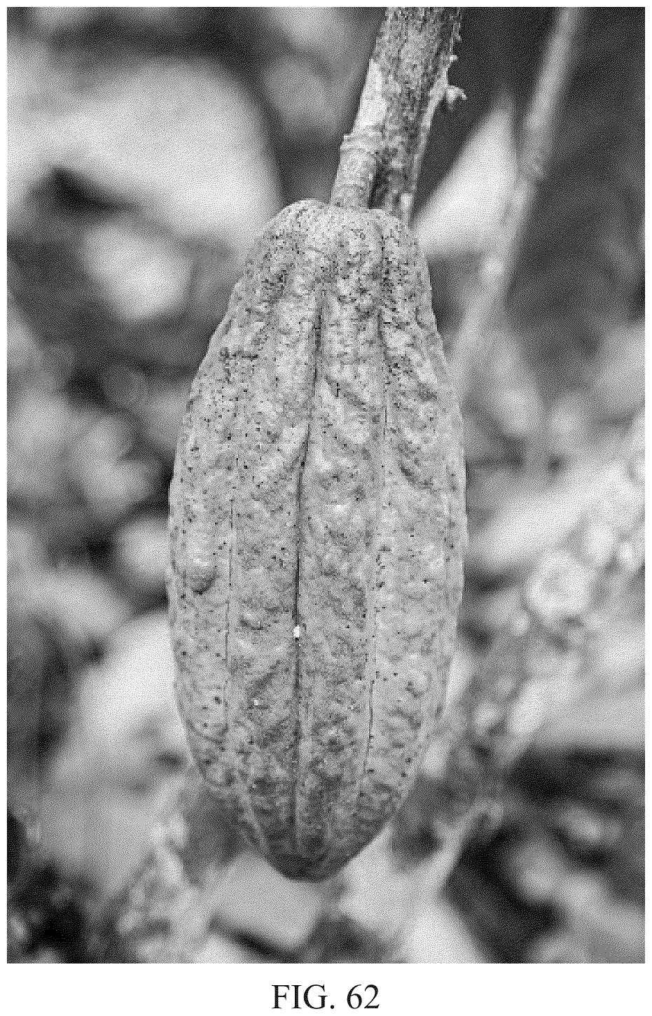

FIG. 63 illustrates flowers and flower buds of variety ‘CMC-011’.

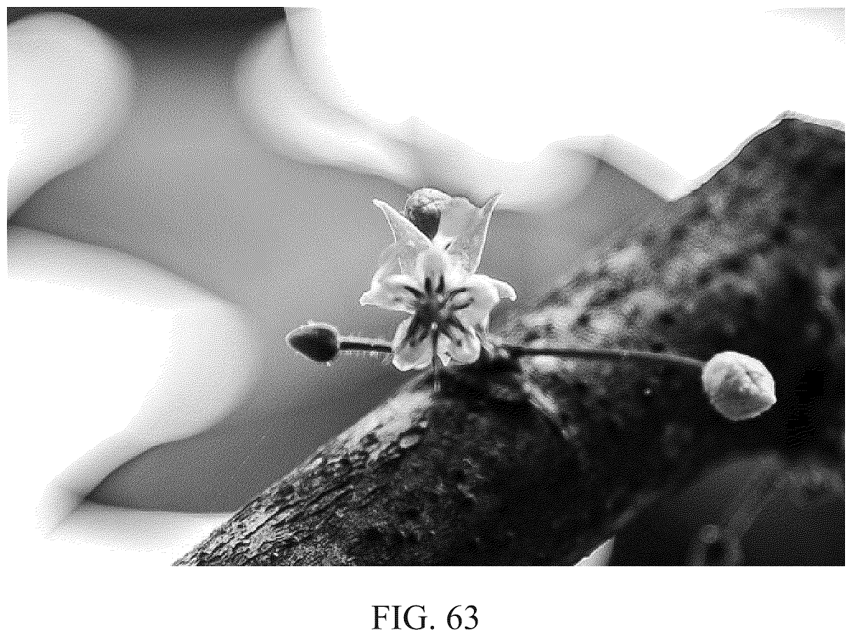

FIG. 64 illustrates flowers and flower buds of variety ‘CMC-011’.

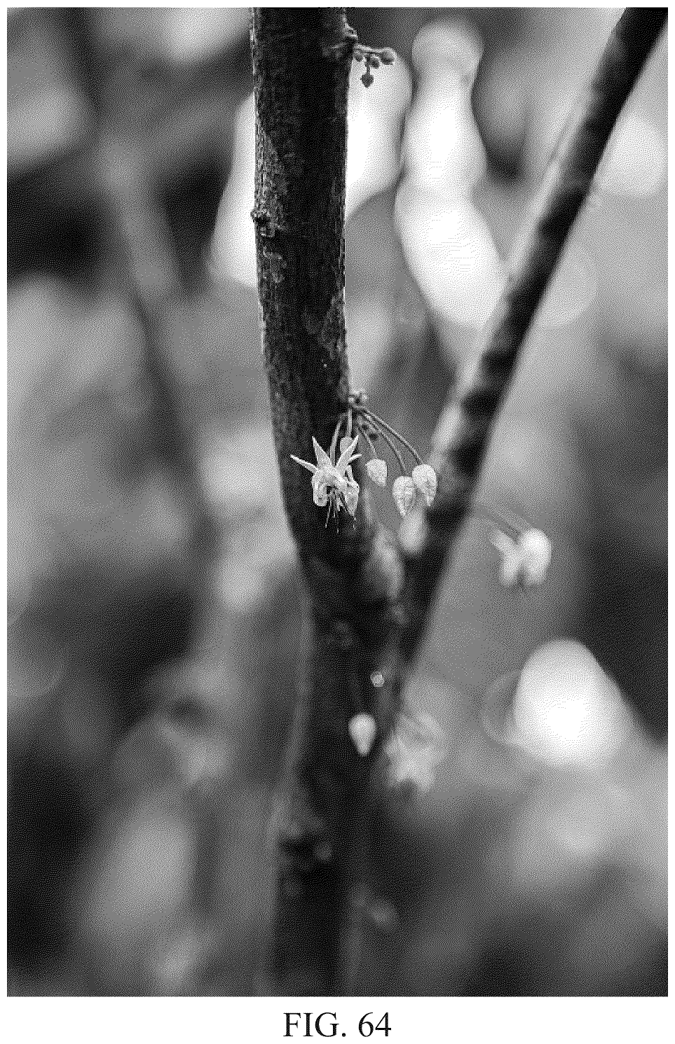

FIG. 65 illustrates flowers and flower buds of variety ‘CMC-011’.

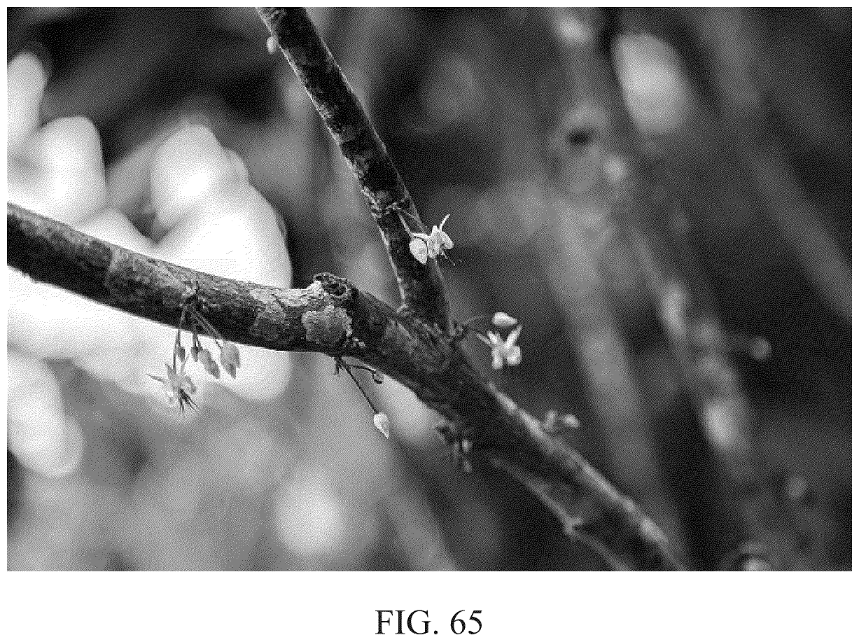

FIG. 66 illustrates leaves of variety ‘CMC-011’.

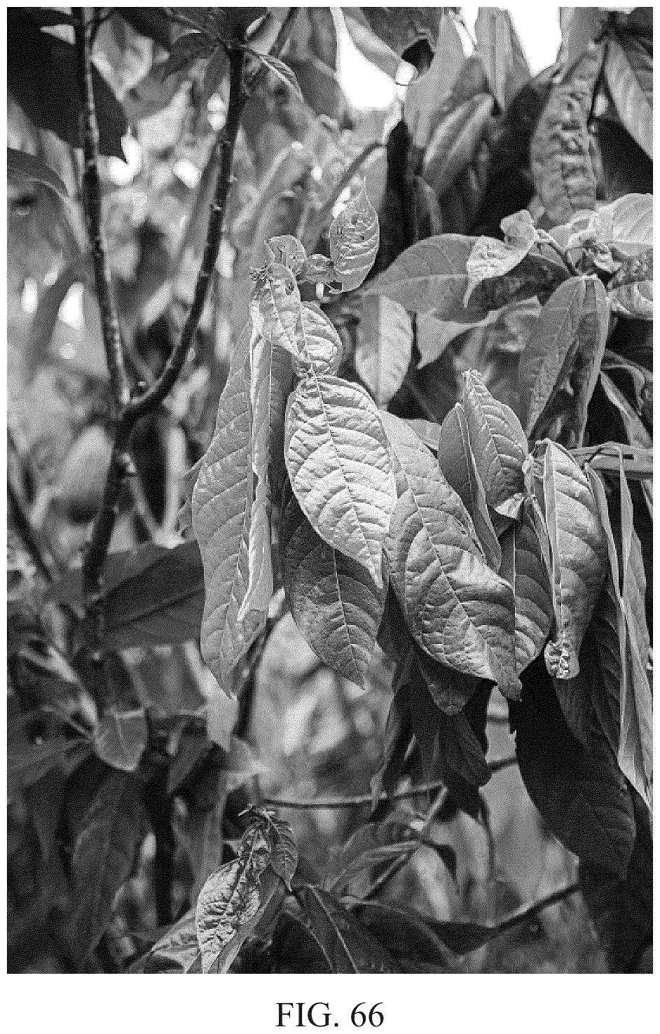

FIG. 67 illustrates a whole tree of variety ‘CMC-012’.

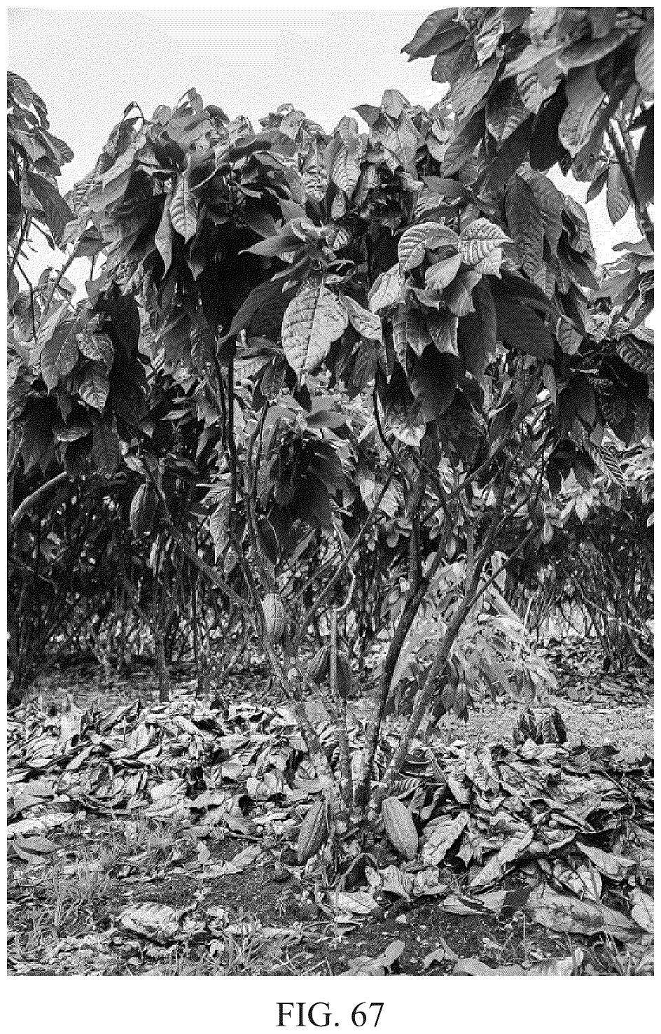

FIG. 68 illustrates a pod of variety ‘CMC-012’.

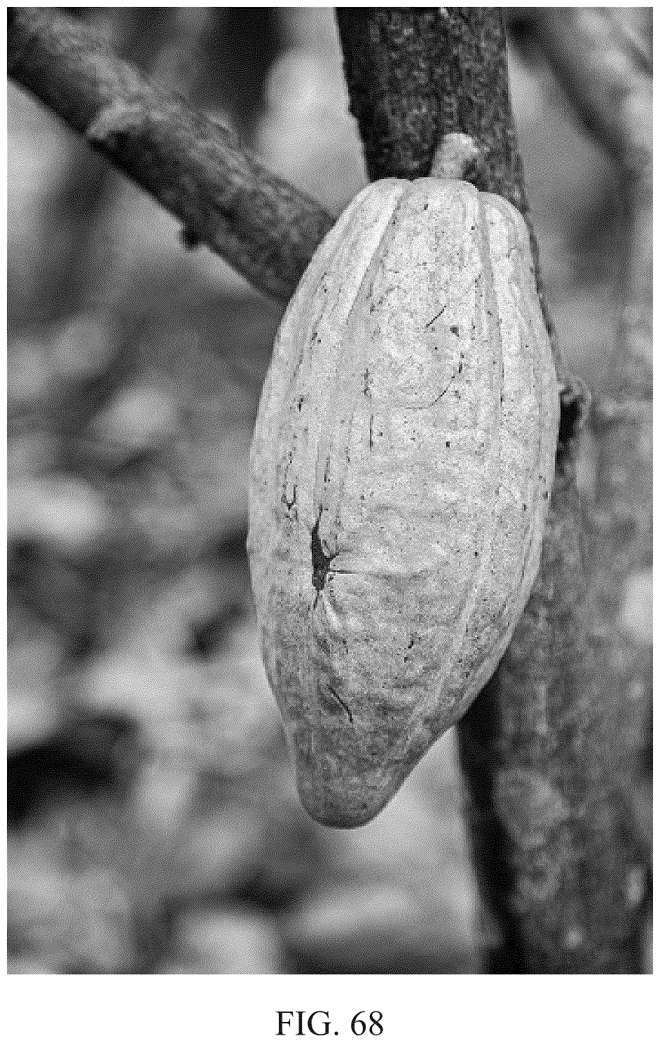

FIG. 69 illustrates a pod of variety ‘CMC-012’.

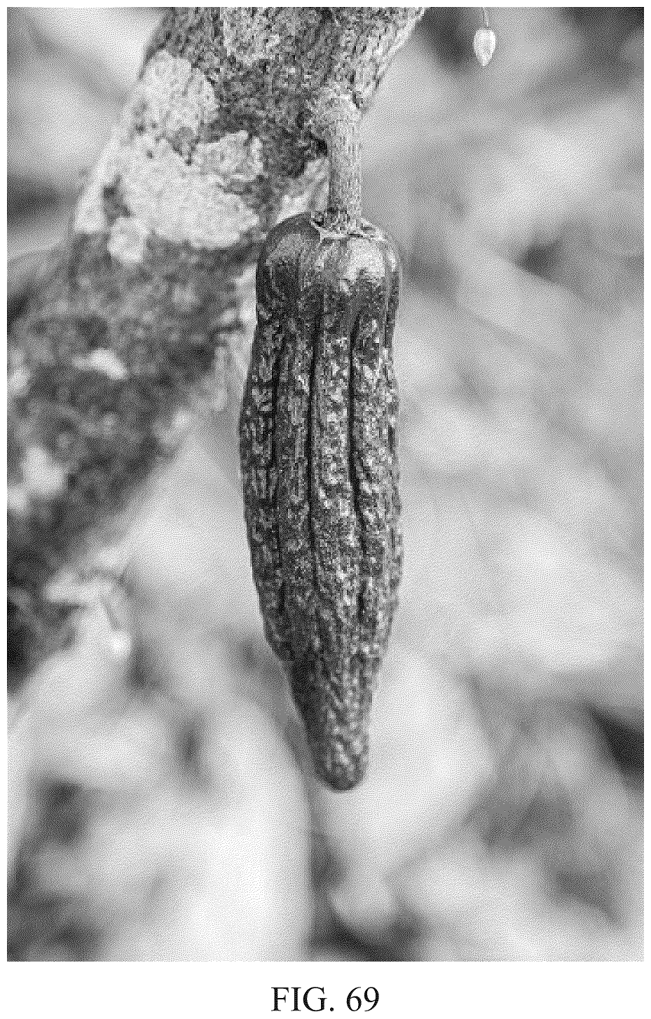

FIG. 70 illustrates a flower of variety ‘CMC-012’.

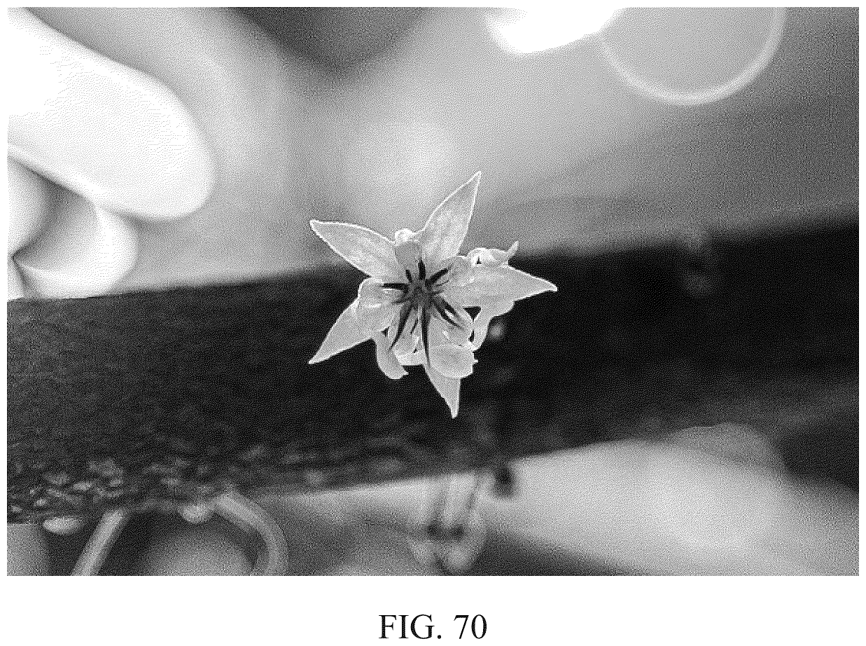

FIG. 71 illustrates flowers and flower buds of variety ‘CMC-012’.

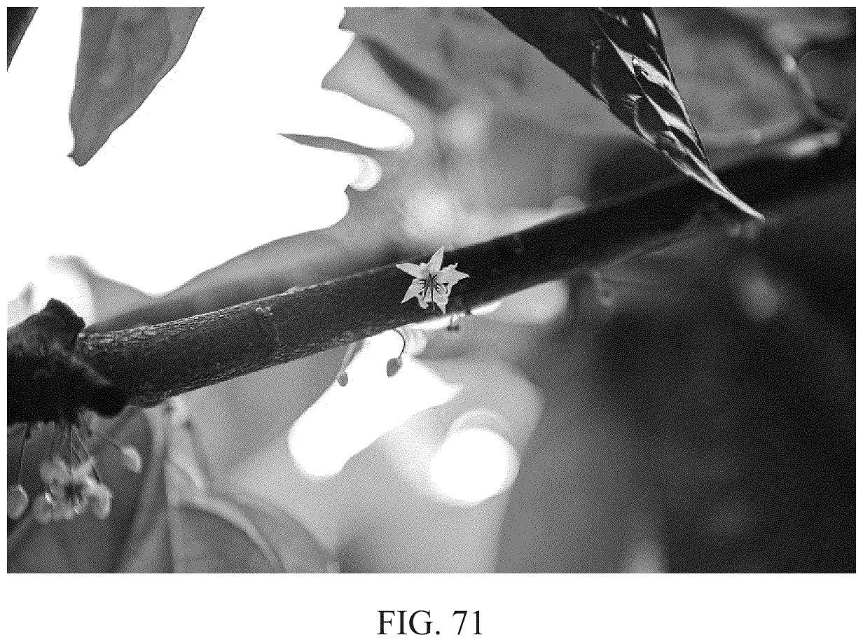

FIG. 72 illustrates flowers and flower buds of variety ‘CMC-012’.

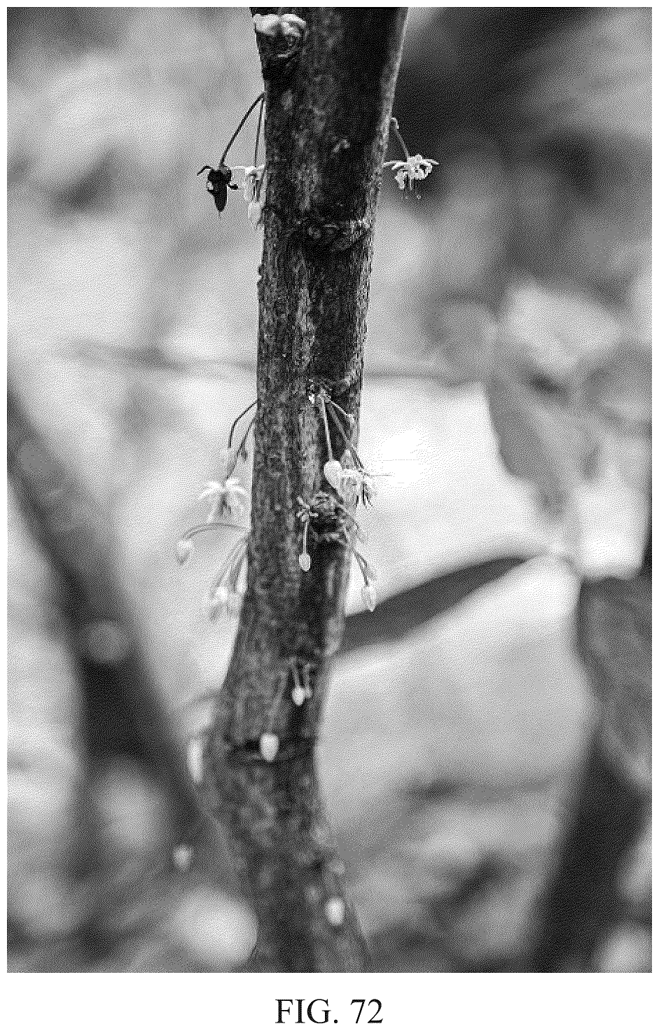

FIG. 73 illustrates leaves of variety ‘CMC-012’.

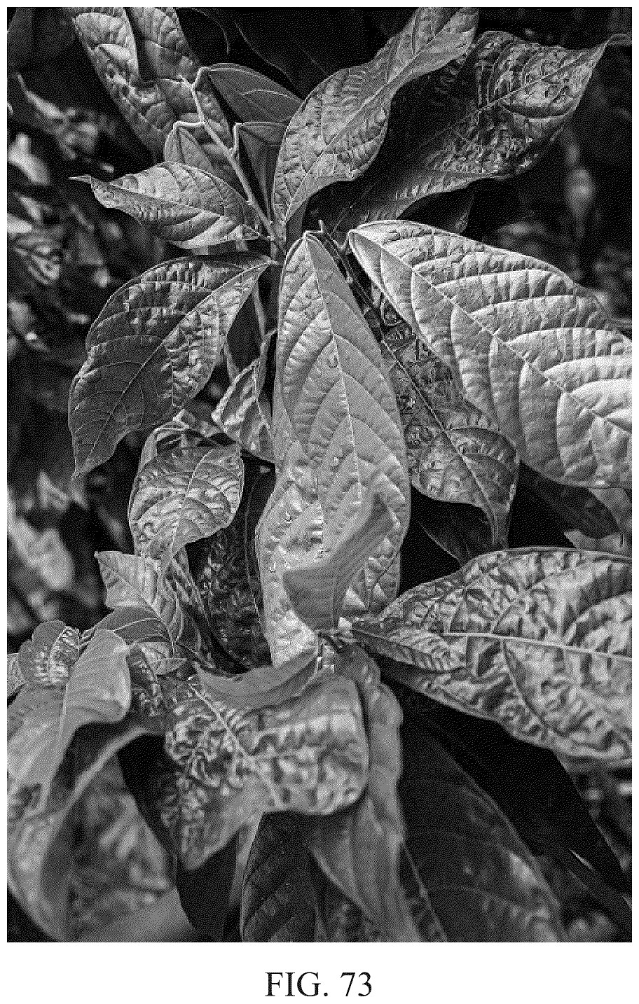

FIG. 74 illustrates a whole tree of variety ‘MC-013’.

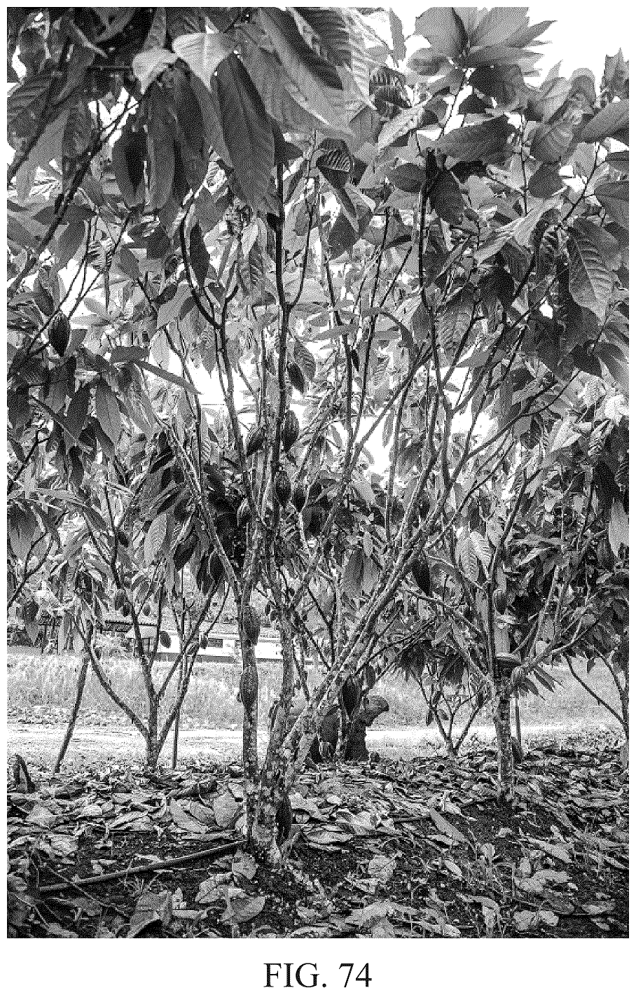

FIG. 75 illustrates a pod of variety ‘CMC-013’.

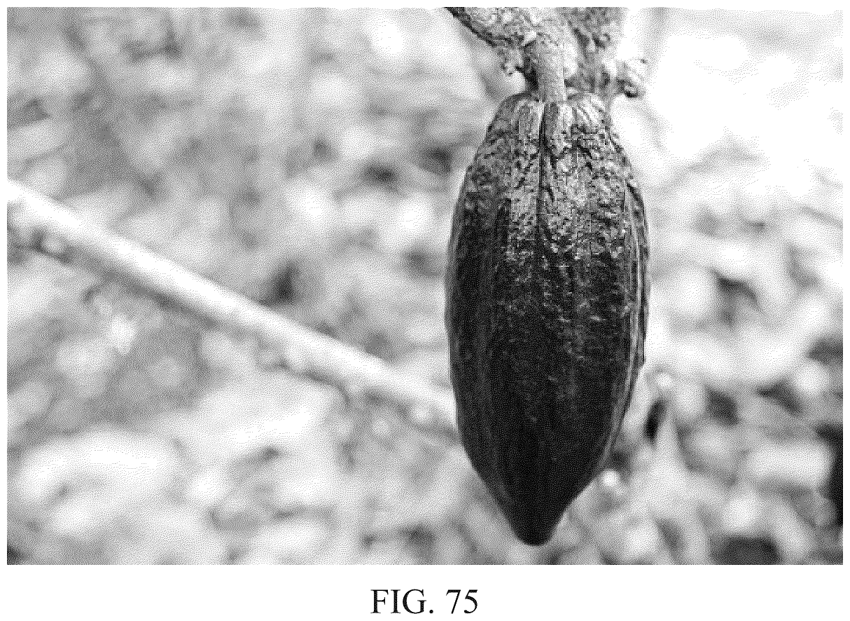

FIG. 76 illustrates a pod of variety ‘CMC-013’.

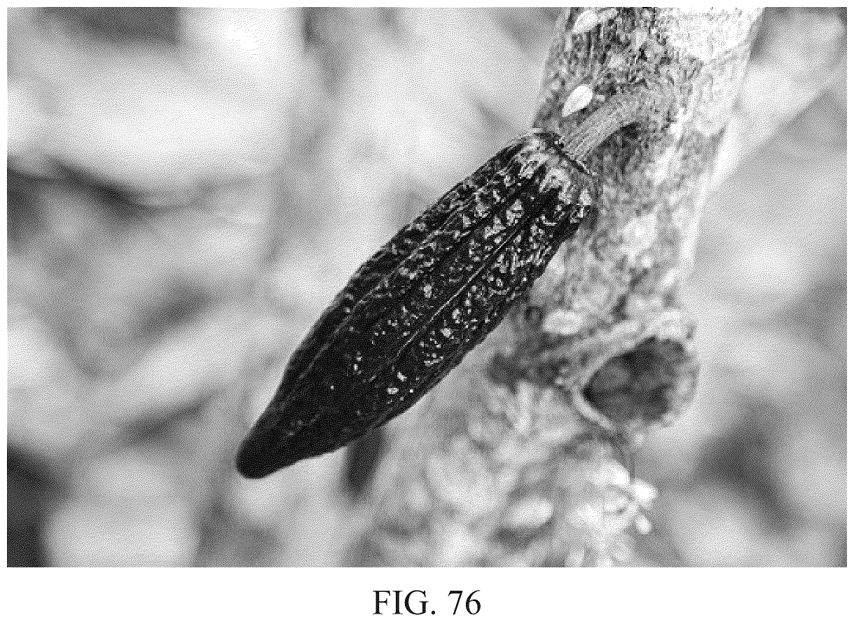

FIG. 77 illustrates a flower of variety ‘CMC-013’.

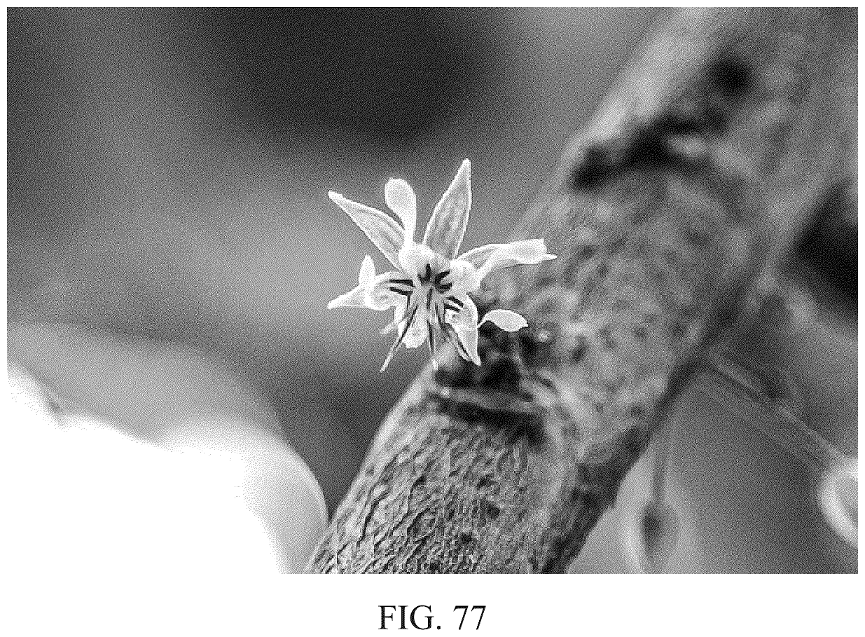

FIG. 78 illustrates flowers and flower buds of variety ‘CMC-013’.

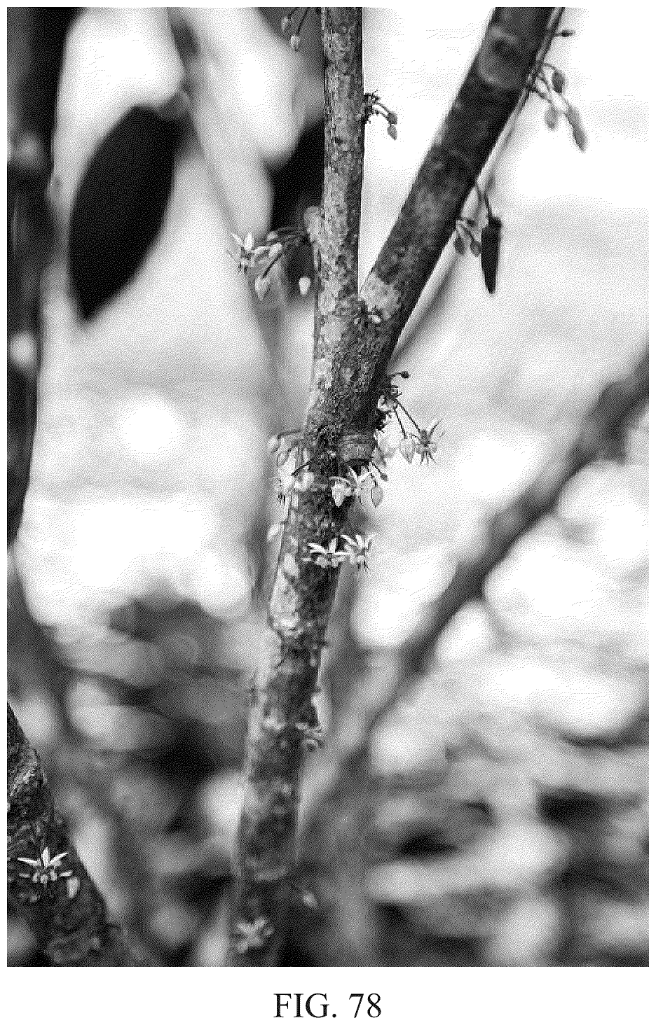

FIG. 79 illustrates flowers and flower buds of variety ‘CMC-013’.

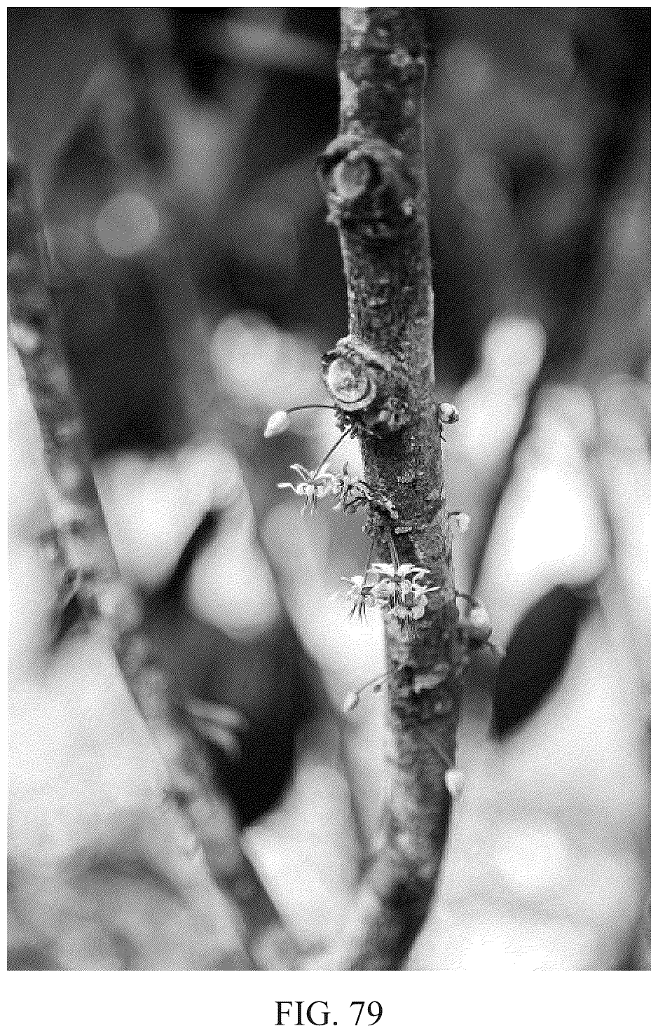

FIG. 80 illustrates leaves of variety ‘CMC-013’.

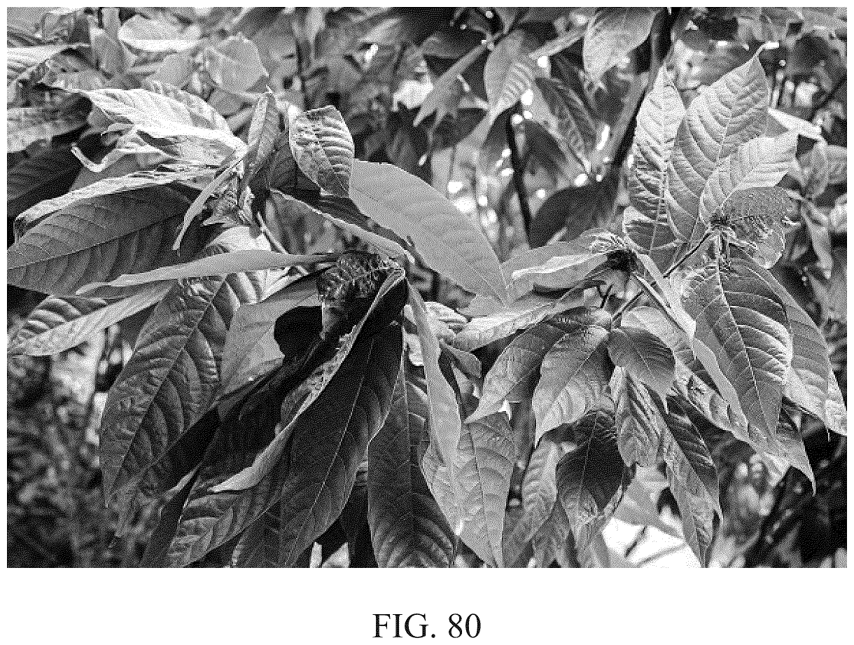

FIG. 81 illustrates a whole tree of variety ‘CMC-014’.

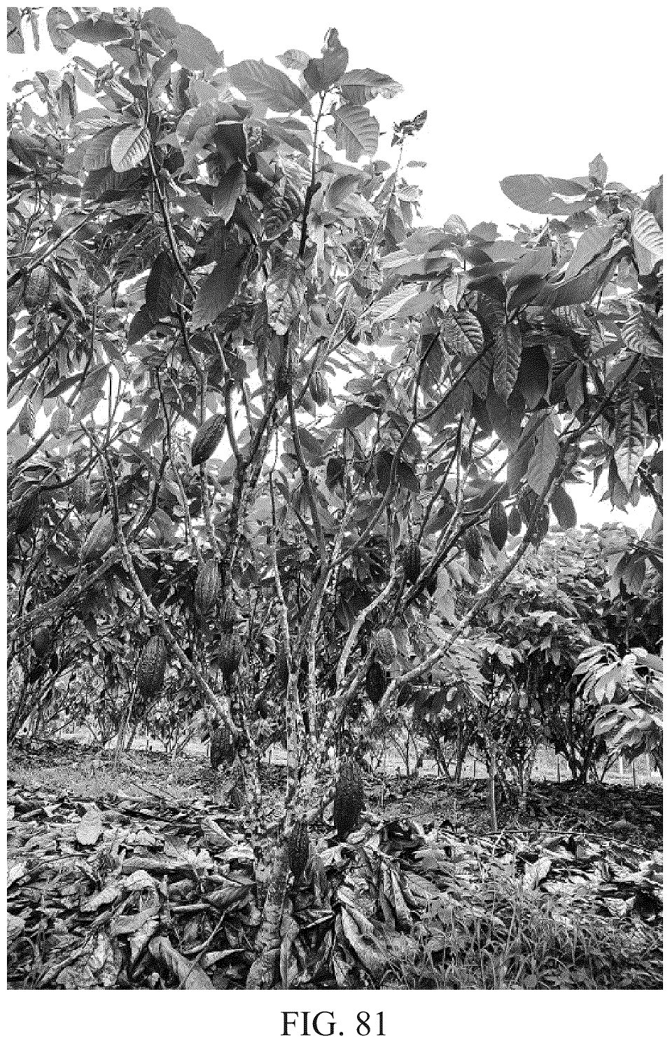

FIG. 82 illustrates a pod of variety ‘CMC-014’.

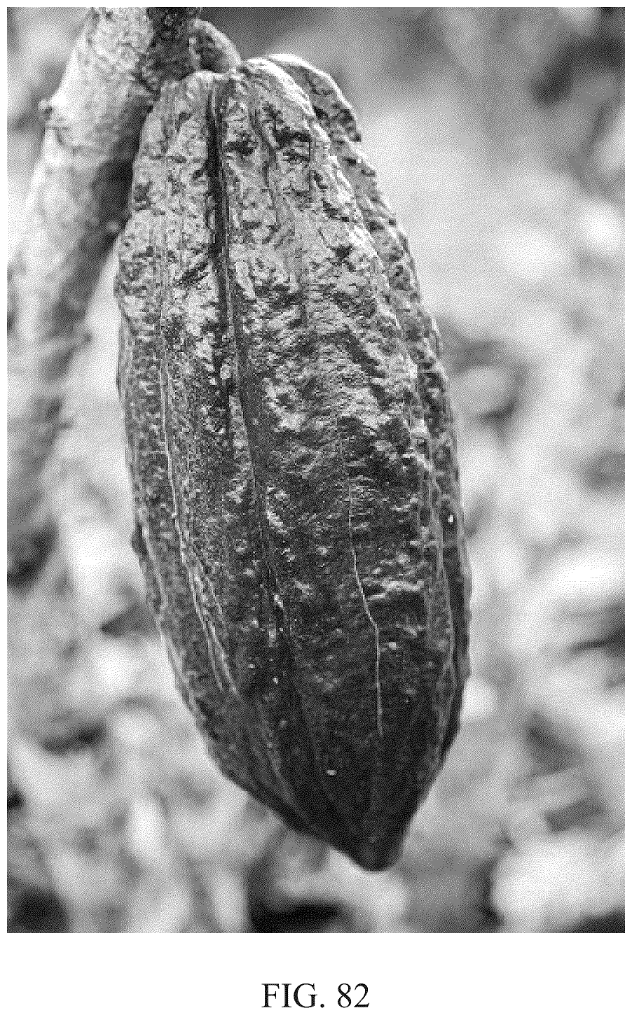

FIG. 83 illustrates flowers of variety ‘CMC-014’.

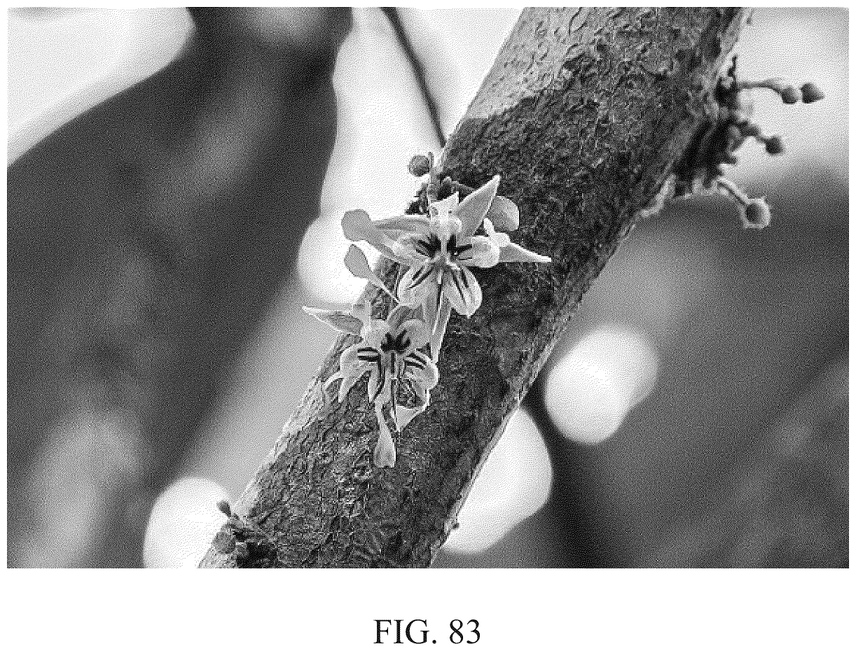

FIG. 84 illustrates flowers and flower buds of variety ‘CMC-014’.

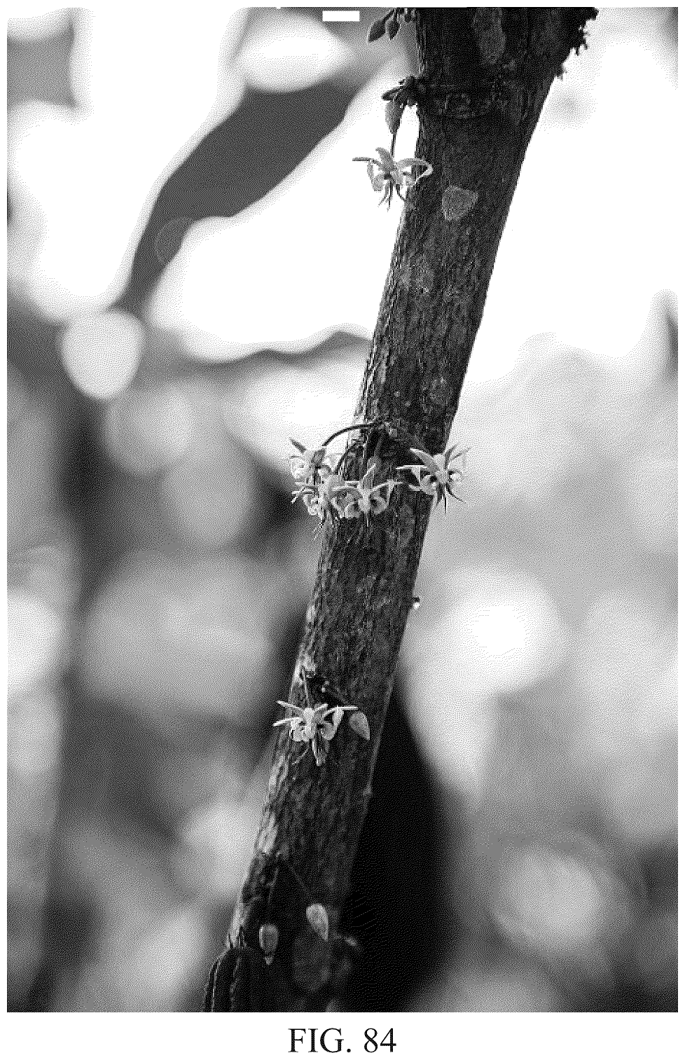

FIG. 85 illustrates leaves of variety ‘CMC-014’.

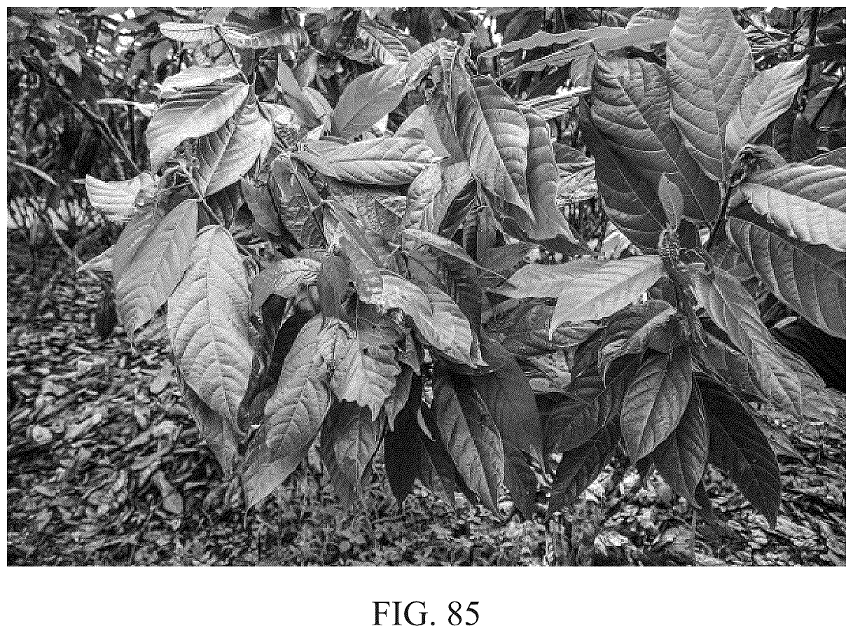

FIG. 86 illustrates a whole tree of variety ‘CMC-015’.

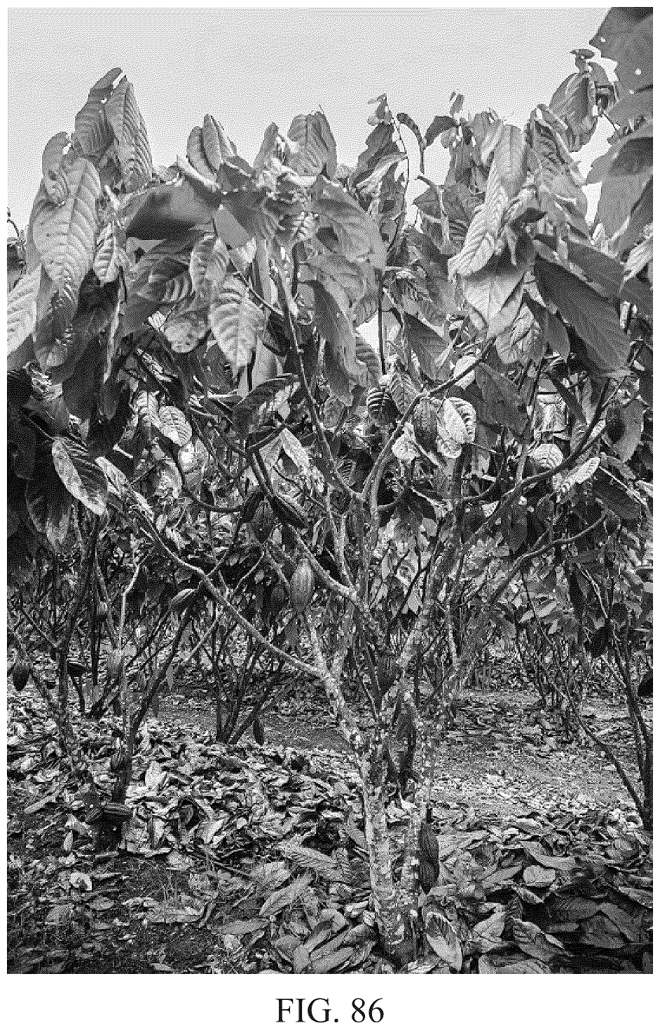

FIG. 87 illustrates a pod of variety ‘CMC-015’.

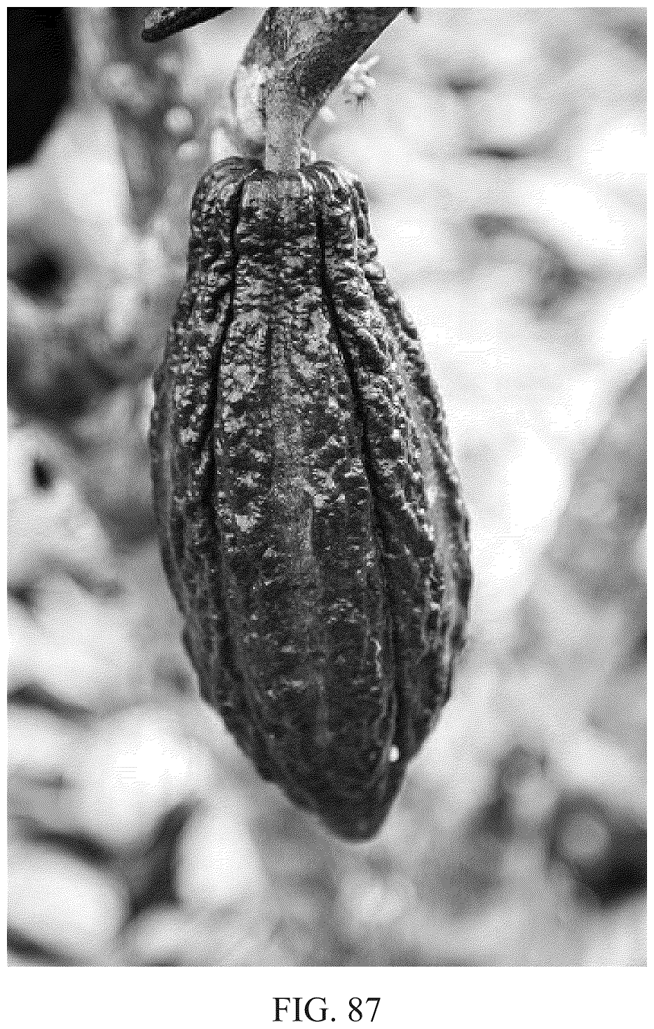

FIG. 88 illustrates a pod of variety ‘CMC-015’.

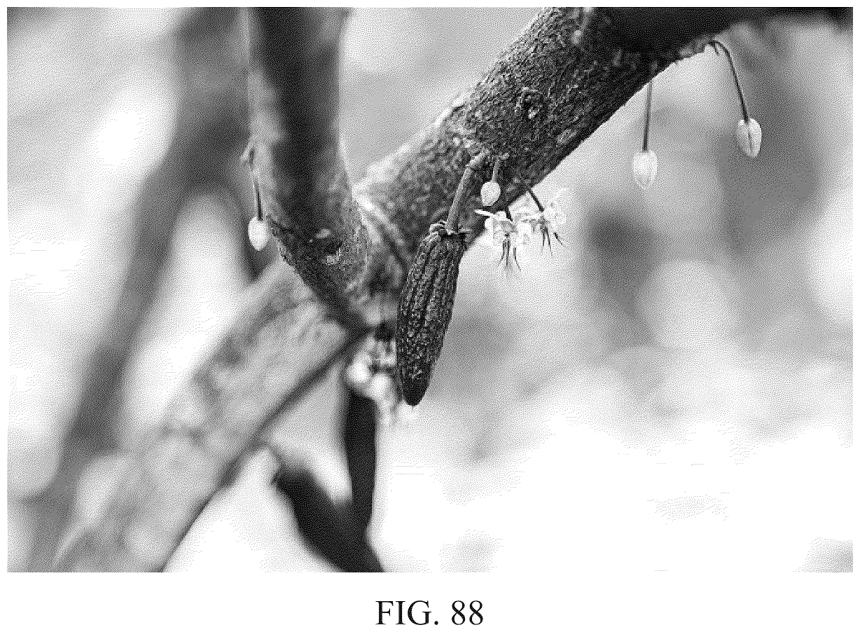

FIG. 89 illustrates a flower of variety ‘CMC-015’.

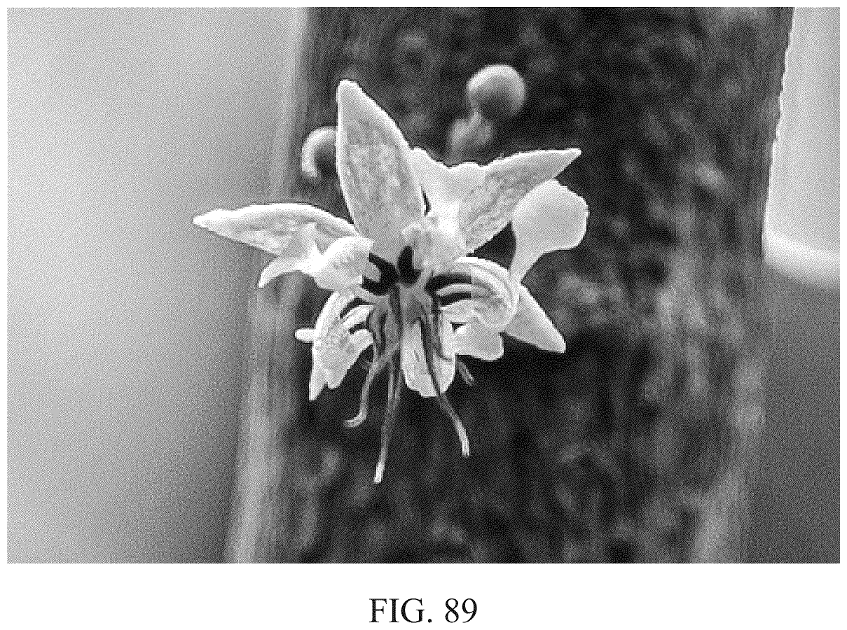

FIG. 90 illustrates flowers and flower buds of variety ‘CMC-015’.

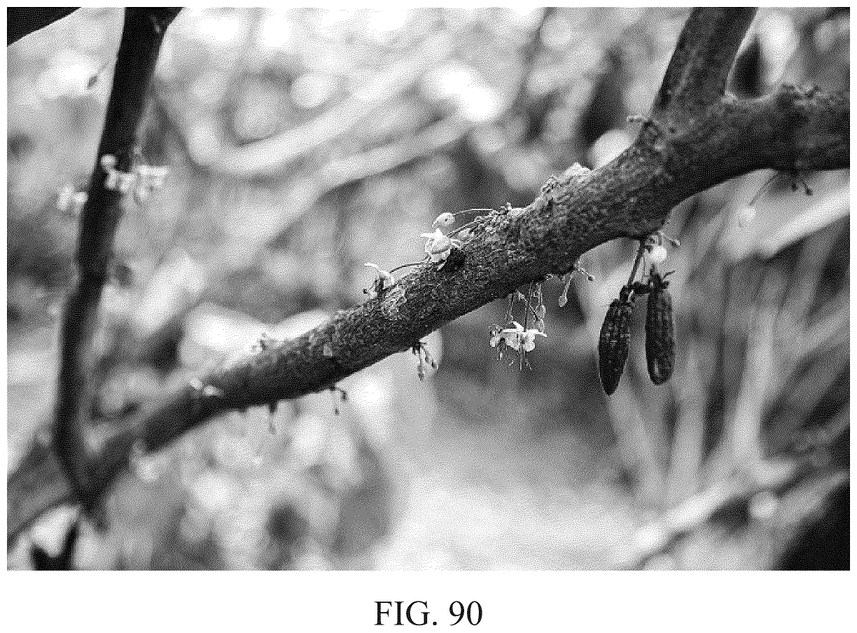

FIG. 91 illustrates leaves of variety ‘CMC-015’.

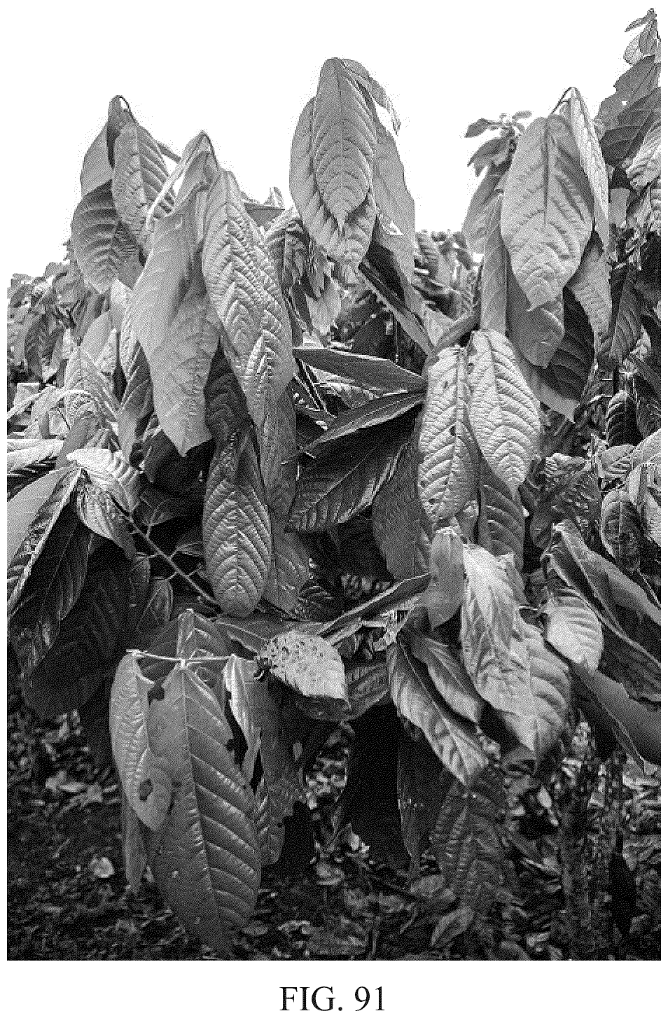

FIG. 92 illustrates a whole tree of variety ‘CMC-016’.

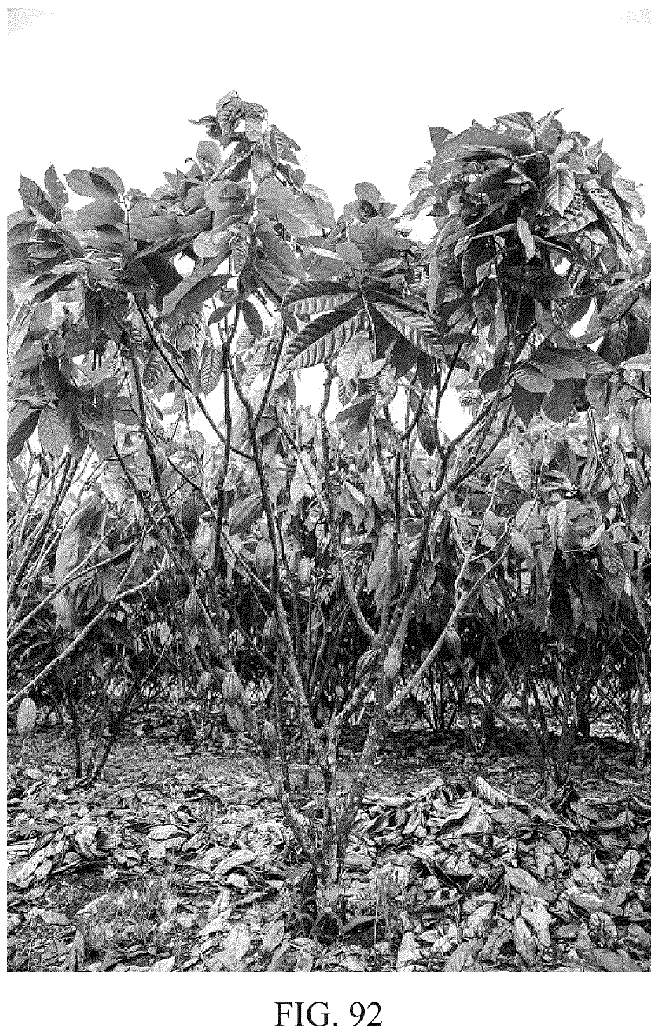

FIG. 93 illustrates a pod of variety ‘CMC-016’.

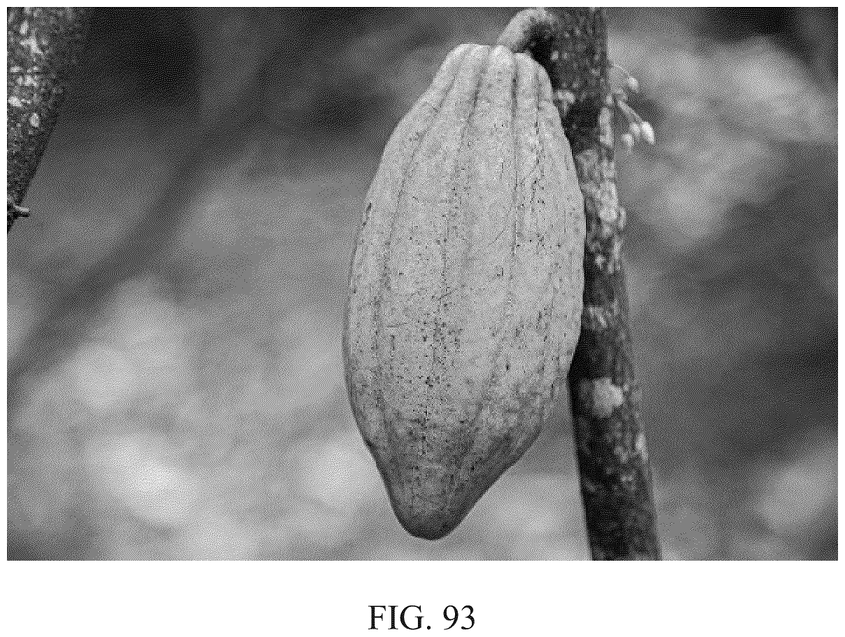

FIG. 94 illustrates a pod of variety ‘CMC-016’.

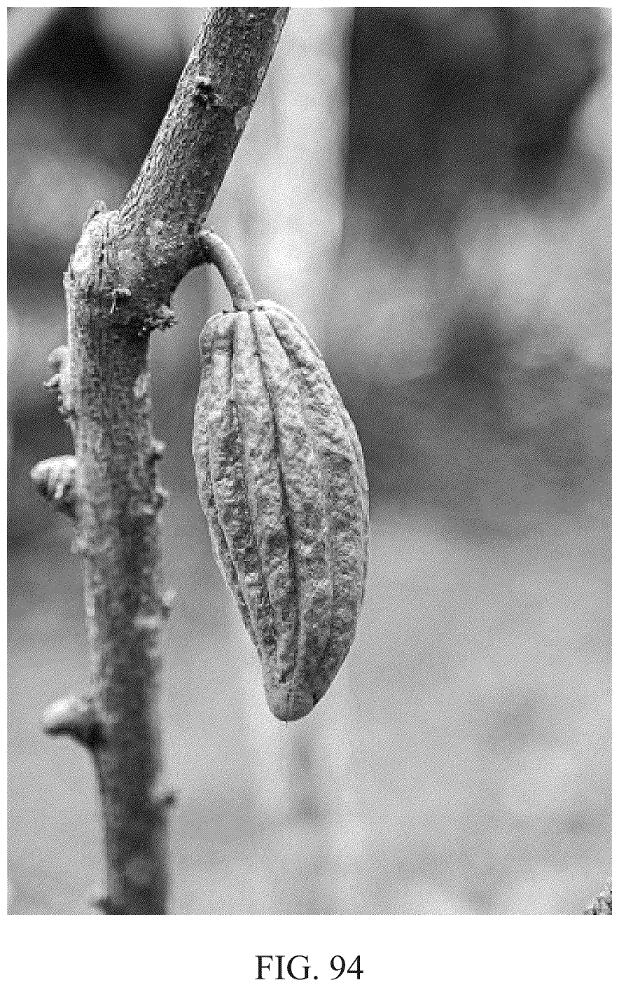

FIG. 95 illustrates a flower of variety ‘CMC-016’.

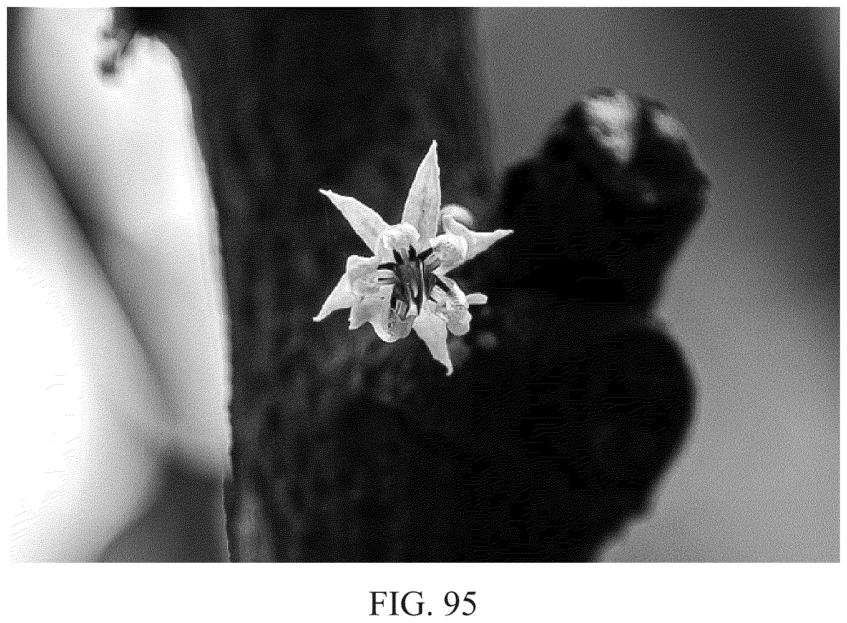

FIG. 96 illustrates flowers and flower buds of variety ‘CMC-016’.

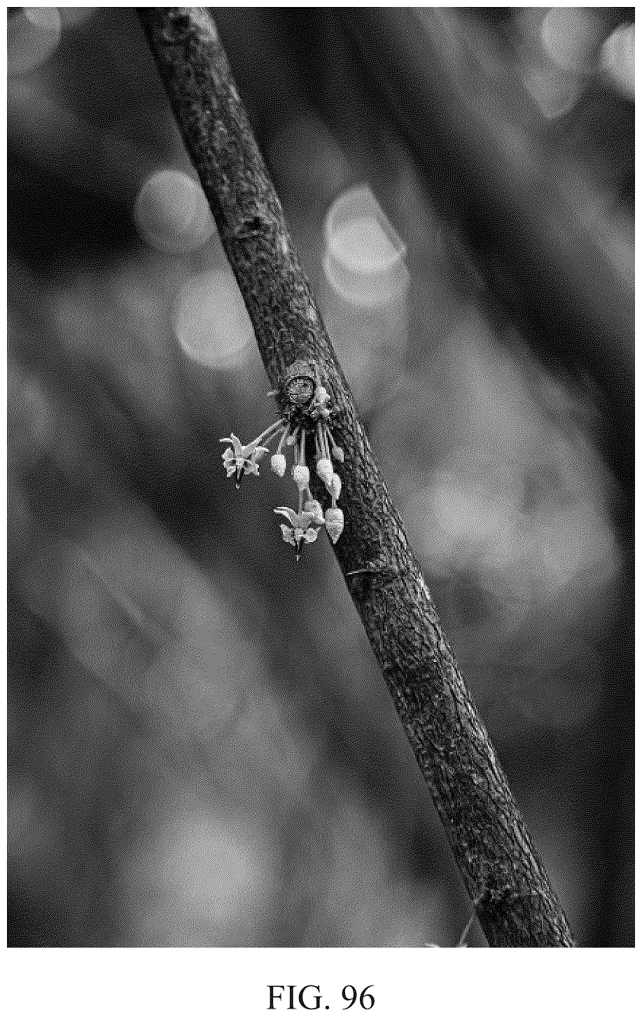

FIG. 97 illustrates leaves of variety ‘CMC-016’.

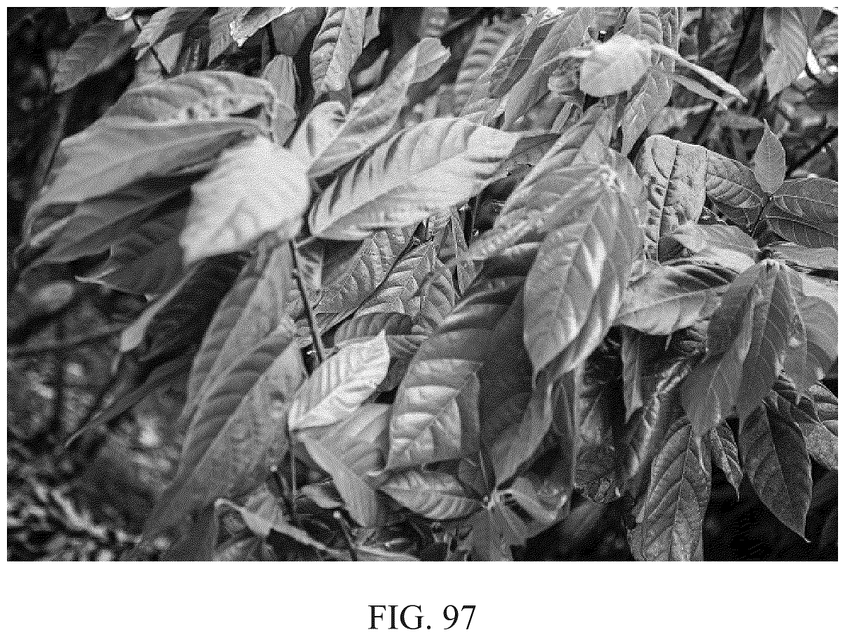

FIG. 98 illustrates a whole tree of variety ‘CMC-017’.

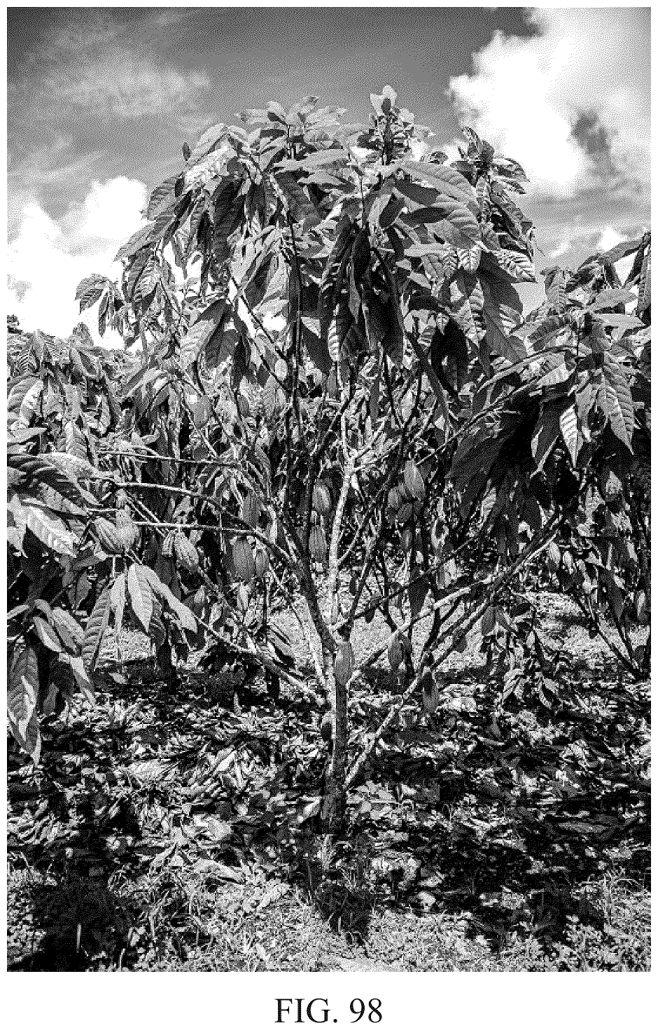

FIG. 99 illustrates a pod of variety ‘CMC-017’.

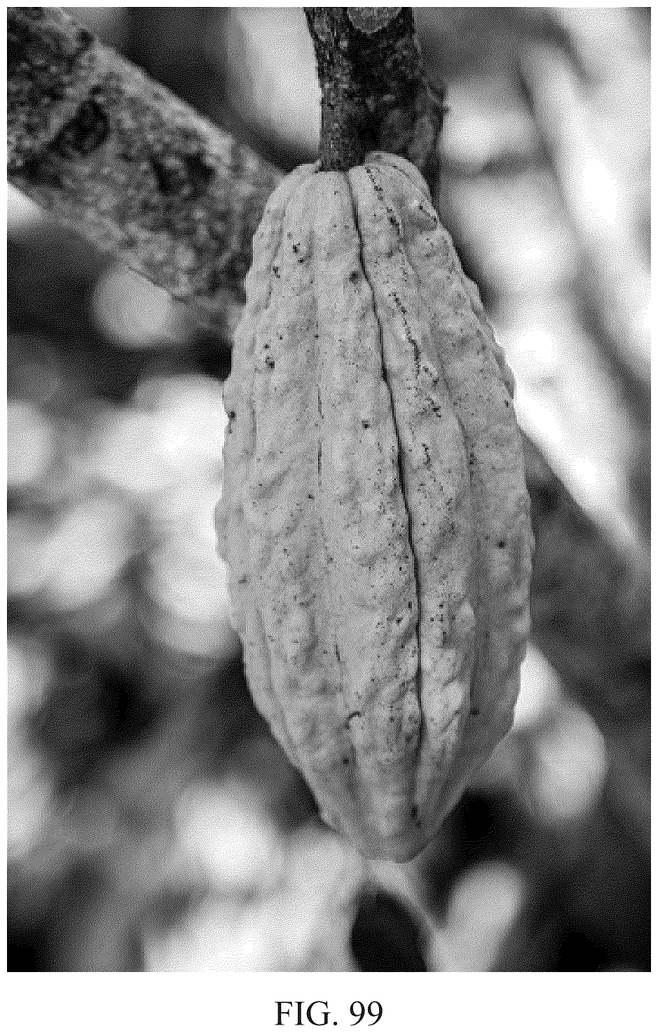

FIG. 100 illustrates a pod of variety ‘CMC-017’.

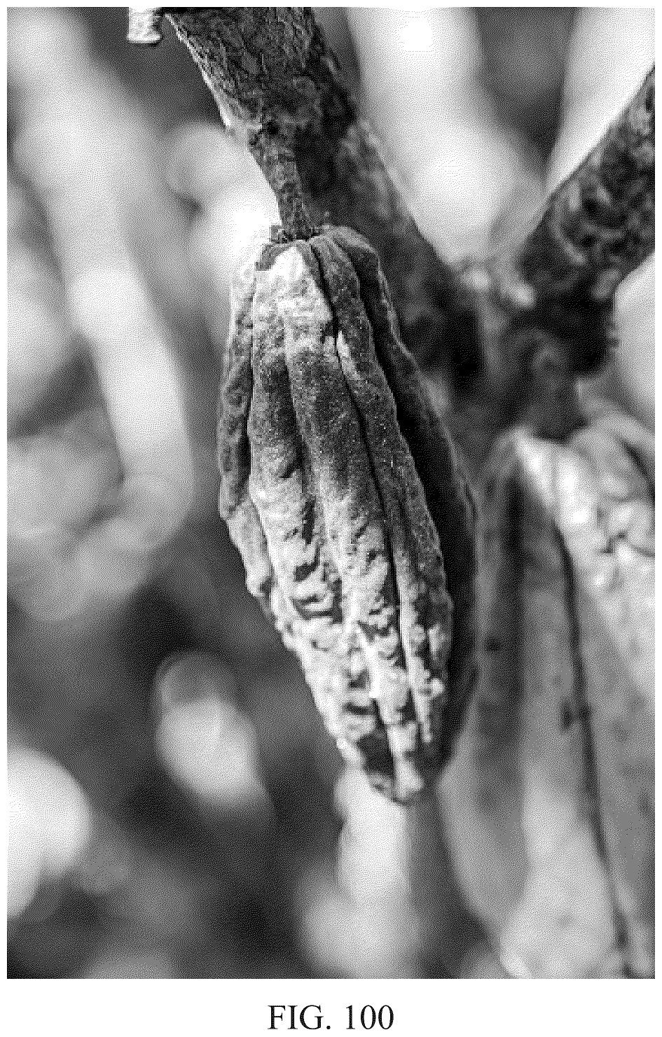

FIG. 101 illustrates a flower of variety ‘CMC-017’.

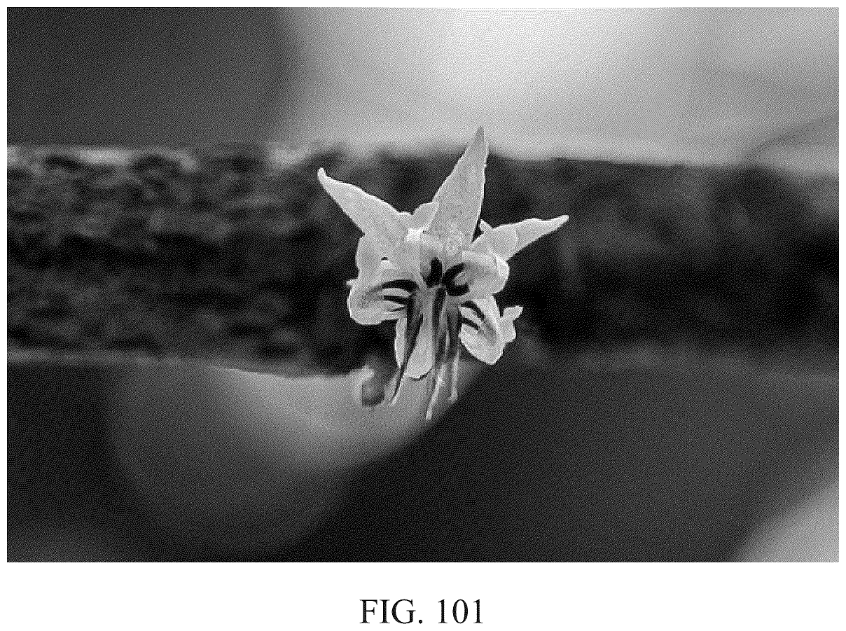

FIG. 102 illustrates a flower and flower buds of variety ‘CMC-017’

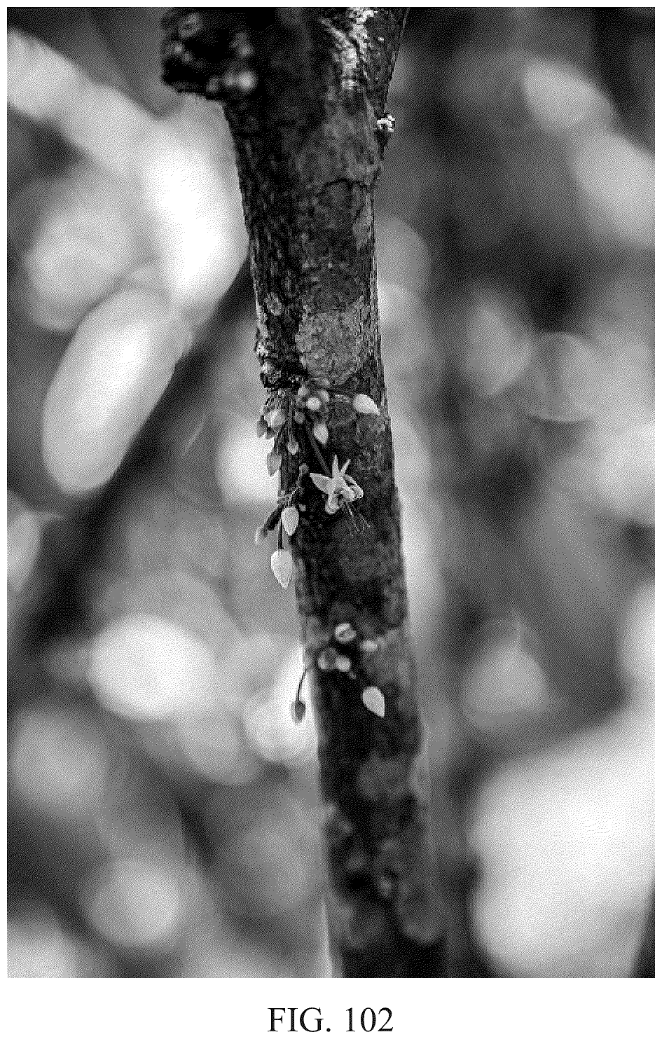

FIG. 103 illustrates leaves of variety ‘CMC-017’.

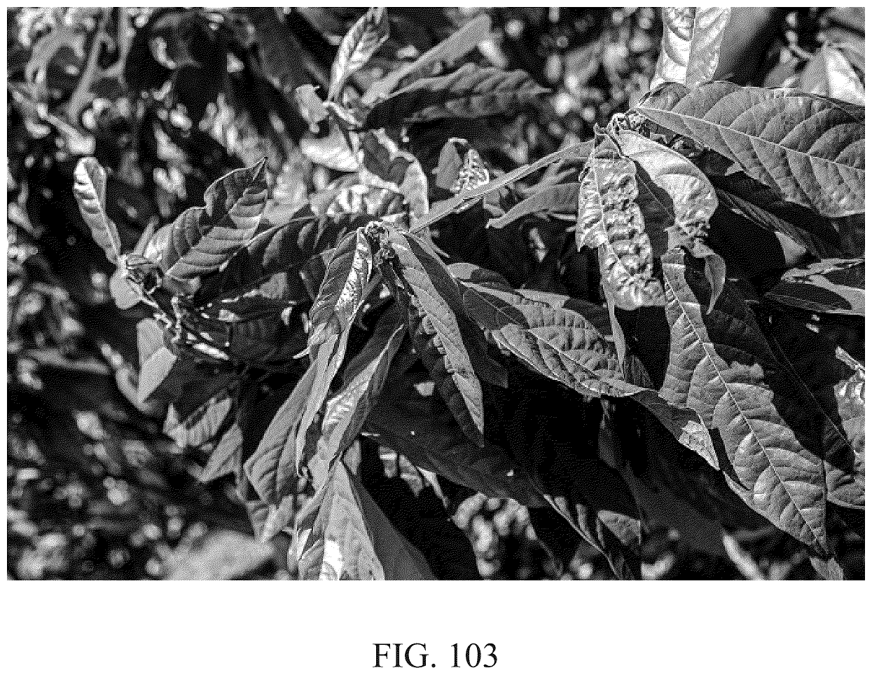

FIG. 104 illustrates a whole tree of variety ‘CMC-018’.

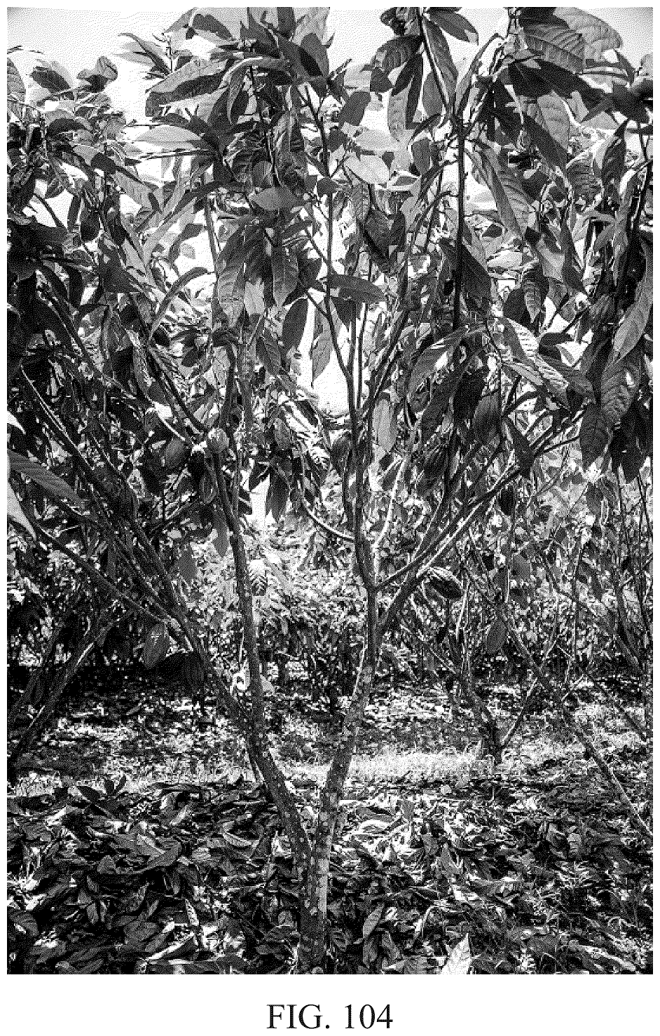

FIG. 105 illustrates a pod of variety ‘CMC-018’.

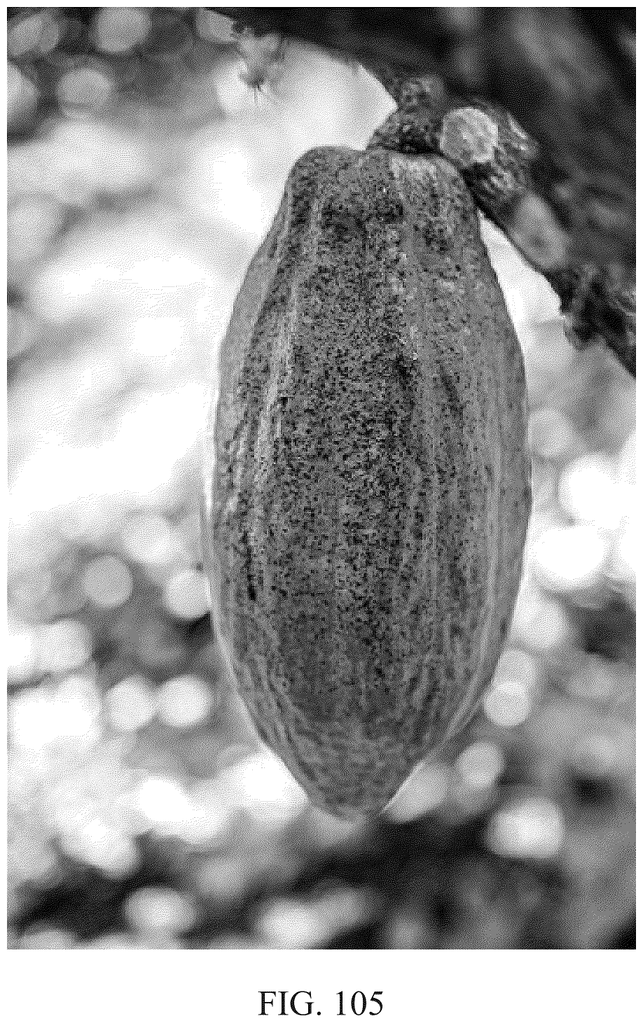

FIG. 106 illustrates a pod of variety ‘CMC-018’.

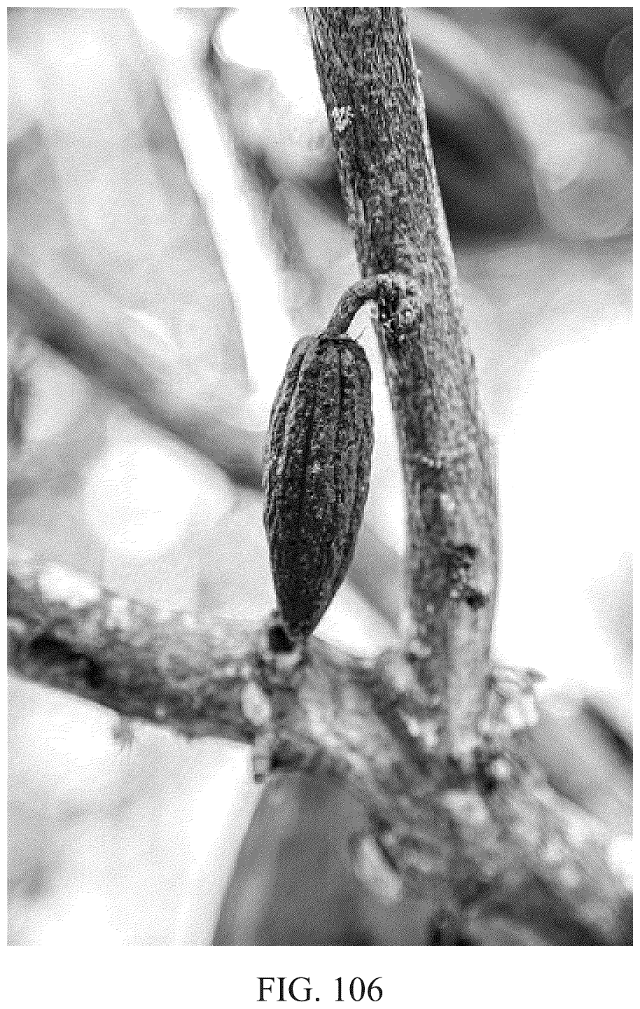

FIG. 107 illustrates a flower of variety ‘CMC-018’.

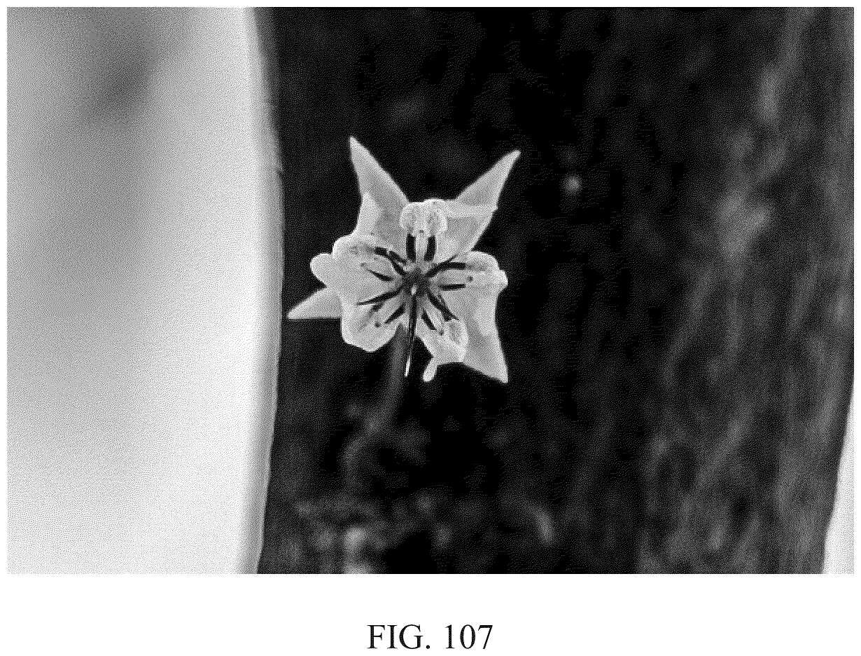

FIG. 108 illustrates flowers and flower buds of variety ‘CMC-018’.

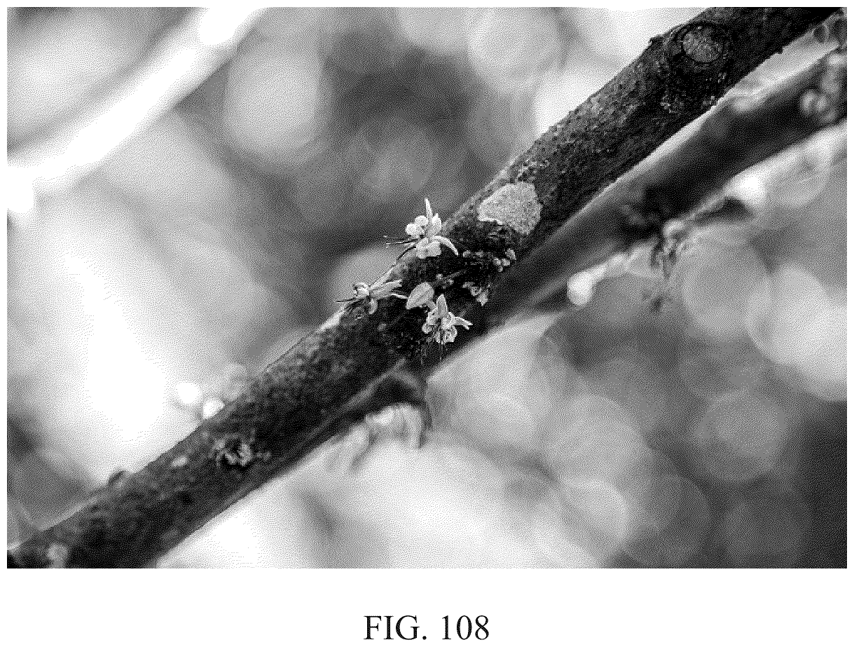

FIG. 109 illustrates leaves of variety ‘CMC-018’.

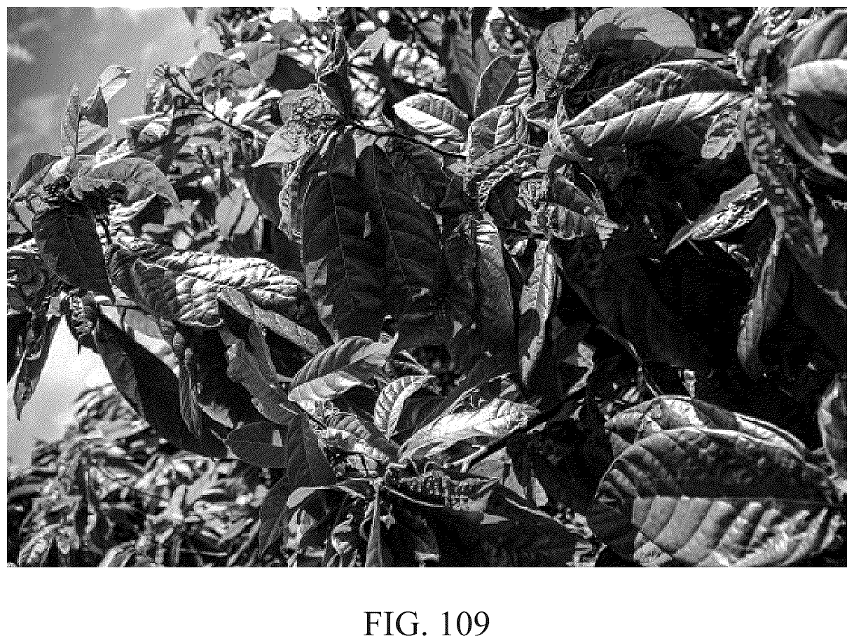

FIG. 110 illustrates a whole tree of variety ‘CMC-019’.

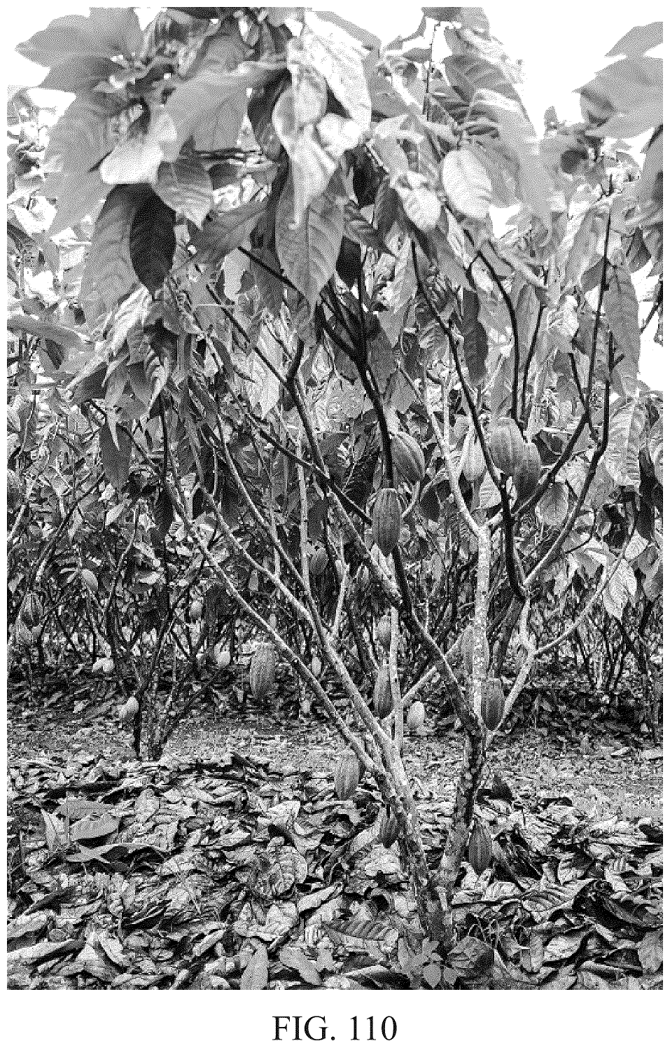

FIG. 111 illustrates a pod of variety ‘CMC-019’.

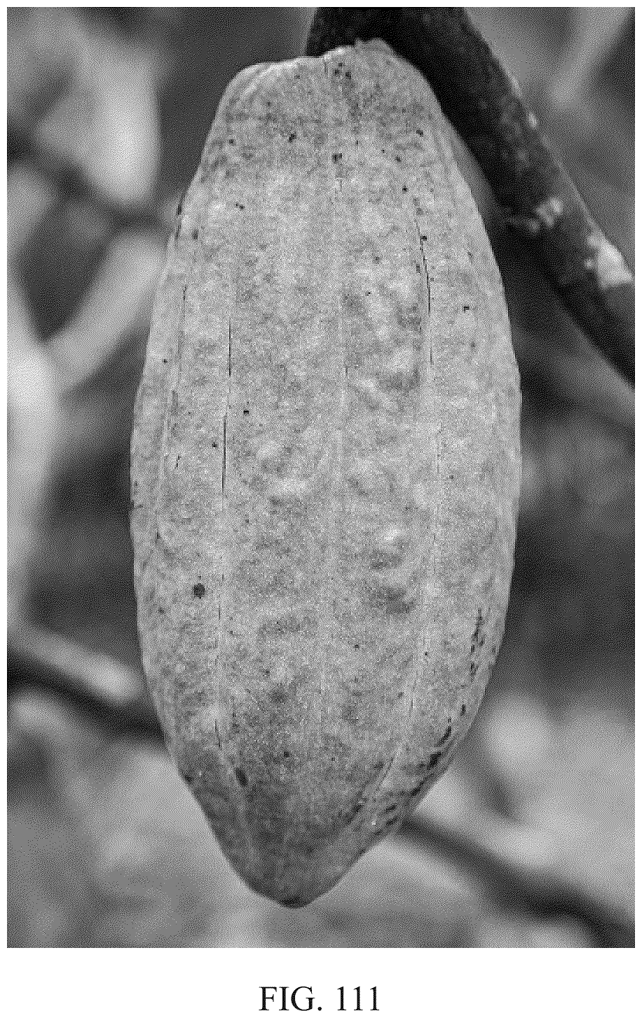

FIG. 112 illustrates a flower of variety ‘CMC-019’.

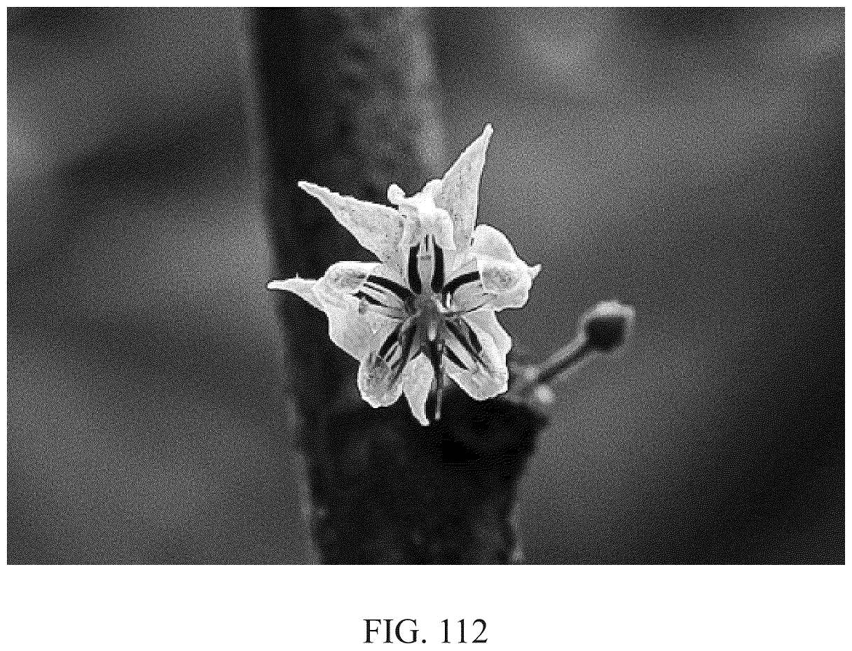

FIG. 113 illustrates flowers and flower buds of variety ‘CMC-019’.

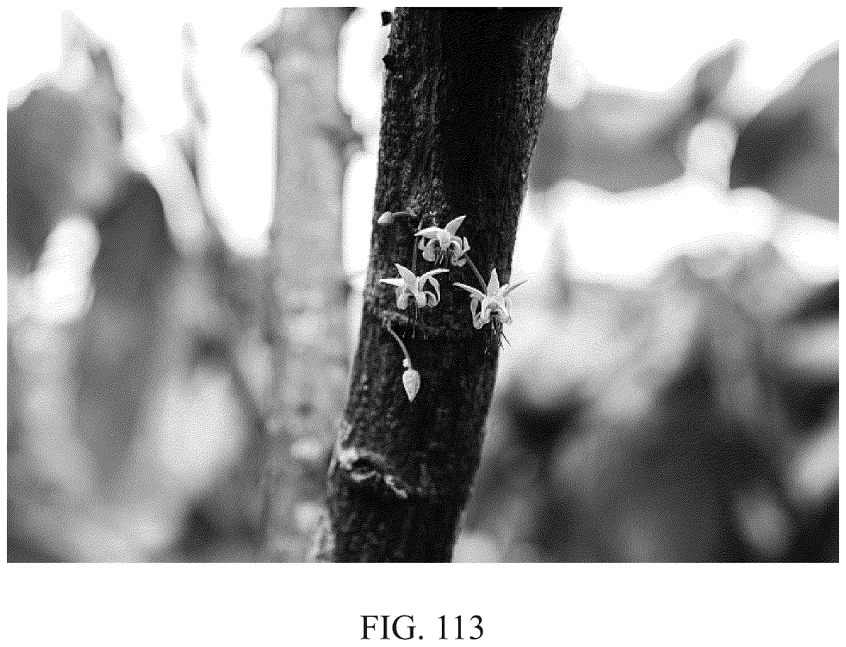

FIG. 114 illustrates leaves of variety ‘CMC-019’.

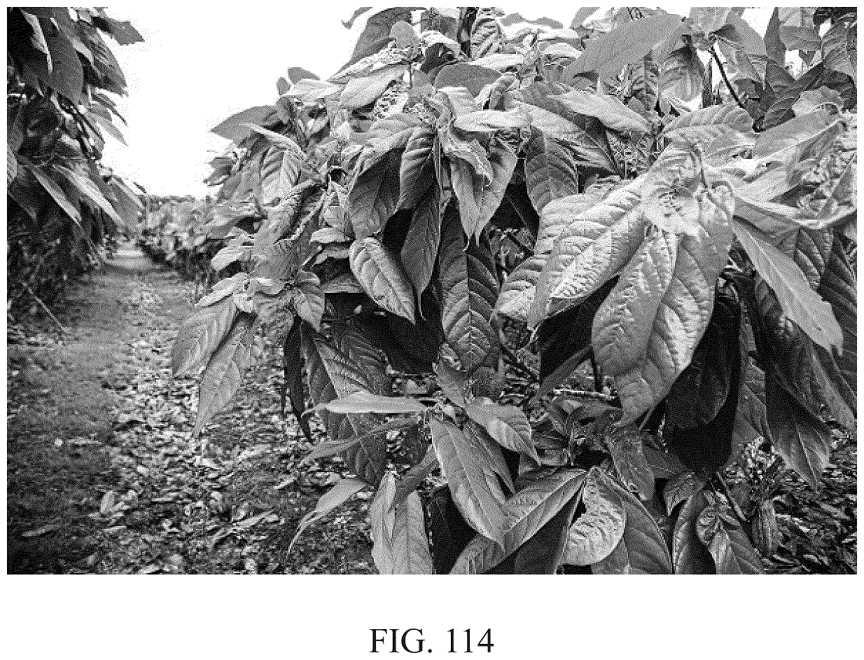

FIG. 115 illustrates leaves of variety ‘CMC-019’.

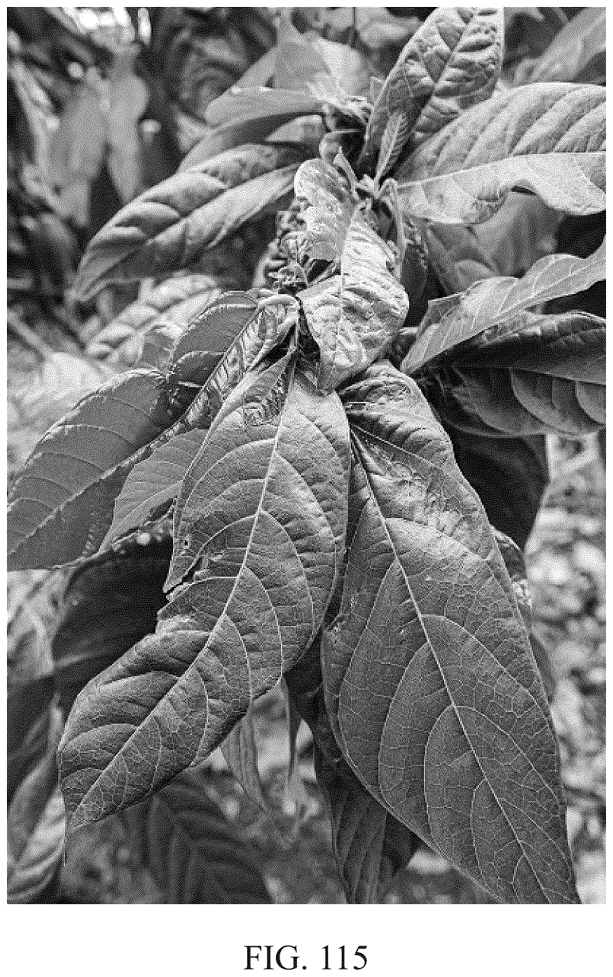

FIG. 116 illustrates a whole tree of variety ‘CMC-020’.

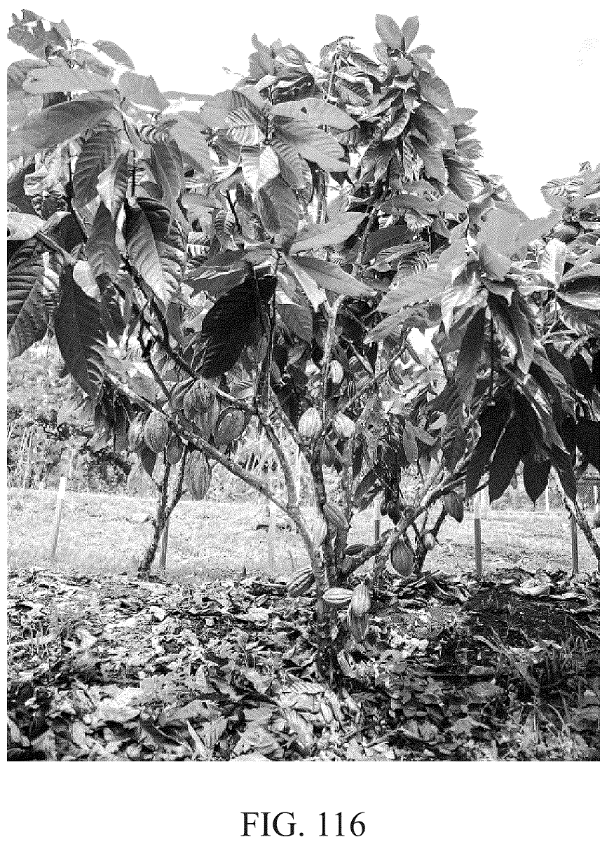

FIG. 117 illustrates a pod of variety ‘CMC-020’.

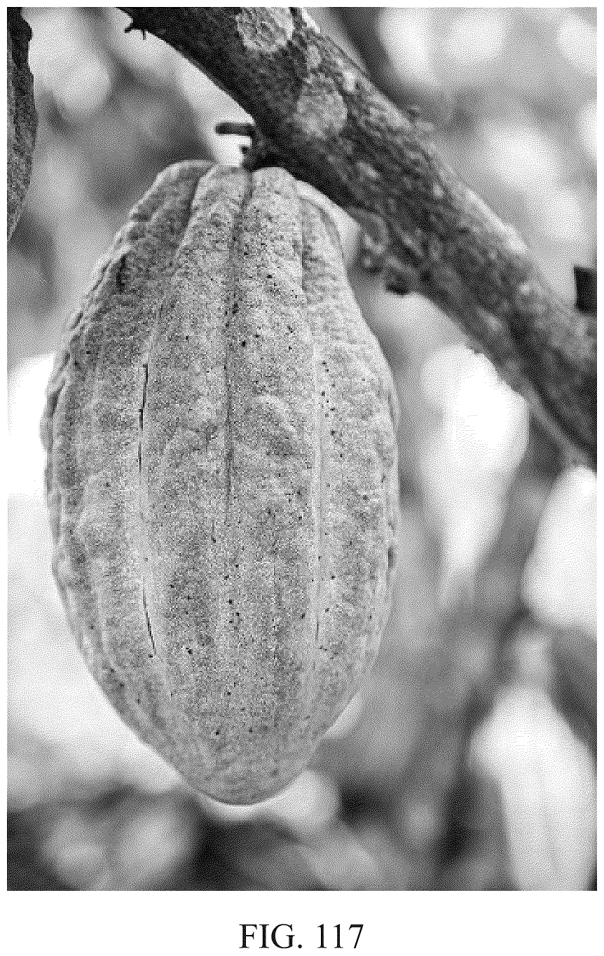

FIG. 118 illustrates a flower of variety ‘CMC-020’.

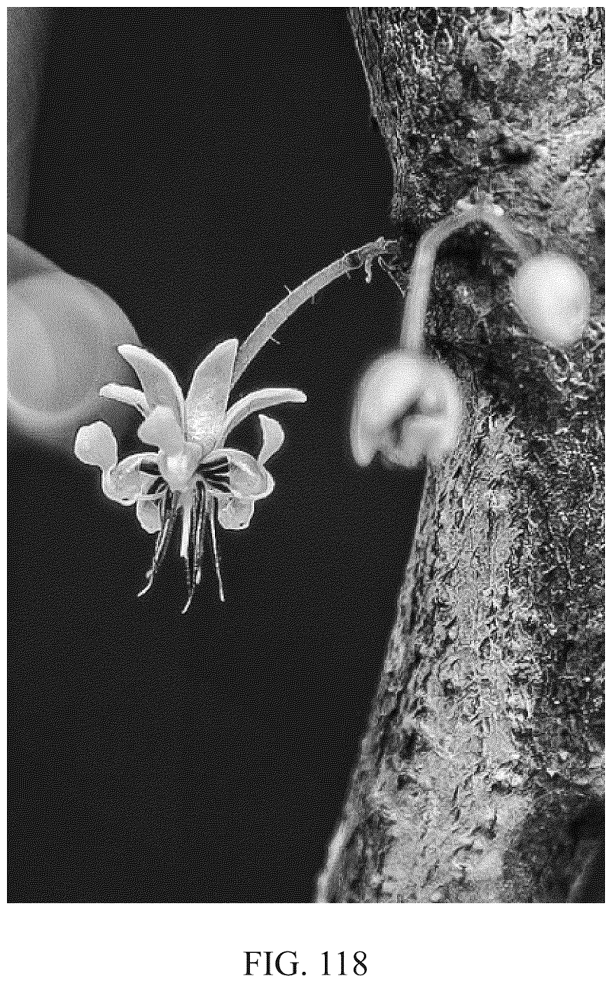

FIG. 119 illustrates flowers and flower buds of variety ‘CMC-020’.

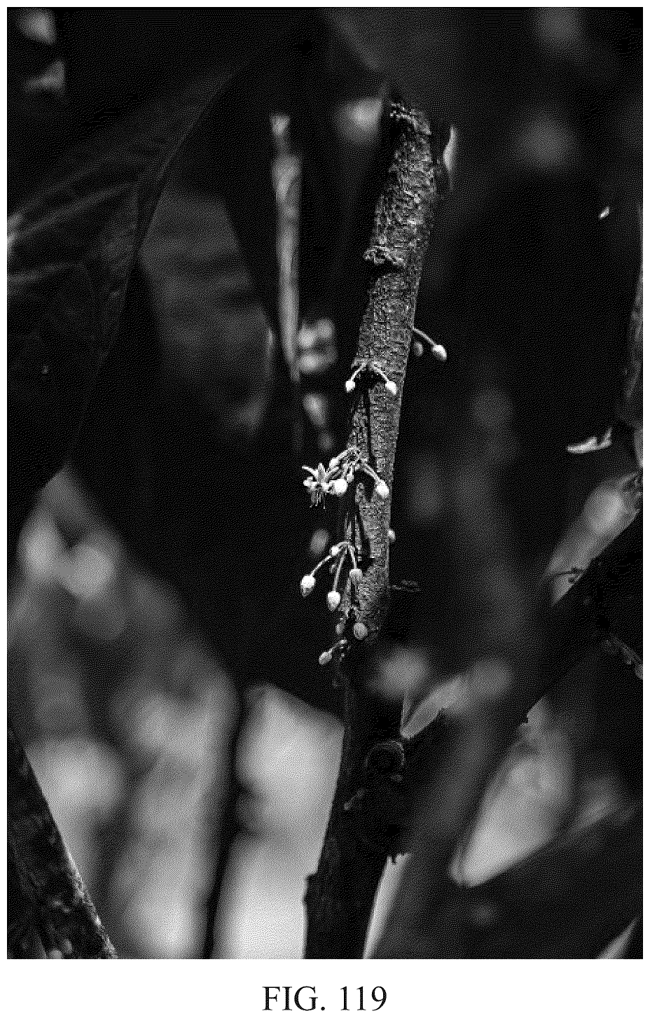

FIG. 120 illustrates flowers and flower buds of variety ‘CMC-020’.

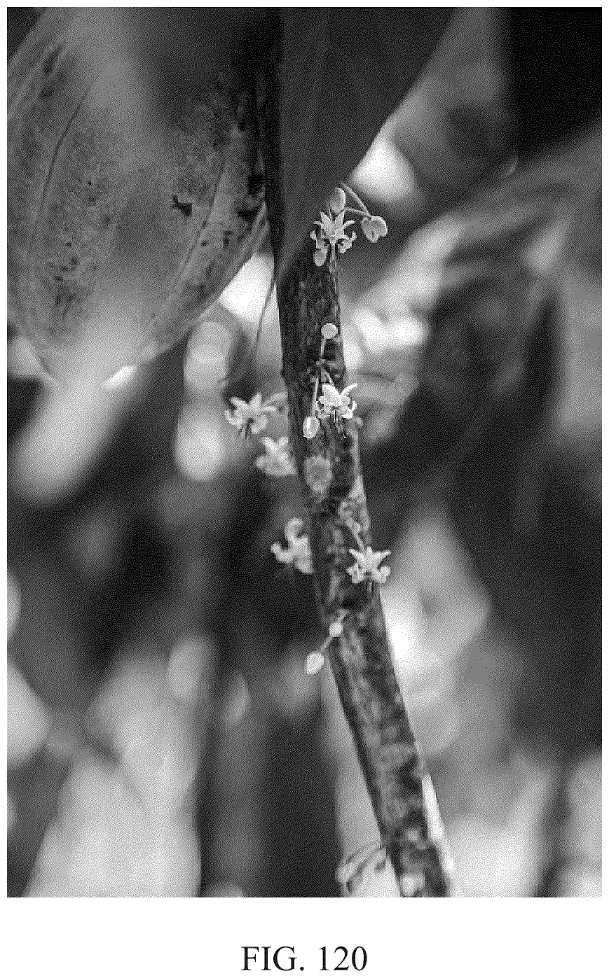

FIG. 121 illustrates leaves of variety ‘CMC-020’.

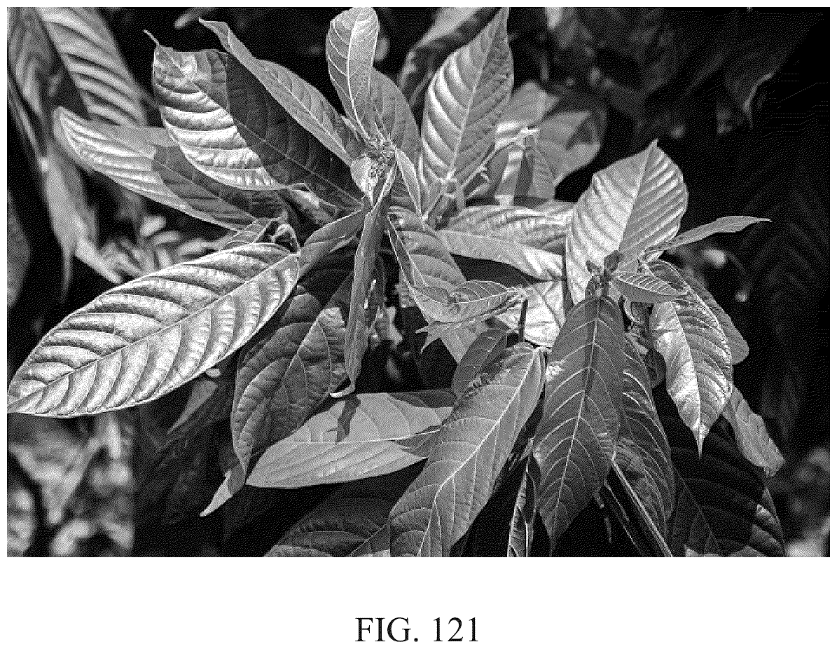

FIG. 122 illustrates a whole tree of variety ‘CMC-021’.

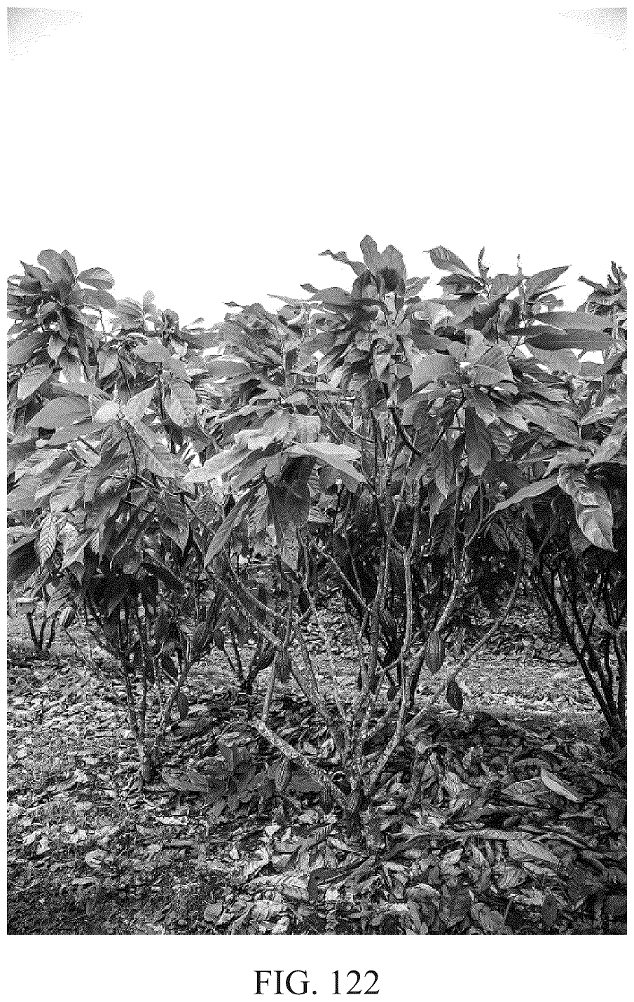

FIG. 123 illustrates a whole tree of variety ‘CMC-021’.

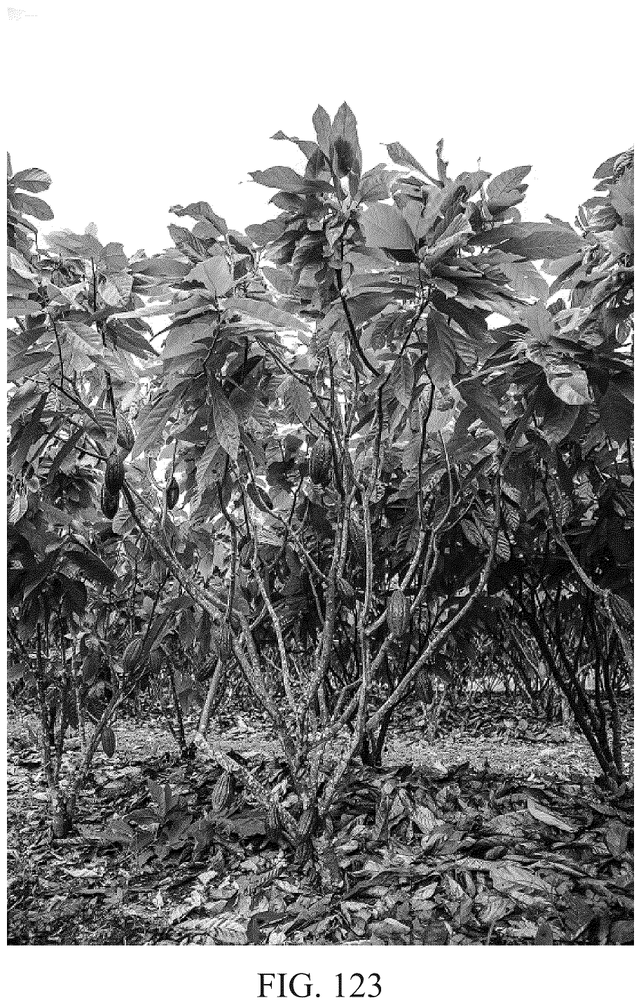

FIG. 124 illustrates a pod of variety ‘CMC-021’.

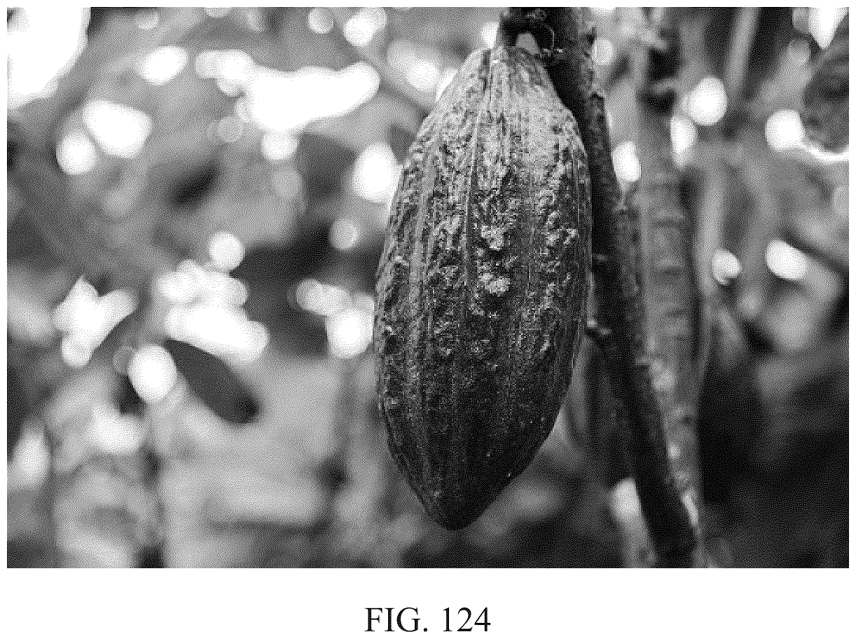

FIG. 125 illustrates a pod of variety ‘CMC-021’.

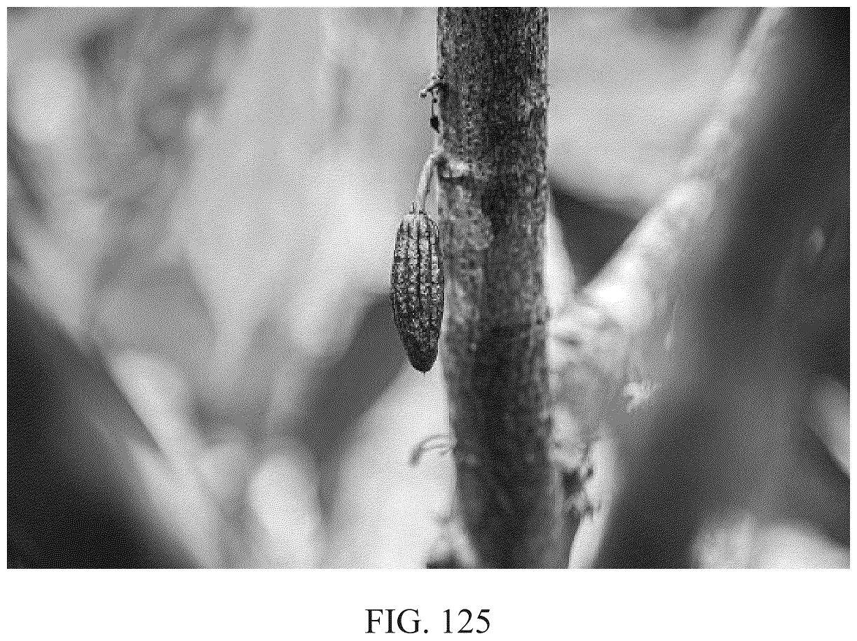

FIG. 126 illustrates a flower of variety ‘CMC-021’.

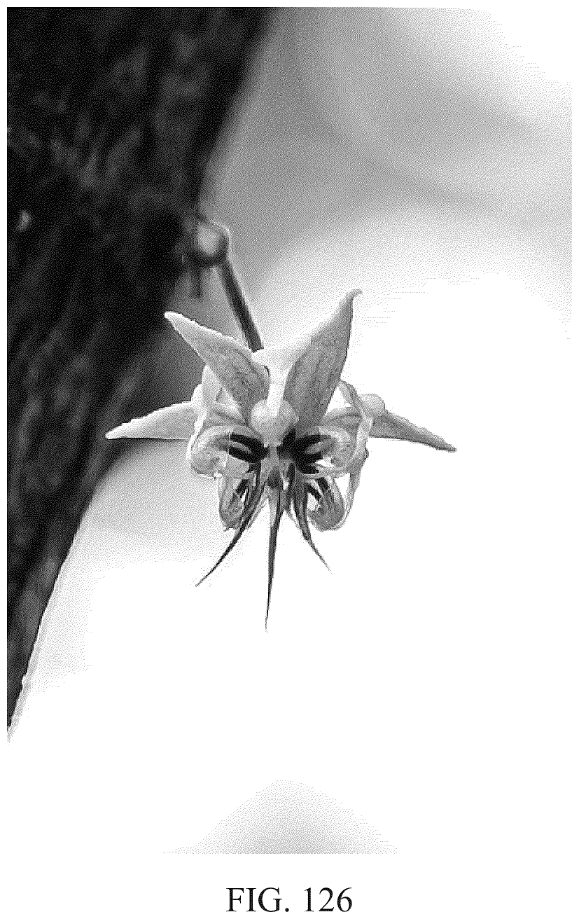

FIG. 127 illustrates a flower of variety ‘CMC-021’.

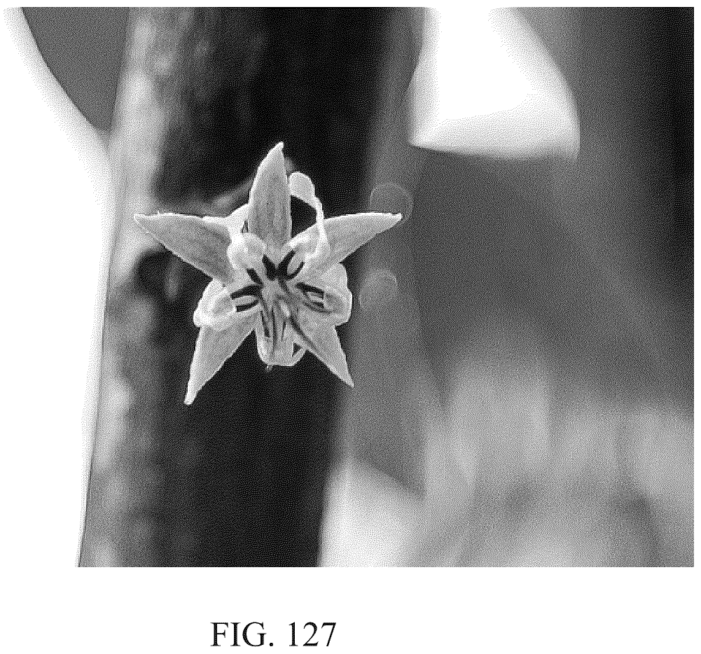

FIG. 128 illustrates flowers and flower buds of variety ‘CMC-021’.

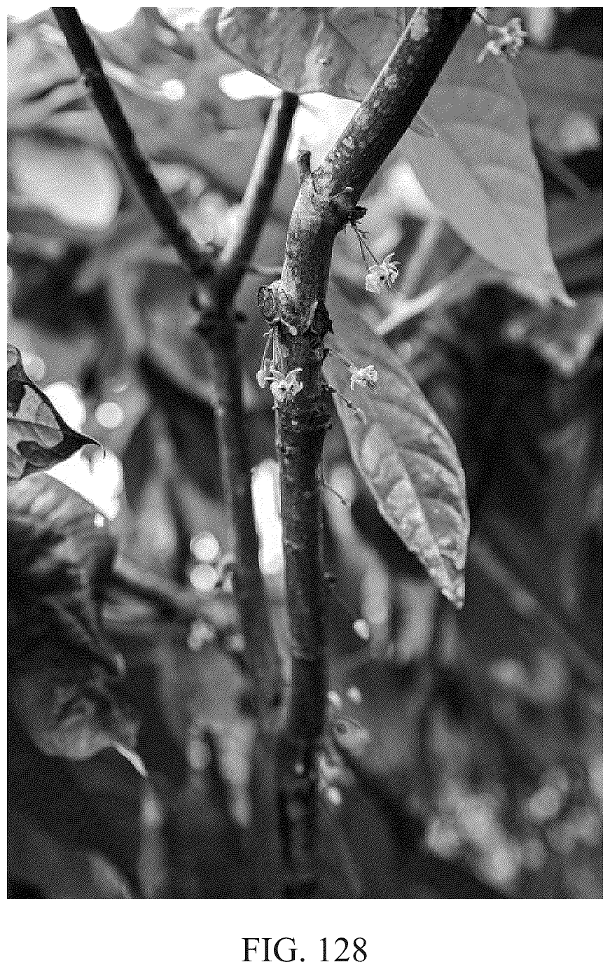

FIG. 129 illustrates flowers and flower buds of variety ‘CMC-021’.

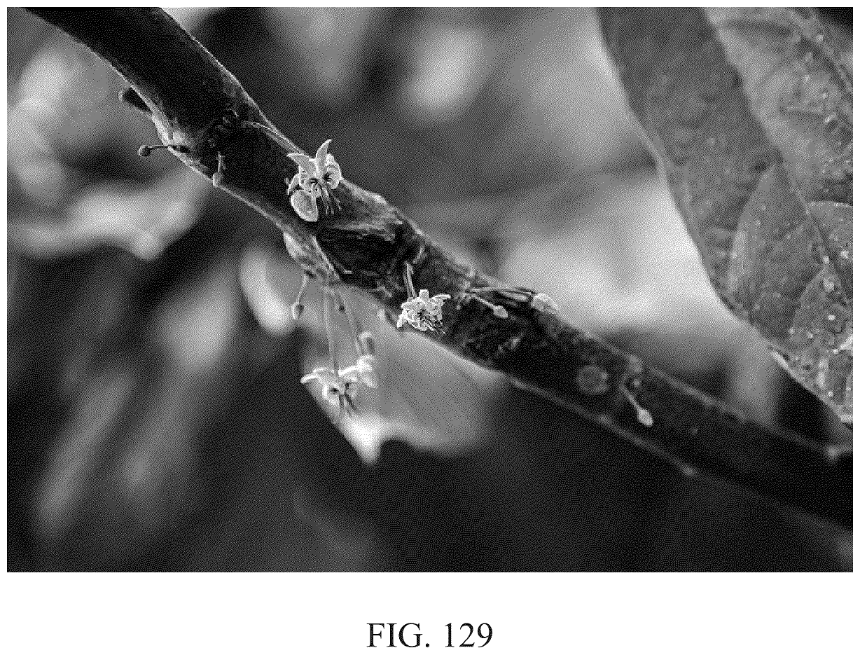

FIG. 130 illustrates flower buds of variety ‘CMC-021’.

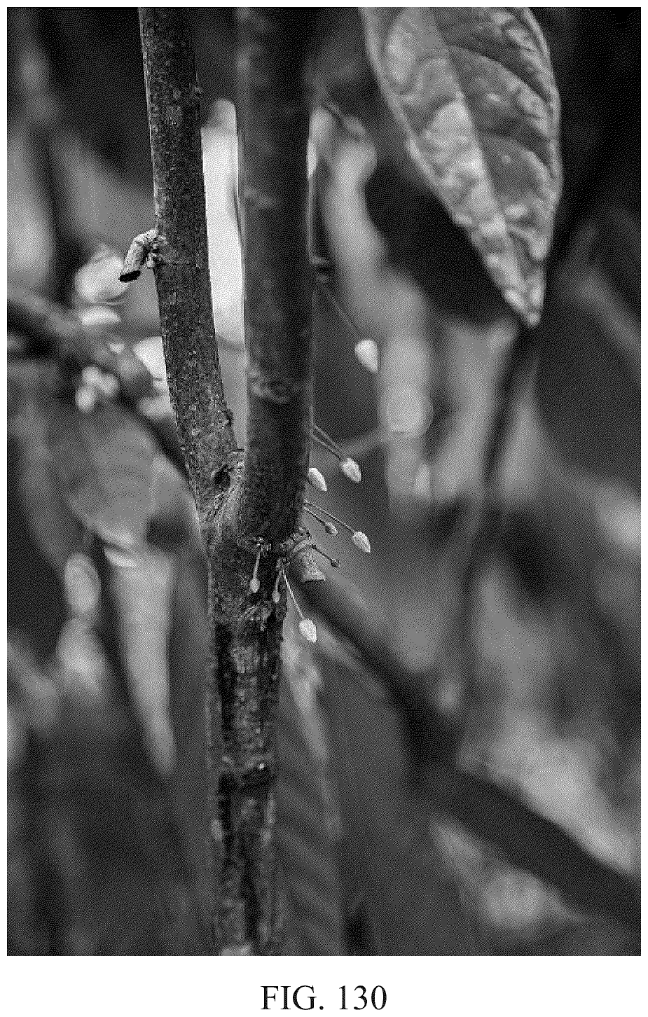

FIG. 131 illustrates leaves of variety ‘CMC-021’.

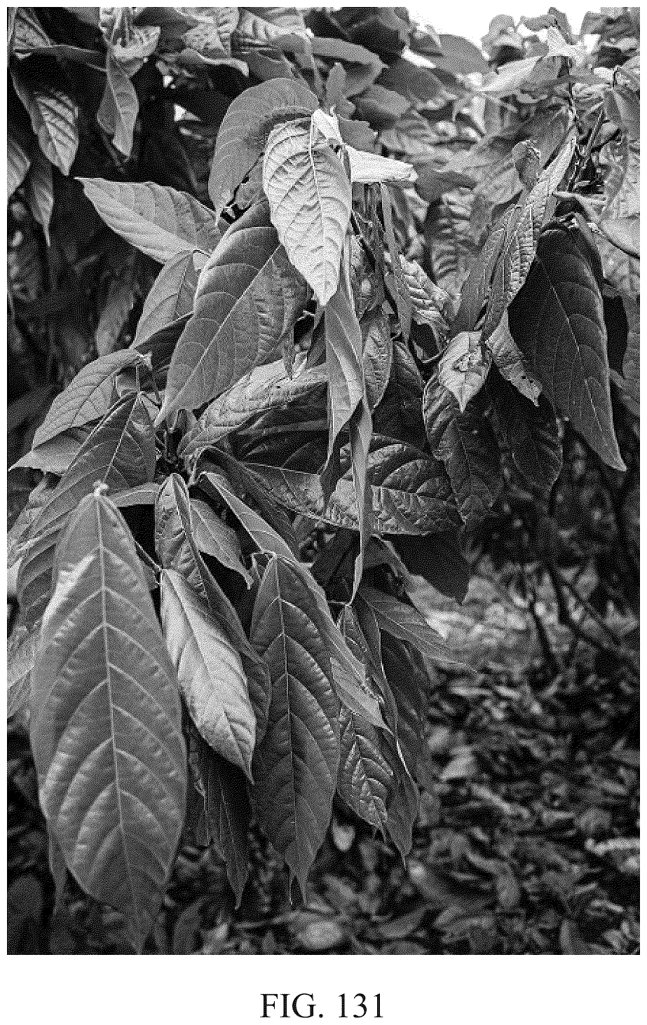

FIG. 132 illustrates a whole tree of variety ‘CMC-023’.

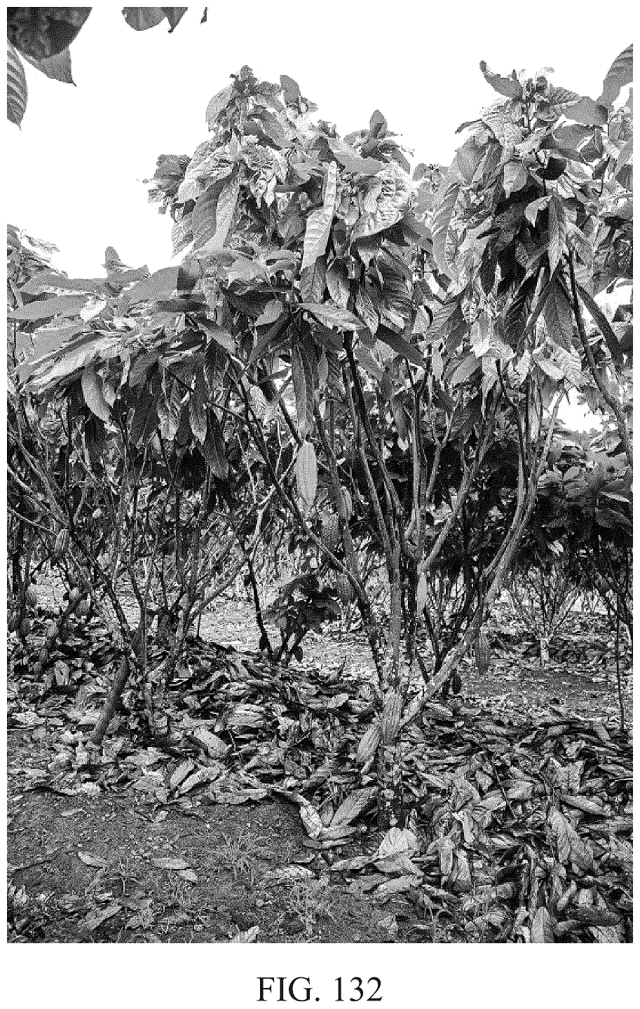

FIG. 133 illustrates a pod of variety ‘CMC-023’.

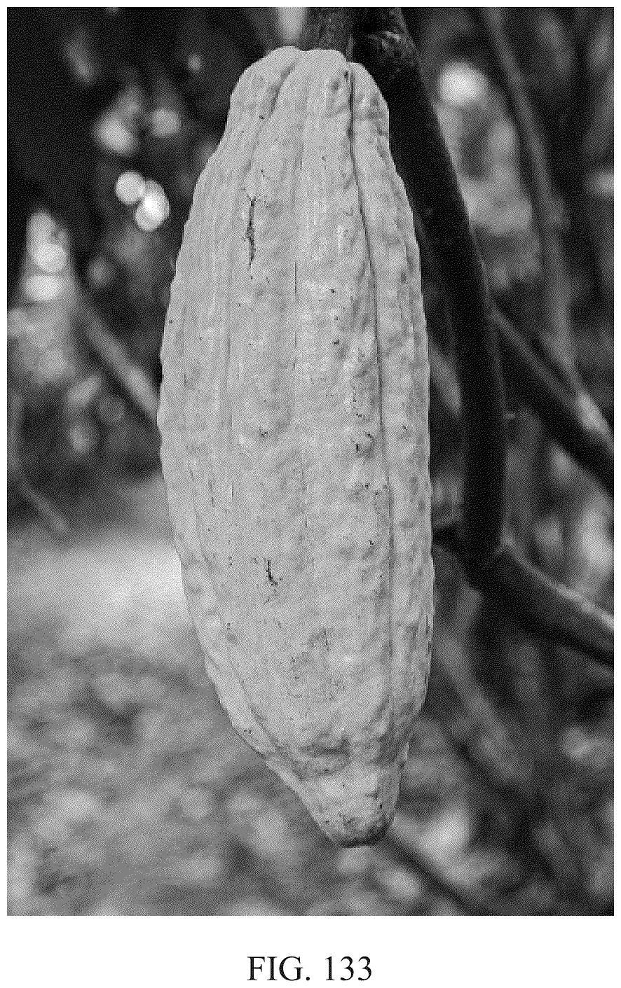

FIG. 134 illustrates a flower of variety ‘CMC-023’.

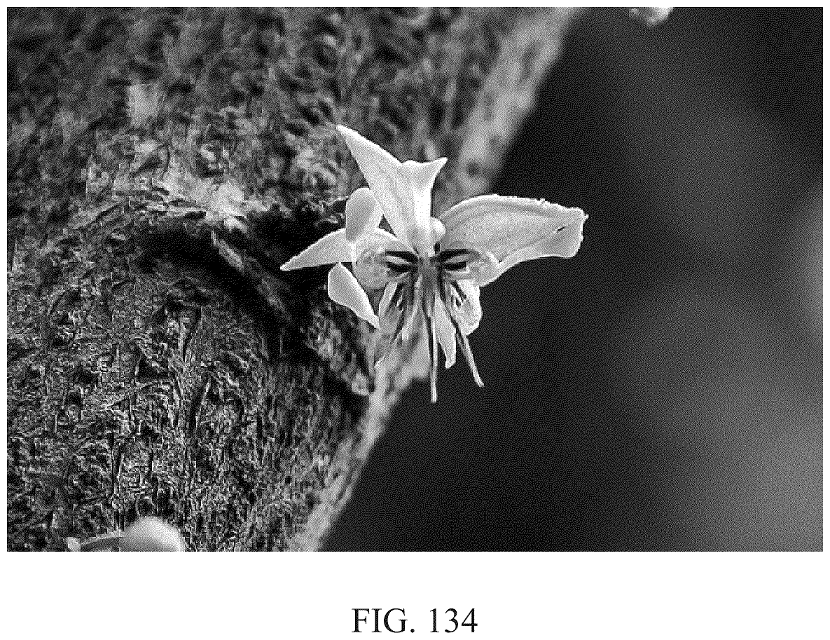

FIG. 135 illustrates flowers and flower buds of variety ‘CMC-023’.

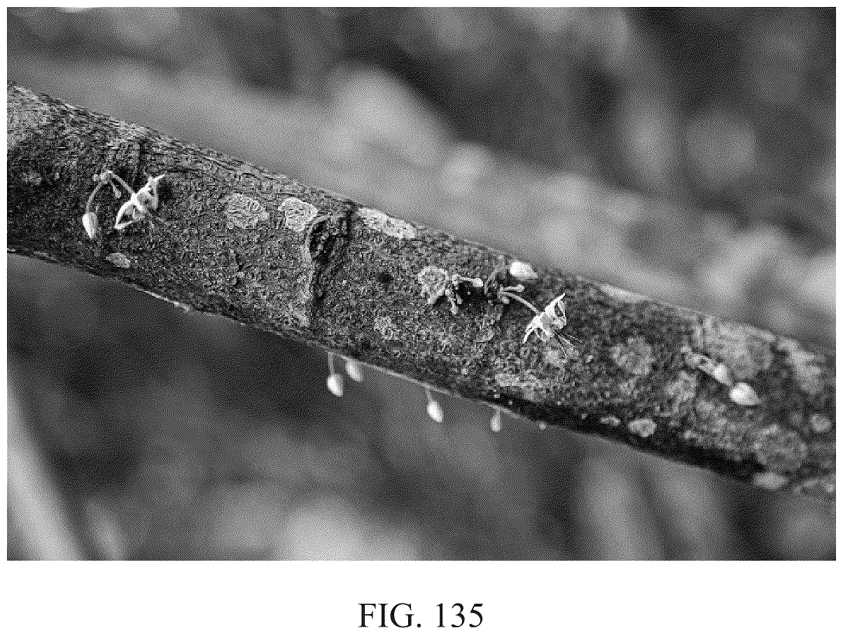

FIG. 136 illustrates flowers and flower buds of variety ‘CMC-023’.

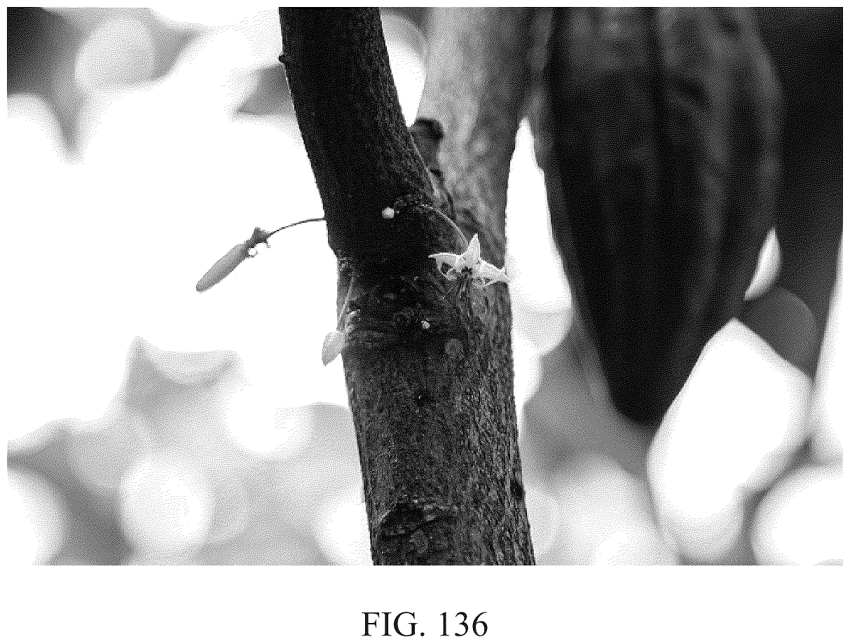

FIG. 137 illustrates flowers and flower buds of variety ‘CMC-023’.

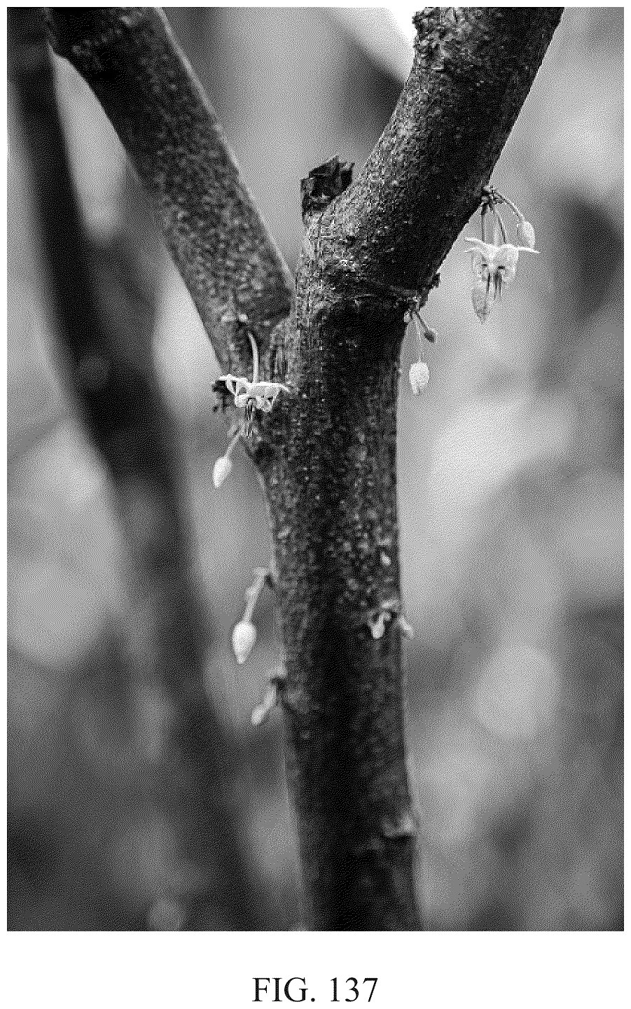

FIG. 138 illustrates leaves of variety ‘CMC-023’.

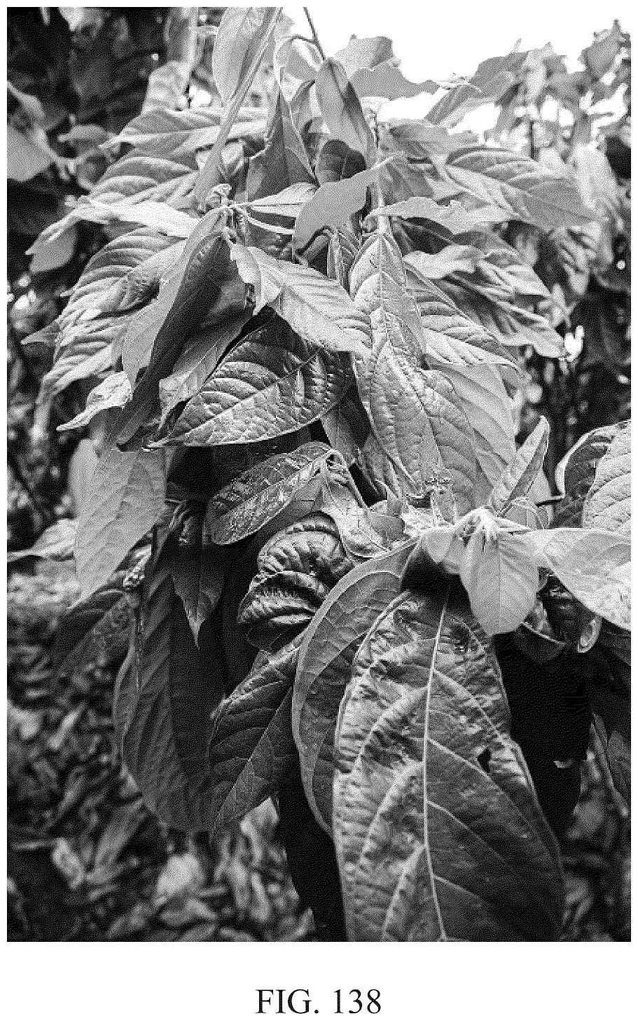

FIG. 139 illustrates a whole tree of variety ‘CMC-024’.

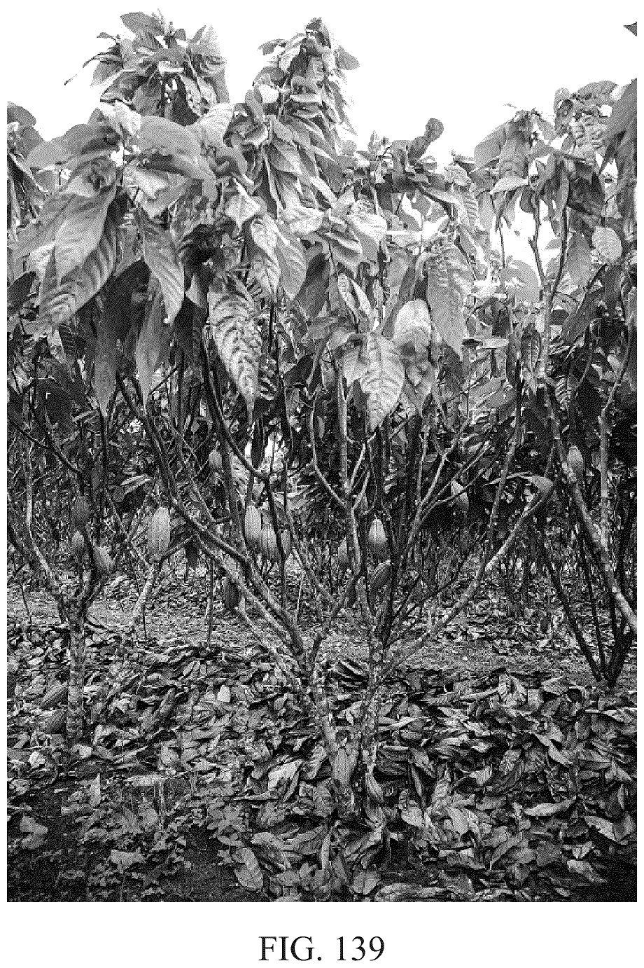

FIG. 140 illustrates a pod of variety ‘CMC-024’.

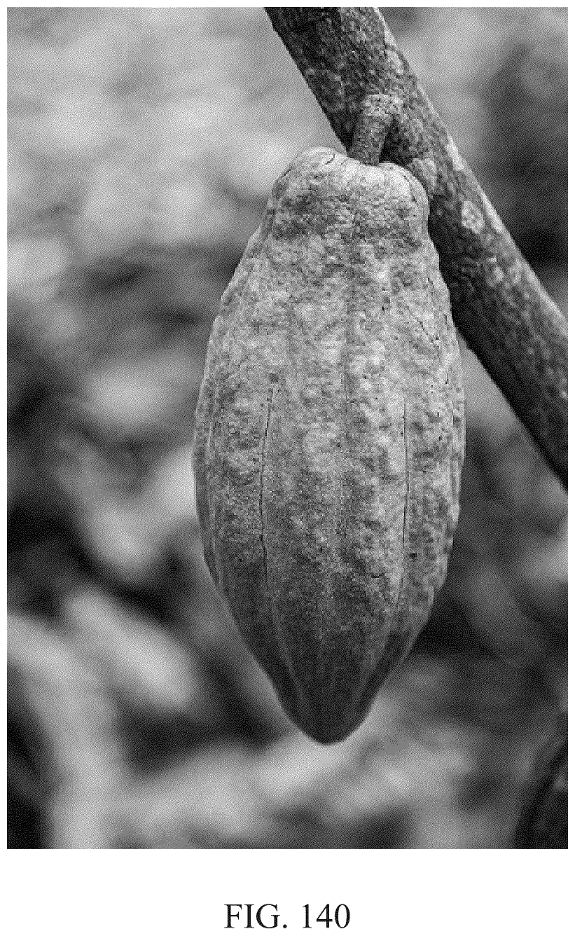

FIG. 141 illustrates a pod of variety ‘CMC-024’.

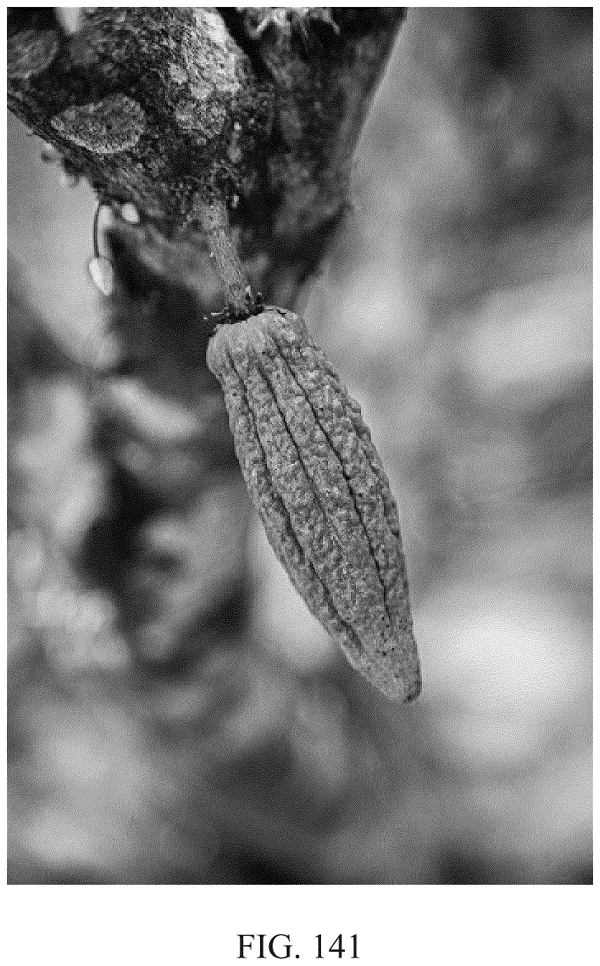

FIG. 142 illustrates a flower of variety ‘CMC-024’.

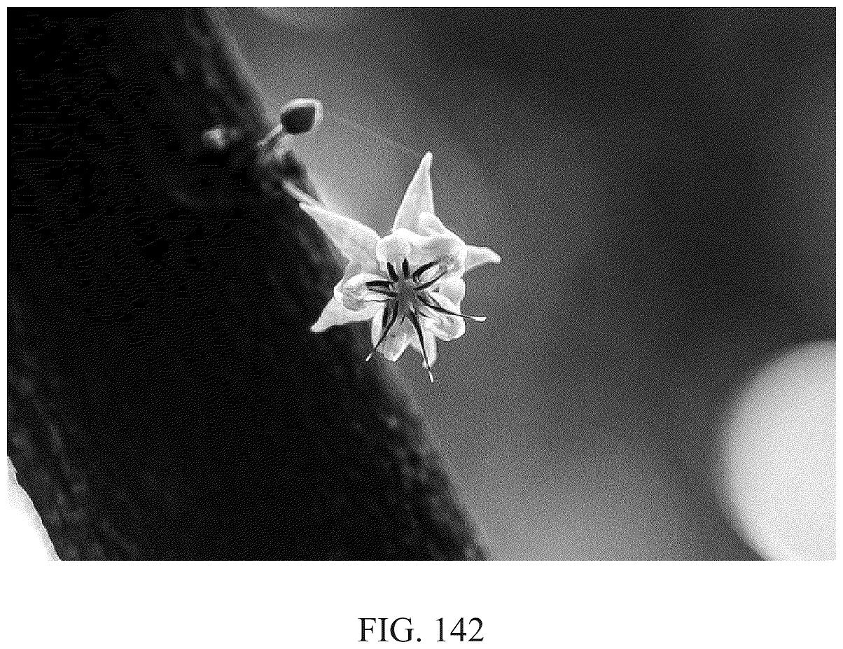

FIG. 143 illustrates flowers and flower buds of variety ‘CMC-024’.

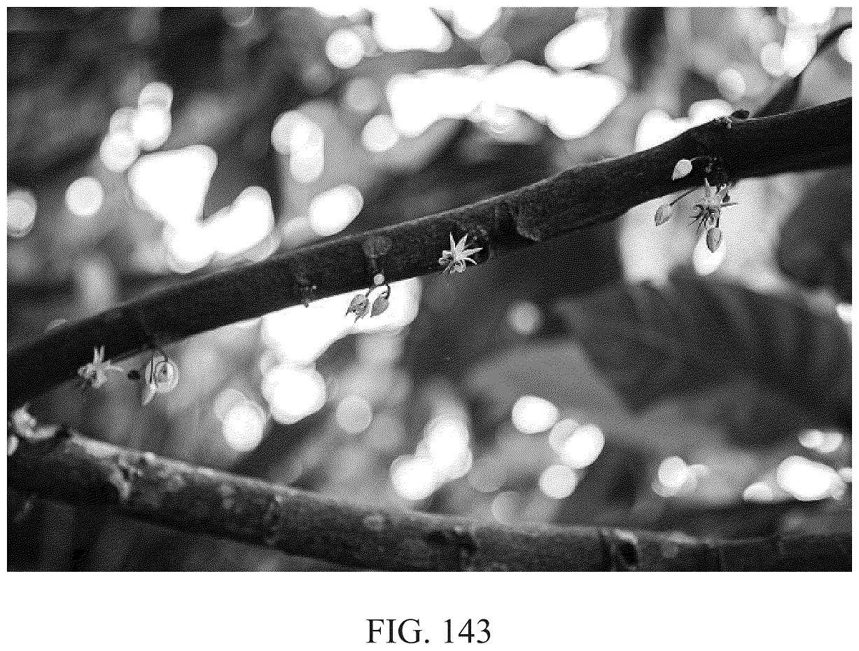

FIG. 144 illustrates flowers and flower buds of variety ‘CMC-024’.

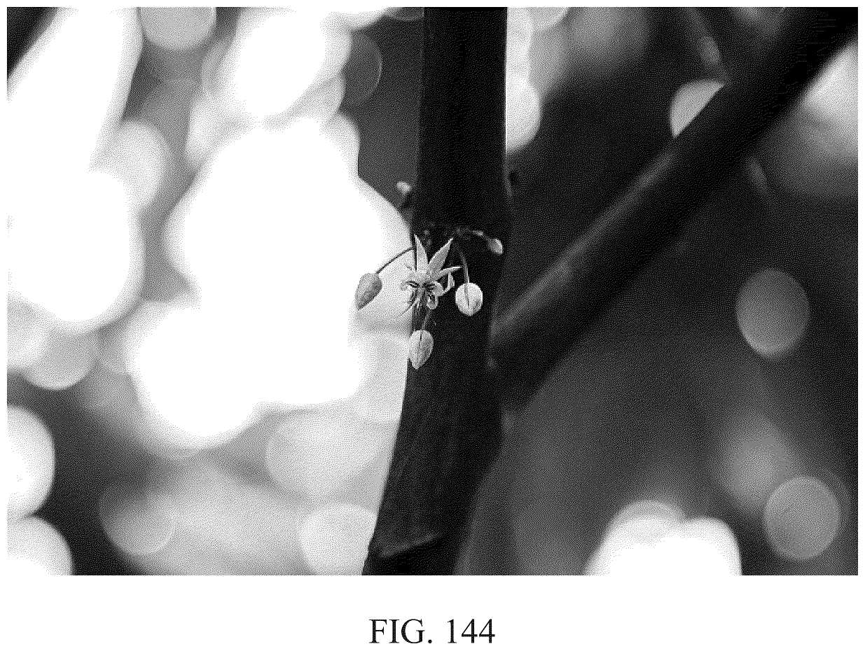

FIG. 145 illustrates leaves of variety ‘CMC-024’.

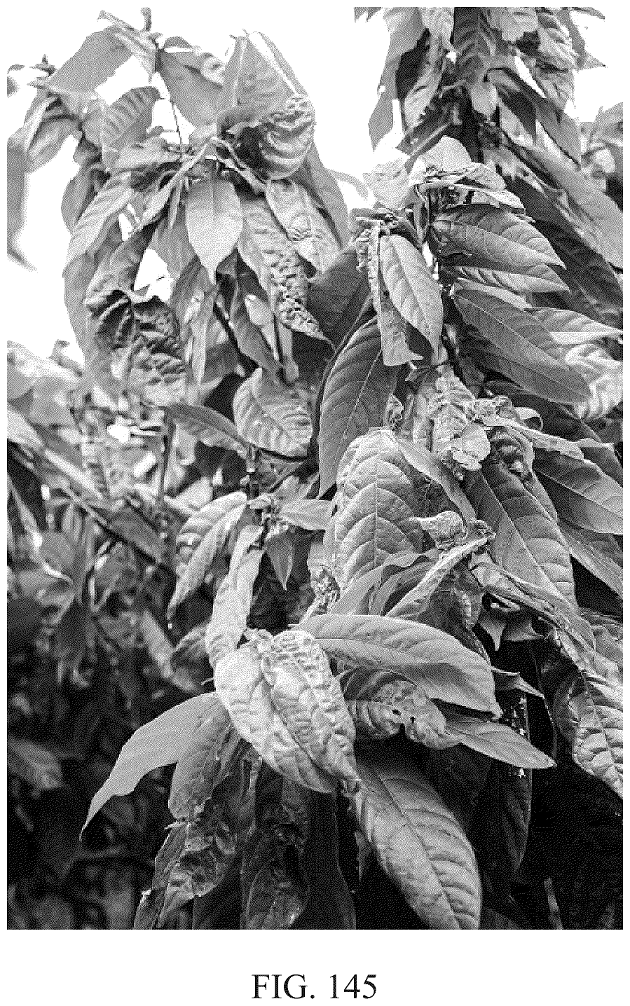

FIG. 146 illustrates leaves of variety ‘CMC-024’.

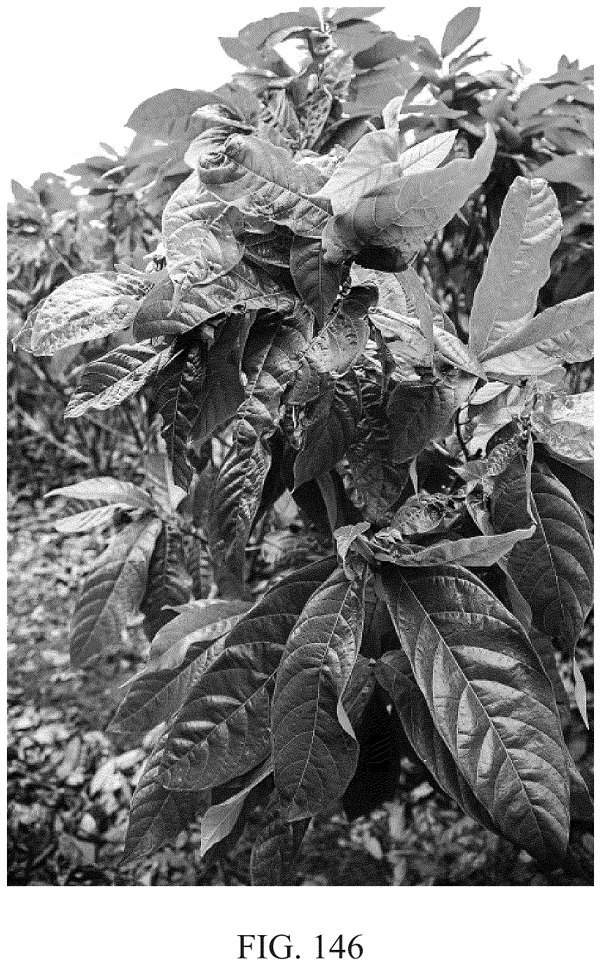

FIG. 147 illustrates a whole tree of variety ‘CMC-025’.

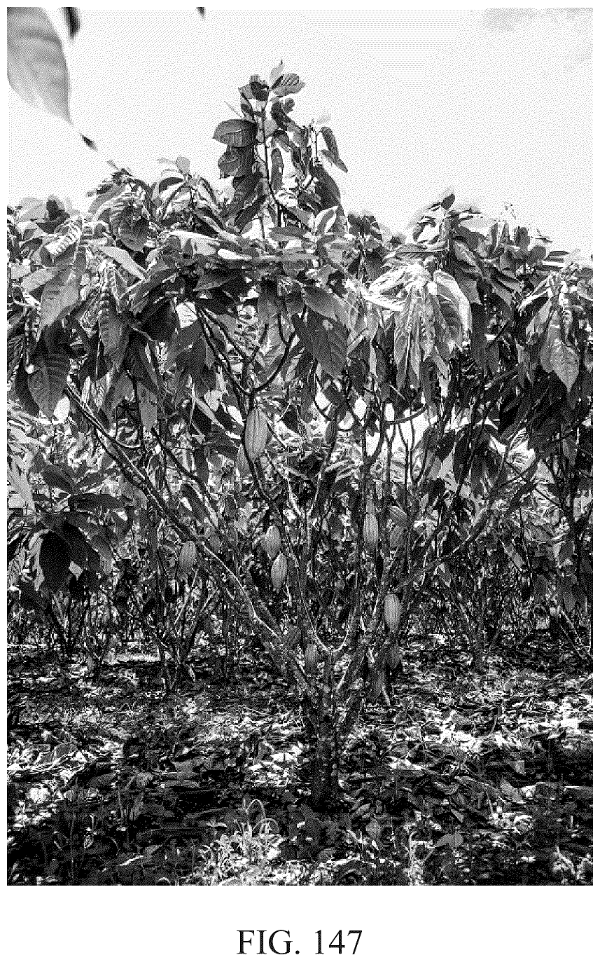

FIG. 148 illustrates a pod of variety ‘CMC-025’.

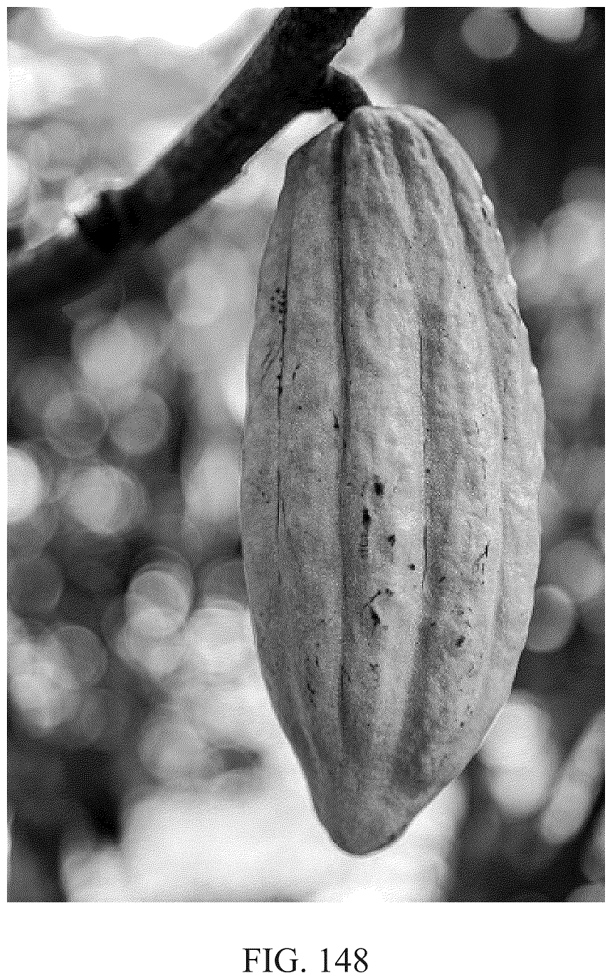

FIG. 149 illustrates a pod of variety ‘CMC-025’.

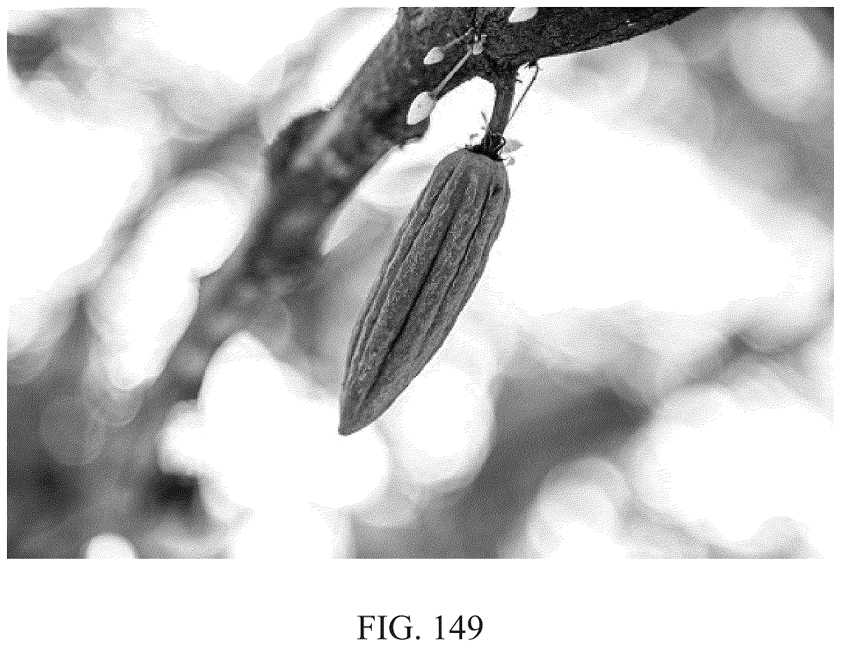

FIG. 150 illustrates a flower of variety ‘CMC-025’.

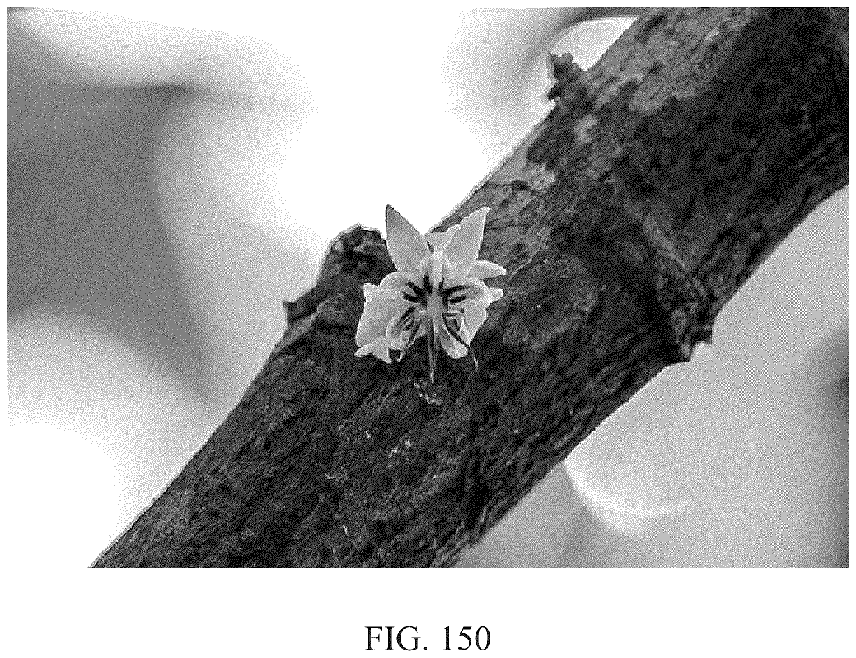

FIG. 151 illustrates flowers and flower buds of variety ‘CMC-025’.

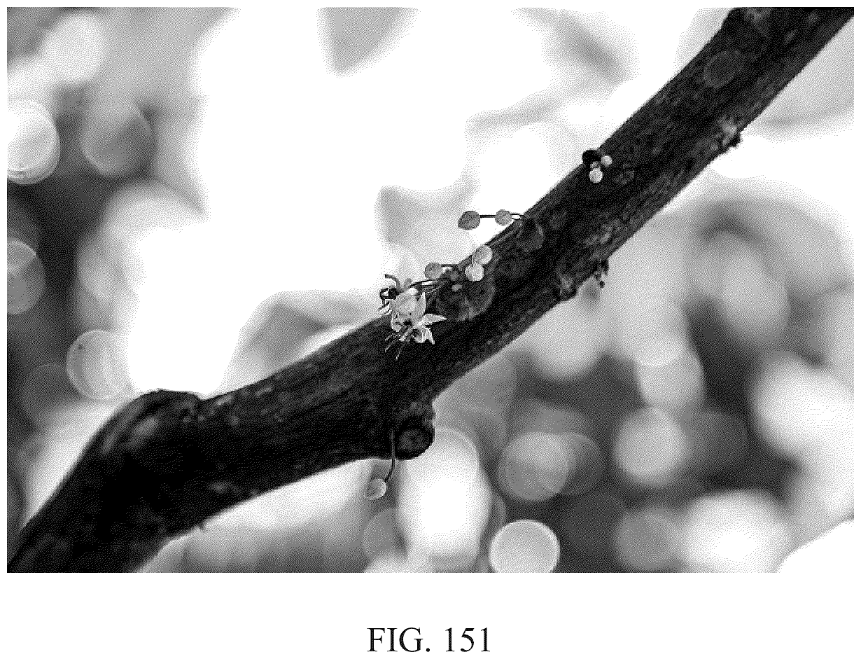

FIG. 152 illustrates leaves of variety ‘CMC-025’.

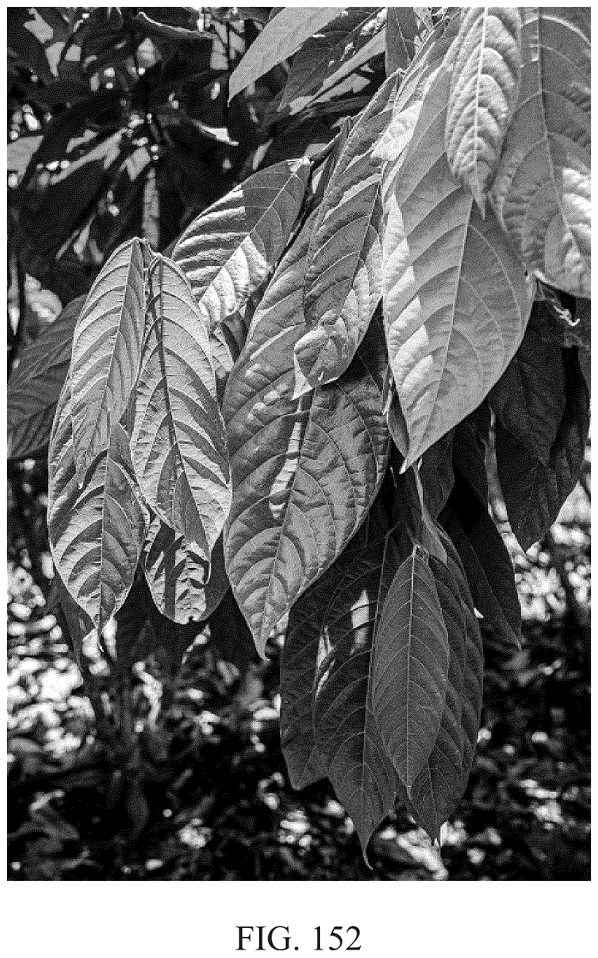

FIG. 153 illustrates a whole tree of variety ‘CMC-026’.

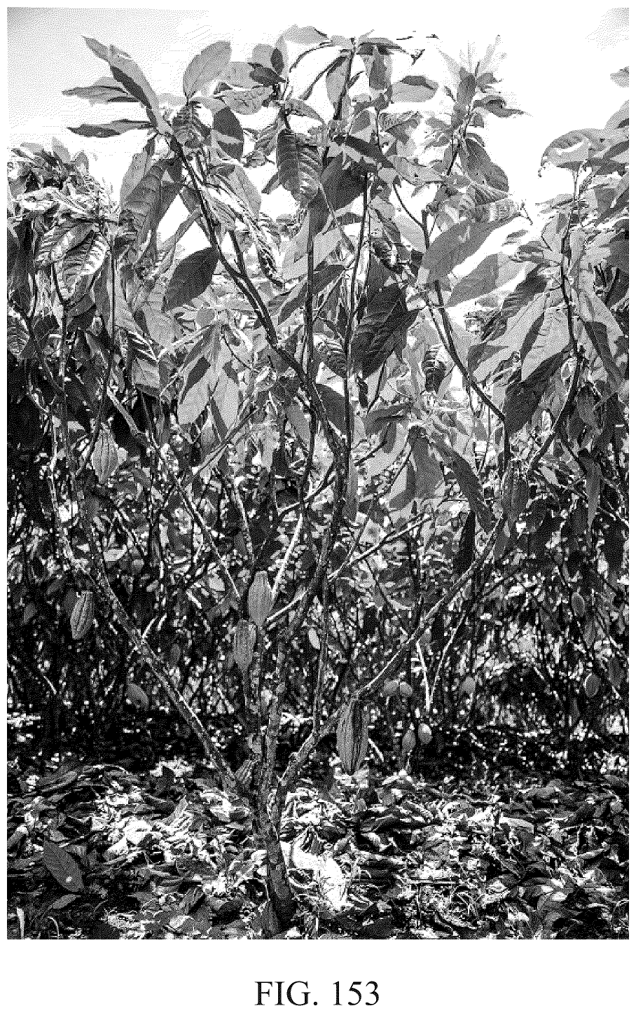

FIG. 154 illustrates a pod of variety ‘CMC-026’.

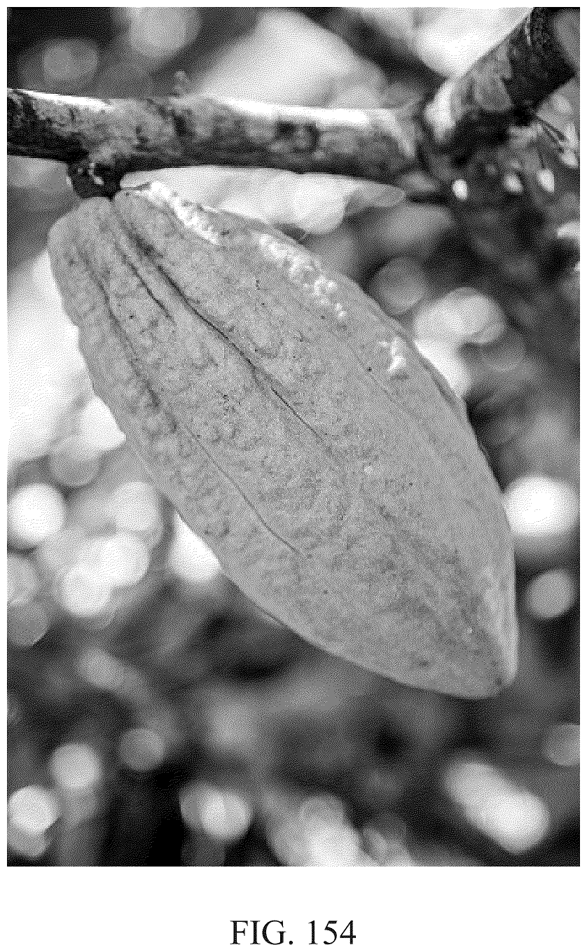

FIG. 155 illustrates a pod of variety ‘CMC-026’.

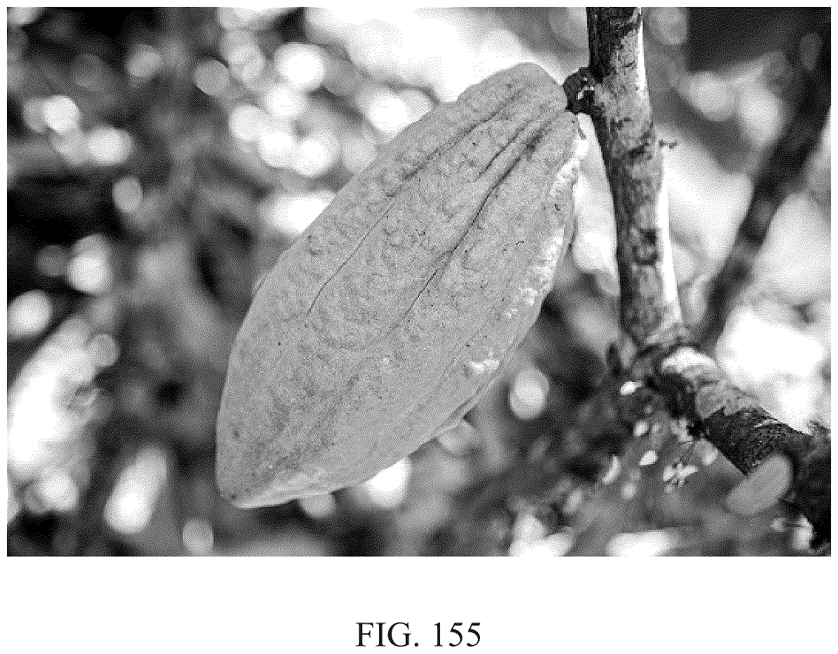

FIG. 156 illustrates a pod of variety ‘CMC-026’.

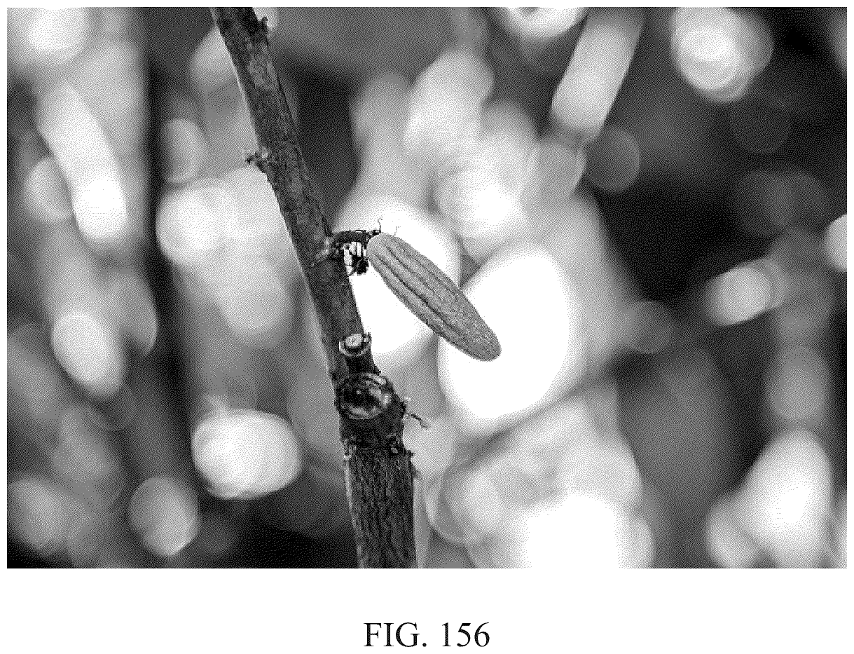

FIG. 157 illustrates a flower of variety ‘CMC-026’.

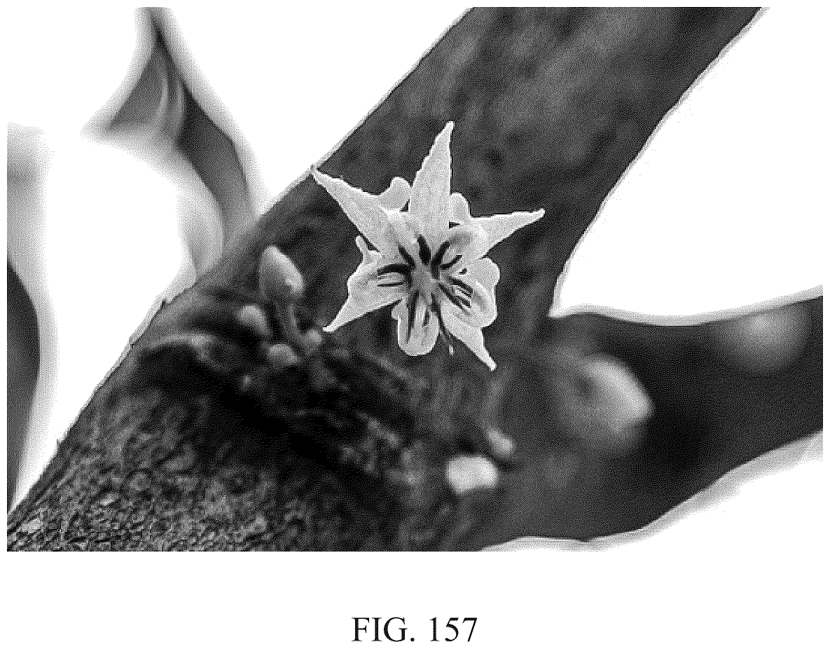

FIG. 158 illustrates flowers and flower buds of variety ‘CMC-026’.

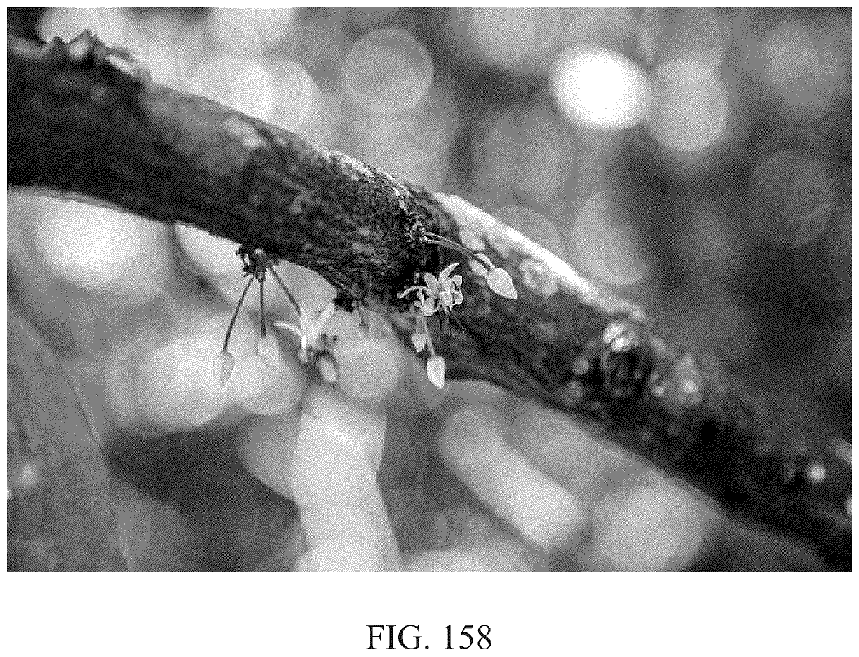

FIG. 159 illustrates flowers and flower buds of variety ‘CMC-026’.

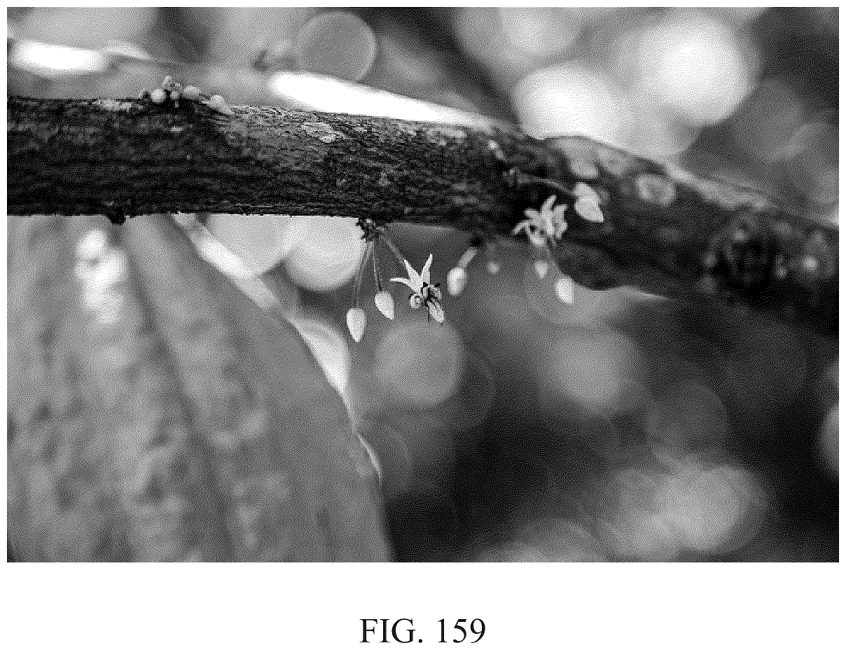

FIG. 160 illustrates leaves of variety ‘CMC-026’.

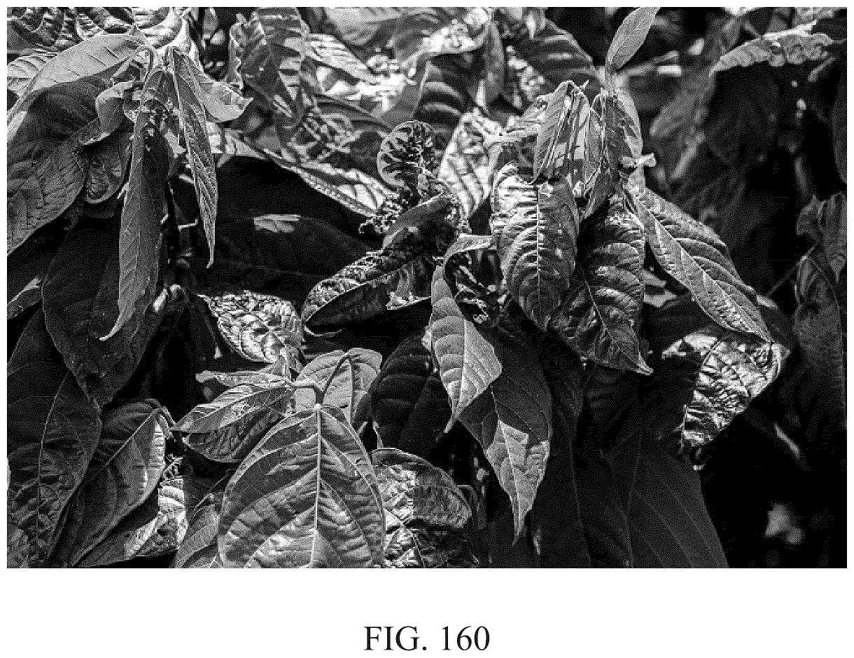

FIG. 161 illustrates a whole tree of variety ‘CMC-027’.

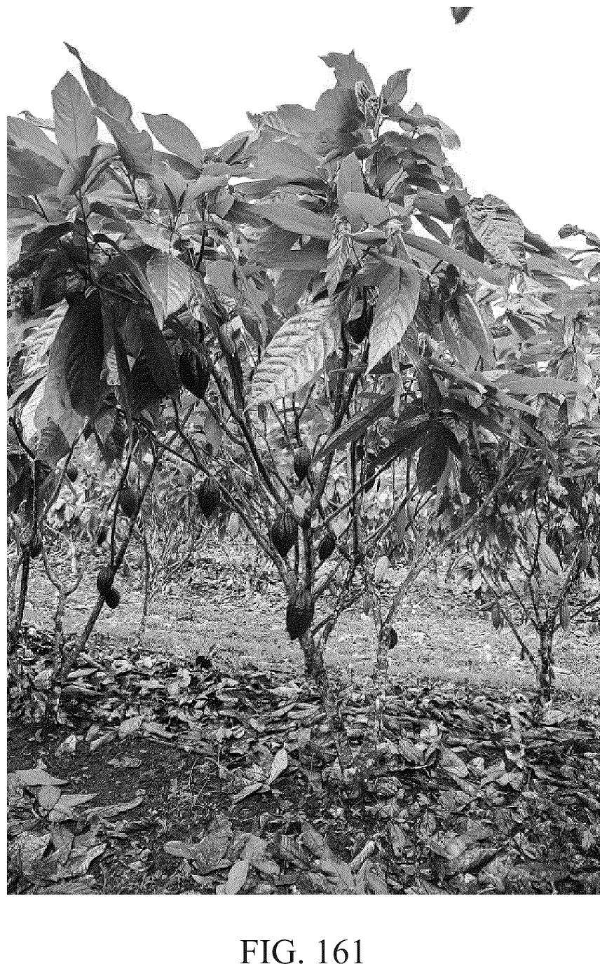

FIG. 162 illustrates a pod of variety ‘CMC-027’.

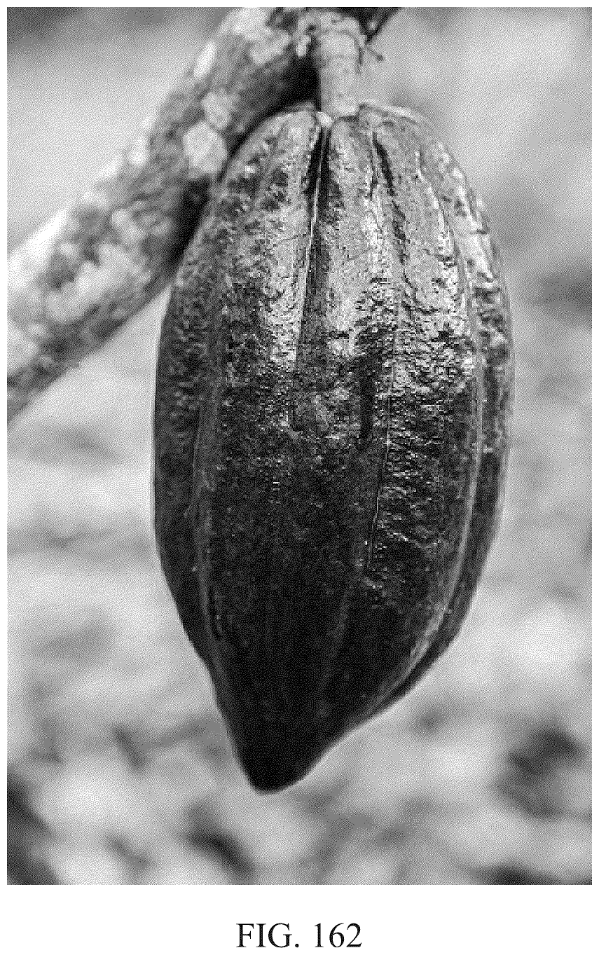

FIG. 163 illustrates a flower of variety ‘CMC-027’.

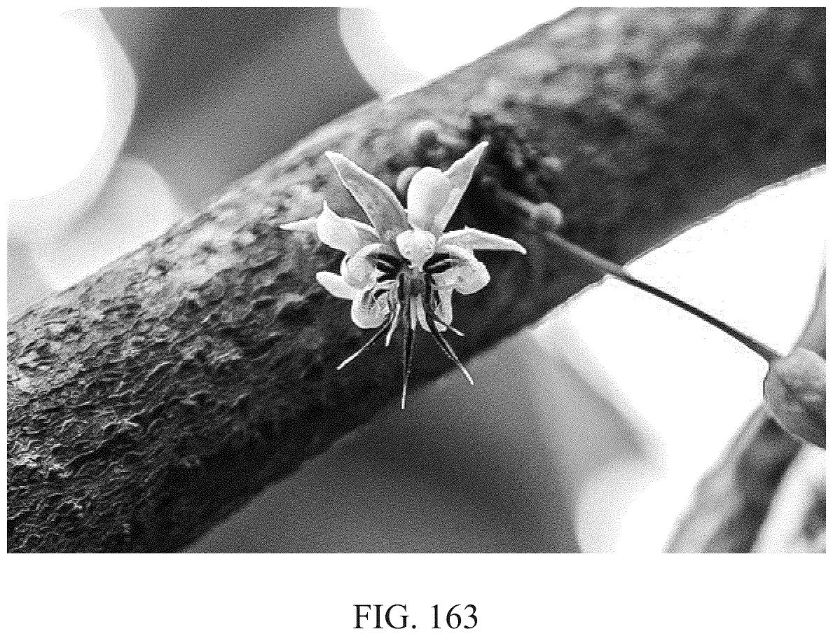

FIG. 164 illustrates a flower and flower buds of variety ‘CMC-027’.

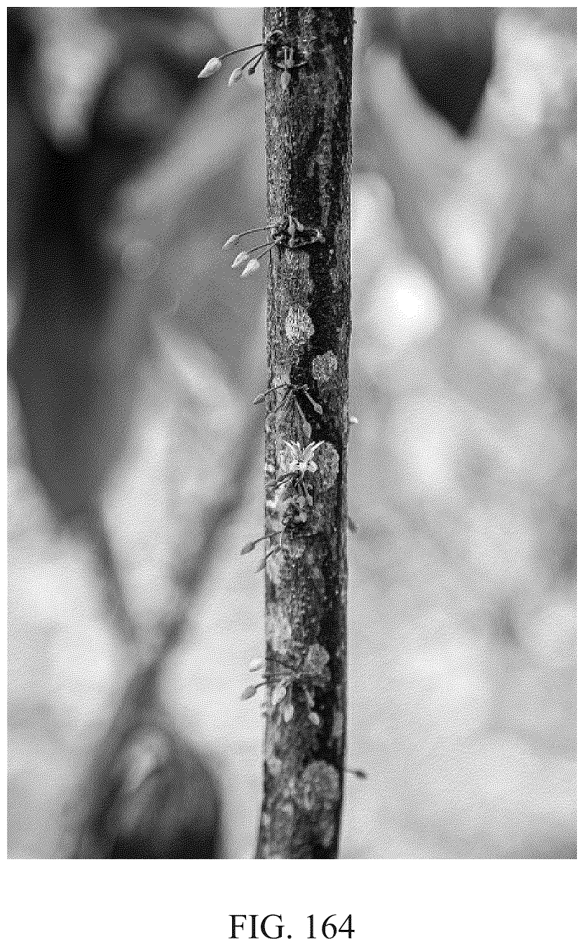

FIG. 165 illustrates leaves of variety ‘CMC-027’.

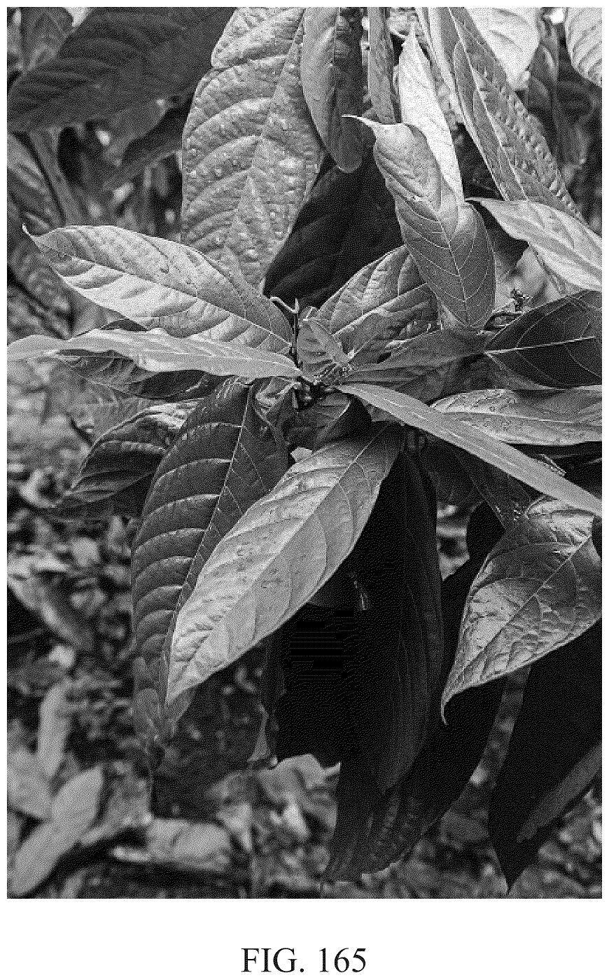

FIG. 166 illustrates a whole tree of variety ‘CMC-028’.

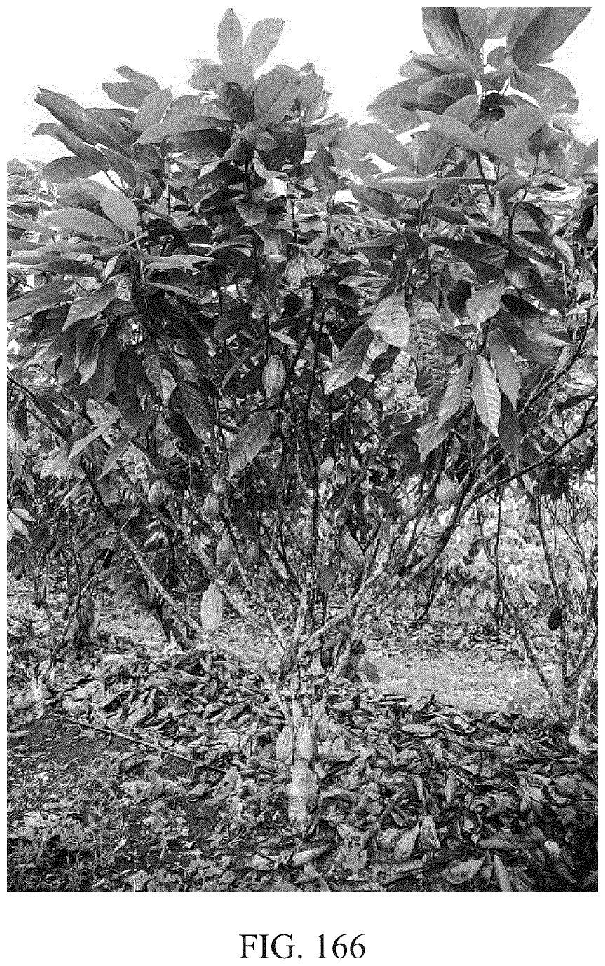

FIG. 167 illustrates a pod of variety ‘CMC-028’.

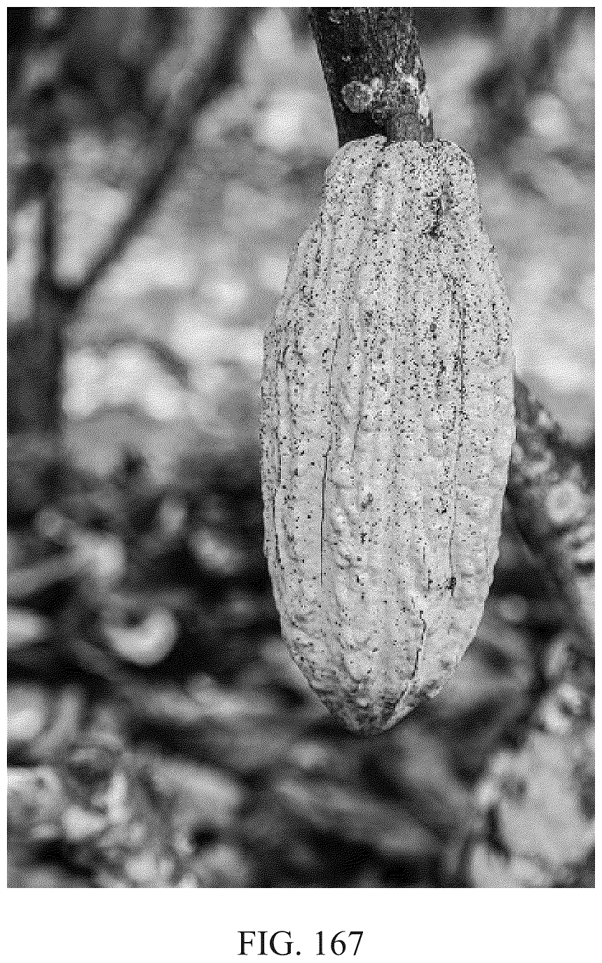

FIG. 168 illustrates a flower of variety ‘CMC-028’.

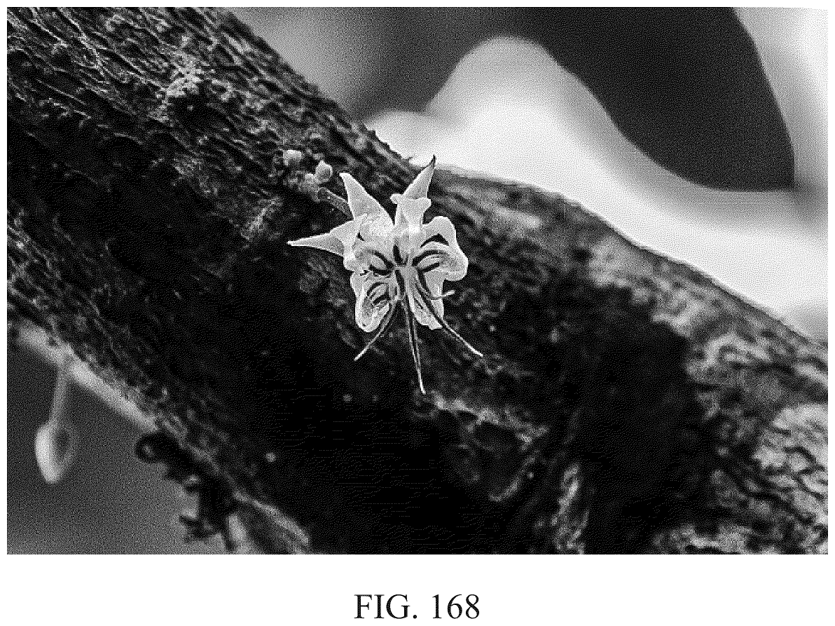

FIG. 169 illustrates flowers of variety ‘CMC-028’.

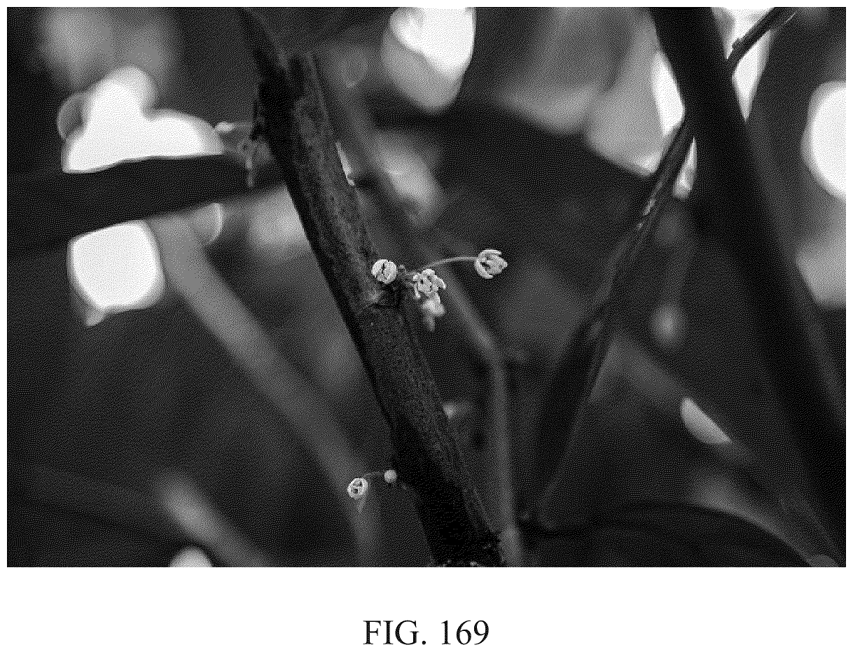

FIG. 170 illustrates flower buds of variety ‘CMC-028’.

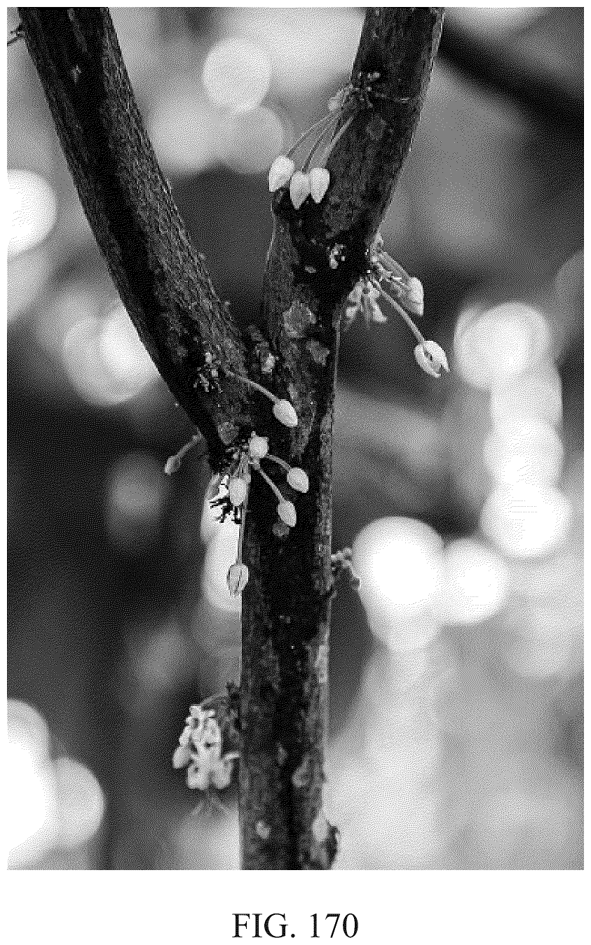

FIG. 171 illustrates leaves of variety ‘CMC-028’.

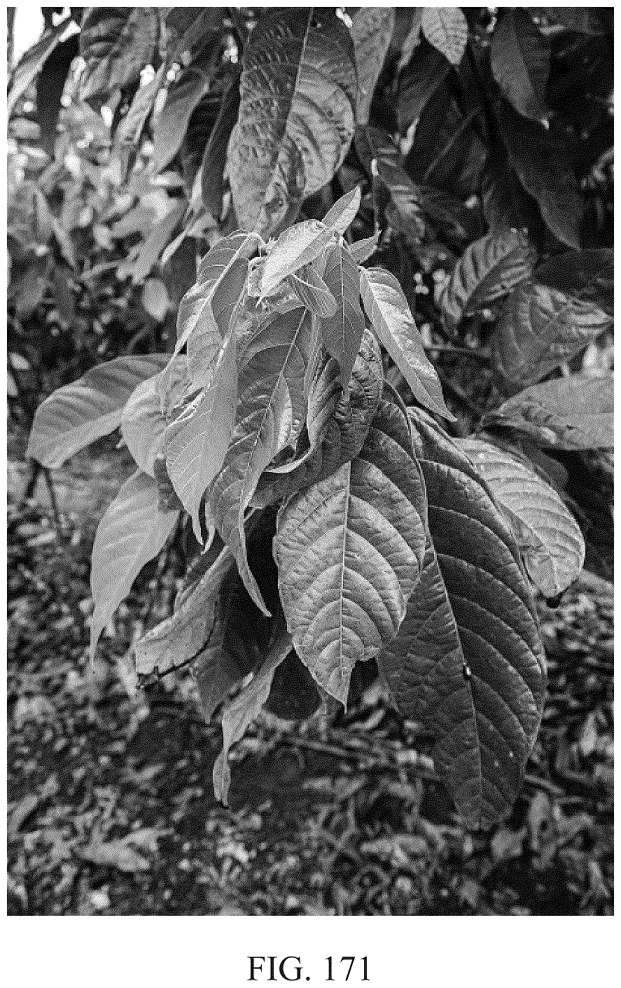

FIG. 172 illustrates a whole tree of variety ‘CMC-029’.

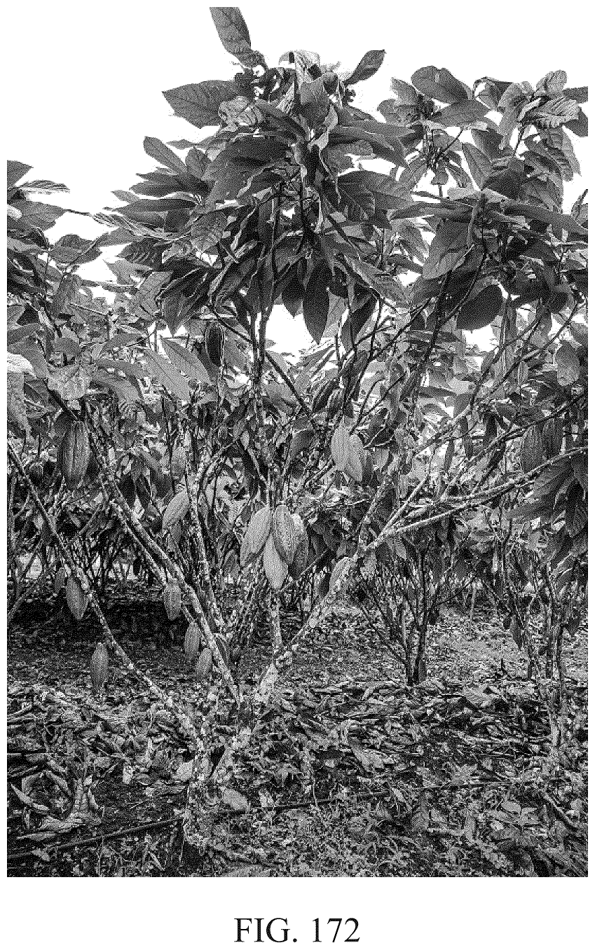

FIG. 173 illustrates a pod of variety ‘CMC-029’.

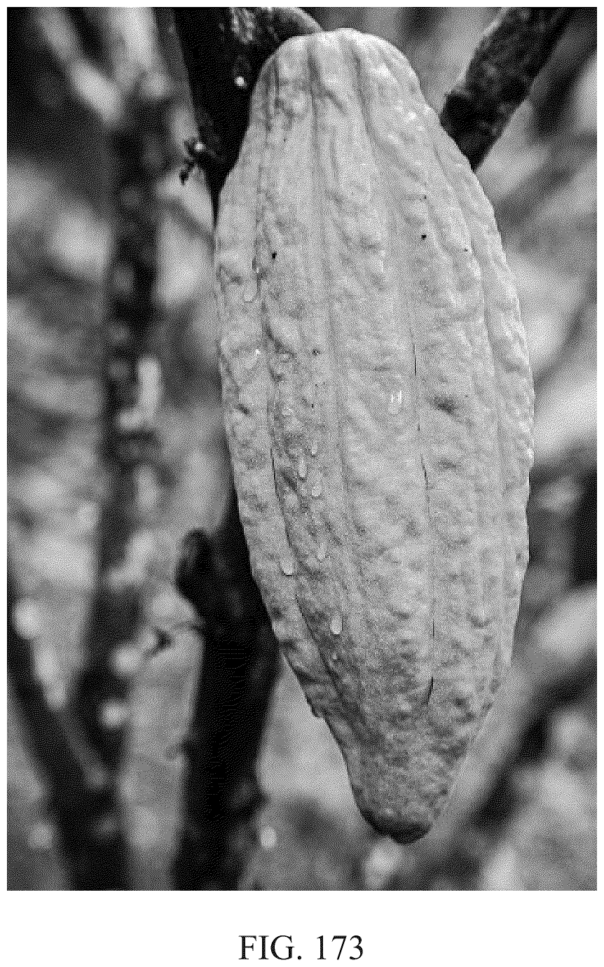

FIG. 174 illustrates a flower and flower buds of variety ‘CMC-029’.

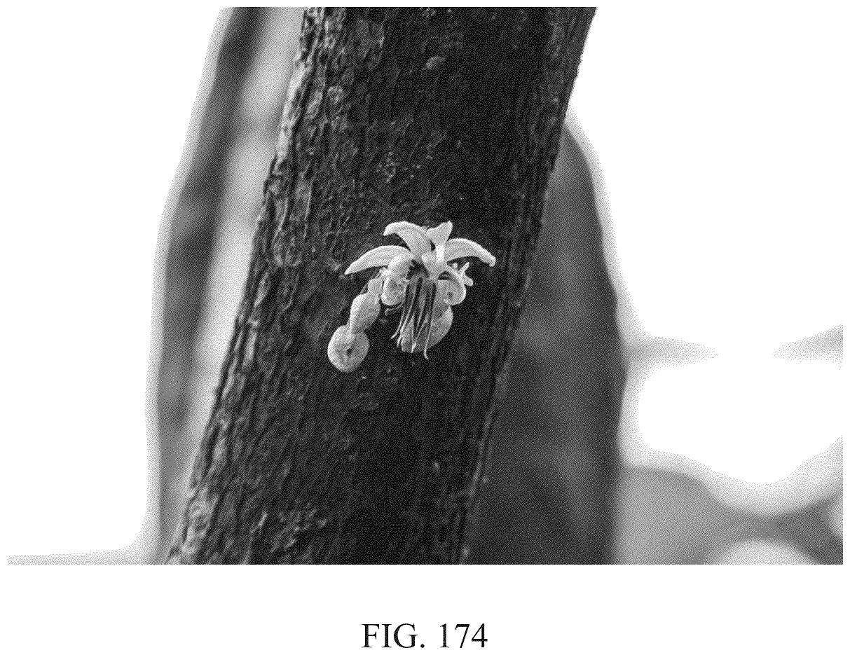

FIG. 175 illustrates a flower and flower buds of variety ‘CMC-029’.

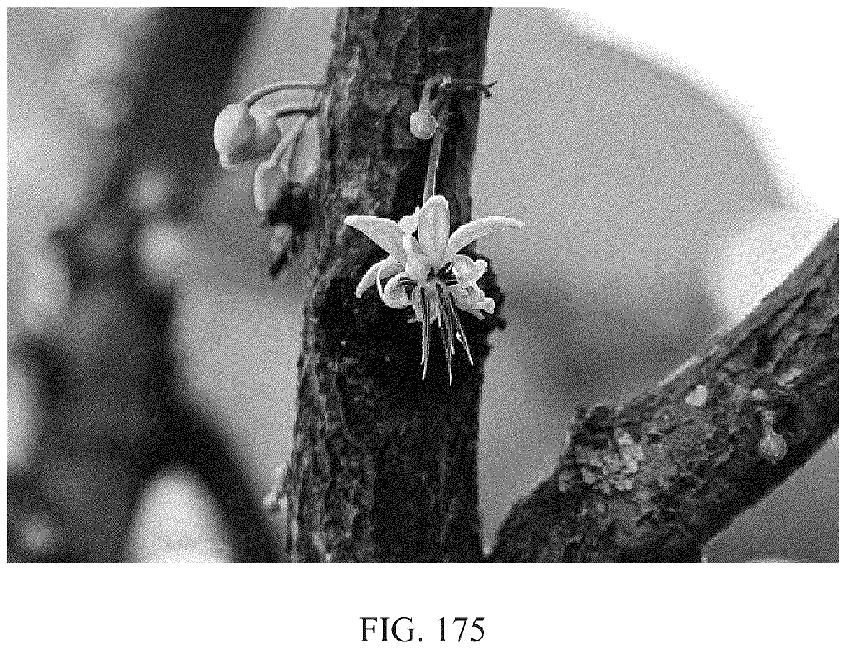

FIG. 176 illustrates a flower and flower buds of variety ‘CMC-029’.

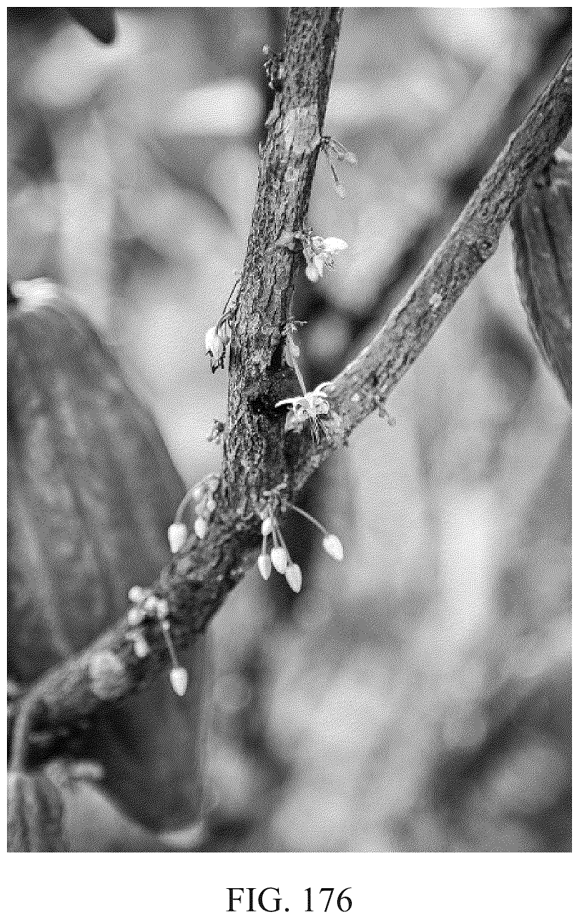

FIG. 177 illustrates leaves of variety ‘CMC-029’.

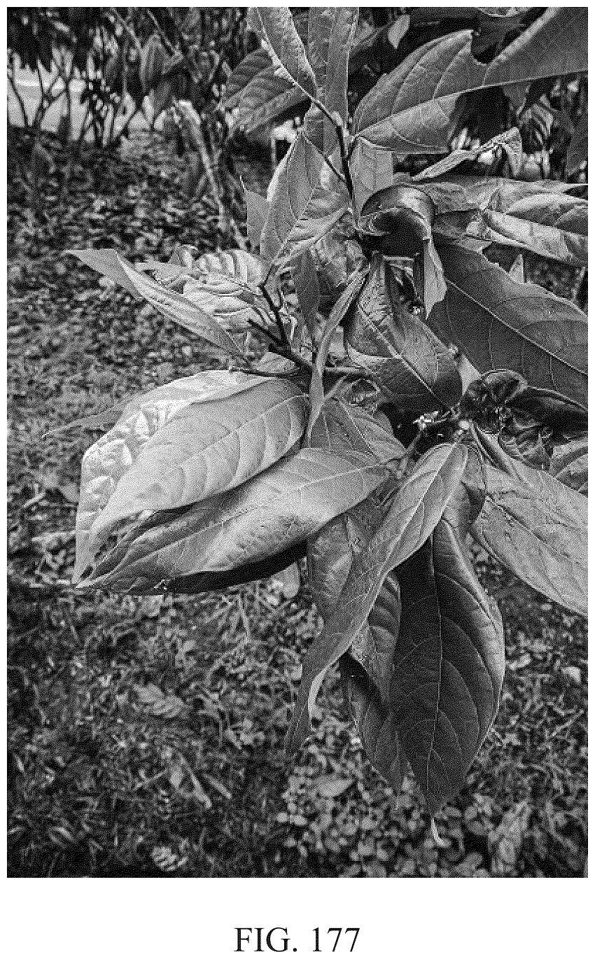

FIG. 178 illustrates a whole tree of variety ‘CMC-030’.

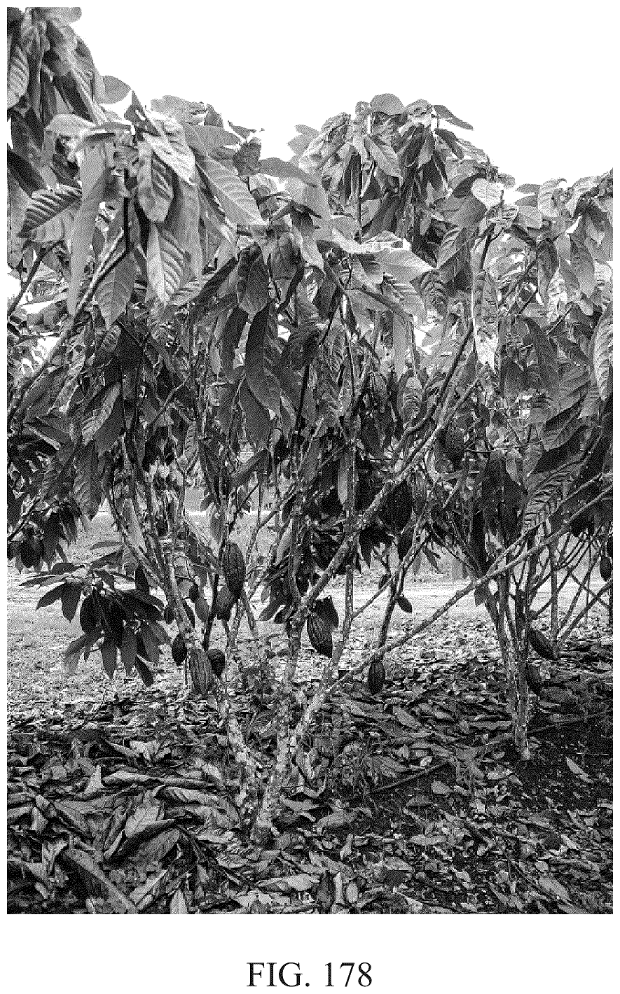

FIG. 179 illustrates a pod of variety ‘CMC-030’.

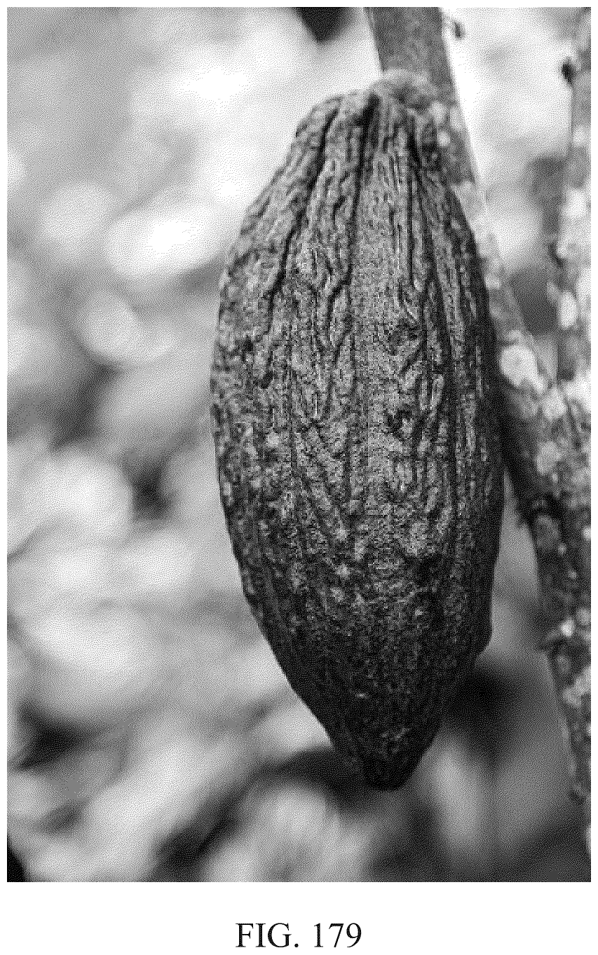

FIG. 180 illustrates a pod of variety ‘CMC-030’.

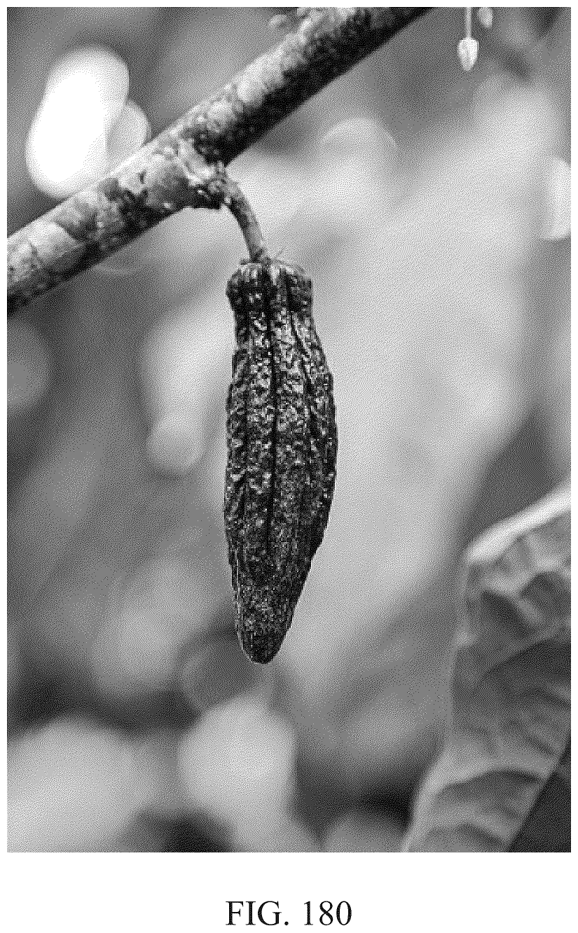

FIG. 181 illustrates a flower of variety ‘CMC-030’.

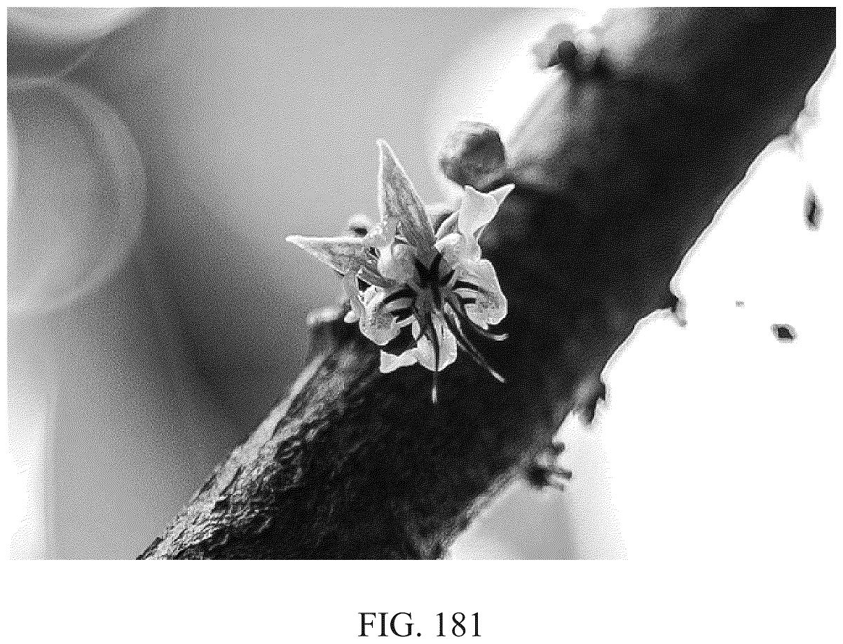

FIG. 182 illustrates flowers and flower buds of variety ‘CMC-030’.

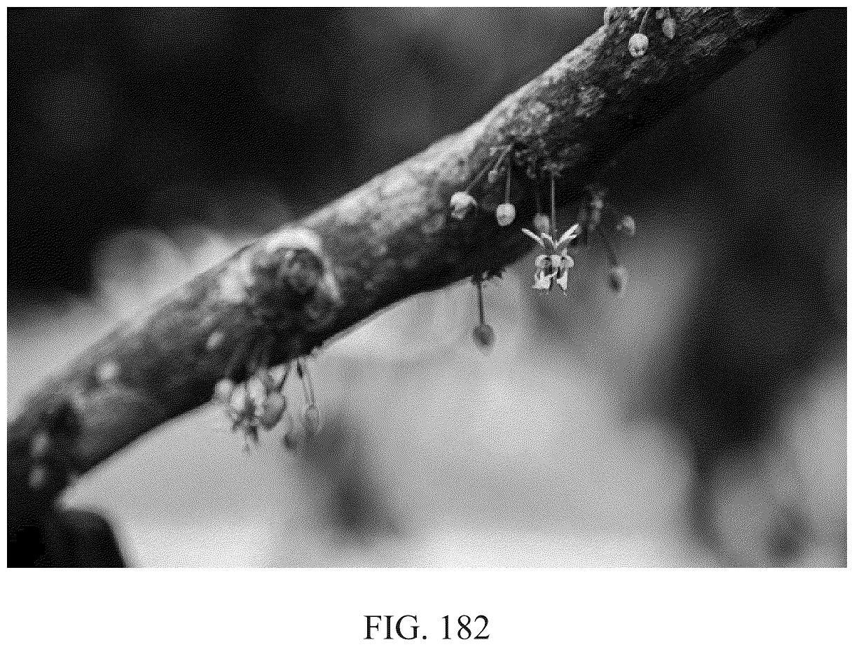

FIG. 183 illustrates flowers and flower buds of variety ‘CMC-030’.

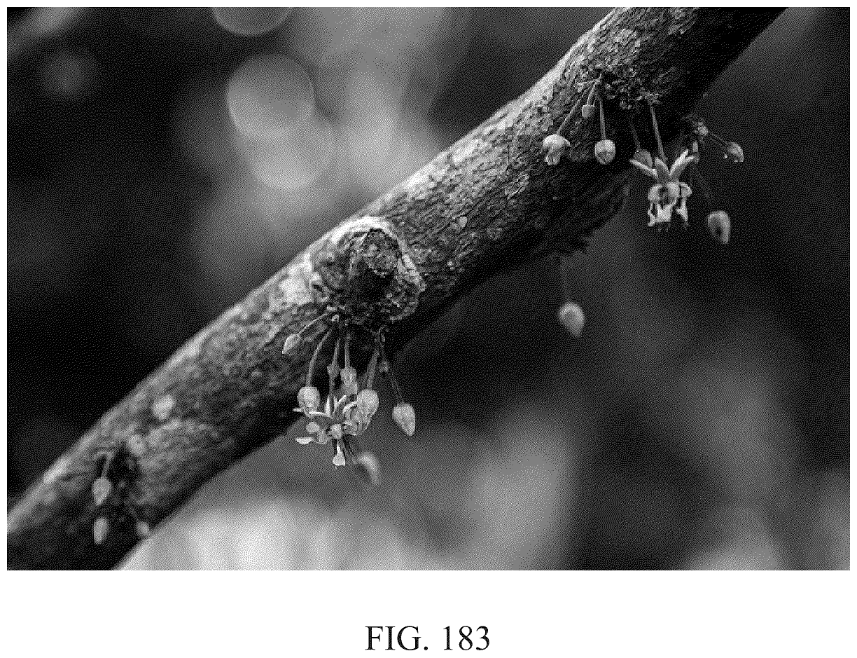

FIG. 184 illustrates leaves of variety ‘CMC-030’.

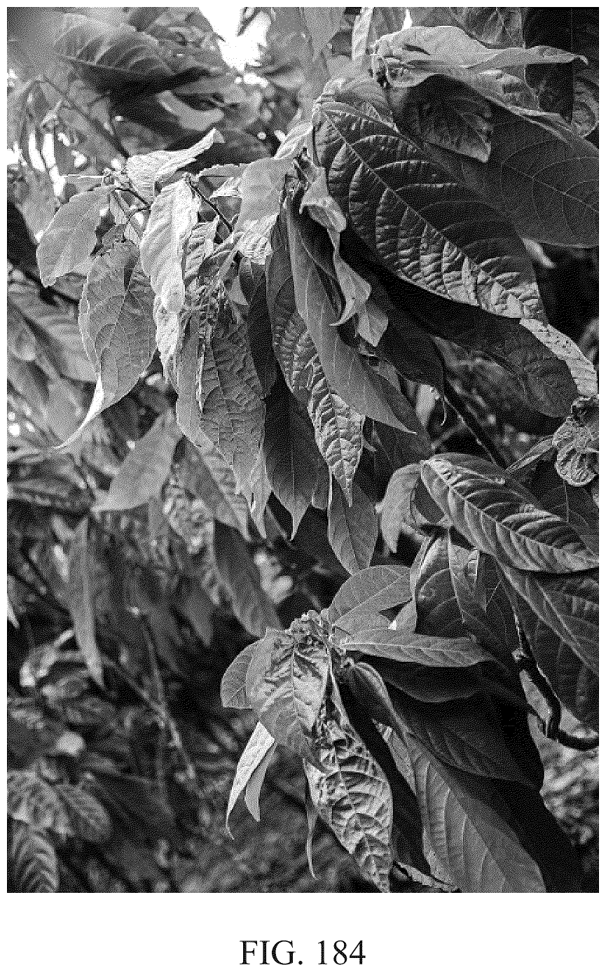

FIG. 185 illustrates a whole tree of variety ‘CMC-031’.

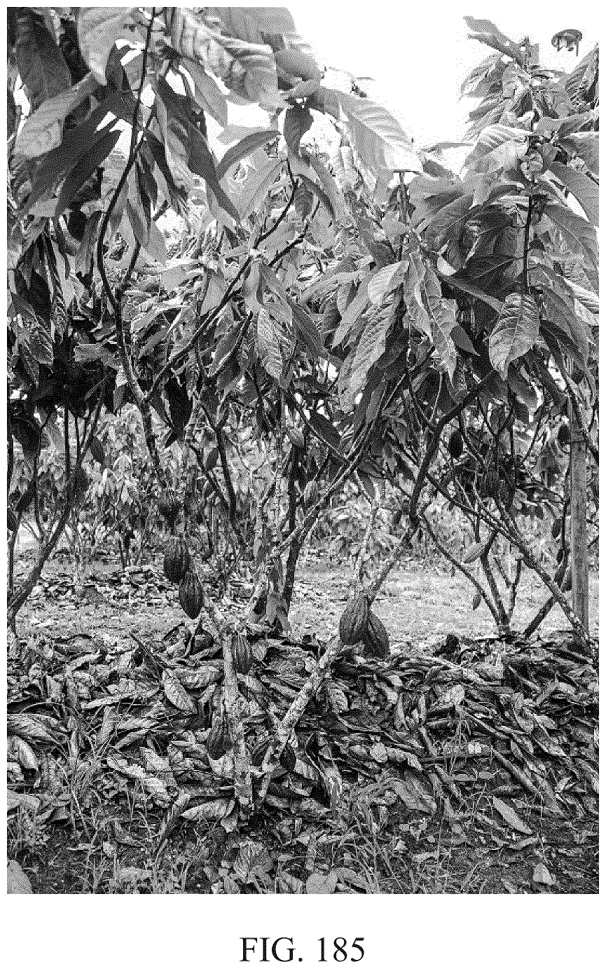

FIG. 186 illustrates a pod of variety ‘CMC-031’.

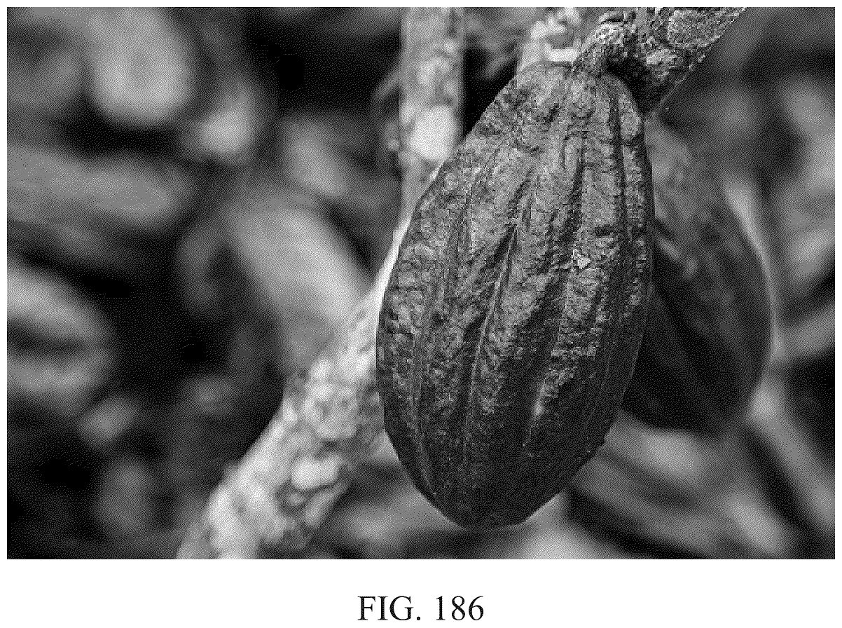

FIG. 187 illustrates a flower of variety ‘CMC-031’.

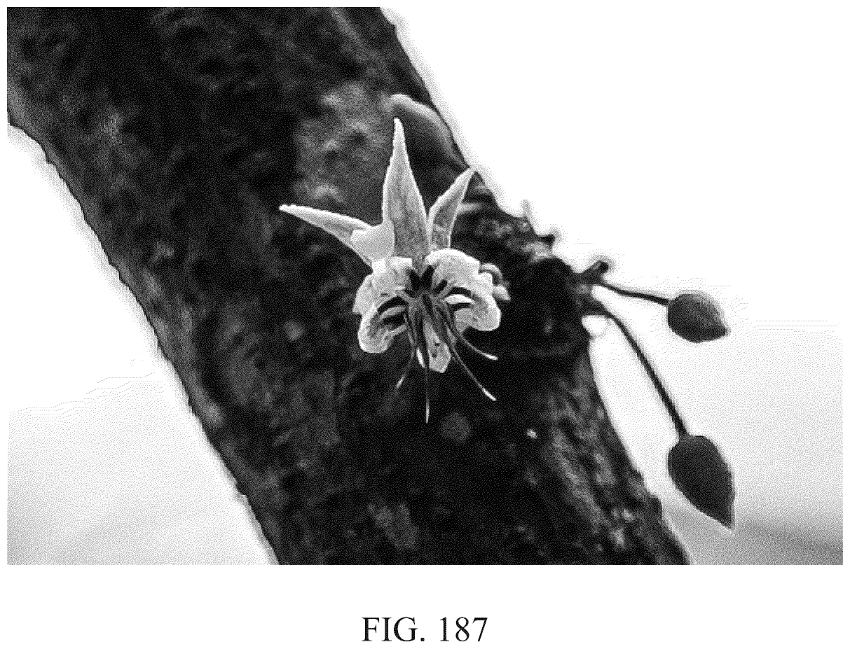

FIG. 188 illustrates a flower and flower buds of variety ‘CMC-031’.

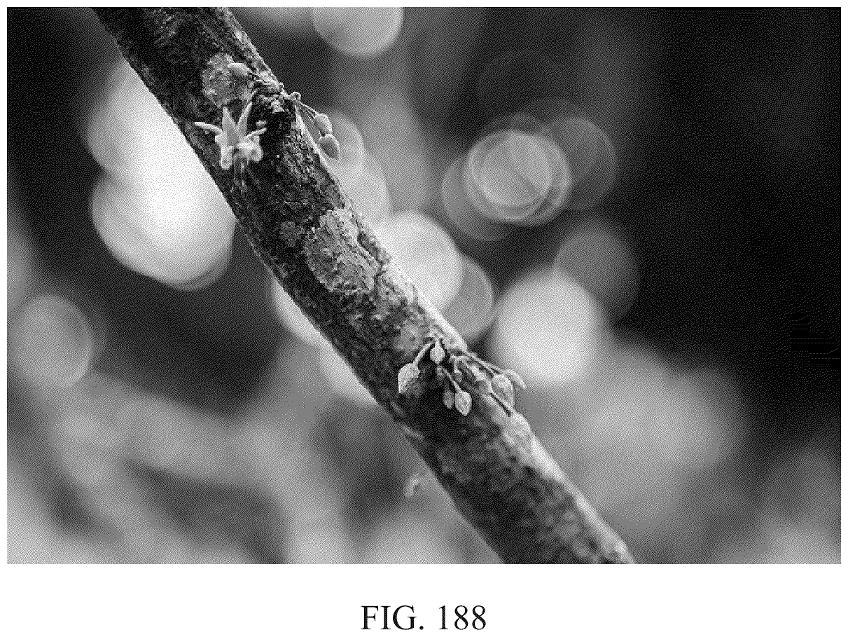

FIG. 189 illustrates flower buds of variety ‘CMC-031’.

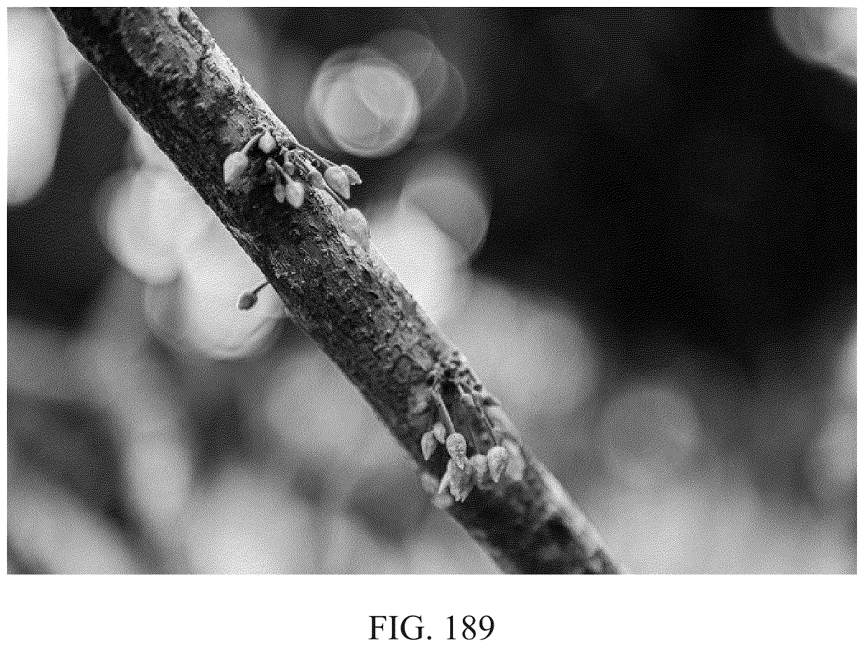

FIG. 190 illustrates leaves of variety ‘CMC-031’.

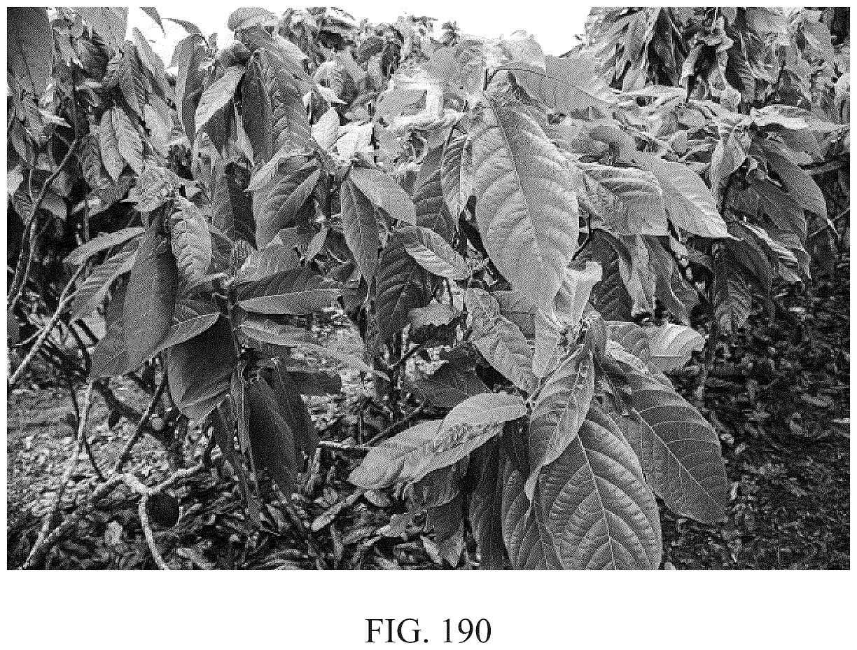

FIG. 191 illustrates leaves of variety ‘CMC-031’.

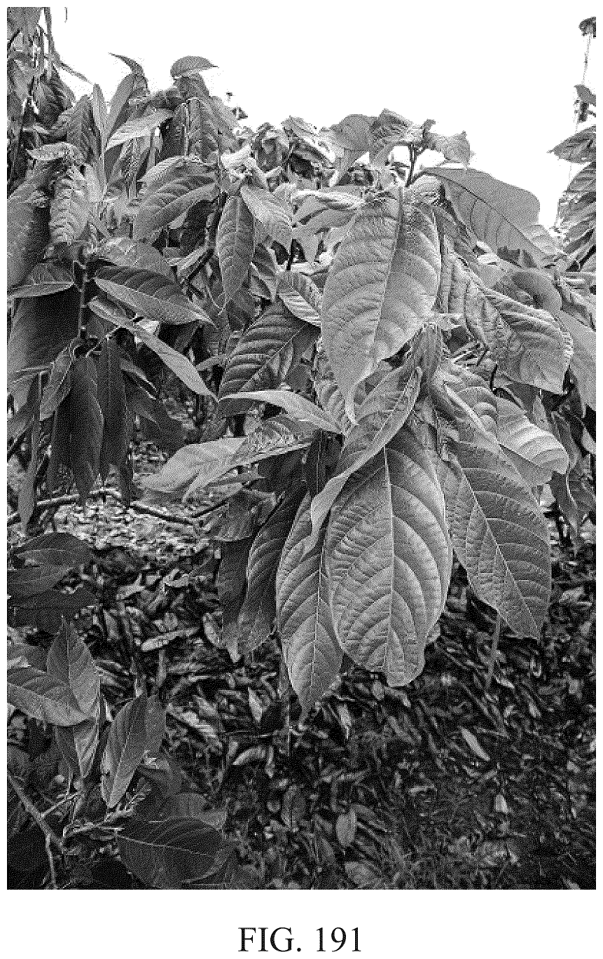

FIG. 192 illustrates a whole tree of variety ‘CMC-032’.

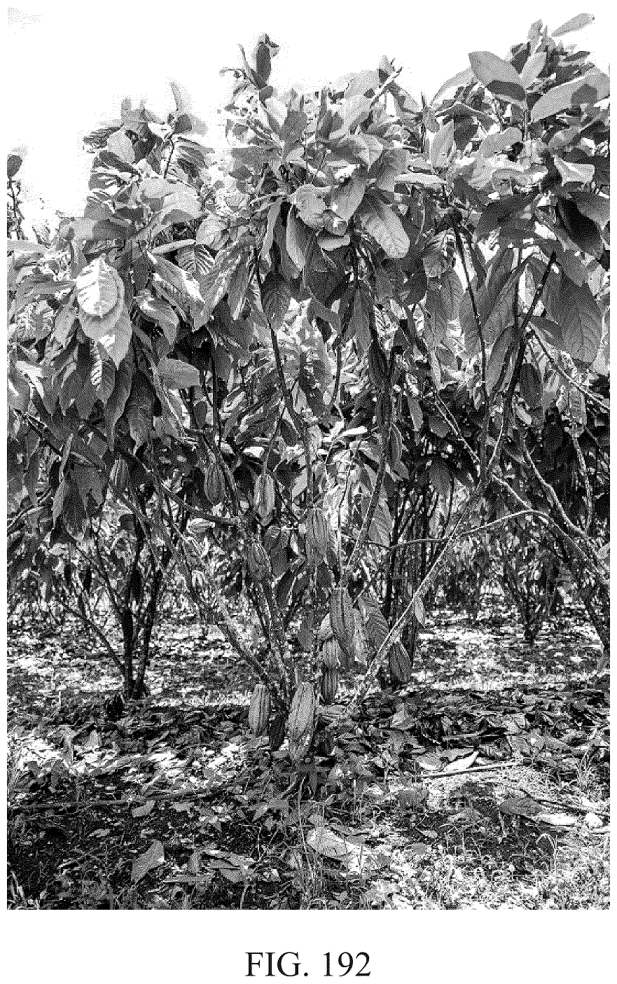

FIG. 193 illustrates a pod of variety ‘CMC-032’

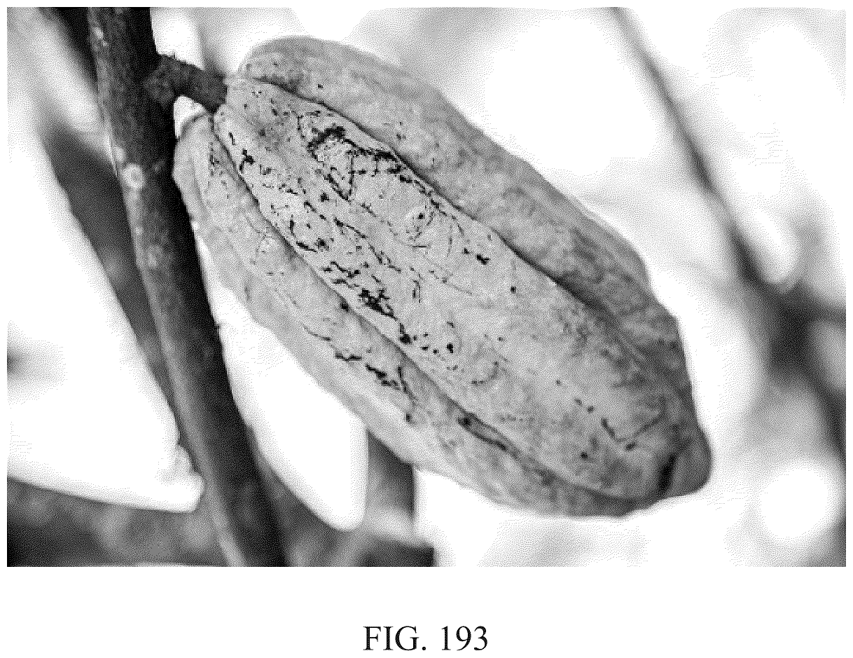

FIG. 194 illustrates a pod of variety ‘CMC-032’.

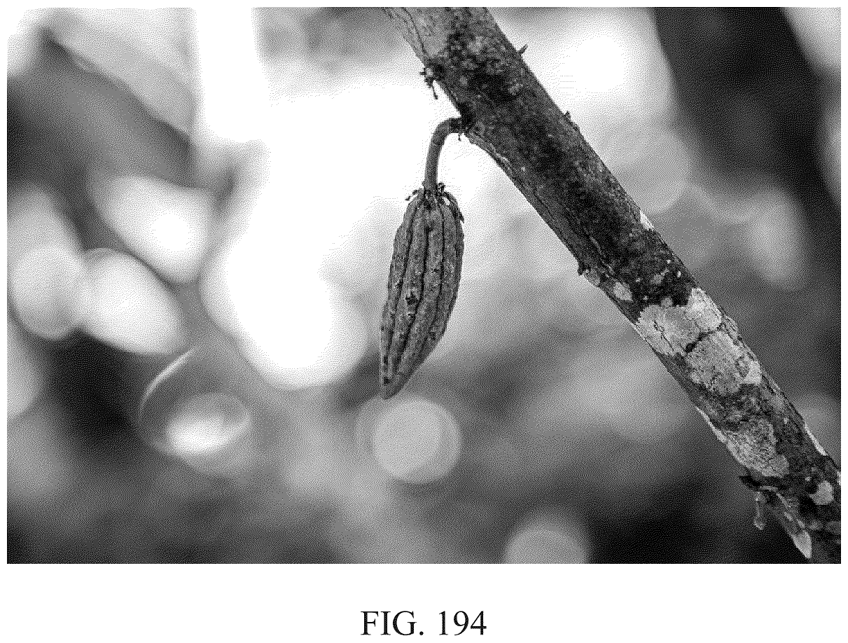

FIG. 195 illustrates a flower of variety ‘CMC-032’.

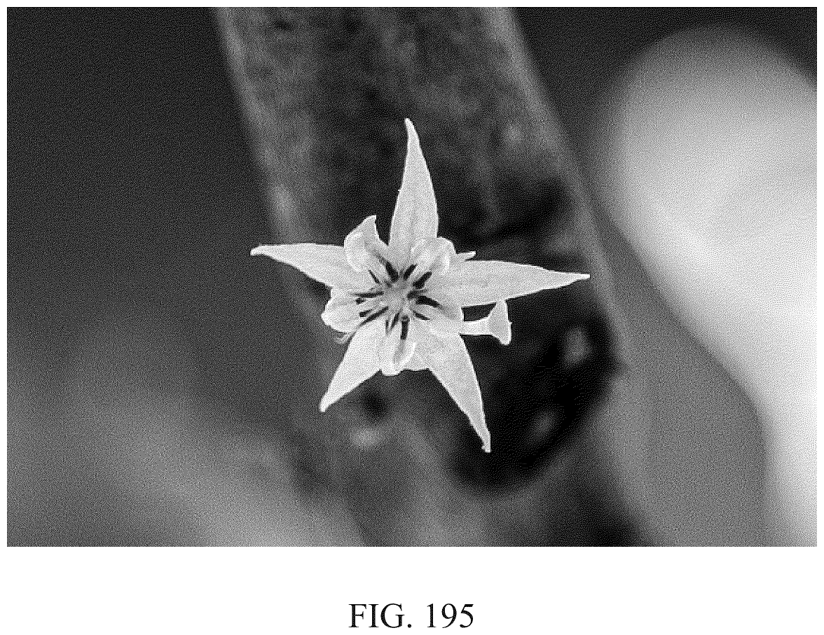

FIG. 196 illustrates flowers and flower buds of variety ‘CMC-032’.

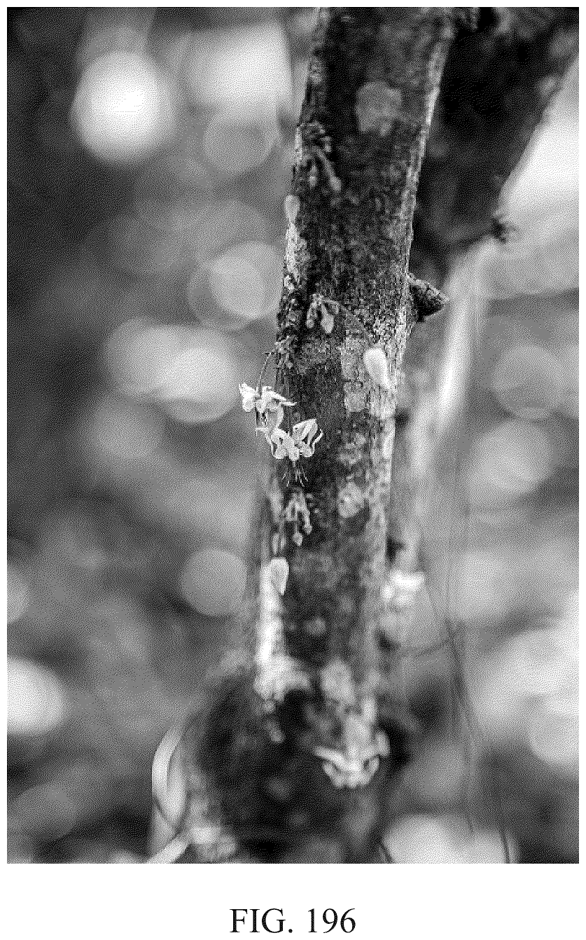

FIG. 197 illustrates leaves of variety ‘CMC-032’.

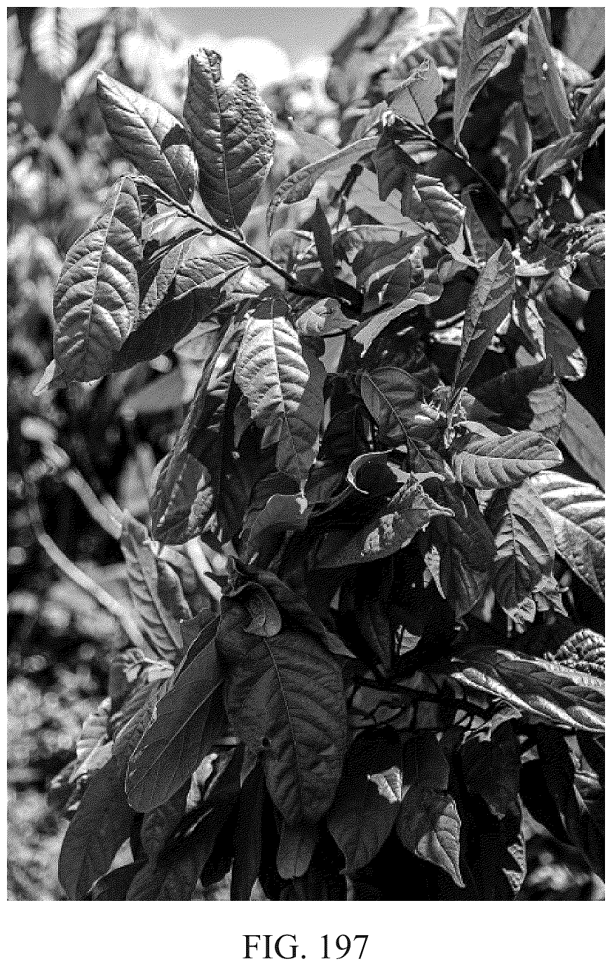

FIG. 198 illustrates a whole tree of variety ‘CMC-033’.

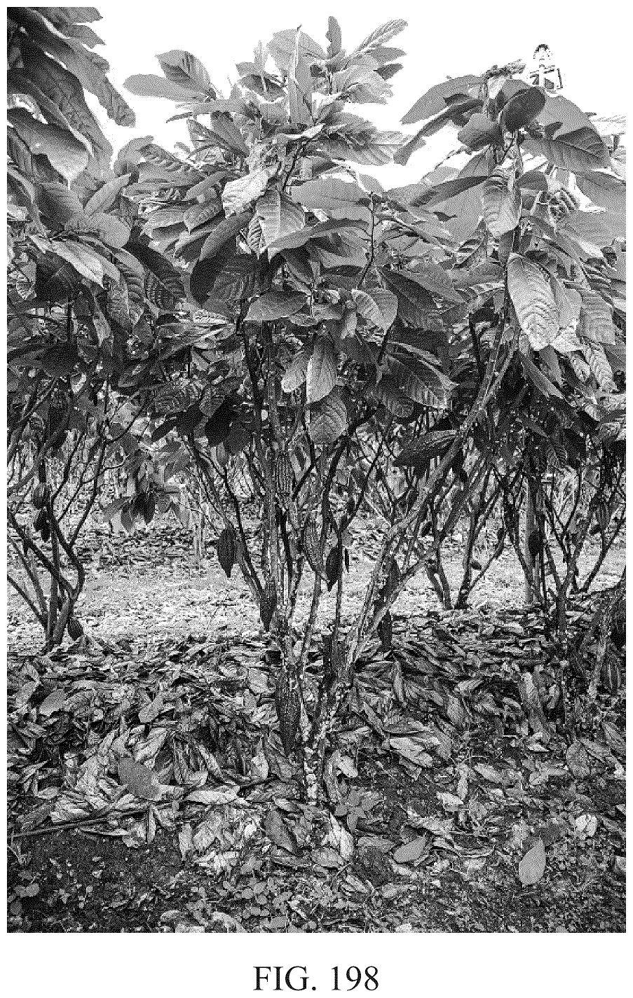

FIG. 199 illustrates a pod of variety ‘CMC-033’.

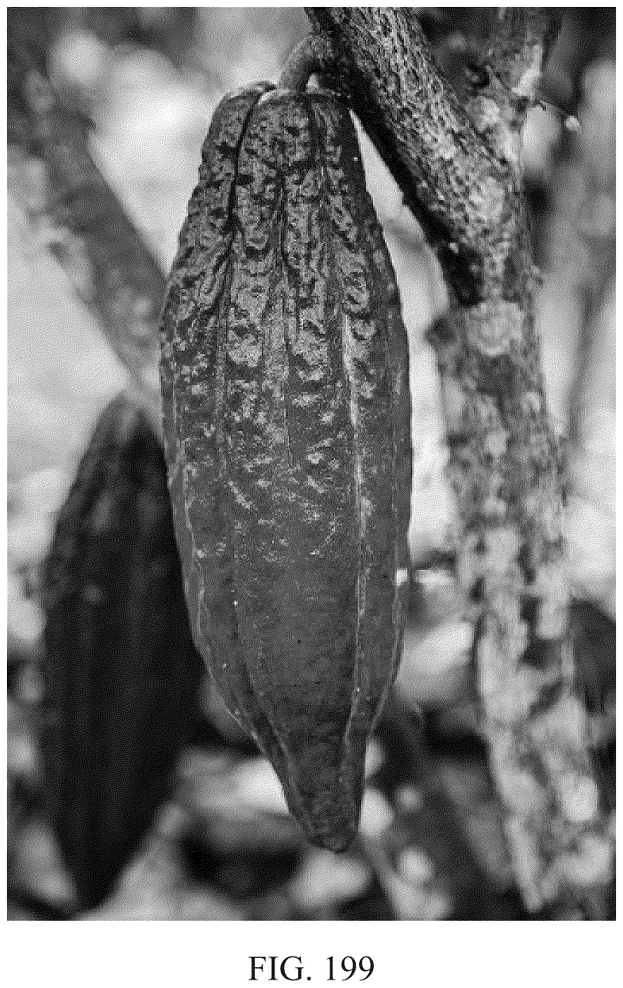

FIG. 200 illustrates a flower of variety ‘CMC-033’.

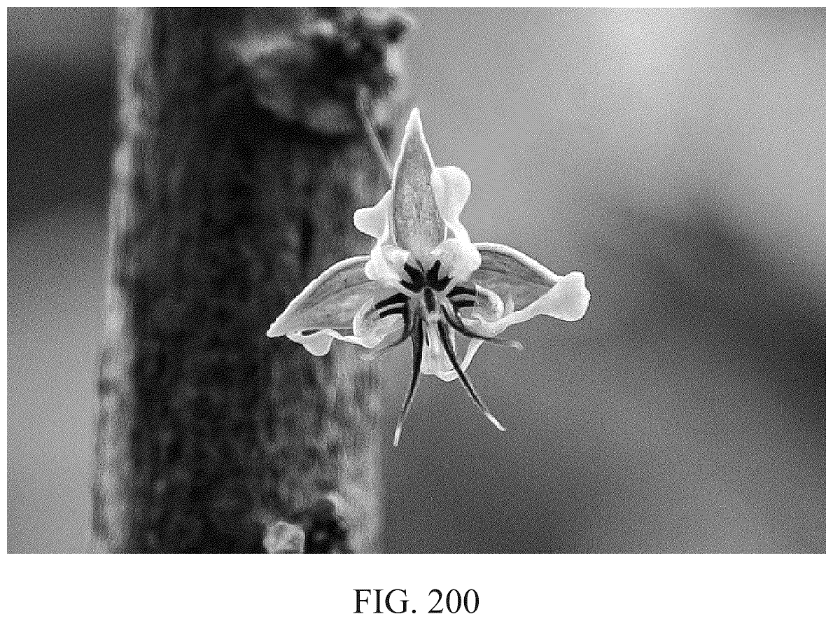

FIG. 201 illustrates flowers and flower buds of variety ‘CMC-033’.

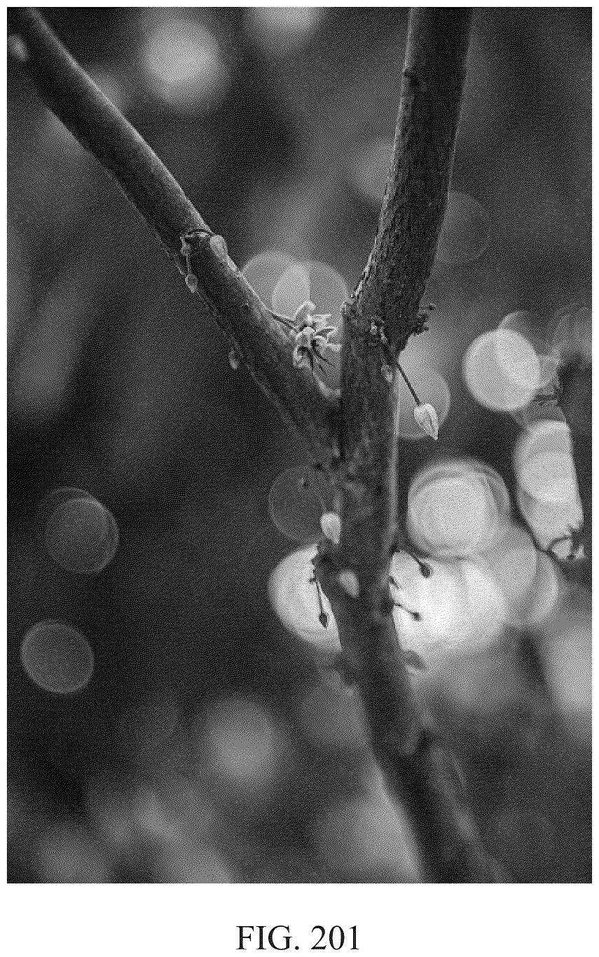

FIG. 202 illustrates flowers and flower buds of variety ‘CMC-033’.

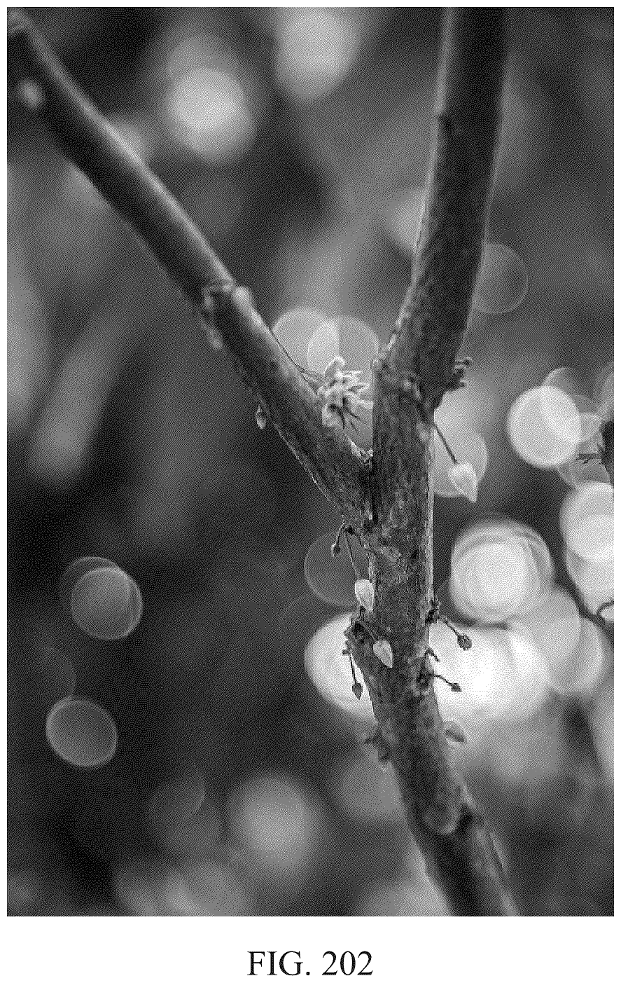

FIG. 203 illustrates flowers and flower buds of variety ‘CMC-033’.

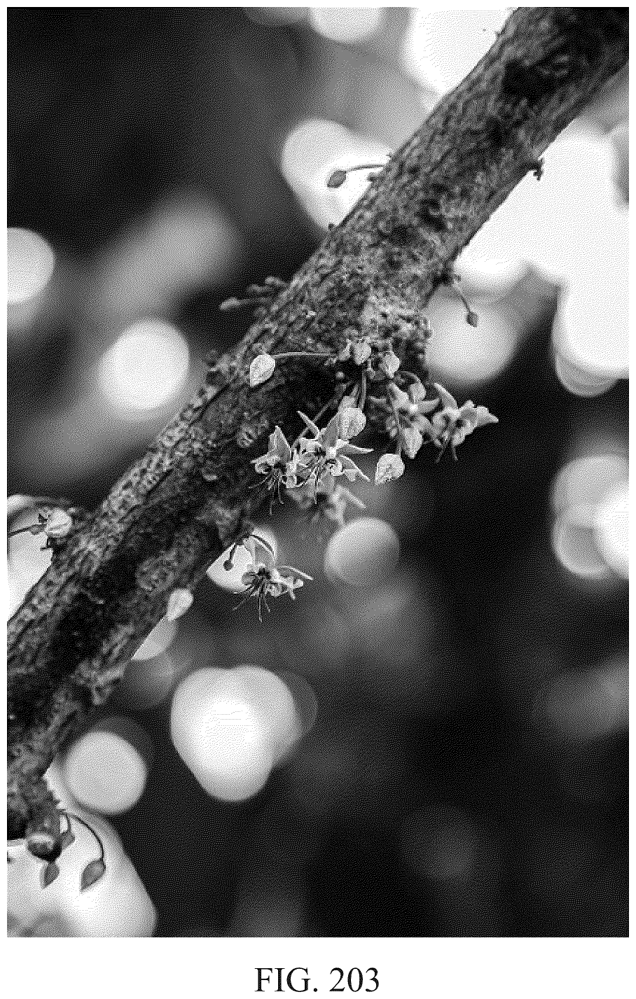

FIG. 204 illustrates leaves of variety ‘CMC-033’.

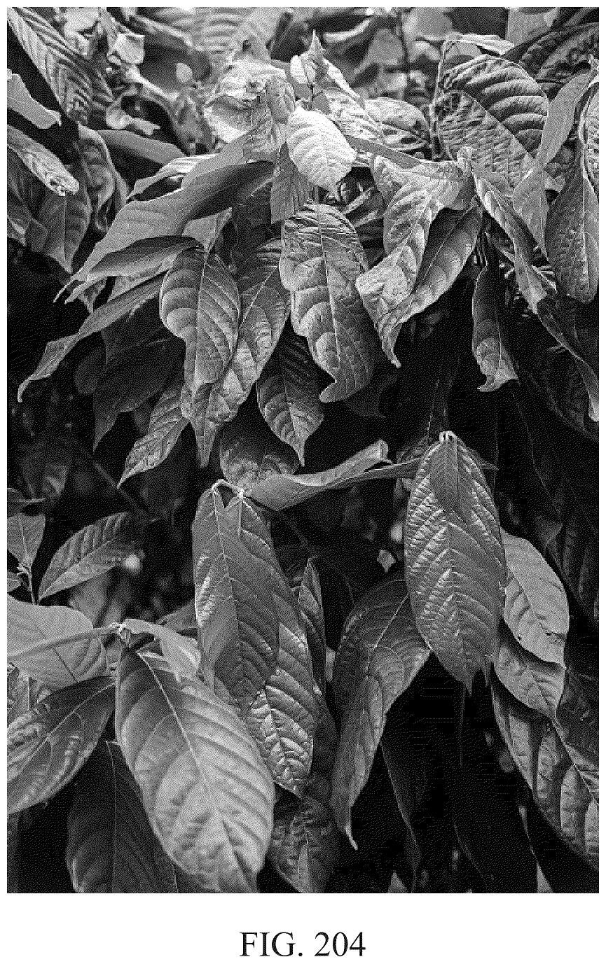

FIG. 205 illustrates a whole tree of variety ‘CMC-034’.

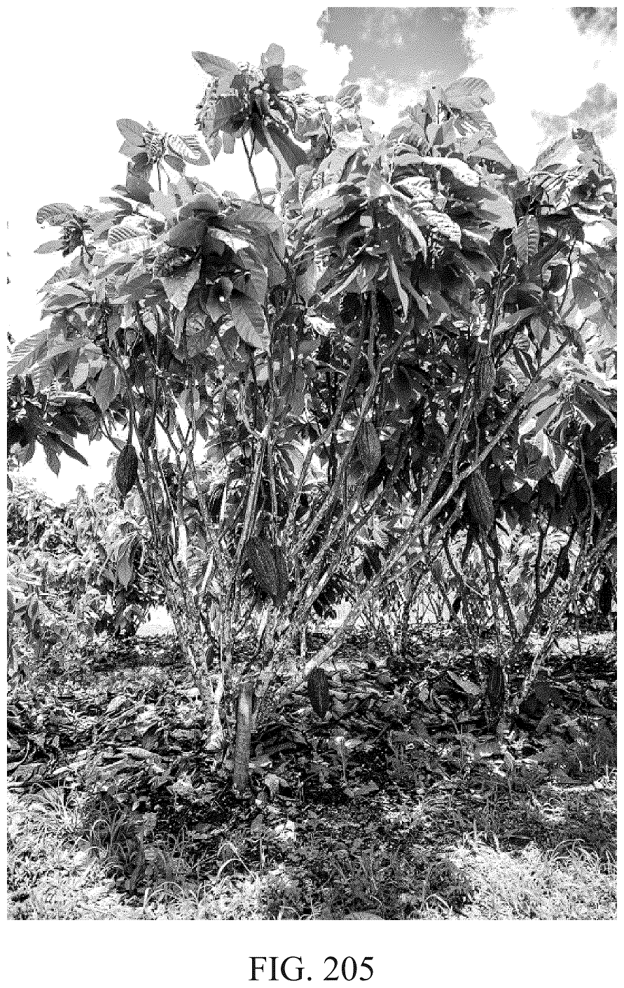

FIG. 206 illustrates a whole tree of variety ‘CMC-034’.

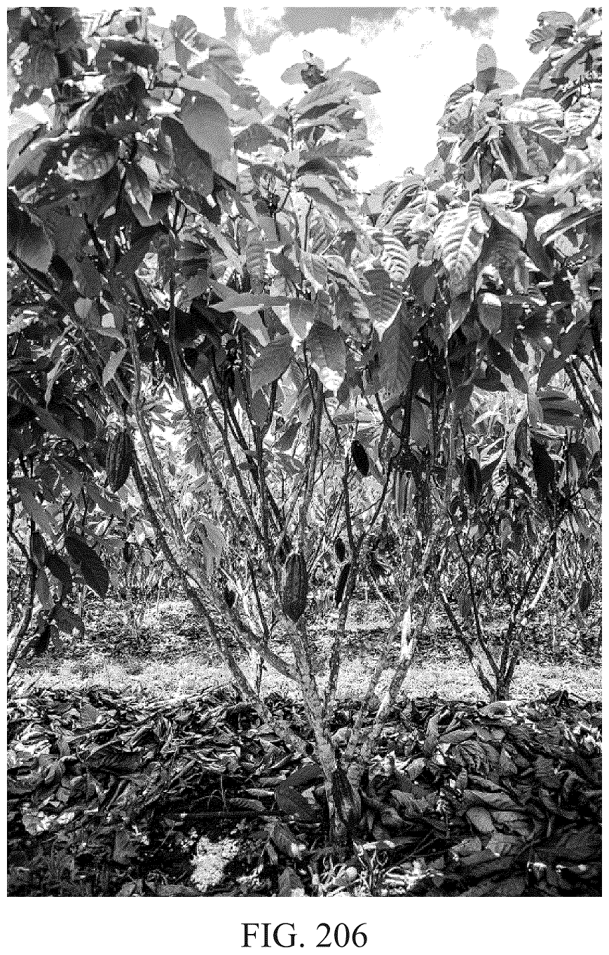

FIG. 207 illustrates a pod of variety ‘CMC-034’.

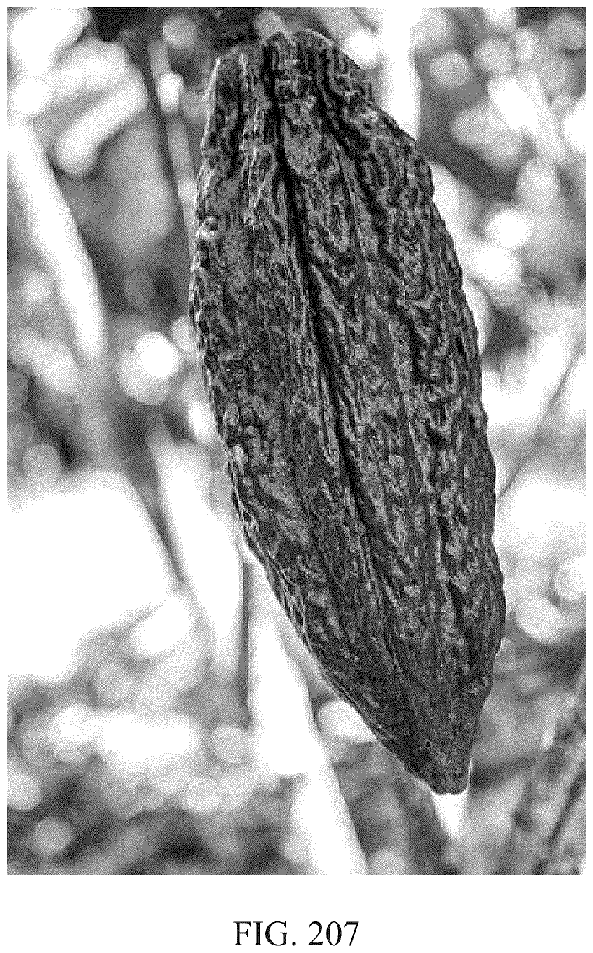

FIG. 208 illustrates a pod of variety ‘CMC-034’.

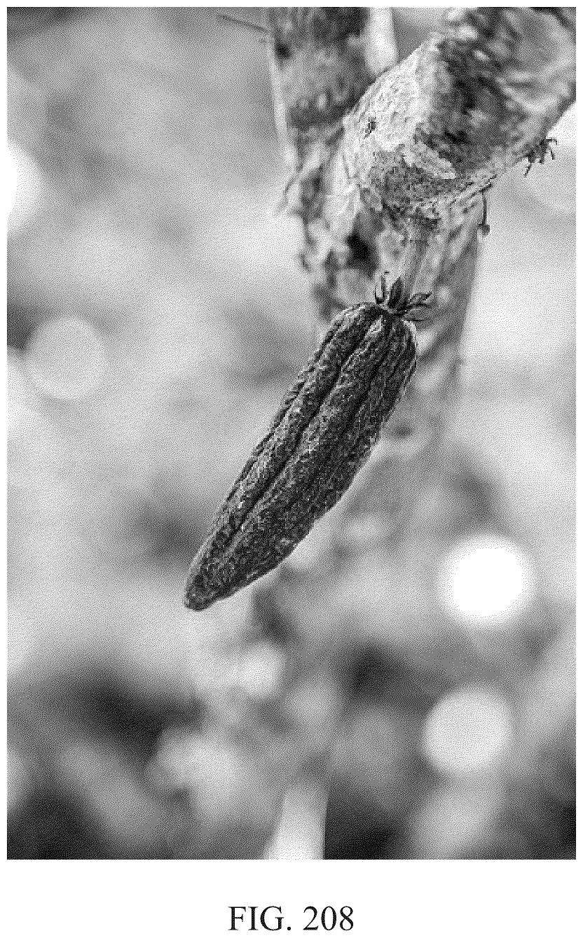

FIG. 209 illustrates a flower of variety ‘CMC-034’.

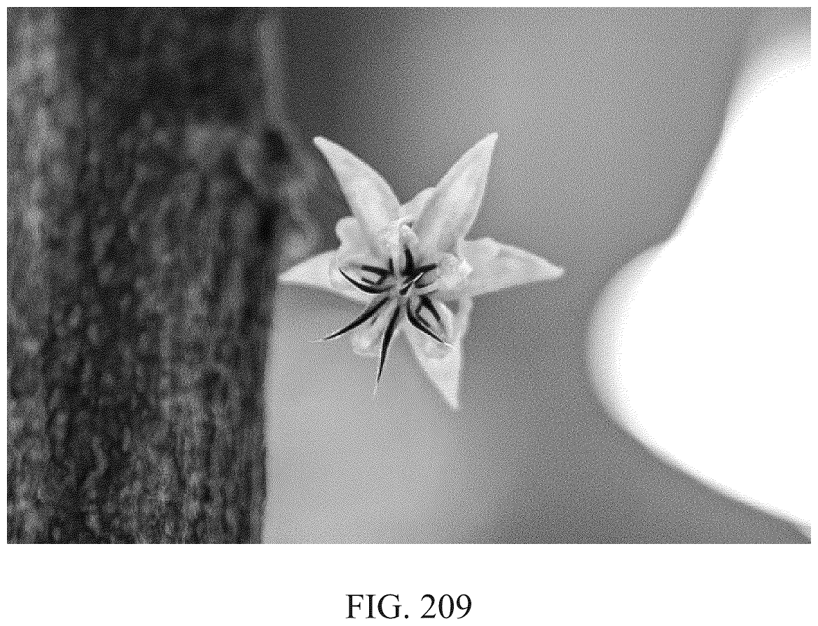

FIG. 210 illustrates a flower of variety ‘CMC-034’.

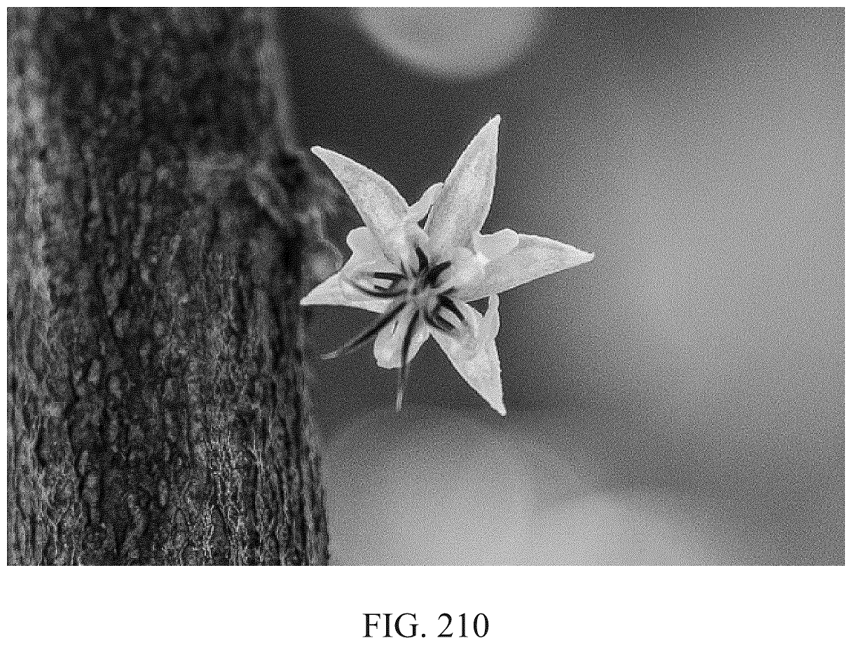

FIG. 211 illustrates flowers and flower buds of variety ‘CMC-034’.

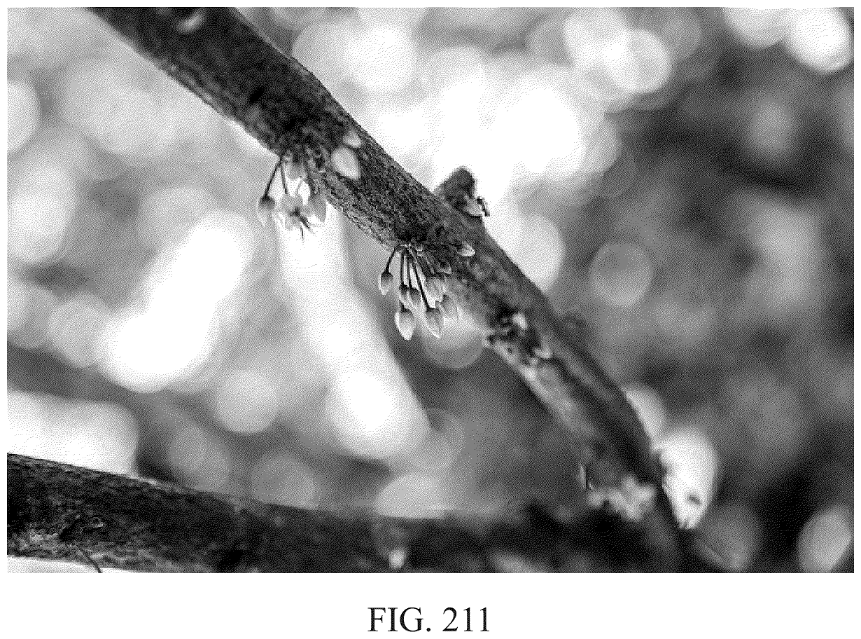

FIG. 212 illustrates flowers and flower buds of variety ‘CMC-034’.

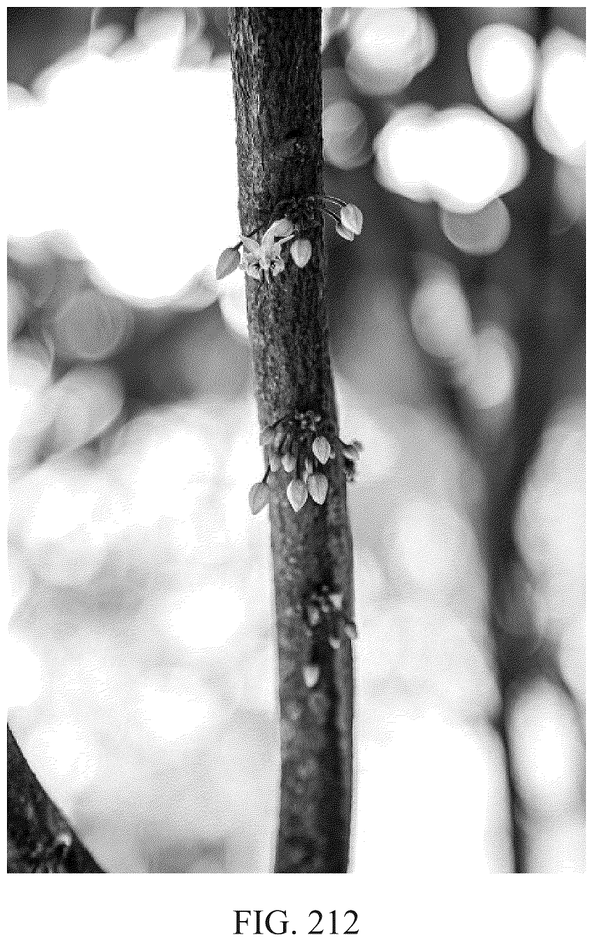

FIG. 213 illustrates leaves of variety ‘CMC-034’.

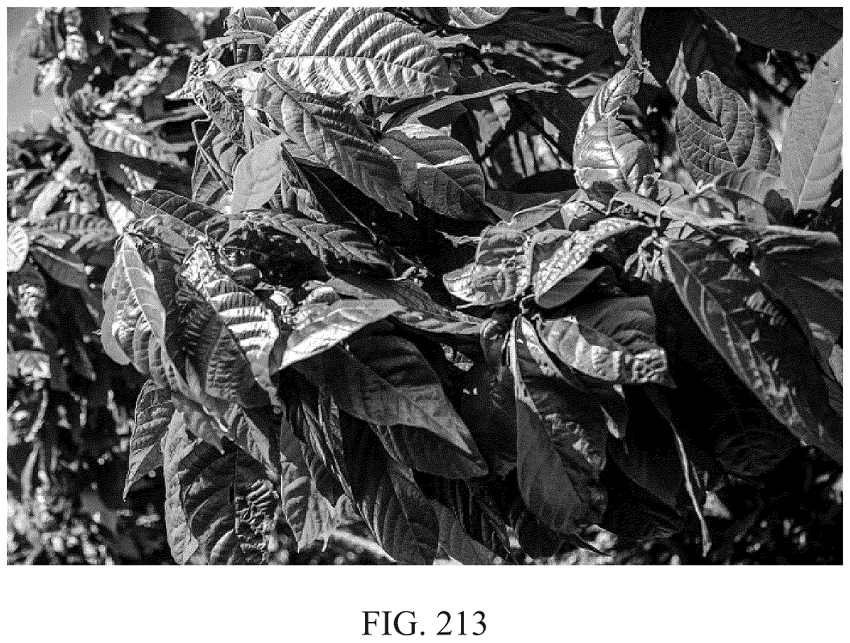

FIG. 214 illustrates a whole tree of variety ‘CMC-035’.

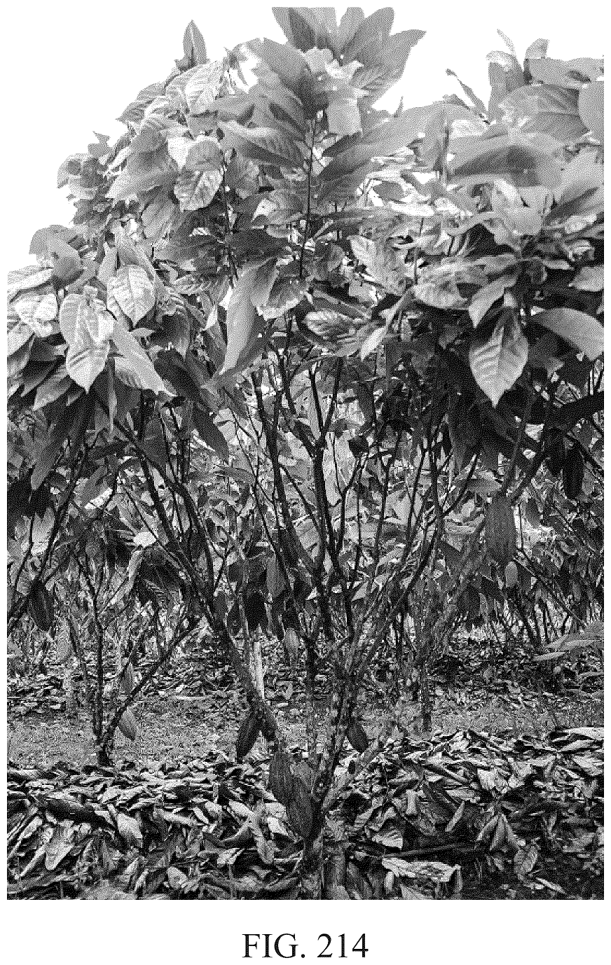

FIG. 215 illustrates a pod of variety ‘CMC-035’.

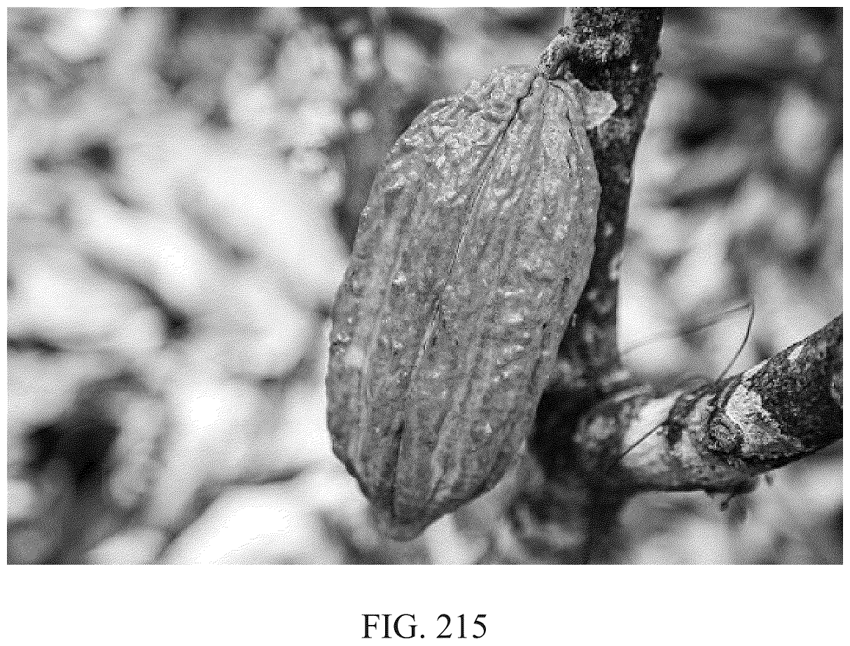

FIG. 216 illustrates a flower of variety ‘CMC-035’.

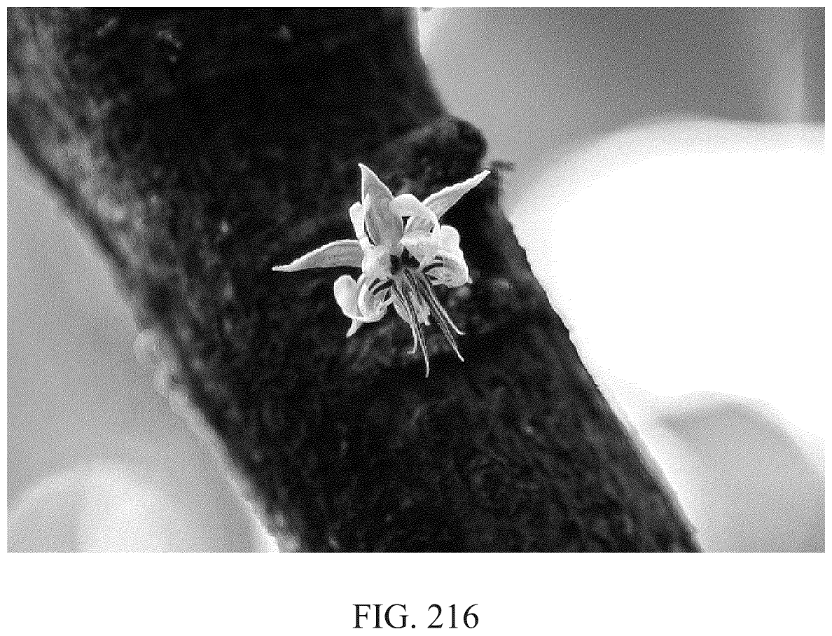

FIG. 217 illustrates flowers and pods of variety ‘CMC-035’.

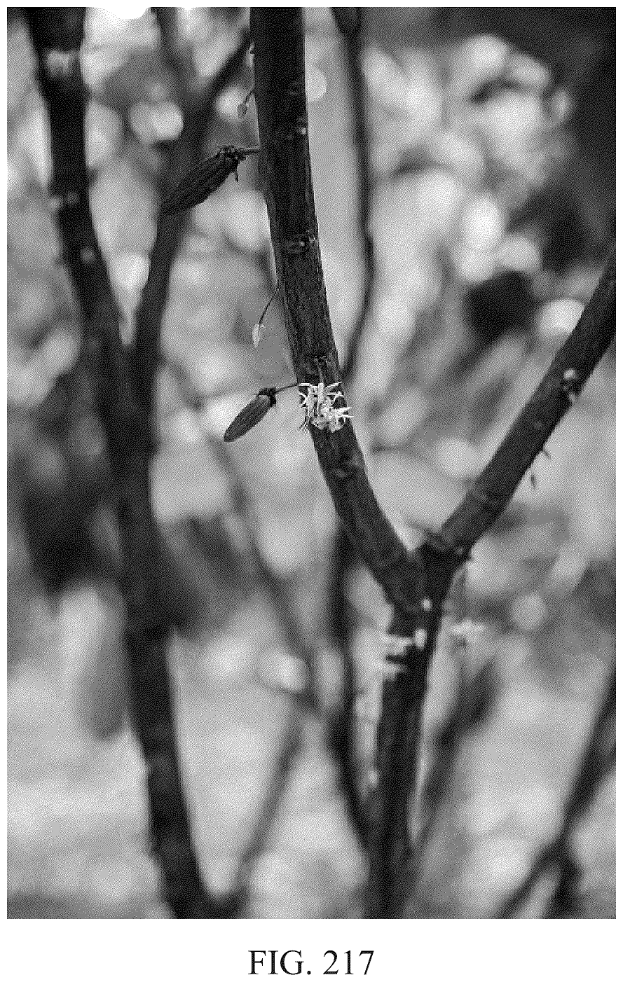

FIG. 218 illustrates flowers and flower buds of variety ‘CMC-035’.

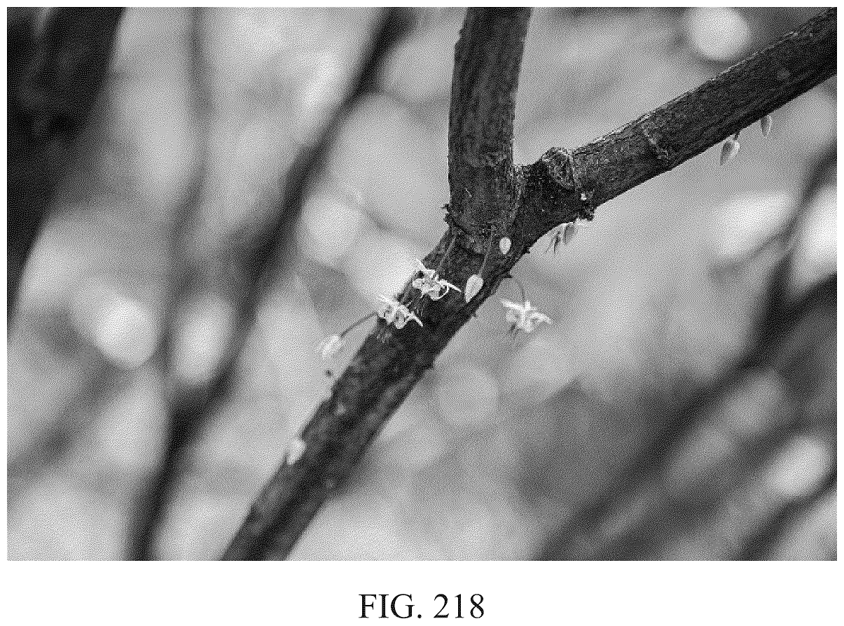

FIG. 219 illustrates leaves of variety ‘CMC-035’.

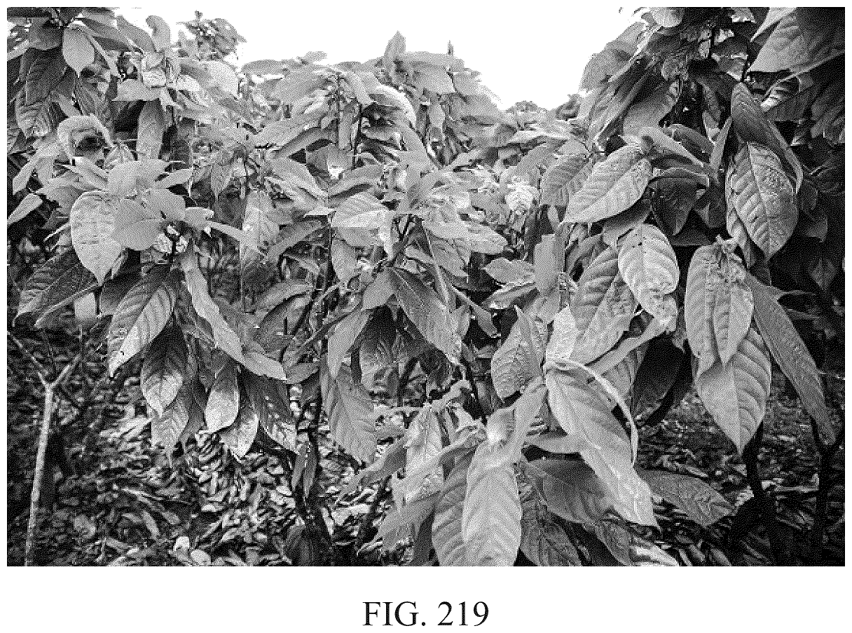

FIG. 220 illustrates a whole tree of variety ‘CMC-036’.

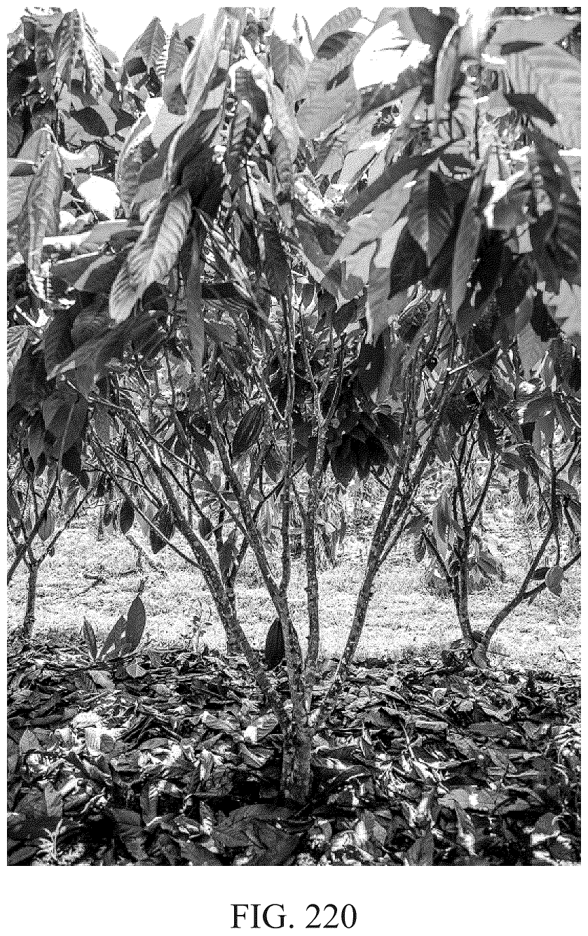

FIG. 221 illustrates a pod of variety ‘CMC-036’.

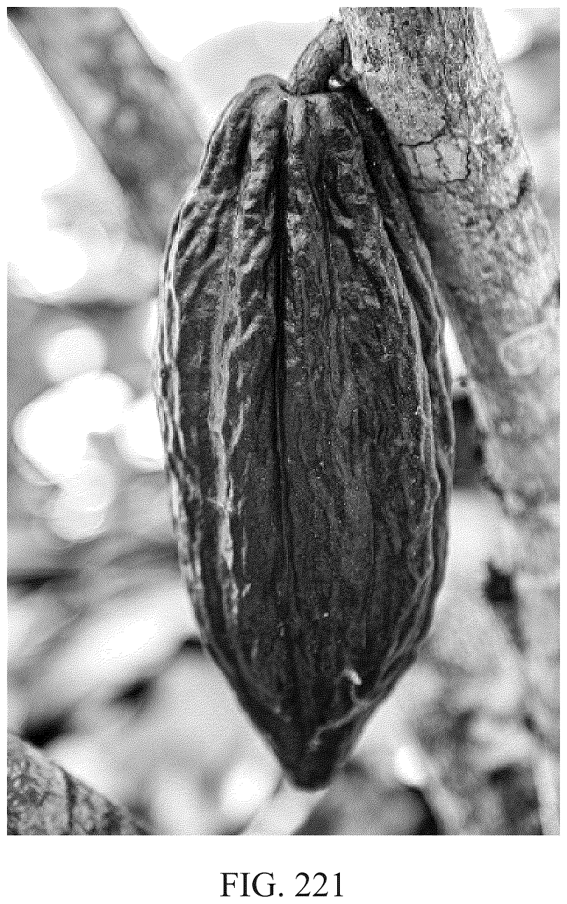

FIG. 222 illustrates a pod of variety ‘CMC-036’.

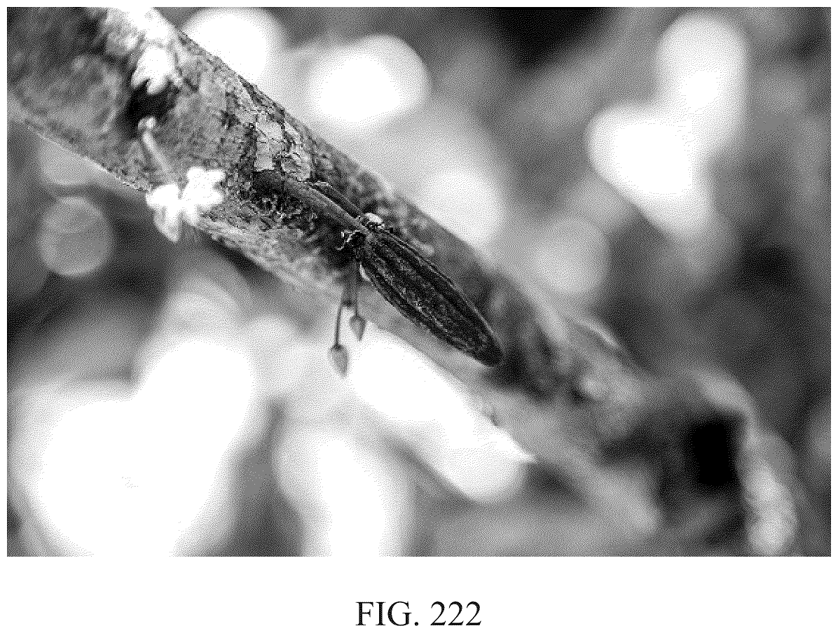

FIG. 223 illustrates a flower of variety ‘CMC-036’.

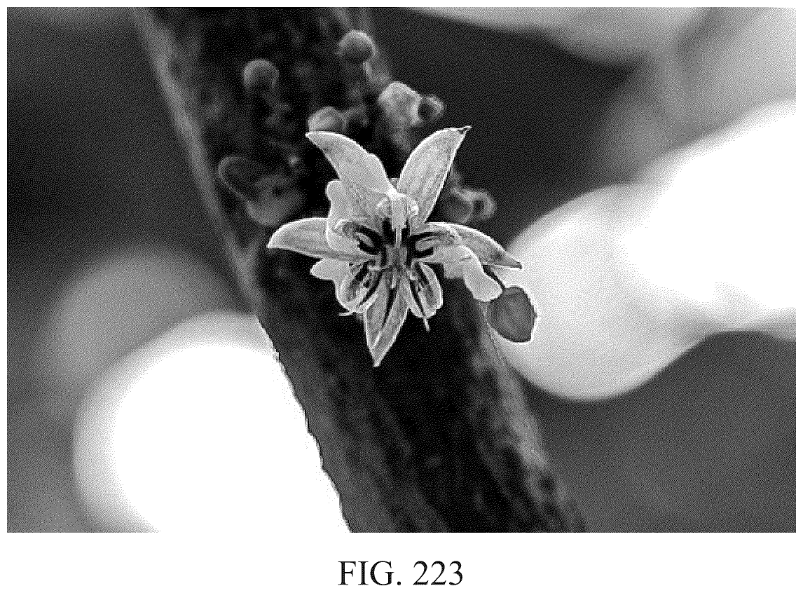

FIG. 224 illustrates flowers and flower buds of variety ‘CMC-036’.

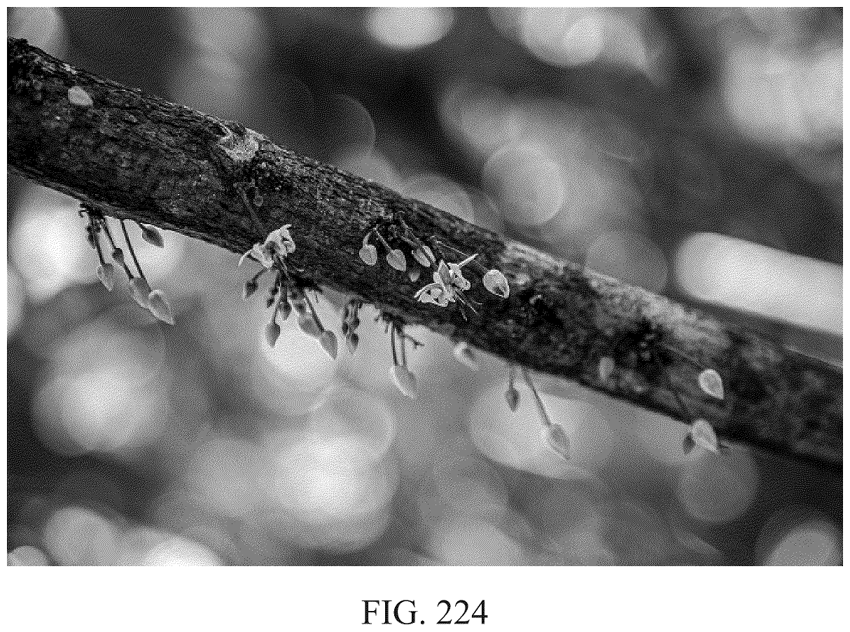

FIG. 225 illustrates leaves of variety ‘CMC-036’.

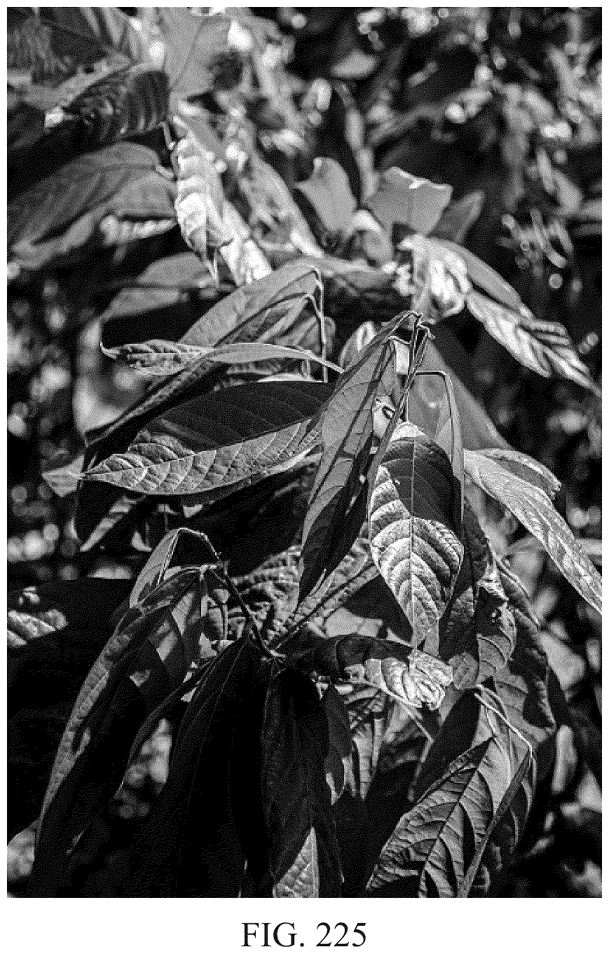

FIG. 226 illustrates a whole tree of variety ‘CMC-022’.

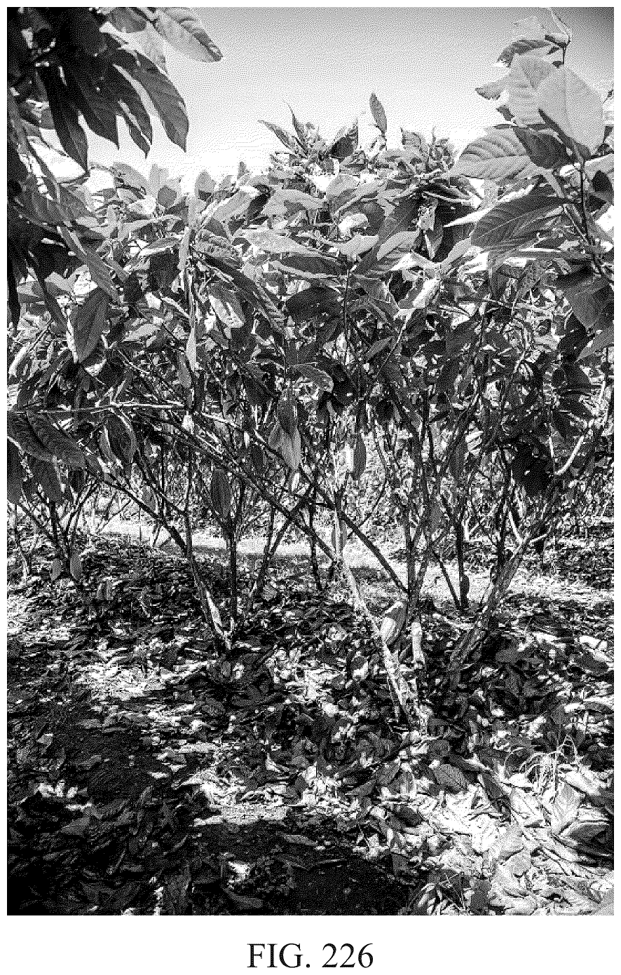

FIG. 227 illustrates a pod of variety ‘CMC-022’.

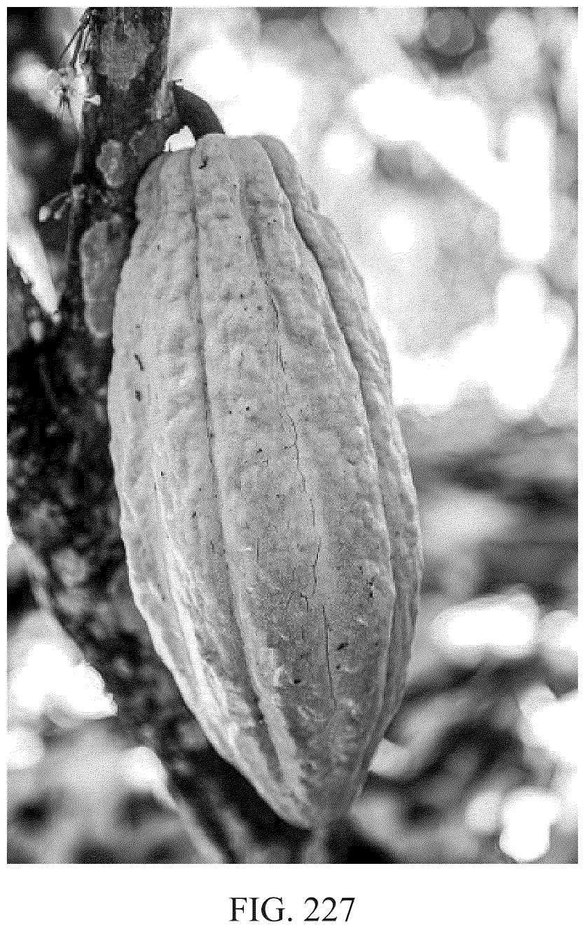

FIG. 228 illustrates a pod of variety ‘CMC-022’.

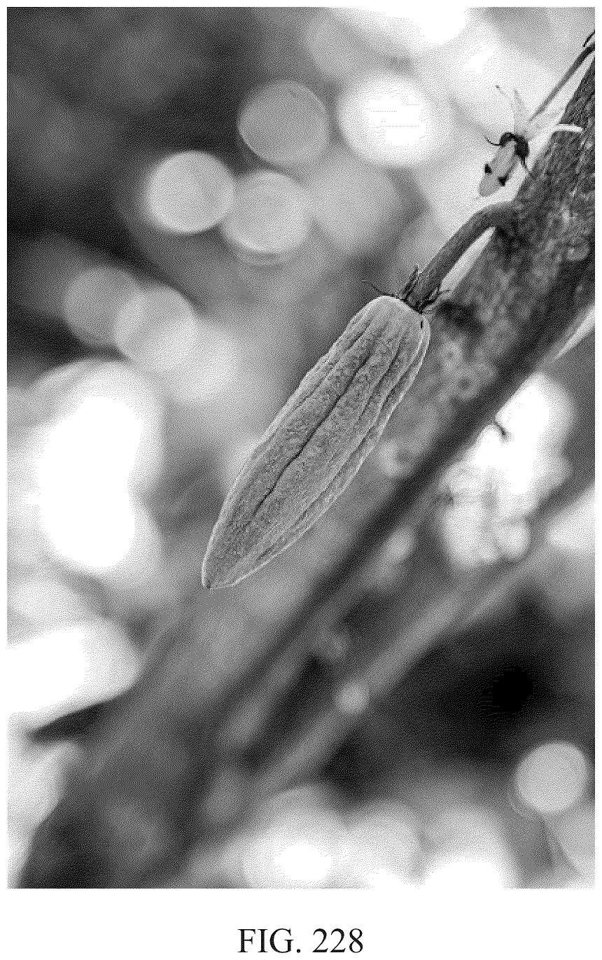

FIG. 229 illustrates a flower of variety ‘CMC-022’.

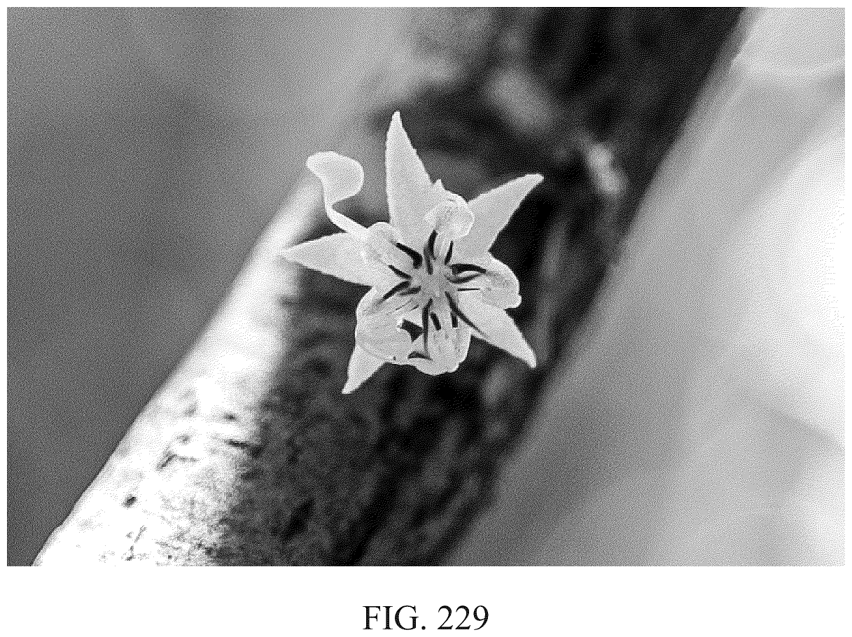

FIG. 230 illustrates flowers and flower buds of variety ‘CMC-022’.

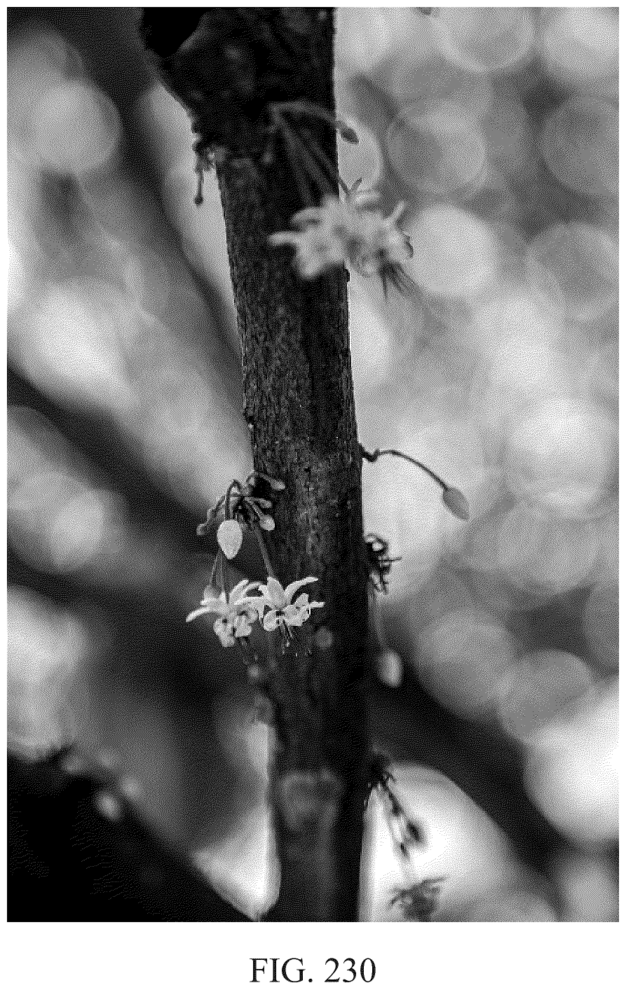

FIG. 231 illustrates leaves of variety ‘CMC-022’.

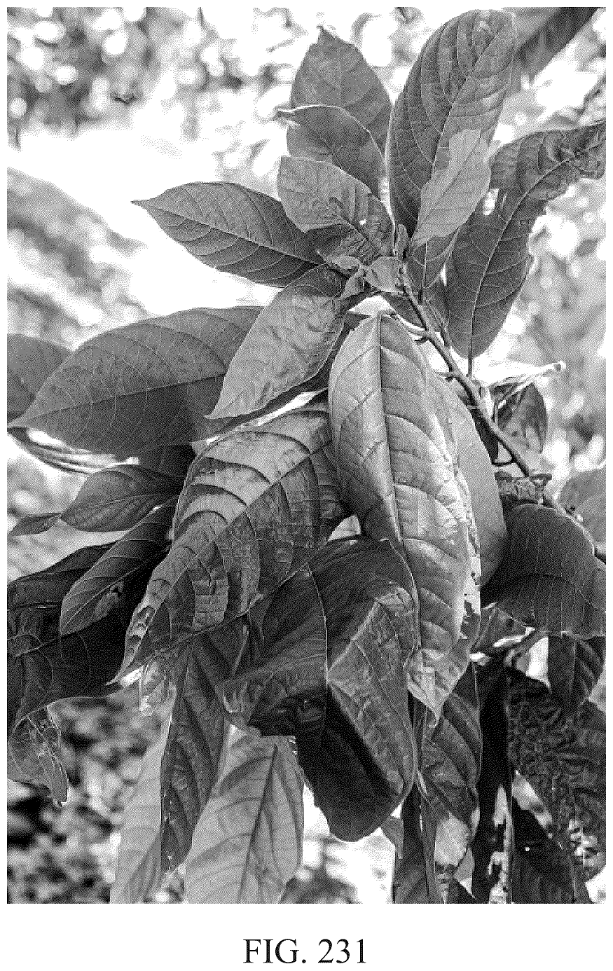

### DETAILED BOTANICAL DESCRIPTION

The following description is based on observations taken of plants and fruits of the new and distinctive cacao varieties of the present invention.

The dry weight of a single bean is calculated by dividing the dry bean weight by the number of healthy beans harvested. The yield is calculated by dividing the total fresh bean weight by the number of reps and multiplying by 1,175 (the plant density in trees/ha). The percentage of pods infected with witches' broom disease is calculated by dividing the number of pods infected with witches' broom disease by the total number of pods harvested. The percentage of pods infected with black pod disease is calculated by dividing the number of pods infected with black pod disease by the total number of pods harvested.

### Variety ‘CMC-001’

In certain aspects, the present invention relates to a new and distinctive cacao plant designated as ‘CMC-001’.

The cacao variety ‘CMC-001’ originated from a controlled cross between varieties ‘GU-144C’ and ‘MC-08091’. The breeder's reference is ‘PT-08-020-1-145-02’, and the variety designation is ‘CMC-001’. Cacao variety ‘CMC-001’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (157.9 cm²), with 27.8 cm in length and 8.7 cm in width. The base leaf shape is obtuse, and the leaf apex is acuminate. The intensity of green in immature leaves is medium red, whereas the intensity of green in the mature leaves is medium.

The average length and width of the sepal are 7.5 mm (medium) and 2.5 mm (medium), with absent anthocyanin coloration. The ligula color is yellow, and the staminode has strong anthocyanin coloration, while the pedicel has weak anthocyanin coloration.

The average length and width of the pod are 174.0 mm (medium) and 78.8 mm (medium), with the yellow color of the mature pod. The pod surface roughness is smooth or slightly rough, with a medium depth of grooves and thick exocarp (e.g., lower and greater exocarps measuring 10.9 and 16.3 mm, respectively). The color of the pulp is white, and the pulp sweetness is medium, with BRIX measuring 15.8. The average number of seeds per pod is 18.6.

The seed has an elliptic shape in longitudinal section, with an average seed length of 23.7 mm (short), and a width of 12.9 mm (medium). The seed thickness is 8.6 mm, and the cotyledon has dark red color.

TABLE 1
Variety ‘CMC-001’ Harvest and Infection Data in 2020 and 2021.

| Characteristic | Value |
|---|---|
| Total reps in 2020 | 12 |
| Total pods harvested in 2020 | 196 |
| Total healthy pods harvested in 2020 | 129 |
| Pods harvested per tree in 2020 | 16 |
| Healthy pods harvested per tree in 2020 | 11 |
| Pods infected with witches' broom disease in 2020 | 22 |
| Pods infected with black pod disease in 2020 | 3 |
| Percentage of pods infected with witches' broom disease in 2020 | 11.22% |
| Percentage of pods infected with black pod disease in 2020 | 1.53% |
| Dry weight of a single bean in 2020 (g) | 0.77 |
| Total dry bean weight in 2020 (g) | 2498.432554 |
| Total dry bean weight per tree in 2020 (g) | 208.20 |
| Yield in 2020 (kg/ha) | 244.6381876 |
| Clone yield divided by CCN 51 yield in 2020 | 1.92 |
| Total reps in 2021 | 12 |
| Total pods harvested in 2021 | 783 |
| Total healthy pods harvested in 2021 | 720 |
| Pods harvested per tree in 2021 | 65 |
| Healthy pods harvested per tree in 2021 | 60 |
| Pods infected with witches' broom disease in 2021 | 6 |
| Pods infected with black pod disease in 2021 | 3 |
| Percentage of pods infected with witches' broom disease in 2021 | 1% |
| Percentage of pods infected with black pod disease in 2021 | 0% |
| Dry weight of a single bean in 2021 (g) | 0.77 |
| Total dry bean weight in 2021 (g) | 14174.6 |
| Total dry bean weight per tree in 2021 (g) | 1181.2 |
| Yield in 2021 (kg/ha) | 1387.9 |
| Clone yield divided by CCN 51 yield in 2021 | 3.8 |
| Accumulated yield (kg/ha) | 1,632.57 |
| Number of dead plants infected with Ceratocystis cacaofunesta | 1 |
| Percentage of seedlings dead after infection with Ceratocystis cacaofunesta | 20% |
| Minimum lesion (mm) | 74.8 |
| Maximum lesion (mm) | 74.8 |
| Average lesion (mm) | 74.8 |
| Fat content (%) | N/A |

### Variety ‘CMC-002’

In certain aspects, the present invention relates to a new and distinctive cacao plant designated as ‘CMC-002’.

The cacao variety ‘CMC-002’ originated from a controlled cross between varieties ‘GU-144C’ and ‘MC-08091’. The breeder's reference is ‘PT-08-020-1-145-12’, and the variety designation is ‘CMC-002’. Cacao variety ‘CMC-002’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Obtuse

The leaf size is medium (170.0 cm²), with 28.3 cm in length and 9.1 cm in width. The base leaf shape is obtuse, and the leaf apex is apiculate. The intensity of green in immature leaves is medium red, whereas the intensity of green in the mature leaves is medium.

The average length and width of the sepals are 7.2 mm (medium) and 2.1 mm (short), respectively, with absent anthocyanin coloration. The ligula color is cream yellow, and the staminode has medium anthocyanin coloration, while the pedicel has weak anthocyanin coloration.

The average length and width of the pod are 179.3 mm (medium) and 76.0 mm (medium), respectively, with the yellow color of the mature pod. The pod surface roughness is moderately rough, with a medium depth of grooves and thick exocarp (e.g. lower and greater exocarps measuring 11.5 and 16.2 mm, respectively). The color of the pulp is light cream, and the pulp sweetness is high, with BRIX measuring 17.1. The average number of seeds per pod is 18.6.

The seed has an elliptic shape in longitudinal section, with an average seed length of 25.6 mm (medium), and a width of 12.9 mm (medium). The seed thickness is 8.8 mm, and the cotyledon has dark purple color.

TABLE 2
Variety ‘CMC-002’ Harvest and Infection Data in 2020 and 2021.

| Characteristic | Value |
|---|---|
| Total reps in 2020 | 12 |
| Total pods harvested in 2020 | 291 |
| Total healthy pods harvested in 2020 | 234 |
| Pods harvested per tree in 2020 | 24 |
| Healthy pods harvested per tree in 2020 | 20 |
| Pods infected with witches' broom disease in 2020 | 11 |
| Pods infected with black pod disease in 2020 | 4 |
| Percentage of pods infected with witches' broom disease in 2020 | 3.78% |
| Percentage of pods infected with black pod disease in 2020 | 1.37% |
| Dry weight of a single bean in 2020 (g) | 0.86 |
| Total dry bean weight in 2020 (g) | 5903.579 |
| Total dry bean weight per tree in 2020 (g) | 491.96 |
| Yield in 2020 (kg/ha) | 578.0588 |
| Clone yield divided by CCN 51 yield in 2020 | 4.53 |
| Total reps in 2021 | 12 |
| Total pods harvested in 2021 | 494 |
| Total healthy pods harvested in 2021 | 451 |
| Pods harvested per tree in 2021 | 41 |
| Healthy pods harvested per tree in 2021 | 38 |
| Pods infected with witches' broom disease in 2021 | 7 |
| Pods infected with black pod disease in 2021 | 2 |
| Percentage of pods infected with witches' broom disease in 2021 | 1% |
| Percentage of pods infected with black pod disease in 2021 | 0% |
| Dry weight of a single bean in 2021 (g) | 1.13 |
| Total dry bean weight in 2021 (g) | 9790.2 |
| Total dry bean weight per tree in 2021 (g) | 815.9 |
| Yield in 2021 (kg/ha) | 958.6 |
| Clone yield divided by CCN 51 yield in 2021 | 2.7 |
| Accumulated yield (kg/ha) | 1,536.68 |
| Number of dead plants infected with Ceratocystis cacaofunesta | 2 |
| Percentage of seedlings dead after infection with Ceratocystis cacaofunesta | 40% |
| Minimum lesion (mm) | 127 |
| Maximum lesion (mm) | 159.9 |
| Average lesion (mm) | 143.5 |
| Fat content (%) | 43.776 |

### Variety ‘CMC-003’

In certain aspects, the present invention relates to a new and distinctive cacao plant designated as ‘CMC-003’.

The cacao variety ‘CMC-003’ originated from a controlled cross between varieties ‘GU-144C’ and ‘MC-08017’. The breeder's reference is ‘PT-08-018-2-084-18’, and the variety designation is ‘CMC-003’. Cacao variety ‘CMC-003’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Elliptic
Basal constriction on fruit: Present
Shape of fruit apex: Caudate

The leaf size is medium (198.8 cm²), with 29.2 cm in length and 10.3 cm in width. The base leaf shape is obtuse, and the leaf apex is acuminate. The intensity of green in immature leaves is medium red, whereas the intensity of green in the mature leaves is medium.

The average length and width of the sepals are 5.9 mm (short) and 1.9 mm (medium), respectively, with absent anthocyanin coloration. The ligula color is cream yellow, and the staminode has medium anthocyanin coloration, while the pedicel has weak anthocyanin coloration.

The average length and width of the pod are 156.7 mm (medium) and 66.3 mm (short), respectively, with the yellow color of the mature pod. The pod surface roughness is very rough, with a medium depth of grooves and medium exocarp (e.g. lower and greater exocarps measuring 7.8 and 13.7 mm, respectively). The color of the pulp is light cream, and the pulp sweetness is low, with BRIX measuring 14.8. The average number of seeds per pod is 13.3.

The seed has an elliptic shape in longitudinal section, with an average seed length of 23.3 mm (short), and a width of 12.8 mm (medium). The seed thickness is 8.5 mm, and the cotyledon has dark purple color.

TABLE 3
Variety ‘CMC-003’ Harvest and Infection Data in 2020 and 2021.

| Characteristic | Value |
|---|---|
| Total reps in 2020 | 15 |
| Total pods harvested in 2020 | 470 |
| Total healthy pods harvested in 2020 | 326 |
| Pods harvested per tree in 2020 | 31 |
| Healthy pods harvested per tree in 2020 | 22 |
| Pods infected with witches' broom disease in 2020 | 20 |
| Pods infected with black pod disease in 2020 | 14 |
| Percentage of pods infected with witches' broom disease in 2020 | 4.26% |
| Percentage of pods infected with black pod disease in 2020 | 2.98% |
| Dry weight of a single bean in 2020 (g) | 0.78 |
| Total dry bean weight in 2020 (g) | 7393.837 |
| Total dry bean weight per tree in 2020 (g) | 492.92 |
| Yield in 2020 (kg/ha) | 579.1839 |
| Clone yield divided by CCN 51 yield in 2020 | 4.54 |
| Total reps in 2021 | 15 |
| Total pods harvested in 2021 | 587 |
| Total healthy pods harvested in 2021 | 551 |
| Pods harvested per tree in 2021 | 39 |
| Healthy pods harvested per tree in 2021 | 37 |
| Pods infected with witches' broom disease in 2021 | 2 |
| Pods infected with black pod disease in 2021 | 0 |
| Percentage of pods infected with witches' broom disease in 2021 | 0% |
| Percentage of pods infected with black pod disease in 2021 | 0% |
| Dry weight of a single bean in 2021 (g) | 1.12 |
| Total dry bean weight in 2021 (g) | 11999.2 |
| Total dry bean weight per tree in 2021 (g) | 799.9 |
| Yield in 2021 (kg/ha) | 939.9 |
| Clone yield divided by CCN 51 yield in 2021 | 2.6 |
| Accumulated yield (kg/ha) | 1,519.12 |
| Number of dead plants infected with Ceratocystis cacaofunesta | 5 |
| Percentage of seedlings dead after infection with Ceratocystis cacaofunesta | 100% |
| Minimum lesion (mm) | 93.4 |
| Maximum lesion (mm) | 165.2 |
| Average lesion (mm) | 137.4 |
| Fat content (%) | 38.46333 |

### Variety ‘CMC-004’

In certain aspects, the present invention relates to a new and distinctive cacao plant designated as ‘CMC-004’.

The cacao variety ‘CMC-004’ originated from a controlled cross between varieties ‘MC-08005’ and ‘MC-08060’ (‘CMC-029’). The breeder's reference is ‘PT-08-038-1-113-09’, and the variety designation is ‘CMC-004’. Cacao variety ‘CMC-004’ has subsequently been asexually propagated via grafting onto open-pollinated ‘VB-1151’ rootstock.

Characteristics of this variety are provided below:
Young flush leaf color: Light red
Axil spot on petiole: Absent
Intensity of anthocyanin on sepal: Absent or very low
Fruit shape: Elliptic
Basal constriction on fruit: Present
Shape of fruit apex: Caudate

The leaf size is medium (201.6 cm²), with 30.9 cm in length and 9.9 cm in width. The base leaf shape is obtuse, and the leaf apex is acuminate. The intensity of green in immature leaves is light red, whereas the intensity of green in the mature leaves is medium.

The average length and width of the sepals are 7.6 mm (medium) and 2.5 mm (medium), respectively, with absent anthocyanin coloration. The ligula color is yellow, and the staminode has medium anthocyanin coloration, while the pedicel has absent anthocyanin coloration.

The average length and width of the pod are 188.5 mm (medium) and 77.3 mm (medium), respectively, with the yellow color of the mature pod. The pod surface roughness is smooth or slightly rough, with a medium depth of grooves and medium exocarp (e.g. lower and greater exocarps measuring 9.9 and 13.7 mm, respectively). The color of the pulp is light cream, and the pulp sweetness is high, with BRIX measuring 18.5. The average number of seeds per pod is 11.7.

The seed has an oblong shape in longitudinal section, with an average seed length of 23.2 mm (short), and a width of 10.2 mm (medium). The seed thickness is 8.9 mm, and the cotyledon has pink color.

TABLE 4
Variety ‘CMC-004’ Harvest and Infection Data in 2020 and 2021.

| Characteristic | Value |
|---|---|
| Total reps in 2020 | 12 |
| Total pods harvested in 2020 | 816 |
| Total healthy pods harvested in 2020 | 538 |
| Pods harvested per tree in 2020 | 68 |
| Healthy pods harvested per tree in 2020 | 45 |
| Pods infected with witches' broom disease in 2020 | 33 |
| Pods infected with black pod disease in 2020 | 11 |
| Percentage of pods infected with witches' broom disease in 2020 | 4.04% |
| Percentage of pods infected with black pod disease in 2020 | 1.35% |
| Dry weight of a single bean in 2020 (g) | 0.52 |
| Total dry bean weight in 2020 (g) | 9054.772 |
| Total dry bean weight per tree in 2020 (g) | 754.56 |
| Yield in 2020 (kg/ha) | 886.6131 |
| Clone yield divided by CCN 51 yield in 2020 | 6.95 |
| Total reps in 2021 | 12 |
| Total pods harvested in 2021 | 632 |
| Total healthy pods harvested in 2021 | 487 |
| Pods harvested per tree in 2021 | 53 |
| Healthy pods harvested per tree in 2021 | 41 |
| Pods infected with witches' broom disease in 2021 | 5 |
| Pods infected with black pod disease in 2021 | 1 |
| Percentage of pods infected with witches' broom disease in 2021 | 1% |
| Percentage of pods infected with black pod disease in 2021 | 0% |
| Dry weight of a single bean in 2021 (g) | 0.64 |
| Total dry bean weight in 2021 (g) | 6323.1 |
| Total dry bean weight per tree in 2021 (g) | 526.9 |
| Yield in 2021 (kg/ha) | 619.1 |
| Clone yield divided by CCN 51 yield in 2021 | 1.7 |
| Accumulated yield (kg/ha) | 1,505.75 |
| Number of dead plants infected with Ceratocystis cacaofunesta | 2 |
| Percentage of seedlings dead after infection with Ceratocystis cacaofunesta | 40% |
| Minimum lesion (mm) | 145.1 |
| Maximum lesion (mm) | 154.9 |
| Average lesion (mm) | 150 |
| Fat content (%) | 37.63 |

### Variety ‘CMC-005’

In certain aspects, the present invention relates to a new and distinctive cacao plant designated as ‘CMC-005’.

The cacao variety ‘CMC-005’ originated from a controlled cross between varieties ‘GU-144C’ and ‘MC-08017’. The breeder's reference is ‘PT-08-018-2-084-12’, and the variety designation is ‘CMC-005’. Cacao variety ‘CMC-005’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Caudate

The leaf size is medium (179.4 cm²), with 28.4 cm in length and 9.6 cm in width. The base leaf shape is obtuse, and the leaf apex is apiculate. The intensity of green in immature leaves is medium red, whereas the intensity of green in the mature leaves is medium.

The average length and width of the sepals are 6.8 mm (medium) and 2.0 mm (short), respectively, with absent anthocyanin coloration. The ligula color is cream yellow, and the staminode has weak anthocyanin coloration, while the pedicel has absent anthocyanin coloration.

The average length and width of the pod are 160.2 mm (medium) and 61.9 mm (short), respectively, with the yellow color of the mature pod. The pod surface roughness is very rough, with a medium depth of grooves and medium exocarp (e.g. lower and greater exocarps measuring 8.4 and 13.2 mm, respectively). The color of the pulp is light cream, and the pulp sweetness is medium, with BRIX measuring 16.7. The average number of seeds per pod is 15.4.

The seed has an irregular shape in longitudinal section, with an average seed length of 22.4 mm (short), and a width of 12.3 mm (medium). The seed thickness is 9.4 mm, and the cotyledon has dark purple color.

TABLE 5
Variety ‘CMC-005’ Harvest and Infection Data in 2020 and 2021.

| Characteristic | Value |
|---|---|
| Total reps in 2020 | 12 |
| Total pods harvested in 2020 | 445 |
| Total healthy pods harvested in 2020 | 308 |
| Pods harvested per tree in 2020 | 37 |
| Healthy pods harvested per tree in 2020 | 26 |
| Pods infected with witches' broom disease in 2020 | 33 |
| Pods infected with black pod disease in 2020 | 43 |
| Percentage of pods infected with witches' broom disease in 2020 | 7.42% |
| Percentage of pods infected with black pod disease in 2020 | 9.66% |
| Dry weight of a single bean in 2020 (g) | 1.00 |
| Total dry bean weight in 2020 (g) | 8279.69 |
| Total dry bean weight per tree in 2020 (g) | 689.97 |
| Yield in 2020 (kg/ha) | 810.7196 |
| Clone yield divided by CCN 51 yield in 2020 | 6.36 |
| Total reps in 2021 | 12 |
| Total pods harvested in 2021 | 319 |
| Total healthy pods harvested in 2021 | 282 |
| Pods harvested per tree in 2021 | 27 |
| Healthy pods harvested per tree in 2021 | 24 |
| Pods infected with witches' broom disease in 2021 | 7 |
| Pods infected with black pod disease in 2021 | 7 |
| Percentage of pods infected with witches' broom disease in 2021 | 2% |
| Percentage of pods infected with black pod disease in 2021 | 2% |
| Dry weight of a single bean in 2021 (g) | 1.09 |
| Total dry bean weight in 2021 (g) | 5891.5 |
| Total dry bean weight per tree in 2021 (g) | 491.0 |
| Yield in 2021 (kg/ha) | 576.9 |
| Clone yield divided by CCN 51 yield in 2021 | 1.6 |
| Accumulated yield (kg/ha) | 1,387.60 |
| Number of dead plants infected with Ceratocystis cacaofunesta | 5 |
| Percentage of seedlings dead after infection with Ceratocystis cacaofunesta | 100% |
| Minimum lesion (mm) | 65.7 |
| Maximum lesion (mm) | 174 |
| Average lesion (mm) | 124.2 |
| Fat content (%) | 41.33286 |

### Variety ‘CMC-006’

In certain aspects, the present invention relates to a new and distinctive cacao plant designated as ‘CMC-006’.

The cacao variety ‘CMC-006’ originated from a controlled cross between varieties ‘MC-08005’ and ‘MC-08060’ (‘CMC-029’). The breeder's reference is ‘PT-08-038-3-096-10’, and the variety designation is ‘CMC-006’. Cacao variety ‘CMC-006’ has subsequently been asexually propagated via grafting onto open-pollinated ‘VB-1151’ rootstock.

Characteristics of this variety are provided below:
Young flush leaf color: Light red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (191.0 cm²), with 28.6 cm in length and 10.1 cm in width. The base leaf shape is obtuse, and the leaf apex is acuminate. The intensity of green in immature leaves is light red, whereas the intensity of green in the mature leaves is medium.

The average length and width of the sepals are 7.2 mm (medium) and 2.6 mm (medium), respectively, with absent anthocyanin coloration. The ligula color is yellow, and the staminode has weak anthocyanin coloration, while the pedicel has absent anthocyanin coloration.

The average length and width of the pod are 169.1 mm (medium) and 83.3 mm (medium), respectively, with the yellow color of the mature pod. The pod surface roughness is moderately rough, with a medium depth of grooves and thick exocarp (e.g. lower and greater exocarps measuring 10.2 and 16.5 mm, respectively). The color of the pulp is light cream, and the pulp sweetness is medium, with BRIX measuring 16.7. The average number of seeds per pod is 21.3.

The seed has an oblong shape in longitudinal section, with an average seed length of 24.6 mm (short), and a width of 11.6 mm (medium). The seed thickness is 8.4 mm, and the cotyledon bas pink color.

TABLE 6
Variety ‘CMC-006’ Harvest and Infection Data in 2020 and 2021.

| Characteristic | Value |
|---|---|
| Total reps in 2020 | 12 |
| Total pods harvested in 2020 | 156 |
| Total healthy pods harvested in 2020 | 103 |
| Pods harvested per tree in 2020 | 13 |
| Healthy pods harvested per tree in 2020 | 9 |
| Pods infected with witches' broom disease in 2020 | 9 |
| Pods infected with black pod disease in 2020 | 17 |
| Percentage of pods infected with witches' broom disease in 2020 | 5.77% |
| Percentage of pods infected with black pod disease in 2020 | 10.90% |
| Dry weight of a single bean in 2020 (g) | 0.88 |
| Total dry bean weight in 2020 (g) | 2647.068 |
| Total dry bean weight per tree in 2020 (g) | 220.59 |
| Yield in 2020 (kg/ha) | 259.1921 |
| Clone yield divided by CCN 51 yield in 2020 | 2.03 |
| Total reps in 2021 | 12 |
| Total pods harvested in 2021 | 779 |
| Total healthy pods harvested in 2021 | 549 |
| Pods harvested per tree in 2021 | 65 |
| Healthy pods harvested per tree in 2021 | 46 |
| Pods infected with witches' broom disease in 2021 | 11 |
| Pods infected with black pod disease in 2021 | 3 |
| Percentage of pods infected with witches' broom disease in 2021 | 1% |
| Percentage of pods infected with black pod disease in 2021 | 0% |
| Dry weight of a single bean in 2021 (g) | 0.88 |
| Total dry bean weight in 2021 (g) | 11185.1 |
| Total dry bean weight per tree in 2021 (g) | 932.1 |
| Yield in 2021 (kg/ha) | 1095.2 |
| Clone yield divided by CCN 51 yield in 2021 | 3.0 |
| Accumulated yield (kg/ha) | 1,354.40 |
| Number of dead plants infected with Ceratocystis cacaofunesta | 5 |
| Percentage of seedlings dead after infection with Ceratocystis cacaofunesta | 100% |
| Minimum lesion (mm) | 70.6 |
| Maximum lesion (mm) | 145.1 |
| Average lesion (mm) | 108.1 |
| Fat content (%) | NA |

### Variety ‘CMC-007’

In certain aspects, the present invention relates to a new and distinctive cacao plant designated as ‘CMC-007’.

The cacao variety ‘CMC-007’ originated from a controlled cross between varieties ‘GC-144C’ and ‘MC-08091’. The breeder's reference is ‘PT-08-020-1-145-15’, and the variety designation is ‘CMC-007’. Cacao variety ‘CMC-007’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Obtuse

The leaf size is medium (182.3 cm²), with 29.3 cm in length and 9.4 cm in width. The base leaf shape is obtuse, and the leaf apex is acuminate. The intensity of green in immature leaves is medium red, whereas the intensity of green in the mature leaves is medium.

The average length and width of the sepals are 8.3 mm (medium) and 2.3 mm (medium), respectively, with absent anthocyanin coloration. The ligula color is yellow, and the staminode has strong anthocyanin coloration, while the pedicel has absent anthocyanin coloration.

The average length and width of the pod are 179.2 mm (medium) and 81.2 mm (medium), respectively, with the yellow color of the mature pod. The pod surface roughness is moderately rough, with a medium depth of grooves and thick exocarp (e.g. lower and greater exocarps measuring 11.3 and 16.3 mm, respectively). The color of the pulp is dark cream, and the pulp sweetness is high, with BRIX measuring 21.0. The average number of seeds per pod is 23.2.

The seed has an elliptic shape in longitudinal section, with an average seed length of 26.5 mm (medium), and a width of 13.7 mm (medium). The seed thickness is 10.2 mm, and the cotyledon has dark red color.

TABLE 7
Variety ‘CMC-007’ Harvest and Infection Data in 2020 and 2021.

| Characteristic | Value |
|---|---|
| Total reps in 2020 | 15 |
| Total pods harvested in 2020 | 237 |
| Total healthy pods harvested in 2020 | 177 |
| Pods harvested per tree in 2020 | 16 |
| Healthy pods harvested per tree in 2020 | 12 |
| Pods infected with witches' broom disease in 2020 | 18 |
| Pods infected with black pod disease in 2020 | 2 |
| Percentage of pods infected with witches' broom disease in 2020 | 7.59% |
| Percentage of pods infected with black pod disease in 2020 | 0.84% |
| Dry weight of a single bean in 2020 (g) | 0.95 |
| Total dry bean weight in 2020 (g) | 5131.389 |
| Total dry bean weight per tree in 2020 (g) | 342.09 |
| Yield in 2020 (kg/ha) | 401.9588 |
| Clone yield divided by CCN 51 yield in 2020 | 3.15 |
| Total reps in 2021 | 15 |
| Total pods harvested in 2021 | 361 |
| Total healthy pods harvested in 2021 | 327 |
| Pods harvested per tree in 2021 | 24 |
| Healthy pods harvested per tree in 2021 | 22 |
| Pods infected with witches' broom disease in 2021 | 3 |
| Pods infected with black pod disease in 2021 | 2 |
| Percentage of pods infected with witches' broom disease in 2021 | 1% |
| Percentage of pods infected with black pod disease in 2021 | 1% |
| Dry weight of a single bean in 2021 (g) | 1.43 |
| Total dry bean weight in 2021 (g) | 10943.7 |
| Total dry bean weight per tree in 2021 (g) | 729.6 |
| Yield in 2021 (kg/ha) | 857.3 |
| Clone yield divided by CCN 51 yield in 2021 | 2.4 |
| Accumulated yield (kg/ha) | 1,259.22 |
| Number of dead plants infected with Ceratocystis cacaofunesta | 2 |
| Percentage of seedlings dead after infection with Ceratocystis cacaofunesta | 40% |
| Minimum lesion (mm) | 67.4 |
| Maximum lesion (mm) | 136.1 |
| Average lesion (mm) | 101.7 |
| Fat content (%) | 42.05333 |

### Variety ‘CMC-008’

In certain aspects, the present invention relates to a new and distinctive cacao plant designated as ‘CMC-008’.

The cacao variety ‘CMC-008’ originated from a controlled cross between varieties ‘MOQ-4.25’ and ‘MC-08017’. The breeder's reference is ‘PT-08-041-2-052-18’, and the variety designation is ‘CMC-008’. Cacao variety ‘CMC-008’ has subsequently been asexually propagated via grafting onto open-pollinated ‘VB-1151’ rootstock.

Characteristics of this variety are provided below:
Young flush leaf color: Light red
Axil spot on petiole: Absent
Intensity of anthocyanin on sepal: Absent or very low
Fruit shape: Elliptic
Basal constriction on fruit: Present
Shape of fruit apex: Caudate

The leaf size is medium (176.3 cm²), with 26.5 cm in length and 10.1 cm in width. The base leaf shape is obtuse, and the leaf apex is apiculate. The intensity of green in immature leaves is light red, whereas the intensity of green in the mature leaves is medium.

The average length and width of the sepals are 8.4 mm (medium) and 2.6 mm (long), respectively, with absent anthocyanin coloration. The ligula color is cream yellow, and the staminode has medium anthocyanin coloration, while the pedicel has absent anthocyanin coloration.

The average length and width of the pod are 177.2 mm (medium) and 82.9 mm (medium), respectively, with the yellow color of the mature pod. The pod surface roughness is moderately rough, with a medium depth of grooves and thick exocarp (e.g. lower and greater exocarps measuring 11.4 and 16.7 mm, respectively). The color of the pulp is light cream, and the pulp sweetness is high, with BRIX measuring 17.3. The average number of seeds per pod is 18.2.

The seed has an elliptic shape in longitudinal section, with an average seed length of 22.7 mm (medium), and a width of 14.8 mm (medium). The seed thickness is 10.9 mm, and the cotyledon has pink color.

TABLE 8
Variety ‘CMC-008’ Harvest and Infection Data in 2020 and 2021.

| Characteristic | Value |
|---|---|
| Total reps in 2020 | 15 |
| Total pods harvested in 2020 | 307 |
| Total healthy pods harvested in 2020 | 211 |
| Pods harvested per tree in 2020 | 20 |
| Healthy pods harvested per tree in 2020 | 14 |
| Pods infected with witches' broom disease in 2020 | 23 |
| Pods infected with black pod disease in 2020 | 9 |
| Percentage of pods infected with witches' broom disease in 2020 | 7.49% |
| Percentage of pods infected with black pod disease in 2020 | 2.93% |
| Dry weight of a single bean in 2020 (g) | 0.89 |
| Total dry bean weight in 2020 (g) | 5093.924 |
| Total dry bean weight per tree in 2020 (g) | 339.59 |
| Yield in 2020 (kg/ha) | 399.0241 |
| Clone yield divided by CCN 51 yield in 2020 | 3.13 |
| Total reps in 2021 | 15 |
| Total pods harvested in 2021 | 503 |
| Total healthy pods harvested in 2021 | 400 |
| Pods harvested per tree in 2021 | 34 |
| Healthy pods harvested per tree in 2021 | 27 |
| Pods infected with witches' broom disease in 2021 | 11 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 1.85 |
| Total dry bean weight in 2021 (g) | 10966.9 |
| Total dry bean weight per tree in 2021 (g) | 731.1 |
| Yield in 2021 (kg/ha) | 859.1 |
| Clone yield divided by CCN 51 yield in 2021 | 2.4 |
| Accumulated yield (kg/ha) | 1,258.10 |
| Number of dead plants infected with Ceratocystis cacaofunesta | 5 |
| Percentage of seedlings dead after infection with Ceratocystis cacaofunesta | 100% |
| Minimum lesion (mm) | 180.1 |
| Maximum lesion (mm) | 244.9 |
| Average lesion (mm) | 216 |
| Fat content (%) | 41.16 |

### Variety ‘CMC-009’

In certain aspects, the present invention relates to a new and distinctive cacao plant designated as ‘Variety ‘CMC-009’.

The cacao variety ‘CMC-009’ originated from a controlled cross between varieties ‘GU-144C’ and ‘MC-08017’. The breeder's reference is ‘PT-08-018-1-034-18’, and the variety designation is ‘CMC-009’. Cacao variety ‘CMC-009’ has subsequently been asexually propagated via grafting onto open-pollinated ‘VB-1151’ rootstock.

Characteristics of this variety are provided below:
Young flush leaf color: Light red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (181.3 cm²), with 26.7 cm in length and 10.3 cm in width. The base leaf shape is obtuse, and the leaf apex is apiculate. The intensity of green in immature leaves is light red, whereas the intensity of green in the mature leaves is medium.

The average length and width of the sepals are 7.5 mm (medium) and 2.3 mm (medium), respectively, with absent anthocyanin coloration. The ligula color is yellow, and the staminode has medium anthocyanin coloration, while the pedicel has absent anthocyanin coloration.

The average length and width of the pod are 161.0 mm (medium) and 84.3 mm (medium), respectively, with the yellow color of the mature pod. The pod surface roughness is very rough, with a medium depth of grooves and thick exocarp (e.g. lower and greater exocarps measuring 12.4 and 18.1 mm, respectively). The pulp sweetness is high, with BRIX measuring 17.2. The average number of seeds per pod is 20.1.

The seed has an elliptic shape in longitudinal section, with an average seed length of 27.9 mm (medium), and a width of 14.4 mm (medium). The seed thickness is 9.7 mm, and the cotyledon has dark red color.

TABLE 9
Variety ‘CMC-009’ Harvest and Infection Data in 2020 and 2021.

| Characteristic | Value |
|---|---|
| Total reps in 2020 | 12 |
| Total pods harvested in 2020 | 263 |
| Total healthy pods harvested in 2020 | 190 |
| Pods harvested per tree in 2020 | 22 |
| Healthy pods harvested per tree in 2020 | 16 |
| Pods infected with witches' broom disease in 2020 | 23 |
| Pods infected with black pod disease in 2020 | 4 |
| Percentage of pods infected with witches' broom disease in 2020 | 8.75% |
| Percentage of pods infected with black pod disease in 2020 | 1.52% |
| Dry weight of a single bean in 2020 (g) | 1.06 |
| Total dry bean weight in 2020 (g) | 5342.102 |
| Total dry bean weight per tree in 2020 (g) | 445.18 |
| Yield in 2020 (kg/ha) | 523.0808 |
| Clone yield divided by CCN 51 yield in 2020 | 4.10 |
| Total reps in 2021 | 12 |
| Total pods harvested in 2021 | 307 |
| Total healthy pods harvested in 2021 | 269 |
| Pods harvested per tree in 2021 | 26 |
| Healthy pods harvested per tree in 2021 | 22 |
| Pods infected with witches' broom disease in 2021 | 4 |
| Pods infected with black pod disease in 2021 | 0 |
| Percentage of pods infected with witches' broom disease in 2021 | 1% |
| Percentage of pods infected with black pod disease in 2021 | 0% |
| Dry weight of a single bean in 2021 (g) | 1.29 |
| Total dry bean weight in 2021 (g) | 7494.4 |
| Total dry bean weight per tree in 2021 (g) | 624.5 |
| Yield in 2021 (kg/ha) | 733.8 |
| Clone yield divided by CCN 51 yield in 2021 | 2.0 |
| Accumulated yield (kg/ha) | 1,256.91 |
| Number of dead plants infected with Ceratocystis cacaofunesta | 5 |
| Percentage of seedlings dead after infection with Ceratocystis cacaofunesta | 100% |
| Minimum lesion (mm) | 157.1 |
| Maximum lesion (mm) | 219.9 |
| Average lesion (mm) | 171.8 |
| Fat content (%) | 39.544 |

### Variety ‘CMC-010’

In certain aspects, the present invention relates to a new and distinctive cacao plant designated as ‘CMC-010’.

The cacao variety ‘CMC-010’ originated from a controlled cross between varieties ‘MC-08005’ and ‘MC-08060’ (‘CMC-029’). The breeder's reference is ‘PT-08-038-1-113-03’, and the variety designation is ‘CMC-010’. Cacao variety ‘CMC-010’ has subsequently been asexually propagated via grafting onto open-pollinated ‘VB-1151’ rootstock.

Characteristics of this variety are provided below:
Young flush leaf color: Light red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (192.0 cm²), with 29.5 cm in length and 9.9 cm in width. The base leaf shape is obtuse, and the leaf apex is apiculate. The intensity of green in immature leaves is light red, whereas the intensity of green in the mature leaves is medium.

The average length and width of the sepals are 8.4 mm (medium) and 2.7 mm (medium), respectively, with absent anthocyanin coloration. The ligula color is yellow, and the staminode has medium anthocyanin coloration, while the pedicel has moderate anthocyanin coloration.

The average length and width of the pod are 171.2 mm (medium) and 82.3 mm (medium), respectively, with the orange/anthocyanin (ridge) color of the mature pod. The pod surface roughness is very rough, with a medium depth of grooves and thick exocarp (e.g. lower and greater exocarps measuring 10.9 and 16.5 mm, respectively). The color of the pulp is dark cream, and the pulp sweetness is low, with BRIX measuring 13.3. The average number of seeds per pod is 18.0.

The seed has an oblong shape in longitudinal section, with an average seed length of 28.4 mm (medium), and a width of 12.6 mm (medium). The seed thickness is 9.1 mm, and the cotyledon has pink color.

TABLE 10
Variety ‘CMC-010’ Harvest and Infection Data in 2020 and 2021.

| Characteristic | Value |
|---|---|
| Total reps in 2020 | 15 |
| Total pods harvested in 2020 | 240 |
| Total healthy pods harvested in 2020 | 202 |
| Pods harvested per tree in 2020 | 16 |
| Healthy pods harvested per tree in 2020 | 13 |
| Pods infected with witches' broom disease in 2020 | 10 |
| Pods infected with black pod disease in 2020 | 1 |
| Percentage of pods infected with witches' broom disease in 2020 | 4.17% |
| Percentage of pods infected with black pod disease in 2020 | 0.42% |
| Dry weight of a single bean in 2020 (g) | 0.76 |
| Total dry bean weight in 2020 (g) | 4460.742 |
| Total dry bean weight per tree in 2020 (g) | 297.38 |
| Yield in 2020 (kg/ha) | 349.4248 |
| Clone yield divided by CCN 51 yield in 2020 | 2.74 |
| Total reps in 2021 | 15 |
| Total pods harvested in 2021 | 537 |
| Total healthy pods harvested in 2021 | 402 |
| Pods harvested per tree in 2021 | 36 |
| Healthy pods harvested per tree in 2021 | 27 |
| Pods infected with witches' broom disease in 2021 | 17 |
| Pods infected with black pod disease in 2021 | 1 |
| Percentage of pods infected with witches' broom disease in 2021 | 3% |
| Percentage of pods infected with black pod disease in 2021 | 0% |
| Dry weight of a single bean in 2021 (g) | 1.47 |
| Total dry bean weight in 2021 (g) | 11120.7 |
| Total dry bean weight per tree in 2021 (g) | 741.4 |
| Yield in 2021 (kg/ha) | 871.1 |
| Clone yield divided by CCN 51 yield in 2021 | 2.4 |
| Accumulated yield (kg/ha) | 1,220.55 |
| Number of dead plants infected with Ceratocystis cacaofunesta | 2 |
| Percentage of seedlings dead after infection with Ceratocystis cacaofunesta | 40% |
| Minimum lesion (mm) | 133.1 |
| Maximum lesion (mm) | 187.4 |
| Average lesion (mm) | 160.2 |
| Fat content (%) | NA |

### Variety ‘CMC-011’

In certain aspects, the present invention relates to a new and distinctive cacao plant designated as ‘CMC-011’.

The cacao variety ‘CMC-011’ originated from a controlled cross between varieties ‘GU-144C’ and ‘MC-08091’. The breeder's reference is ‘PT-08-020-3-092-11’, and the variety designation is ‘CMC-011’. Cacao variety ‘CMC-011’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Obtuse

The leaf size is medium (169.0 cm²), with 28.3 cm in length and 9.0 cm in width. The base leaf shape is obtuse, and the leaf apex is apiculate. The intensity of green in immature leaves is medium red, whereas the intensity of green in the mature leaves is medium.

The average length and width of the sepals are 8.8 mm (medium) and 2.5 mm (medium), respectively, with absent anthocyanin coloration. The ligula color is yellow, and the staminode has strong anthocyanin coloration, while the pedicel has moderate anthocyanin coloration.

The average length and width of the pod are 150.7 mm (medium) and 74.3 mm (short), respectively, with the yellow color of the mature pod. The pod surface roughness is very rough, with a medium depth of grooves and medium exocarp (e.g. lower and greater exocarps measuring 10.3 and 13.9 mm, respectively). The color of the pulp is light cream, and the pulp sweetness is low, with BRIX measuring 14.1. The average number of seeds per pod is 20.5.

The seed has an irregular shape in longitudinal section, with an average seed length of 24.3 mm (short), and a width of 12.9 mm (medium). The seed thickness is 8.9 mm, and the cotyledon has dark red color.

TABLE 11
Variety ‘CMC-011’ Harvest and Infection Data in 2020 and 2021.

| Characteristic | Value |
|---|---|
| Total reps in 2020 | 12 |
| Total pods harvested in 2020 | 364 |
| Total healthy pods harvested in 2020 | 210 |
| Pods harvested per tree in 2020 | 30 |
| Healthy pods harvested per tree in 2020 | 18 |
| Pods infected with witches' broom disease in 2020 | 11 |
| Pods infected with black pod disease in 2020 | 11 |
| Percentage of pods infected with witches' broom disease in 2020 | 3.02% |
| Percentage of pods infected with black pod disease in 2020 | 3.02% |
| Dry weight of a single bean in 2020 (g) | 0.62 |
| Total dry bean weight in 2020 (g) | 4067.864 |
| Total dry bean weight per tree in 2020 (g) | 338.99 |
| Yield in 2020 (kg/ha) | 398.3117 |
| Clone yield divided by CCN 51 yield in 2020 | 3.12 |
| Total reps in 2021 | 12 |
| Total pods harvested in 2021 | 505 |
| Total healthy pods harvested in 2021 | 488 |
| Pods harvested per tree in 2021 | 42 |
| Healthy pods harvested per tree in 2021 | 41 |
| Pods infected with witches' broom disease in 2021 | 0 |
| Pods infected with black pod disease in 2021 | 0 |
| Percentage of pods infected with witches' broom disease in 2021 | 0% |
| Percentage of pods infected with black pod disease in 2021 | 0% |
| Dry weight of a single bean in 2021 (g) | 0.88 |
| Total dry bean weight in 2021 (g) | 8245.1 |
| Total dry bean weight per tree in 2021 (g) | 687.1 |
| Yield in 2021 (kg/ha) | 807.3 |
| Clone yield divided by CCN 51 yield in 2021 | 2.2 |
| Accumulated yield (kg/ha) | 1,205.64 |
| Number of dead plants infected with Ceratocystis cacaofunesta | 3 |
| Percentage of seedlings dead after infection with Ceratocystis cacaofunesta | 60% |
| Minimum lesion (mm) | 87.2 |
| Maximum lesion (mm) | 149.3 |
| Average lesion (mm) | 119.9 |
| Fat content (%) | 41.42 |

### Variety ‘CMC-012’

In certain aspects, the present invention relates to a new and distinctive cacao plant designated as ‘CMC-012’.

The cacao variety ‘CMC-012’ originated from a controlled cross between varieties ‘PH-111’ and ‘CCN-51’. The breeder's reference is ‘MC-13-202’, and the variety designation is ‘CMC-012’. Cacao variety ‘CMC-012’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Low
Fruit shape: Elliptic
Basal constriction on fruit: Present
Shape of fruit apex: Acute

The leaf size is medium (177.0 cm²), with 26.1 cm in length and 10.3 cm in width. The base leaf shape is obtuse, and the leaf apex is apiculate. The intensity of green in immature leaves is medium red, whereas the intensity of green in the mature leaves is medium.

The average length and width of the sepals are 7.3 mm (medium) and 2.3 mm (short), respectively, with weak anthocyanin coloration. The ligula color is yellow, and the staminode has medium anthocyanin coloration, while the pedicel has moderate anthocyanin coloration.

The average length and width of the pod are 179.6 mm (medium) and 84.2 mm (medium), respectively, with the yellow color of the mature pod. The pod surface roughness is smooth or slightly rough, with a shallow depth of grooves and thick exocarp (e.g. lower and greater exocarps measuring 13.3 and 16.3 mm, respectively). The color of the pulp is light cream, and the pulp sweetness is medium, with BRIX measuring 16.1. The average number of seeds per pod is 27.9.

The seed has an elliptic shape in longitudinal section, with an average seed length of 26.8 mm (medium), and a width of 14.7 mm (medium). The seed thickness is 9.9 mm, and the cotyledon has dark purple color.

TABLE 12
Variety ‘CMC-012’ Harvest and Infection Data in 2020 and 2021.

| Characteristic | Value |
|---|---|
| Total reps in 2020 | 15 |
| Total pods harvested in 2020 | 60 |
| Total healthy pods harvested in 2020 | 36 |
| Pods harvested per tree in 2020 | 4 |
| Healthy pods harvested per tree in 2020 | 2 |
| Pods infected with witches' broom disease in 2020 | 10 |
| Pods infected with black pod disease in 2020 | 1 |
| Percentage of pods infected with witches' broom disease in 2020 | 16.67% |
| Percentage of pods infected with black pod disease in 2020 | 1.67% |
| Dry weight of a single bean in 2020 (g) | 1.09 |
| Total dry bean weight in 2020 (g) | 1122.676 |
| Total dry bean weight per tree in 2020 (g) | 74.85 |
| Yield in 2020 (kg/ha) | 87.94296 |
| Clone yield divided by CCN 51 yield in 2020 | 0.69 |
| Total reps in 2021 | 14 |
| Total pods harvested in 2021 | 485 |
| Total healthy pods harvested in 2021 | 408 |
| Pods harvested per tree in 2021 | 35 |
| Healthy pods harvested per tree in 2021 | 29 |
| Pods infected with witches' broom disease in 2021 | 11 |
| Pods infected with black pod disease in 2021 | 5 |
| Percentage of pods infected with witches' broom disease in 2021 | 2% |
| Percentage of pods infected with black pod disease in 2021 | 1% |
| Dry weight of a single bean in 2021 (g) | 1.64 |
| Total dry bean weight in 2021 (g) | 13302 |
| Total dry bean weight per tree in 2021 (g) | 950.1 |
| Yield in 2021 (kg/ha) | 1116.4 |
| Clone yield divided by CCN 51 yield in 2021 | 3.1 |
| Accumulated yield (kg/ha) | 1,204.36 |
| Number of dead plants infected with Ceratocystis cacaofunesta | 3 |
| Percentage of seedlings dead after infection with Ceratocystis cacaofunesta | 75% |
| Minimum lesion (mm) | 117.1 |
| Maximum lesion (mm) | 162.8 |
| Average lesion (mm) | 139.8 |
| Fat content (%) | NA |

### Variety ‘CMC-013’

In certain aspects, the present invention relates to a new and distinctive cacao plant designated as ‘CMC-013’.

The cacao variety ‘CMC-013’ originated from a controlled cross between varieties ‘CCN-51’ and ‘MC-08017’. The breeder's reference is ‘PT-08-003-3-155-18’, and the variety designation is ‘CMC-013’. Cacao variety ‘CMC-013’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Low
Fruit shape: Elliptic
Basal constriction on fruit: Present
Shape of fruit apex: Caudate

The leaf size is medium (150.8 cm²), with 25.1 cm in length and 9.1 cm in width. The base leaf shape is obtuse, and the leaf apex is acuminate. The intensity of green in immature leaves is dark red, whereas the intensity of green in the mature leaves is medium.

The average length and width of the sepals are 8.4 mm (medium) and 2.6 mm (medium), respectively, with weak anthocyanin coloration. The ligula color is yellow, and the staminode has medium anthocyanin coloration, while the pedicel has weak anthocyanin coloration.

The average length and width of the pod are 159.6 mm (medium) and 74.3 mm (short), respectively, with the orange/anthocyanin (ridge) color of the mature pod. The pod surface roughness is smooth or slightly rough, with a shallow depth of grooves and medium exocarp (e.g. lower and greater exocarps measuring 9.4 and 13.5 mm, respectively). The color of the pulp is light cream, and the pulp sweetness is medium, with BRIX measuring 16.1. The average number of seeds per pod is 21.9.

The seed has an elliptic shape in longitudinal section, with an average seed length of 24.0 mm (short), and a width of 12.4 mm (medium). The seed thickness is 8.8 mm, and the cotyledon has dark purple color.

TABLE 13
Variety ‘CMC-013’ Harvest and Infection Data in 2020 and 2021.

| Characteristic | Value |
|---|---|
| Total reps in 2020 | 15 |
| Total pods harvested in 2020 | 569 |
| Total healthy pods harvested in 2020 | 309 |
| Pods harvested per tree in 2020 | 38 |
| Healthy pods harvested per tree in 2020 | 21 |
| Pods infected with witches' broom disease in 2020 | 74 |
| Pods infected with black pod disease in 2020 | 16 |
| Percentage of pods infected with witches' broom disease in 2020 | 13.01% |
| Percentage of pods infected with black pod disease in 2020 | 2.81% |
| Dry weight of a single bean in 2020 (g) | 0.83 |
| Total dry bean weight in 2020 (g) | 7364.798 |
| Total dry bean weight per tree in 2020 (g) | 490.99 |
| Yield in 2020 (kg/ha) | 576.9091 |
| Clone yield divided by CCN 51 yield in 2020 | 4.52 |
| Total reps in 2021 | 15 |
| Total pods harvested in 2021 | 662 |
| Total healthy pods harvested in 2021 | 393 |
| Pods harvested per tree in 2021 | 44 |
| Healthy pods harvested per tree in 2021 | 26 |
| Pods infected with witches' broom disease in 2021 | 16 |
| Pods infected with black pod disease in 2021 | 10 |
| Percentage of pods infected with witches' broom disease in 2021 | 2% |
| Percentage of pods infected with black pod disease in 2021 | 2% |
| Dry weight of a single bean in 2021 (g) | 0.96 |
| Total dry bean weight in 2021 (g) | 7677.9 |
| Total dry bean weight per tree in 2021 (g) | 511.9 |
| Yield in 2021 (kg/ha) | 601.4 |
| Clone yield divided by CCN 51 yield in 2021 | 1.7 |
| Accumulated yield (kg/ha) | 1,178.34 |
| Number of dead plants infected with Ceratocystis cacaofunesta | 4 |
| Percentage of seedlings dead after infection with Ceratocystis cacaofunesta | 80% |
| Minimum lesion (mm) | 124.2 |
| Maximum lesion (mm) | 163.2 |
| Average lesion (mm) | 146 |
| Fat content (%) | 42.01714 |

### Variety ‘CMC-014’

In certain aspects, the present invention relates to a new and distinctive cacao plant designated as ‘CMC-014’.

The cacao variety ‘CMC-014’ originated from a controlled cross between varieties ‘CCN-51’ and ‘GU-171C’. The breeder's reference is ‘MC-08006’, and the variety designation is ‘CMC-014’. Cacao variety ‘CMC-014’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Low
Fruit shape: Oblong
Basal constriction on fruit: Present
Shape of fruit apex: Obtuse

The leaf size is medium (201.2 cm²), with 29.9 cm in length and 10.2 cm in width. The base leaf shape is obtuse, and the leaf apex is acuminate. The intensity of green in immature leaves is dark red, whereas the intensity of green in the mature leaves is medium.

The average length and width of the sepals are 7.4 mm (medium) and 2.1 mm (short), respectively, with weak anthocyanin coloration. The ligula color is yellow, and the staminode has weak anthocyanin coloration, while the pedicel has weak anthocyanin coloration.

The average length and width of the pod are 186.3 mm (medium) and 81.7 mm (medium), respectively, with the orange/anthocyanin (ridge) color of the mature pod. The pod surface roughness is moderately rough, with a medium depth of grooves and thick exocarp (e.g. lower and greater exocarps measuring 10.9 and 16.1 mm, respectively). The color of the pulp is dark cream, and the pulp sweetness is high, with BRIX measuring 20.3. The average number of seeds per pod is 24.1.

The seed has an oblong shape in longitudinal section, with an average seed length of 25.7 mm (medium), and a width of 11.9 mm (medium). The seed thickness is 7.9 mm, and the cotyledon has dark red color.

TABLE 14
Variety ‘CMC-014’ Harvest and Infection Data in 2020 and 2021.

| Characteristic | Value |
|---|---|
| Total reps in 2020 | 15 |
| Total pods harvested in 2020 | 235 |
| Total healthy pods harvested in 2020 | 168 |
| Pods harvested per tree in 2020 | 16 |
| Healthy pods harvested per tree in 2020 | 11 |
| Pods infected with witches' broom disease in 2020 | 39 |
| Pods infected with black pod disease in 2020 | 3 |
| Percentage of pods infected with witches' broom disease in 2020 | 16.60% |
| Percentage of pods infected with black pod disease in 2020 | 1.28% |
| Dry weight of a single bean in 2020 (g) | 0.79 |
| Total dry bean weight in 2020 (g) | 4053.172 |
| Total dry bean weight per tree in 2020 (g) | 270.21 |
| Yield in 2020 (kg/ha) | 317.4985 |
| Clone yield divided by CCN 51 yield in 2020 | 2.49 |
| Total reps in 2021 | 13 |
| Total pods harvested in 2021 | 364 |
| Total healthy pods harvested in 2021 | 303 |
| Pods harvested per tree in 2021 | 28 |
| Healthy pods harvested per tree in 2021 | 23 |
| Pods infected with witches' broom disease in 2021 | 6 |
| Pods infected with black pod disease in 2021 | 1 |
| Percentage of pods infected with witches' broom disease in 2021 | 2% |
| Percentage of pods infected with black pod disease in 2021 | 0% |
| Dry weight of a single bean in 2021 (g) | 1.36 |
| Total dry bean weight in 2021 (g) | 9055 |
| Total dry bean weight per tree in 2021 (g) | 696.5 |
| Yield in 2021 (kg/ha) | 818.4 |
| Clone yield divided by CCN 51 yield in 2021 | 2.3 |
| Accumulated yield (kg/ha) | 1,135.93 |
| Number of dead plants infected with Ceratocystis cacaofunesta | 2 |
| Percentage of seedlings dead after infection with Ceratocystis cacaofunesta | 40% |
| Minimum lesion (mm) | 113.7 |
| Maximum lesion (mm) | 133.4 |
| Average lesion (mm) | 123.5 |
| Fat content (%) | NA |

### Variety ‘CMC-015’

In certain aspects, the present invention relates to a new and distinctive cacao plant designated as ‘CMC-015’.

The cacao variety ‘CMC-015’ originated from a controlled cross between varieties ‘MOQ-4.25’ and ‘MC-08017’. The breeder's reference is ‘PT-08-041-1-037-17’, and the variety designation is ‘CMC-015’. Cacao variety ‘CM15’ has subsequently been asexually propagated via grafting onto open-pollinated ‘VB-1151’ rootstock.

Characteristics of this variety are provided below:
Young flush leaf color: Brown
Axil spot on petiole: Absent
Intensity of anthocyanin on sepal: Low
Fruit shape: Elliptic
Basal constriction on fruit: Present
Shape of fruit apex: Caudate

The leaf size is medium (221.9 cm²), with 28.9 cm in length and 11.6 cm in width. The base leaf shape is obtuse, and the leaf apex is apiculate. The intensity of green in immature leaves is brown, whereas the intensity of green in the mature leaves is medium.

The average length and width of the sepals are 7.3 mm (medium) and 2.7 mm (medium), respectively, with weak anthocyanin coloration. The ligula color is yellow, and the staminode has medium anthocyanin coloration, while the pedicel has weak anthocyanin coloration.

The average length and width of the pod are 222.0 mm (long) and 86.6 mm (medium), respectively, with the orange/anthocyanin (ridge) color of the mature pod. The pod surface roughness is very rough, with a medium depth of grooves and thick exocarp (e.g. lower and greater exocarps measuring 10.4 and 15.7 mm, respectively). The color of the pulp is dark cream, and the pulp sweetness is high, with BRIX measuring 17.1. The average number of seeds per pod is 16.3.

The seed has an elliptic shape in longitudinal section, with an average seed length of 25.2 mm (medium), and a width of 14.4 mm (medium). The seed thickness is 9.6 mm, and the cotyledon has dark red color.

TABLE 15
Variety ‘CMC-015’ Harvest and Infection Data in 2020 and 2021.

| Characteristic | Value |
|---|---|
| Total reps in 2020 | 15 |
| Total pods harvested in 2020 | 294 |
| Total healthy pods harvested in 2020 | 193 |
| Pods harvested per tree in 2020 | 20 |
| Healthy pods harvested per tree in 2020 | 13 |
| Pods infected with witches' broom disease in 2020 | 27 |
| Pods infected with black pod disease in 2020 | 0 |
| Percentage of pods infected with witches' broom disease in 2020 | 9.18% |
| Percentage of pods infected with black pod disease in 2020 | 0.00% |
| Dry weight of a single bean in 2020 (g) | 1.01 |
| Total dry bean weight in 2020 (g) | 4291.414 |
| Total dry bean weight per tree in 2020 (g) | 286.09 |
| Yield in 2020 (kg/ha) | 336.1608 |
| Clone yield divided by CCN 51 yield in 2020 | 2.64 |
| Total reps in 2021 | 15 |
| Total pods harvested in 2021 | 532 |
| Total healthy pods harvested in 2021 | 446 |
| Pods harvested per tree in 2021 | 35 |
| Healthy pods harvested per tree in 2021 | 30 |
| Pods infected with witches' broom disease in 2021 | 11 |
| Pods infected with black pod disease in 2021 | 2 |
| Percentage of pods infected with witches' broom disease in 2021 | 2% |
| Percentage of pods infected with black pod disease in 2021 | 0% |
| Dry weight of a single bean in 2021 (g) | 1.20 |
| Total dry bean weight in 2021 (g) | 10127 |
| Total dry bean weight per tree in 2021 (g) | 675.1 |
| Yield in 2021 (kg/ha) | 793.3 |
| Clone yield divided by CCN 51 yield in 2021 | 2.2 |
| Accumulated yield (kg/ha) | 1,129.44 |
| Number of dead plants infected with Ceratocystis cacaofunesta | 2 |
| Percentage of seedlings dead after infection with Ceratocystis cacaofunesta | 40% |
| Minimum lesion (mm) | 90.6 |
| Maximum lesion (mm) | 128.1 |
| Average lesion (mm) | 104.4 |
| Fat content (%) | NA |

### Variety ‘CMC-016’

In certain aspects, the present invention relates to a new and distinctive cacao plant designated as ‘CMC-016’.

The cacao variety ‘CMC-016’ originated from a controlled cross between varieties ‘IPIRANGA-1’ and ‘MC-08114’. The breeder's reference is ‘PT-08-034-3-154-07’, and the variety designation is ‘CMC-016’. Cacao variety ‘CMC-016’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Acute

The leaf size is medium (159.4 cm²), with 26.4 cm in length and 9.1 cm in width. The base leaf shape is obtuse, and the leaf apex is apiculate. The intensity of green in immature leaves is medium red, whereas the intensity of green in the mature leaves is medium.

The average length and width of the sepals are 7.7 mm (medium) and 2.4 mm (medium), respectively, with absent anthocyanin coloration. The ligula color is cream yellow, and the staminode has medium anthocyanin coloration, while the pedicel has absent anthocyanin coloration.

The average length and width of the pod are 151.6 mm (medium) and 71.8 mm (short), respectively, with the yellow color of the mature pod. The pod surface roughness is smooth or slightly rough, with a medium depth of grooves and medium exocarp (e.g. lower and greater exocarps measuring 7.8 and 12.6 mm, respectively). The color of the pulp is light cream, and the pulp sweetness is low, with BRIX measuring 13.1. The average number of seeds per pod is 19.6.

The seed has an irregular shape in longitudinal section, with an average seed length of 20.6 mm (short), and a width of 12.6 mm (medium). The seed thickness is 7.5 mm, and the cotyledon has pink color.

TABLE 16
Variety ‘CMC-016’ Harvest and Infection Data in 2020 and 2021.

| Characteristic | Value |
|---|---|
| Total reps in 2020 | 12 |
| Total pods harvested in 2020 | 297 |
| Total healthy pods harvested in 2020 | 201 |
| Pods harvested per tree in 2020 | 25 |
| Healthy pods harvested per tree in 2020 | 17 |
| Pods infected with witches' broom disease in 2020 | 24 |
| Pods infected with black pod disease in 2020 | 1 |
| Percentage of pods infected with witches' broom disease in 2020 | 8.08% |
| Percentage of pods infected with black pod disease in 2020 | 0.34% |
| Dry weight of a single bean in 2020 (g) | 0.72 |
| Total dry bean weight in 2020 (g) | 3893.067 |
| Total dry bean weight per tree in 2020 (g) | 324.42 |
| Yield in 2020 (kg/ha) | 381.1962 |
| Clone yield divided by CCN 51 yield in 2020 | 2.99 |
| Total reps in 2021 | 11 |
| Total pods harvested in 2021 | 396 |
| Total healthy pods harvested in 2021 | 296 |
| Pods harvested per tree in 2021 | 36 |
| Healthy pods harvested per tree in 2021 | 27 |
| Pods infected with witches' broom disease in 2021 | 7 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 1.12 |
| Total dry bean weight in 2021 (g) | 6834.5 |
| Total dry bean weight per tree in 2021 (g) | 621.3 |
| Yield in 2021 (kg/ha) | 730.0 |
| Clone yield divided by CCN 51 yield in 2021 | 2.0 |
| Accumulated yield (kg/ha) | 1,111.25 |
| Number of dead plants infected with Ceratocystis cacaofunesta | 4 |
| Percentage of seedlings dead after infection with Ceratocystis cacaofunesta | 80% |
| Minimum lesion (mm) | 99.9 |
| Maximum lesion (mm) | 236.4 |
| Average lesion (mm) | 164.7 |
| Fat content (%) | 36.445 |

### Variety ‘CMC-017’

In certain aspects, the present invention relates to a new and distinctive cacao plant designated as ‘CMC-017’.

The cacao variety ‘CMC-017’ originated from a controlled cross between varieties ‘MC-08005’ and ‘MC-08060’ (‘CMC-029’). The breeder's reference is ‘PT-08-038-3-096-06’, and the variety designation is ‘CMC-017’. Cacao variety ‘CMC-017’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Obtuse

The leaf size is medium (170.9 cm²), with 27.4 cm in length and 9.5 cm in width. The base leaf shape is obtuse, and the leaf apex is apiculate. The intensity of green in immature leaves is medium red, whereas the intensity of green in the mature leaves is medium.

The average length and width of the sepals are 7.5 mm (medium) and 2.3 mm (medium), respectively, with absent anthocyanin coloration. The ligula color is yellow, and the staminode has medium anthocyanin coloration, while the pedicel has weak anthocyanin coloration.

The average length and width of the pod are 152.5 mm (medium) and 69.2 mm (short), respectively, with the yellow color of the mature pod. The pod surface roughness is moderately rough, with a deep depth of grooves and thick exocarp (e.g. lower and greater exocarps measuring 8.9 and 15.6 mm, respectively). The color of the pulp is light cream, and the pulp sweetness is high, with BRIX measuring 18.9. The average number of seeds per pod is 14.5.

The seed has an irregular shape in longitudinal section, with an average seed length of 23.9 mm (short), and a width of 11.8 mm (medium). The seed thickness is 7.9 mm, and the cotyledon has dark purple color.

TABLE 17
Variety ‘CMC-017’ Harvest and Infection Data in 2020 and 2021.

| Characteristic | Value |
|---|---|
| Total reps in 2020 | 12 |
| Total pods harvested in 2020 | 718 |
| Total healthy pods harvested in 2020 | 429 |
| Pods harvested per tree in 2020 | 60 |
| Healthy pods harvested per tree in 2020 | 36 |
| Pods infected with witches' broom disease in 2020 | 19 |
| Pods infected with black pod disease in 2020 | 2 |
| Percentage of pods infected with witches' broom disease in 2020 | 2.65% |
| Percentage of pods infected with black pod disease in 2020 | 0.28% |
| Dry weight of a single bean in 2020 (g) | 0.56 |
| Total dry bean weight in 2020 (g) | 6037.46 |
| Total dry bean weight per tree in 2020 (g) | 503.12 |
| Yield in 2020 (kg/ha) | 591.168 |
| Clone yield divided by CCN 51 yield in 2020 | 4.63 |
| Total reps in 2021 | 12 |
| Total pods harvested in 2021 | 463 |
| Total healthy pods harvested in 2021 | 368 |
| Pods harvested per tree in 2021 | 39 |
| Healthy pods harvested per tree in 2021 | 31 |
| Pods infected with witches' broom disease in 2021 | 4 |
| Pods infected with black pod disease in 2021 | 0 |
| Percentage of pods infected with witches' broom disease in 2021 | 1% |
| Percentage of pods infected with black pod disease in 2021 | 0% |
| Dry weight of a single bean in 2021 (g) | 0.92 |
| Total dry bean weight in 2021 (g) | 5175.8 |
| Total dry bean weight per tree in 2021 (g) | 431.3 |
| Yield in 2021 (kg/ha) | 506.8 |
| Clone yield divided by CCN 51 yield in 2021 | 1.4 |
| Accumulated yield (kg/ha) | 1,097.97 |
| Number of dead plants infected with Ceratocystis cacaofunesta | 4 |
| Percentage of seedlings dead after infection with Ceratocystis cacaofunesta | 80% |
| Minimum lesion (mm) | 88.1 |
| Maximum lesion (mm) | 186.5 |
| Average lesion (mm) | 144.9 |
| Fat content (%) | NA |

### Variety ‘CMC-018’

In certain aspects, the present invention relates to a new and distinctive cacao plant designated as ‘CMC-018’.

The cacao variety ‘CMC-018’ originated from a controlled cross between varieties ‘PH-111’ and ‘GU-144C’. The breeder's reference is ‘PT-08-057-2-173-09’, and the variety designation is ‘CMC-018’. Cacao variety ‘CMC-018’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (174.4 cm²), with 28.7 cm in length and 9.2 cm in width. The base leaf shape is obtuse, and the leaf apex is acuminate. The intensity of green in immature leaves is dark red, whereas the intensity of green in the mature leaves is medium.

The average length and width of the sepals are 6.2 mm (short) and 1.9 mm (short), respectively, with absent anthocyanin coloration. The ligula color is cream yellow, and the staminode has medium anthocyanin coloration, while the pedicel has weak anthocyanin coloration.

The average length and width of the pod are 149.4 mm (short) and 77.0 mm (medium), respectively, with the orange/anthocyanin (ridge) color of the mature pod. The pod surface roughness is smooth or slightly rough, with an absent or very shallow depth of grooves and medium exocarp (e.g. lower and greater exocarps measuring 10.7 and 13.7 mm, respectively). The color of the pulp is dark cream, and the pulp sweetness is high, with BRIX measuring 17.7. The average number of seeds per pod is 14.2.

The seed has an elliptic shape in longitudinal section, with an average seed length of 27.7 mm (medium), and a width of 14.7 mm (medium). The seed thickness is 9.5 mm, and the cotyledon has dark red color.

TABLE 18
Variety ‘CMC-018’ Harvest and Infection Data in 2020 and 2021.

| Characteristic | Value |
|---|---|
| Total reps in 2020 | 15 |
| Total pods harvested in 2020 | 490 |
| Total healthy pods harvested in 2020 | 310 |
| Pods harvested per tree in 2020 | 33 |
| Healthy pods harvested per tree in 2020 | 21 |
| Pods infected with witches' broom disease in 2020 | 34 |
| Pods infected with black pod disease in 2020 | 4 |
| Percentage of pods infected with witches' broom disease in 2020 | 6.94% |
| Percentage of pods infected with black pod disease in 2020 | 0.82% |
| Dry weight of a single bean in 2020 (g) | 0.85 |
| Total dry bean weight in 2020 (g) | 6142.235 |
| Total dry bean weight per tree in 2020 (g) | 409.48 |
| Yield in 2020 (kg/ha) | 481.1417 |
| Clone yield divided by CCN 51 yield in 2020 | 3.77 |
| Total reps in 2021 | 14 |
| Total pods harvested in 2021 | 552 |
| Total healthy pods harvested in 2021 | 456 |
| Pods harvested per tree in 2021 | 39 |
| Healthy pods harvested per tree in 2021 | 33 |
| Pods infected with witches' broom disease in 2021 | 10 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 1.17 |
| Total dry bean weight in 2021 (g) | 7155.7 |
| Total dry bean weight per tree in 2021 (g) | 511.1 |
| Yield in 2021 (kg/ha) | 600.6 |
| Clone yield divided by CCN 51 yield in 2021 | 1.7 |
| Accumulated yield (kg/ha) | 1,081.71 |
| Number of dead plants infected with Ceratocystis cacaofunesta | 5 |
| Percentage of seedlings dead after infection with Ceratocystis cacaofunesta | 100% |
| Minimum lesion (mm) | 99.4 |
| Maximum lesion (mm) | 137.9 |
| Average lesion (mm) | 126.1 |
| Fat content (%) | 40.46 |

### Variety ‘CMC-019’

In certain aspects, the present invention relates to a new and distinctive cacao plant designated as ‘CMC-019’.

The cacao variety ‘CMC-019’ originated from a controlled cross between varieties ‘GU-144C’ and ‘MC-08091’. The breeder's reference is ‘PT-08-020-2-057-12’, and the variety designation is ‘CMC-019’. Cacao variety ‘CMC-019’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Obtuse

The leaf size is medium (213.9 cm²), with 30.7 cm in length and 10.6 cm in width. The base leaf shape is obtuse, and the leaf apex is apiculate. The intensity of green in immature leaves is medium red, whereas the intensity of green in the mature leaves is medium.

The average length and width of the sepals are 7.2 mm (medium) and 2.1 mm (medium), respectively, with absent anthocyanin coloration. The ligula color is yellow, and the staminode has strong anthocyanin coloration, while the pedicel has weak anthocyanin coloration.

The average length and width of the pod are 168.7 mm (medium) and 84.4 mm (medium), respectively, with the yellow color of the mature pod. The pod surface roughness is smooth or slightly rough, with a medium depth of grooves and thick exocarp (e.g. lower and greater exocarps measuring 12.2 and 17.9 mm, respectively). The color of the pulp is dark cream, and the pulp sweetness is high, with BRIX measuring 17.4. The average number of seeds per pod is 24.0.

The seed has an elliptic shape in longitudinal section, with an average seed length of 24.1 mm (short), and a width of 12.5 mm (medium). The seed thickness is 9.4 mm, and the cotyledon has dark purple color.

TABLE 19
Variety ‘CMC-019’ Harvest and Infection Data in 2020 and 2021.

| Characteristic | Value |
|---|---|
| Total reps in 2020 | 12 |
| Total pods harvested in 2020 | 114 |
| Total healthy pods harvested in 2020 | 74 |
| Pods harvested per tree in 2020 | 10 |
| Healthy pods harvested per tree in 2020 | 6 |
| Pods infected with witches' broom disease in 2020 | 6 |
| Pods infected with black pod disease in 2020 | 2 |
| Percentage of pods infected with witches' broom disease in 2020 | 5.26% |
| Percentage of pods infected with black pod disease in 2020 | 1.75% |
| Dry weight of a single bean in 2020 (g) | 0.65 |
| Total dry bean weight in 2020 (g) | 1380.046 |
| Total dry bean weight per tree in 2020 (g) | 115.00 |
| Yield in 2020 (kg/ha) | 135.1295 |
| Clone yield divided by CCN 51 yield in 2020 | 1.06 |
| Total reps in 2021 | 12 |
| Total pods harvested in 2021 | 389 |
| Total healthy pods harvested in 2021 | 350 |
| Pods harvested per tree in 2021 | 32 |
| Healthy pods harvested per tree in 2021 | 29 |
| Pods infected with witches' broom disease in 2021 | 8 |
| Pods infected with black pod disease in 2021 | 7 |
| Percentage of pods infected with witches' broom disease in 2021 | 2% |
| Percentage of pods infected with black pod disease in 2021 | 2% |
| Dry weight of a single bean in 2021 (g) | 0.65 |
| Total dry bean weight in 2021 (g) | 9646.4 |
| Total dry bean weight per tree in 2021 (g) | 803.9 |
| Yield in 2021 (kg/ha) | 944.5 |
| Clone yield divided by CCN 51 yield in 2021 | 2.6 |
| Accumulated yield (kg/ha) | 1,079.67 |
| Number of dead plants infected with Ceratocystis cacaofunesta | 4 |
| Percentage of seedlings dead after infection with Ceratocystis cacaofunesta | 80% |
| Minimum lesion (mm) | 84.3 |
| Maximum lesion (mm) | 183 |
| Average lesion (mm) | 139.3 |
| Fat content (%) | NA |

### Variety ‘CMC-020’

In certain aspects, the present invention relates to a new and distinctive cacao plant designated as ‘CMC-020’.

The cacao variety ‘CMC-020’ originated from a controlled cross between varieties ‘NA-33’ and ‘GU-144C’. The breeder's reference is ‘PT-08-045-2-172-10’, and the variety designation is ‘CMC-020’. Cacao variety ‘CMC-020’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Elliptic
Basal constriction on fruit: Present
Shape of fruit apex: Indented

The leaf size is medium (181.0 cm²), with 27.7 cm in length and 9.9 cm in width. The base leaf shape is obtuse, and the leaf apex is acuminate. The intensity of green in immature leaves is medium red, whereas the intensity of green in the mature leaves is medium.

The average length and width of the sepals are 6.5 mm (medium) and 2.3 mm (medium), respectively, with absent anthocyanin coloration. The ligula color is yellow, and the staminode has medium anthocyanin coloration, while the pedicel has absent anthocyanin coloration.

The average length and width of the pod are 134.2 mm (short) and 78.3 mm (medium), respectively, with the yellow color of the mature pod. The pod surface roughness is moderately rough, with a medium depth of grooves and medium exocarp (e.g. lower and greater exocarps measuring 8.6 and 12.7 mm, respectively). The color of the pulp is light cream, and the pulp sweetness is high, with BRIX measuring 17.2. The average number of seeds per pod is 14.0.

The seed has an elliptic shape in longitudinal section, with an average seed length of 26.6 mm (medium), and a width of 14.3 mm (medium). The seed thickness is 8.2 mm, and the cotyledon has dark red color.

TABLE 20
Variety ‘CMC-020’ Harvest and Infection Data in 2020 and 2021.

| Characteristic | Value |
|---|---|
| Total reps in 2020 | 15 |
| Total pods harvested in 2020 | 286 |
| Total healthy pods harvested in 2020 | 200 |
| Pods harvested per tree in 2020 | 19 |
| Healthy pods harvested per tree in 2020 | 13 |
| Pods infected with witches' broom disease in 2020 | 59 |
| Pods infected with black pod disease in 2020 | 2 |
| Percentage of pods infected with witches' broom disease in 2020 | 20.63% |
| Percentage of pods infected with black pod disease in 2020 | 0.70% |
| Dry weight of a single bean in 2020 (g) | 0.87 |
| Total dry bean weight in 2020 (g) | 3948.464 |
| Total dry bean weight per tree in 2020 (g) | 263.23 |
| Yield in 2020 (kg/ha) | 309.2964 |
| Clone yield divided by CCN 51 yield in 2020 | 2.42 |
| Total reps in 2021 | 15 |
| Total pods harvested in 2021 | 509 |
| Total healthy pods harvested in 2021 | 452 |
| Pods harvested per tree in 2021 | 34 |
| Healthy pods harvested per tree in 2021 | 30 |
| Pods infected with witches' broom disease in 2021 | 8 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 1.29 |
| Total dry bean weight in 2021 (g) | 9371.4 |
| Total dry bean weight per tree in 2021 (g) | 624.8 |
| Yield in 2021 (kg/ha) | 734.1 |
| Clone yield divided by CCN 51 yield in 2021 | 2.0 |
| Accumulated yield (kg/ha) | 1,043.39 |
| Number of dead plants infected with Ceratocystis cacaofunesta | 5 |
| Percentage of seedlings dead after infection with Ceratocystis cacaofunesta | 100% |
| Minimum lesion (mm) | 60.2 |
| Maximum lesion (mm) | 162.2 |
| Average lesion (mm) | 116.6 |
| Fat content (%) | NA |

### Variety ‘CMC-021’

In certain aspects, the present invention relates to a new and distinctive cacao plant designated as ‘CMC-021’.

The cacao variety ‘CMC-021’ originated from a controlled cross between varieties ‘GU-144C’ and ‘MC-08024’. The breeder's reference is ‘PT-08-019-3-164-18’, and the variety designation is ‘CMC-021’. Cacao variety ‘CMC-021’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Low
Fruit shape: Oblong
Basal constriction on fruit: Present
Shape of fruit apex: Obtuse

The leaf size is medium (184.8 cm²), with 27.9 cm in length and 10.0 cm in width. The base leaf shape is obtuse, and the leaf apex is apiculate. The intensity of green in immature leaves is dark red, whereas the intensity of green in the mature leaves is medium.

The average length and width of the sepals are 7.6 mm (medium) and 2.4 mm (medium), respectively, with weak anthocyanin coloration. The ligula color is yellow, and the staminode has medium anthocyanin coloration, while the pedicel has moderate anthocyanin coloration.

The average length and width of the pod are 182.6 mm (medium) and 79.1 mm (medium), respectively, with the orange/anthocyanin (ridge) color of the mature pod. The pod surface roughness is moderately rough, with a medium depth of grooves and thick exocarp (e.g. lower and greater exocarps measuring 11.8 and 15.7 mm, respectively). The color of the pulp is dark cream, and the pulp sweetness is low, with BRIX measuring 11.6. The average number of seeds per pod is 18.4.

The seed has an irregular shape in longitudinal section, with an average seed length of 22.8 mm (short), and a width of 12.8 mm (medium). The seed thickness is 8.1 mm, and the cotyledon has dark purple color.

TABLE 21
Variety ‘CMC-021’ Harvest and Infection Data in 2020 and 2021.

| Characteristic | Value |
|---|---|
| Total reps in 2020 | 12 |
| Total pods harvested in 2020 | 336 |
| Total healthy pods harvested in 2020 | 237 |
| Pods harvested per tree in 2020 | 28 |
| Healthy pods harvested per tree in 2020 | 20 |
| Pods infected with witches' broom disease in 2020 | 28 |
| Pods infected with black pod disease in 2020 | 20 |
| Percentage of pods infected with witches' broom disease in 2020 | 8.33% |
| Percentage of pods infected with black pod disease in 2020 | 5.95% |
| Dry weight of a single bean in 2020 (g) | 0.67 |
| Total dry bean weight in 2020 (g) | 4492.967 |
| Total dry bean weight per tree in 2020 (g) | 374.41 |
| Yield in 2020 (kg/ha) | 439.9363 |
| Clone yield divided by CCN 51 yield in 2020 | 3.45 |
| Total reps in 2021 | 12 |
| Total pods harvested in 2021 | 363 |
| Total healthy pods harvested in 2021 | 269 |
| Pods harvested per tree in 2021 | 30 |
| Healthy pods harvested per tree in 2021 | 22 |
| Pods infected with witches' broom disease in 2021 | 7 |
| Pods infected with black pod disease in 2021 | 1 |
| Percentage of pods infected with witches' broom disease in 2021 | 2% |
| Percentage of pods infected with black pod disease in 2021 | 0% |
| Dry weight of a single bean in 2021 (g) | 1.12 |
| Total dry bean weight in 2021 (g) | 6128.7 |
| Total dry bean weight per tree in 2021 (g) | 510.7 |
| Yield in 2021 (kg/ha) | 600.1 |
| Clone yield divided by CCN 51 yield in 2021 | 1.7 |
| Accumulated yield (kg/ha) | 1,040.04 |
| Number of dead plants infected with Ceratocystis cacaofunesta | 5 |
| Percentage of seedlings dead after infection with Ceratocystis cacaofunesta | 100% |
| Minimum lesion (mm) | 37.1 |
| Maximum lesion (mm) | 112.9 |
| Average lesion (mm) | 92.4 |
| Fat content (%) | 40.14 |

### Variety ‘CMC-023’

In certain aspects, the present invention relates to a new and distinctive cacao plant designated as ‘CMC-023’.

The cacao variety ‘CMC-023’ originated from a controlled cross between varieties ‘MC-08005’ and ‘MC-08060’ (‘CMC-029’). The breeder's reference is ‘PT-08-038-2-050-11’, and the variety designation is ‘CMC-023’. Cacao variety ‘CMC-023’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Obtuse

The leaf size is medium (194.3 cm²), with 30.2 cm in length and 9.7 cm in width. The base leaf shape is obtuse, and the leaf apex is acuminate. The intensity of green in immature leaves is medium red, whereas the intensity of green in the mature leaves is medium.

The average length and width of the sepals are 8.5 mm (medium) and 2.3 mm (medium), respectively, with absent anthocyanin coloration. The ligula color is yellow, and the staminode has weak anthocyanin coloration, while the pedicel has absent anthocyanin coloration.

The average length and width of the pod are 201.1 mm (long) and 76.8 mm (medium), respectively, with the yellow color of the mature pod. The pod surface roughness is moderately rough, with a deep depth of grooves and medium exocarp (e.g. lower and greater exocarps measuring 9.0 and 14.9 mm, respectively). The color of the pulp is dark cream, and the pulp sweetness is high, with BRIX measuring 18.2. The average number of seeds per pod is 22.8.

The seed has an oblong shape in longitudinal section, with an average seed length of 25.2 mm (short), and a width of 10.2 mm (medium). The seed thickness is 7.9 mm, and the cotyledon has pink color.

TABLE 22
Variety ‘CMC-023’ Harvest and Infection Data in 2020 and 2021.

| Characteristic | Value |
|---|---|
| Total reps in 2020 | 12 |
| Total pods harvested in 2020 | 90 |
| Total healthy pods harvested in 2020 | 63 |
| Pods harvested per tree in 2020 | 8 |
| Healthy pods harvested per tree in 2020 | 5 |
| Pods infected with witches' broom disease in 2020 | 9 |
| Pods infected with black pod disease in 2020 | 1 |
| Percentage of pods infected with witches' broom disease in 2020 | 10.00% |
| Percentage of pods infected with black pod disease in 2020 | 1.11% |
| Dry weight of a single bean in 2020 (g) | 0.86 |
| Total dry bean weight in 2020 (g) | 1723.102 |
| Total dry bean weight per tree in 2020 (g) | 143.59 |
| Yield in 2020 (kg/ha) | 168.7204 |
| Clone yield divided by CCN 51 yield in 2020 | 1.32 |
| Total reps in 2021 | 12 |
| Total pods harvested in 2021 | 494 |
| Total healthy pods harvested in 2021 | 390 |
| Pods harvested per tree in 2021 | 41 |
| Healthy pods harvested per tree in 2021 | 33 |
| Pods infected with witches' broom disease in 2021 | 12 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 0.96 |
| Total dry bean weight in 2021 (g) | 8868.6 |
| Total dry bean weight per tree in 2021 (g) | 739.1 |
| Yield in 2021 (kg/ha) | 868.4 |
| Clone yield divided by CCN 51 yield in 2021 | 2.4 |
| Accumulated yield (kg/ha) | 1,037.10 |
| Number of dead plants infected with Ceratocystis cacaofunesta | 4 |
| Percentage of seedlings dead after infection with Ceratocystis cacaofunesta | 80% |
| Minimum lesion (mm) | 115.5 |
| Maximum lesion (mm) | 256.8 |
| Average lesion (mm) | 173.9 |
| Fat content (%) | NA |

### Variety ‘CMC-024’

In certain aspects, the present invention relates to a new and distinctive cacao plant designated as ‘CMC-024’.

The cacao variety ‘CMC-024’ originated from a controlled cross between varieties ‘GU-144C’ and ‘MC-08091’. The breeder's reference is ‘PT-08-020-2-057-16’, and the variety designation is ‘CMC-024’. Cacao variety ‘CMC-024’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (170.8 cm²), with 27.9 cm in length and 9.3 cm in width. The base leaf shape is obtuse, and the leaf apex is apiculate. The intensity of green in immature leaves is medium red, whereas the intensity of green in the mature leaves is medium.

The average length and width of the sepals are 7.9 mm (medium) and 2.0 mm (short), respectively, with absent anthocyanin coloration. The ligula color is yellow, and the staminode has medium anthocyanin coloration, while the pedicel has absent anthocyanin coloration.

The average length and width of the pod are 198.8 mm (medium) and 85.9 mm (medium), respectively, with the yellow color of the mature pod. The pod surface roughness is moderately rough, with a medium depth of grooves and thick exocarp (e.g. lower and greater exocarps measuring 11.4 and 18.1 mm, respectively). The color of the pulp is dark cream, and the pulp sweetness is medium, with BRIX measuring 17. The average number of seeds per pod is 26.2.

The seed has an elliptic shape in longitudinal section, with an average seed length of 27.1 mm (medium), and a width of 14.2 mm (medium). The seed thickness is 10.4 mm, and the cotyledon has dark red color.

TABLE 23
Variety ‘CMC-024’ Harvest and Infection Data in 2020 and 2021.

| Characteristic | Value |
|---|---|
| Total reps in 2020 | 12 |
| Total pods harvested in 2020 | 53 |
| Total healthy pods harvested in 2020 | 26 |
| Pods harvested per tree in 2020 | 4 |
| Healthy pods harvested per tree in 2020 | 2 |
| Pods infected with witches' broom disease in 2020 | 6 |
| Pods infected with black pod disease in 2020 | 8 |
| Percentage of pods infected with witches' broom disease in 2020 | 11.32% |
| Percentage of pods infected with black pod disease in 2020 | 15.09% |
| Dry weight of a single bean in 2020 (g) | 0.83 |
| Total dry bean weight in 2020 (g) | 578.0616 |
| Total dry bean weight per tree in 2020 (g) | 48.17 |
| Yield in 2020 (kg/ha) | 56.60186 |
| Clone yield divided by CCN 51 yield in 2020 | 0.44 |
| Total reps in 2021 | 12 |
| Total pods harvested in 2021 | 460 |
| Total healthy pods harvested in 2021 | 379 |
| Pods harvested per tree in 2021 | 38 |
| Healthy pods harvested per tree in 2021 | 32 |
| Pods infected with witches' broom disease in 2021 | 2 |
| Pods infected with black pod disease in 2021 | 11 |
| Percentage of pods infected with witches' broom disease in 2021 | 0% |
| Percentage of pods infected with black pod disease in 2021 | 2% |
| Dry weight of a single bean in 2021 (g) | 1.32 |
| Total dry bean weight in 2021 (g) | 9779 |
| Total dry bean weight per tree in 2021 (g) | 814.9 |
| Yield in 2021 (kg/ha) | 957.5 |
| Clone yield divided by CCN 51 yield in 2021 | 2.7 |
| Accumulated yield (kg/ha) | 1,014.13 |
| Number of dead plants infected with Ceratocystis cacaofunesta | 5 |
| Percentage of seedlings dead after infection with Ceratocystis cacaofunesta | 100% |
| Minimum lesion (mm) | 103.8 |
| Maximum lesion (mm) | 160.5 |
| Average lesion (mm) | 126.8 |
| Fat content (%) | NA |

### Variety ‘CMC-025’

In certain aspects, the present invention relates to a new and distinctive cacao plant designated as ‘CMC-025’.

The cacao variety ‘CMC-025’ originated from a controlled cross between varieties ‘MC-08005’ and ‘MC-08060’ (‘CMC-029’). The breeder's reference is ‘PT-08-038-3-096-02’, and the variety designation is ‘CMC-025’. Cacao variety ‘CMC-025’ has subsequently been asexually propagated via grafting onto open-pollinated ‘VB-1151’ rootstock.

Characteristics of this variety are provided below:
Young flush leaf color: Light red
Axil spot on petiole: Absent
Intensity of anthocyanin on sepal: Absent or very low
Fruit shape: Oblong
Basal constriction on fruit: Present
Shape of fruit apex: Acute

The leaf size is medium (197.3 cm²), with 28.9 cm in length and 10.3 cm in width. The base leaf shape is obtuse, and the leaf apex is apiculate. The intensity of green in immature leaves is light red, whereas the intensity of green in the mature leaves is medium.

The average length and width of the sepals are 6.5 mm (medium) and 2.1 mm (short), respectively, with absent anthocyanin coloration. The ligula color is yellow, and the staminode has weak anthocyanin coloration, while the pedicel has weak anthocyanin coloration.

The average length and width of the pod are 202.4 mm (long) and 87.2 mm (medium), respectively, with the yellow color of the mature pod. The pod surface roughness is smooth or slightly rough, with a medium depth of grooves and thick exocarp (e.g. lower and greater exocarps measuring 10.7 and 15.2 mm, respectively). The color of the pulp is light cream, and the pulp sweetness is medium, with BRIX measuring 16.5. The average number of seeds per pod is 21.9.

The seed has an oblong shape in longitudinal section, with an average seed length of 26.7 mm (medium), and a width of 11.7 mm (medium). The seed thickness is 8.7 mm, and the cotyledon has dark purple color.

TABLE 24
Variety ‘CMC-025’ Harvest and Infection Data in 2020 and 2021.

| Characteristic | Value |
|---|---|
| Total reps in 2020 | 15 |
| Total pods harvested in 2020 | 189 |
| Total healthy pods harvested in 2020 | 117 |
| Pods harvested per tree in 2020 | 13 |
| Healthy pods harvested per tree in 2020 | 8 |
| Pods infected with witches' broom disease in 2020 | 5 |
| Pods infected with black pod disease in 2020 | 3 |
| Percentage of pods infected with witches' broom disease in 2020 | 2.65% |
| Percentage of pods infected with black pod disease in 2020 | 1.59% |
| Dry weight of a single bean in 2020 (g) | 0.73 |
| Total dry bean weight in 2020 (g) | 2719.86 |
| Total dry bean weight per tree in 2020 (g) | 181.32 |
| Yield in 2020 (kg/ha) | 213.0557 |
| Clone yield divided by CCN 51 yield in 2020 | 1.67 |
| Total reps in 2021 | 15 |
| Total pods harvested in 2021 | 514 |
| Total healthy pods harvested in 2021 | 381 |
| Pods harvested per tree in 2021 | 34 |
| Healthy pods harvested per tree in 2021 | 25 |
| Pods infected with witches' broom disease in 2021 | 12 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 1.17 |
| Total dry bean weight in 2021 (g) | 10194.4 |
| Total dry bean weight per tree in 2021 (g) | 679.6 |
| Yield in 2021 (kg/ha) | 798.6 |
| Clone yield divided by CCN 51 yield in 2021 | 2.2 |
| Accumulated yield (kg/ha) | 1,011.62 |
| Number of dead plants infected with Ceratocystis cacaofunesta | 3 |
| Percentage of seedlings dead after infection with Ceratocystis cacaofunesta | 60% |
| Minimum lesion (mm) | 139.7 |
| Maximum lesion (mm) | 189.3 |
| Average lesion (mm) | 163.4 |
| Fat content (%) | NA |

### Variety ‘CMC-026’

In certain aspects, the present invention relates to a new and distinctive cacao plant designated as ‘CMC-026’.

The cacao variety ‘CMC-026’ originated from a controlled cross between varieties ‘GU-144C’ and ‘MC-08017’. The breeder's reference is ‘PT-08-018-2-084-08’, and the variety designation is ‘CMC-026’. Cacao variety ‘CMC-026’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (164.3 cm²), with 26.1 cm in length and 9.5 cm in width. The base leaf shape is obtuse, and the leaf apex is apiculate. The intensity of green in immature leaves is medium red, whereas the intensity of green in the mature leaves is medium.

The average length and width of the sepals are 7.6 mm (medium) and 2.0 mm (medium), respectively, with absent anthocyanin coloration. The ligula color is cream yellow, and the staminode has medium anthocyanin coloration, while the pedicel has absent anthocyanin coloration.

The average length and width of the pod are 195.8 mm (medium) and 85.5 mm (medium), respectively, with the yellow color of the mature pod. The pod surface roughness is moderately rough, with a medium depth of grooves and thick exocarp (e.g. lower and greater exocarps measuring 11.8 and 18.7 mm, respectively). The color of the pulp is light cream, and the pulp sweetness is medium, with BRIX measuring 15.8. The average number of seeds per pod is 20.3.

The seed has an oblong shape in longitudinal section, with an average seed length of 27.8 mm (medium), and a width of 12.7 mm (medium). The seed thickness is 9.3 mm, and the cotyledon has dark purple color.

TABLE 25
Variety ‘CMC-026’ Harvest and Infection Data in 2020 and 2021.

| Characteristic | Value |
|---|---|
| Total reps in 2020 | 15 |
| Total pods harvested in 2020 | 206 |
| Total healthy pods harvested in 2020 | 128 |
| Pods harvested per tree in 2020 | 14 |
| Healthy pods harvested per tree in 2020 | 9 |
| Pods infected with witches' broom disease in 2020 | 18 |
| Pods infected with black pod disease in 2020 | 1 |
| Percentage of pods infected with witches' broom disease in 2020 | 8.74% |
| Percentage of pods infected with black pod disease in 2020 | 0.49% |
| Dry weight of a single bean in 2020 (g) | 1.00 |
| Total dry bean weight in 2020 (g) | 2782.76 |
| Total dry bean weight per tree in 2020 (g) | 185.52 |
| Yield in 2020 (kg/ha) | 217.983 |
| Clone yield divided by CCN 51 yield in 2020 | 1.71 |
| Total reps in 2021 | 15 |
| Total pods harvested in 2021 | 465 |
| Total healthy pods harvested in 2021 | 432 |
| Pods harvested per tree in 2021 | 31 |
| Healthy pods harvested per tree in 2021 | 29 |
| Pods infected with witches' broom disease in 2021 | 5 |
| Pods infected with black pod disease in 2021 | 0 |
| Percentage of pods infected with witches' broom disease in 2021 | 1% |
| Percentage of pods infected with black pod disease in 2021 | 0% |
| Dry weight of a single bean in 2021 (g) | 1.23 |
| Total dry bean weight in 2021 (g) | 9709.5 |
| Total dry bean weight per tree in 2021 (g) | 647.3 |
| Yield in 2021 (kg/ha) | 760.6 |
| Clone yield divided by CCN 51 yield in 2021 | 2.1 |
| Accumulated yield (kg/ha) | 978.56 |
| Number of dead plants infected with Ceratocystis cacaofunesta | 3 |
| Percentage of seedlings dead after infection with Ceratocystis cacaofunesta | 60% |
| Minimum lesion (mm) | 51.6 |
| Maximum lesion (mm) | 164.7 |
| Average lesion (mm) | 104.3 |
| Fat content (%) | NA |

### Variety ‘CMC-027’

In certain aspects, the present invention relates to a new and distinctive cacao plant designated as ‘CMC-027’.

The cacao variety ‘CMC-027’ originated from a controlled cross between varieties ‘PS-13.19’ and ‘MC-08017’. The breeder's reference is ‘PT-08-064-1-107-18’, and the variety designation is ‘CMC-027’. Cacao variety ‘CMC-027’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Medium
Fruit shape: Elliptic
Basal constriction on fruit: Present
Shape of fruit apex: Obtuse

The leaf size is medium (187.9 cm²), with 28.4 cm in length and 10.0 cm in width.

The base leaf shape is obtuse, and the leaf apex is apiculate. The intensity of green in immature leaves is dark red, whereas the intensity of green in the mature leaves is medium.

The average length and width of the sepals are 8.0 mm (medium) and 2.3 mm (medium), respectively, with moderate anthocyanin coloration. The ligula color is cream yellow, and the staminode has medium anthocyanin coloration, while the pedicel has strong anthocyanin coloration.

The average length and width of the pod are 160.6 mm (medium) and 84.2 mm (medium), respectively, with the orange/anthocyanin (ridge) color of the mature pod. The pod surface roughness is smooth or slightly rough, with a shallow depth of grooves and thick exocarp (e.g. lower and greater exocarps measuring 11.2 and 16.2 mm, respectively). The color of the pulp is light cream, and the pulp sweetness is medium, with BRIX measuring 16.4. The average number of seeds per pod is 20.2.

The seed has an irregular shape in longitudinal section, with an average seed length of 23.9 mm (medium), and a width of 13.9 mm (medium). The seed thickness is 7.7 mm, and the cotyledon has dark purple color.

TABLE 26
Variety ‘CMC-027’ Harvest and Infection Data in 2020 and 2021.

| Characteristic | Value |
|---|---|
| Total reps in 2020 | 12 |
| Total pods harvested in 2020 | 141 |
| Total healthy pods harvested in 2020 | 101 |
| Pods harvested per tree in 2020 | 12 |
| Healthy pods harvested per tree in 2020 | 8 |
| Pods infected with witches' broom disease in 2020 | 11 |
| Pods infected with black pod disease in 2020 | 0 |
| Percentage of pods infected with witches' broom disease in 2020 | 7.80% |
| Percentage of pods infected with black pod disease in 2020 | 0.00% |
| Dry weight of a single bean in 2020 (g) | 1.05 |
| Total dry bean weight in 2020 (g) | 3262.75 |
| Total dry bean weight per tree in 2020 (g) | 271.90 |
| Yield in 2020 (kg/ha) | 319.478 |
| Clone yield divided by CCN 51 yield in 2020 | 2.50 |
| Total reps in 2021 | 12 |
| Total pods harvested in 2021 | 250 |
| Total healthy pods harvested in 2021 | 207 |
| Pods harvested per tree in 2021 | 21 |
| Healthy pods harvested per tree in 2021 | 17 |
| Pods infected with witches' broom disease in 2021 | 3 |
| Pods infected with black pod disease in 2021 | 0 |
| Percentage of pods infected with witches' broom disease in 2021 | 1% |
| Percentage of pods infected with black pod disease in 2021 | 0% |
| Dry weight of a single bean in 2021 (g) | 1.53 |
| Total dry bean weight in 2021 (g) | 6348.1 |
| Total dry bean weight per tree in 2021 (g) | 529.0 |
| Yield in 2021 (kg/ha) | 621.6 |
| Clone yield divided by CCN 51 yield in 2021 | 1.7 |
| Accumulated yield (kg/ha) | 941.06 |
| Number of dead plants infected with Ceratocystis cacaofunesta | 3 |
| Percentage of seedlings dead after infection with Ceratocystis cacaofunesta | 60% |
| Minimum lesion (mm) | 122.3 |
| Maximum lesion (mm) | 140.3 |
| Average lesion (mm) | 132.8 |
| Fat content (%) | NA |

### Variety ‘CMC-028’

In certain aspects, the present invention relates to a new and distinctive cacao plant designated as ‘CMC-028’.

The cacao variety ‘CMC-028’ originated from a controlled cross between varieties ‘GU-144C’ and ‘MC-08017’. The breeder's reference is ‘PT-08-018-1-034-01’, and the variety designation is ‘CMC-028’. Cacao variety ‘CMC-028’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (173.2 cm²), with 26.9 cm in length and 9.8 cm in width. The base leaf shape is obtuse, and the leaf apex is apiculate. The intensity of green in immature leaves is medium red, whereas the intensity of green in the mature leaves is medium.

The average length and width of the sepals are 6.8 mm (medium) and 2.0 mm (medium), respectively, with absent anthocyanin coloration. The ligula color is yellow, and the staminode has weak anthocyanin coloration, while the pedicel has absent anthocyanin coloration.

The average length and width of the pod are 178.5 mm (medium) and 79.2 mm (medium), respectively, with the yellow color of the mature pod. The pod surface roughness is smooth or slightly rough, with a medium depth of grooves and thick exocarp (e.g. lower and greater exocarps measuring 11.1 and 16.2 mm, respectively). The color of the pulp is light cream, and the pulp sweetness is medium, with BRIX measuring 15.0. The average number of seeds per pod is 15.8.

The seed has an elliptic shape in longitudinal section, with an average seed length of 24.1 mm (short), and a width of 12.7 mm (medium). The seed thickness is 8.4 mm, and the cotyledon has pink color.

TABLE 27
Variety ‘CMC-028’ Harvest and Infection Data in 2020 and 2021.

| Characteristic | Value |
|---|---|
| Total reps in 2020 | 15 |
| Total pods harvested in 2020 | 273 |
| Total healthy pods harvested in 2020 | 221 |
| Pods harvested per tree in 2020 | 18 |
| Healthy pods harvested per tree in 2020 | 15 |
| Pods infected with witches' broom disease in 2020 | 22 |
| Pods infected with black pod disease in 2020 | 0 |
| Percentage of pods infected with witches' broom disease in 2020 | 8.06% |
| Percentage of pods infected with black pod disease in 2020 | 0.00% |
| Dry weight of a single bean in 2020 (g) | 0.86 |
| Total dry bean weight in 2020 (g) | 4953.78 |
| Total dry bean weight per tree in 2020 (g) | 330.25 |
| Yield in 2020 (kg/ha) | 388.046 |
| Clone yield divided by CCN 51 yield in 2020 | 3.04 |
| Total reps in 2021 | 15 |
| Total pods harvested in 2021 | 349 |
| Total healthy pods harvested in 2021 | 320 |
| Pods harvested per tree in 2021 | 23 |
| Healthy pods harvested per tree in 2021 | 21 |
| Pods infected with witches' broom disease in 2021 | 3 |
| Pods infected with black pod disease in 2021 | 1 |
| Percentage of pods infected with witches' broom disease in 2021 | 1% |
| Percentage of pods infected with black pod disease in 2021 | 0% |
| Dry weight of a single bean in 2021 (g) | 0.92 |
| Total dry bean weight in 2021 (g) | 6677.4 |
| Total dry bean weight per tree in 2021 (g) | 445.2 |
| Yield in 2021 (kg/ha) | 523.1 |
| Clone yield divided by CCN 51 yield in 2021 | 1.4 |
| Accumulated yield (kg/ha) | 911.11 |
| Number of dead plants infected with Ceratocystis cacaofunesta | 3 |
| Percentage of seedlings dead after infection with Ceratocystis cacaofunesta | 60% |
| Minimum lesion (mm) | 81.2 |
| Maximum lesion (mm) | 186.1 |
| Average lesion (mm) | 147.6 |
| Fat content (%) | 39.0533 |

### Variety ‘CMC-029’

In certain aspects, the present invention relates to a new and distinctive cacao plant designated as ‘CMC-029’.

The cacao variety ‘CMC-029’ originated from a controlled cross between varieties ‘VB-514’ and ‘CCN-51’. The breeder's reference is ‘MC-08060’, and the variety designation is ‘CMC-029’. Cacao variety ‘CMC-029’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Acute

The leaf size is medium (179.2 cm²), with 27.6 cm in length and 9.8 cm in width. The base leaf shape is obtuse, and the leaf apex is acuminate. The intensity of green in immature leaves is medium red, whereas the intensity of green in the mature leaves is medium.

The average length and width of the sepals are 7.9 mm (medium) and 2.6 mm (medium), respectively, with absent anthocyanin coloration. The ligula color is yellow, and the staminode has medium anthocyanin coloration, while the pedicel has weak anthocyanin coloration.

The average length and width of the pod are 199.9 mm (medium) and 82.3 mm (medium), respectively, with the yellow color of the mature pod. The pod surface roughness is smooth or slightly rough, with a medium depth of grooves and thick exocarp (e.g. lower and greater exocarps measuring 9.2 and 16.1 mm, respectively). The color of the pulp is light cream, and the pulp sweetness is medium, with BRIX measuring 15.4. The average number of seeds per pod is 19.9.

The seed has an oblong shape in longitudinal section, with an average seed length of 27.1 mm (short), and a width of 10.5 mm (medium). The seed thickness is 8.7 mm, and the cotyledon has dark red color.

TABLE 28
Variety ‘CMC-029’ Harvest and Infection Data in 2020 and 2021.

| Characteristic | Value |
|---|---|
| Total reps in 2020 | 12 |
| Total pods harvested in 2020 | 388 |
| Total healthy pods harvested in 2020 | 250 |
| Pods harvested per tree in 2020 | 32 |
| Healthy pods harvested per tree in 2020 | 21 |
| Pods infected with witches' broom disease in 2020 | 47 |
| Pods infected with black pod disease in 2020 | 10 |
| Percentage of pods infected with witches' broom disease in 2020 | 12.11% |
| Percentage of pods infected with black pod disease in 2020 | 2.58% |
| Dry weight of a single bean in 2020 (g) | 0.50 |
| Total dry bean weight in 2020 (g) | 4221.94 |
| Total dry bean weight per tree in 2020 (g) | 351.83 |
| Yield in 2020 (kg/ha) | 413.399 |
| Clone yield divided by CCN 51 yield in 2020 | 3.24 |
| Total reps in 2021 | 12 |
| Total pods harvested in 2021 | 386 |
| Total healthy pods harvested in 2021 | 316 |
| Pods harvested per tree in 2021 | 32 |
| Healthy pods harvested per tree in 2021 | 26 |
| Pods infected with witches' broom disease in 2021 | 8 |
| Pods infected with black pod disease in 2021 | 2 |
| Percentage of pods infected with witches' broom disease in 2021 | 2% |
| Percentage of pods infected with black pod disease in 2021 | 1% |
| Dry weight of a single bean in 2021 (g) | 0.85 |
| Total dry bean weight in 2021 (g) | 4982.4 |
| Total dry bean weight per tree in 2021 (g) | 415.2 |
| Yield in 2021 (kg/ha) | 487.9 |
| Clone yield divided by CCN 51 yield in 2021 | 1.4 |
| Accumulated yield (kg/ha) | 901.26 |
| Number of dead plants infected with Ceratocystis cacaofunesta | 5 |
| Percentage of seedlings dead after infection with Ceratocystis cacaofunesta | 100% |
| Minimum lesion (mm) | 106.8 |
| Maximum lesion (mm) | 154.6 |
| Average lesion (mm) | 124.1 |
| Fat content (%) | 37.76 |

### Variety ‘CMC-030’

In certain aspects, the present invention relates to a new and distinctive cacao plant designated as ‘CMC-030’.

The cacao variety ‘CMC-030’ originated from a controlled cross between varieties ‘GU-144C’ and ‘MC-08017’. The breeder's reference is ‘PT-08-018-2-084-05’, and the variety designation is ‘CMC-030’. Cacao variety ‘CMC-030’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Low
Fruit shape: Elliptic
Basal constriction on fruit: Present
Shape of fruit apex: Obtuse

The leaf size is medium (194.8 cm²), with 28.3 cm in length and 10.4 cm in width. The base leaf shape is obtuse, and the leaf apex is apiculate. The intensity of green in immature leaves is medium red, whereas the intensity of green in the mature leaves is medium.

The average length and width of the sepals are 7.1 mm (medium) and 2.5 mm (medium), respectively, with weak anthocyanin coloration. The ligula color is cream yellow, and the staminode has medium anthocyanin coloration, while the pedicel has moderate anthocyanin coloration.

The average length and width of the pod are 164.6 mm (medium) and 74.6 mm (short), respectively, with the orange/anthocyanin (ridge) color of the mature pod. The pod surface roughness is very rough, with a medium depth of grooves and thick exocarp (e.g. lower and greater exocarps measuring 9.9 and 15.3 mm, respectively). The color of the pulp is light cream, and the pulp sweetness is high, with BRIX measuring 17.1. The average number of seeds per pod is 27.1.

The seed has an elliptic shape in longitudinal section, with an average seed length of 23.5 mm (short), and a width of 12.3 mm (medium). The seed thickness is 8.5 mm, and the cotyledon has dark purple color.

TABLE 29
Variety ‘CMC-030’ Harvest and Infection Data in 2020 and 2021.

| Characteristic | Value |
|---|---|
| Total reps in 2020 | 12 |
| Total pods harvested in 2020 | 188 |
| Total healthy pods harvested in 2020 | 133 |
| Pods harvested per tree in 2020 | 16 |
| Healthy pods harvested per tree in 2020 | 11 |
| Pods infected with witches' broom disease in 2020 | 11 |
| Pods infected with black pod disease in 2020 | 18 |
| Percentage of pods infected with witches' broom disease in 2020 | 5.85% |
| Percentage of pods infected with black pod disease in 2020 | 9.57% |
| Dry weight of a single bean in 2020 (g) | 0.74 |
| Total dry bean weight in 2020 (g) | 2973.68 |
| Total dry bean weight per tree in 2020 (g) | 247.81 |
| Yield in 2020 (kg/ha) | 291.173 |
| Clone yield divided by CCN 51 yield in 2020 | 2.28 |
| Total reps in 2021 | 12 |
| Total pods harvested in 2021 | 296 |
| Total healthy pods harvested in 2021 | 200 |
| Pods harvested per tree in 2021 | 25 |
| Healthy pods harvested per tree in 2021 | 17 |
| Pods infected with witches' broom disease in 2021 | 7 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 0.99 |
| Total dry bean weight in 2021 (g) | 6037.1 |
| Total dry bean weight per tree in 2021 (g) | 503.1 |
| Yield in 2021 (kg/ha) | 591.1 |
| Clone yield divided by CCN 51 yield in 2021 | 1.6 |
| Accumulated yield (kg/ha) | 882.31 |
| Number of dead plants infected with Ceratocystis cacaofunesta | 3 |
| Percentage of seedlings dead after infection with Ceratocystis cacaofunesta | 60% |
| Minimum lesion (mm) | 105.8 |
| Maximum lesion (mm) | 165.3 |
| Average lesion (mm) | 130.9 |
| Fat content (%) | NA |

### Variety ‘CMC-031’

In certain aspects, the present invention relates to a new and distinctive cacao plant designated as ‘CMC-031’.

The cacao variety ‘CMC-031’ originated from a controlled cross between varieties ‘GU-144C’ and ‘MC-08017’. The breeder's reference is ‘PT-08-018-1-034-12’, and the variety designation is ‘CMC-031’. Cacao variety ‘CMC-031’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Absent or very low
Fruit shape: Elliptic
Basal constriction on fruit: Present
Shape of fruit apex: Obtuse

The leaf size is medium (172.5 cm²), with 27.8 cm in length and 9.4 cm in width. The base leaf shape is obtuse, and the leaf apex is acuminate. The intensity of green in immature leaves is dark red, whereas the intensity of green in the mature leaves is medium.

The average length and width of the sepals are 6.9 mm (medium) and 2.1 mm (short), respectively, with absent anthocyanin coloration. The ligula color is yellow, and the staminode has medium anthocyanin coloration, while the pedicel has weak anthocyanin coloration.

The average length and width of the pod are 128.2 mm (short) and 75.5 mm (medium), respectively, with the orange/anthocyanin (ridge) color of the mature pod. The pod surface roughness is smooth or slightly rough, with a shallow depth of grooves and medium exocarp (e.g. lower and greater exocarps measuring 10.7 and 14.2 mm, respectively). The color of the pulp is white, and the pulp sweetness is medium, with BRIX measuring 15.2. The average number of seeds per pod is 12.9.

The seed has an elliptic shape in longitudinal section, with an average seed length of 23.2 mm (short), and a width of 12.5 mm (medium). The seed thickness is 8.1 mm, and the cotyledon has pink color.

TABLE 30
Variety ‘CMC-031’ Harvest and Infection Data in 2020 and 2021.

| Characteristic | Value |
|---|---|
| Total reps in 2020 | 12 |
| Total pods harvested in 2020 | 345 |
| Total healthy pods harvested in 2020 | 253 |
| Pods harvested per tree in 2020 | 29 |
| Healthy pods harvested per tree in 2020 | 21 |
| Pods infected with witches' broom disease in 2020 | 19 |
| Pods infected with black pod disease in 2020 | 35 |
| Percentage of pods infected with witches' broom disease in 2020 | 5.51% |
| Percentage of pods infected with black pod disease in 2020 | 10.14% |
| Dry weight of a single bean in 2020 (g) | 0.76 |
| Total dry bean weight in 2020 (g) | 4433.83 |
| Total dry bean weight per tree in 2020 (g) | 369.49 |
| Yield in 2020 (kg/ha) | 434.146 |
| Clone yield divided by CCN 51 yield in 2020 | 3.40 |
| Total reps in 2021 | 12 |
| Total pods harvested in 2021 | 305 |
| Total healthy pods harvested in 2021 | 264 |
| Pods harvested per tree in 2021 | 25 |
| Healthy pods harvested per tree in 2021 | 22 |
| Pods infected with witches' broom disease in 2021 | 4 |
| Pods infected with black pod disease in 2021 | 0 |
| Percentage of pods infected with witches' broom disease in 2021 | 1% |
| Percentage of pods infected with black pod disease in 2021 | 0% |
| Dry weight of a single bean in 2021 (g) | 0.91 |
| Total dry bean weight in 2021 (g) | 4513.5 |
| Total dry bean weight per tree in 2021 (g) | 376.1 |
| Yield in 2021 (kg/ha) | 441.9 |
| Clone yield divided by CCN 51 yield in 2021 | 1.2 |
| Accumulated yield (kg/ha) | 876.09 |
| Number of dead plants infected with Ceratocystis cacaofunesta | 5 |
| Percentage of seedlings dead after infection with Ceratocystis cacaofunesta | 100% |
| Minimum lesion (mm) | 114.7 |
| Maximum lesion (mm) | 212 |
| Average lesion (mm) | 152.4 |
| Fat content (%) | 35.86 |

### Variety ‘CMC-032’

In certain aspects, the present invention relates to a new and distinctive cacao plant designated as ‘CMC-032’.

The cacao variety ‘CMC-032’ originated from a controlled cross between varieties ‘IAC-1’ and ‘CCN-51’. The breeder's reference is ‘MC-08092’, and the variety designation is ‘CMC-032’. Cacao variety ‘CMC-032’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Oblong
Basal constriction on fruit: Present
Shape of fruit apex: Obtuse

The leaf size is medium (194.9 cm²), with 28.4 cm in length and 10.4 cm in width. The base leaf shape is obtuse, and the leaf apex is apiculate. The intensity of green in immature leaves is medium red, whereas the intensity of green in the mature leaves is medium.

The average length and width of the sepals are 8.6 mm (medium) and 2.4 mm (medium), respectively, with absent anthocyanin coloration. The ligula color is cream yellow, and the staminode has medium anthocyanin coloration, while the pedicel has weak anthocyanin coloration.

The average length and width of the pod are 188.6 mm (medium) and 82.2 mm (medium), respectively, with the yellow color of the mature pod. The pod surface roughness is very rough, with a deep depth of grooves and thick exocarp (e.g. lower and greater exocarps measuring 11.5 and 17.5 mm, respectively). The color of the pulp is light cream, and the pulp sweetness is high, with BRIX measuring 19.3. The average number of seeds per pod is 19.4.

The seed has a ovada shape in longitudinal section, with an average seed length of 25.9 mm (medium), and a width of 13.4 mm (medium). The seed thickness is 9.2 mm, and the cotyledon has pink color.

TABLE 31
Variety ‘CMC-032’ Harvest and Infection Data in 2020 and 2021.

| Characteristic | Value |
|---|---|
| Total reps in 2020 | 15 |
| Total pods harvested in 2020 | 325 |
| Total healthy pods harvested in 2020 | 242 |
| Pods harvested per tree in 2020 | 22 |
| Healthy pods harvested per tree in 2020 | 16 |
| Pods infected with witches' broom disease in 2020 | 18 |
| Pods infected with black pod disease in 2020 | 21 |
| Percentage of pods infected with witches' broom disease in 2020 | 5.54% |
| Percentage of pods infected with black pod disease in 2020 | 6.46% |
| Dry weight of a single bean in 2020 (g) | 0.72 |
| Total dry bean weight in 2020 (g) | 4346.53 |
| Total dry bean weight per tree in 2020 (g) | 289.77 |
| Yield in 2020 (kg/ha) | 340.478 |
| Clone yield divided by CCN 51 yield in 2020 | 2.67 |
| Total reps in 2021 | 12 |
| Total pods harvested in 2021 | 275 |
| Total healthy pods harvested in 2021 | 254 |
| Pods harvested per tree in 2021 | 23 |
| Healthy pods harvested per tree in 2021 | 21 |
| Pods infected with witches' broom disease in 2021 | 4 |
| Pods infected with black pod disease in 2021 | 5 |
| Percentage of pods infected with witches' broom disease in 2021 | 1% |
| Percentage of pods infected with black pod disease in 2021 | 2% |
| Dry weight of a single bean in 2021 (g) | 1.26 |
| Total dry bean weight in 2021 (g) | 5357 |
| Total dry bean weight per tree in 2021 (g) | 446.4 |
| Yield in 2021 (kg/ha) | 524.5 |
| Clone yield divided by CCN 51 yield in 2021 | 1.5 |
| Accumulated yield (kg/ha) | 865.02 |
| Number of dead plants infected with Ceratocystis cacaofunesta | 5 |
| Percentage of seedlings dead after infection with Ceratocystis cacaofunesta | 100% |
| Minimum lesion (mm) | 108.5 |
| Maximum lesion (mm) | 203 |
| Average lesion (mm) | 143.5 |
| Fat content (%) | NA |

### Variety ‘CMC-033’

In certain aspects, the present invention relates to a new and distinctive cacao plant designated as ‘CMC-033’.

The cacao variety ‘CMC-033’ originated from a controlled cross between varieties ‘TSH-1188’ and ‘CCN-51’. The breeder's reference is ‘MCCS_MP01-718’, and the variety designation is ‘CMC-033’. Cacao variety ‘CMC-033’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Low
Fruit shape: Oblong
Basal constriction on fruit: Present
Shape of fruit apex: Attenuate

The leaf size is medium (204.9 cm²), with 27.8 cm in length and 11.2 cm in width. The base leaf shape is obtuse, and the leaf apex is apiculate. The intensity of green in immature leaves is dark red, whereas the intensity of green in the mature leaves is medium.

The average length and width of the sepals are 8.9 mm (medium) and 2.7 mm (long), respectively, with weak anthocyanin coloration. The ligula color is yellow, and the staminode has strong anthocyanin coloration, while the pedicel has moderate anthocyanin coloration.

The average length and width of the pod are 257.7 mm (long) and 80.9 mm (medium), respectively, with the orange/anthocyanin (ridge) color of the mature pod. The pod surface roughness is moderately rough, with a medium depth of grooves and thick exocarp (e.g. lower and greater exocarps measuring 11.1 and 15.5 mm, respectively). The color of the pulp is light cream, and the pulp sweetness is high, with BRIX measuring 17.8. The average number of seeds per pod is 24.2.

The seed has an oblong shape in longitudinal section, with an average seed length of 25.2 mm (medium), and a width of 13.2 mm (medium). The seed thickness is 9.3 mm, and the cotyledon has dark red color.

TABLE 32
Variety ‘CMC-033’ Harvest and Infection Data in 2020 and 2021.

| Characteristic | Value |
|---|---|
| Total reps in 2020 | 12 |
| Total pods harvested in 2020 | 161 |
| Total healthy pods harvested in 2020 | 101 |
| Pods harvested per tree in 2020 | 13 |
| Healthy pods harvested per tree in 2020 | 8 |
| Pods infected with witches' broom disease in 2020 | 5 |
| Pods infected with black pod disease in 2020 | 7 |
| Percentage of pods infected with witches' broom disease in 2020 | 3.11% |
| Percentage of pods infected with black pod disease in 2020 | 4.35% |
| Dry weight of a single bean in 2020 (g) | 0.81 |
| Total dry bean weight in 2020 (g) | 2975.22 |
| Total dry bean weight per tree in 2020 (g) | 247.93 |
| Yield in 2020 (kg/ha) | 291.323 |
| Clone yield divided by CCN 51 yield in 2020 | 2.28 |
| Total reps in 2021 | 12 |
| Total pods harvested in 2021 | 220 |
| Total healthy pods harvested in 2021 | 155 |
| Pods harvested per tree in 2021 | 18 |
| Healthy pods harvested per tree in 2021 | 13 |
| Pods infected with witches' broom disease in 2021 | 5 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 1.18 |
| Total dry bean weight in 2021 (g) | 5816.5 |
| Total dry bean weight per tree in 2021 (g) | 484.7 |
| Yield in 2021 (kg/ha) | 569.5 |
| Clone yield divided by CCN 51 yield in 2021 | 1.6 |
| Accumulated yield (kg/ha) | 860.86 |
| Number of dead plants infected with Ceratocystis cacaofunesta | 3 |
| Percentage of seedlings dead after infection with Ceratocystis cacaofunesta | 60% |
| Minimum lesion (mm) | 34.5 |
| Maximum lesion (mm) | 121.4 |
| Average lesion (mm) | 73.2 |
| Fat content (%) | NA |

### Variety ‘CMC-034’

In certain aspects, the present invention relates to a new and distinctive cacao plant designated as ‘CMC-034’.

The cacao variety ‘CMC-034’ originated from a controlled cross between varieties ‘TSH-1188’ and ‘CCN-51’. The breeder's reference is ‘MCCS_MP01-192’, and the variety designation is ‘CMC-034’. Cacao variety ‘CMC-034’ has subsequently been asexually propagated via grafting onto open-pollinated ‘VB-1151’ rootstock.

Characteristics of this variety are provided below:
Young flush leaf color: Brown
Axil spot on petiole: Absent
Intensity of anthocyanin on sepal: Medium
Fruit shape: Oblong
Basal constriction on fruit: Present
Shape of fruit apex: Caudate

The leaf size is medium (174.2 cm²), with 26.5 cm in length and 9.9 cm in width. The base leaf shape is obtuse, and the leaf apex is apiculate. The intensity of green in immature leaves is brown, whereas the intensity of green in the mature leaves is medium.

The average length and width of the sepals are 7.6 mm (medium) and 2.9 mm (long), respectively, with moderate anthocyanin coloration. The ligula color is cream yellow, and the staminode has medium anthocyanin coloration, while the pedicel has moderate anthocyanin coloration.

The average length and width of the pod are 212.8 mm (long) and 87.6 mm (medium), respectively, with the orange/anthocyanin (ridge) color of the mature pod. The pod surface roughness is very rough, with a deep depth of grooves and thick exocarp (e.g. lower and greater exocarps measuring 9.8 and 17.1 mm, respectively). The color of the pulp is dark cream, and the pulp sweetness is medium, with BRIX measuring 16.0. The average number of seeds per pod is 36.7.

The seed has an elliptic shape in longitudinal section, with an average seed length of 24.5 mm (medium), and a width of 14.6 mm (medium). The seed thickness is 10.3 mm, and the cotyledon has dark purple color.

TABLE 33
Variety ‘CMC-034’ Harvest and Infection Data in 2020 and 2021.

| Characteristic | Value |
|---|---|
| Total reps in 2020 | 15 |
| Total pods harvested in 2020 | 29 |
| Total healthy pods harvested in 2020 | 12 |
| Pods harvested per tree in 2020 | 2 |
| Healthy pods harvested per tree in 2020 | 1 |
| Pods infected with witches' broom disease in 2020 | 1 |
| Pods infected with black pod disease in 2020 | 2 |
| Percentage of pods infected with witches' broom disease in 2020 | 3.45% |
| Percentage of pods infected with black pod disease in 2020 | 6.90% |
| Dry weight of a single bean in 2020 (g) | 1.34 |
| Total dry bean weight in 2020 (g) | 589.198 |
| Total dry bean weight per tree in 2020 (g) | 39.28 |
| Yield in 2020 (kg/ha) | 46.1538 |
| Clone yield divided by CCN 51 yield in 2020 | 0.36 |
| Total reps in 2021 | 15 |
| Total pods harvested in 2021 | 281 |
| Total healthy pods harvested in 2021 | 208 |
| Pods harvested per tree in 2021 | 19 |
| Healthy pods harvested per tree in 2021 | 14 |
| Pods infected with witches' broom disease in 2021 | 3 |
| Pods infected with black pod disease in 2021 | 0 |
| Percentage of pods infected with witches' broom disease in 2021 | 1% |
| Percentage of pods infected with black pod disease in 2021 | 0% |
| Dry weight of a single bean in 2021 (g) | 1.48 |
| Total dry bean weight in 2021 (g) | 9786.1 |
| Total dry bean weight per tree in 2021 (g) | 652.4 |
| Yield in 2021 (kg/ha) | 766.6 |
| Clone yield divided by CCN 51 yield in 2021 | 2.1 |
| Accumulated yield (kg/ha) | 812.73 |
| Number of dead plants infected with Ceratocystis cacaofunesta | 5 |
| Percentage of seedlings dead after infection with Ceratocystis cacaofunesta | 100% |
| Minimum lesion (mm) | 104.7 |
| Maximum lesion (mm) | 158.2 |
| Average lesion (mm) | 133.9 |
| Fat content (%) | NA |

### Variety ‘CMC-035’

In certain aspects, the present invention relates to a new and distinctive cacao plant designated as ‘CMC-035’.

The cacao variety ‘CMC-035’ originated from a controlled cross between varieties ‘MC-08005’ and ‘MC-08060’ (‘CMC-029’). The breeder's reference is ‘PT-08-038-1-113-18’, and the variety designation is ‘CMC-035’. Cacao variety ‘CMC-035’ has subsequently been asexually propagated via grafting onto open-pollinated ‘VB-1151’ rootstock.

Characteristics of this variety are provided below:
Young flush leaf color: Light red
Axil spot on petiole: Absent
Intensity of anthocyanin on sepal: Low
Fruit shape: Oblong
Basal constriction on fruit: Present
Shape of fruit apex: Obtuse

The leaf size is medium (166.6 cm²), with 27.1 cm in length and 9.3 cm in width. The base leaf shape is obtuse, and the leaf apex is apiculate. The intensity of green in immature leaves is light red, whereas the intensity of green in the mature leaves is medium.

The average length and width of the sepals are 7.4 mm (medium) and 2.1 mm (medium), respectively, with weak anthocyanin coloration. The ligula color is yellow, and the staminode has weak anthocyanin coloration, while the pedicel has weak anthocyanin coloration.

The average length and width of the pod are 171.2 mm (medium) and 72.5 mm (short), respectively, with the orange/anthocyanin (ridge) color of the mature pod. The pod surface roughness is moderately rough, with a medium depth of grooves and medium exocarp (e.g. lower and greater exocarps measuring 9.4 and 13.9 mm, respectively). The color of the pulp is light cream, and the pulp sweetness is low, with BRIX measuring 13.7. The average number of seeds per pod is 19.6.

The seed has an oblong shape in longitudinal section, with an average seed length of 24.2 mm (short), and a width of 11.4 mm (medium). The seed thickness is 9.5 mm, and the cotyledon has pink color.

TABLE 34
Variety ‘CMC-035’ Harvest and Infection Data in 2020 and 2021.

| Characteristic | Value |
|---|---|
| Total reps in 2020 | 15 |
| Total pods harvested in 2020 | 240 |
| Total healthy pods harvested in 2020 | 171 |
| Pods harvested per tree in 2020 | 16 |
| Healthy pods harvested per tree in 2020 | 11 |
| Pods infected with witches' broom disease in 2020 | 28 |
| Pods infected with black pod disease in 2020 | 6 |
| Percentage of pods infected with witches' broom disease in 2020 | 11.67% |
| Percentage of pods infected with black pod disease in 2020 | 2.50% |
| Dry weight of a single bean in 2020 (g) | 0.85 |
| Total dry bean weight in 2020 (g) | 4240.94 |
| Total dry bean weight per tree in 2020 (g) | 282.73 |
| Yield in 2020 (kg/ha) | 332.207 |
| Clone yield divided by CCN 51 yield in 2020 | 2.60 |
| Total reps in 2021 | 15 |
| Total pods harvested in 2021 | 297 |
| Total healthy pods harvested in 2021 | 229 |
| Pods harvested per tree in 2021 | 20 |
| Healthy pods harvested per tree in 2021 | 15 |
| Pods infected with witches' broom disease in 2021 | 7 |
| Pods infected with black pod disease in 2021 | 4 |
| Percentage of pods infected with witches' broom disease in 2021 | 2% |
| Percentage of pods infected with black pod disease in 2021 | 1% |
| Dry weight of a single bean in 2021 (g) | 1.04 |
| Total dry bean weight in 2021 (g) | 5519.1 |
| Total dry bean weight per tree in 2021 (g) | 367.9 |
| Yield in 2021 (kg/ha) | 432.3 |
| Clone yield divided by CCN 51 yield in 2021 | 1.2 |
| Accumulated yield (kg/ha) | 764.54 |
| Number of dead plants infected with Ceratocystis cacaofunesta | 4 |
| Percentage of seedlings dead after infection with Ceratocystis cacaofunesta | 80% |
| Minimum lesion (mm) | 115.6 |
| Maximum lesion (mm) | 192.9 |
| Average lesion (mm) | 167.5 |
| Fat content (%) | NA |

### Variety ‘CMC-036’

In certain aspects, the present invention relates to a new and distinctive cacao plant designated as ‘CMC-036’.

The cacao variety ‘CMC-036’ originated from a controlled cross between varieties ‘CCN-51’ and ‘MC-08017’. The breeder's reference is ‘PT-08-003-1-009-05’, and the variety designation is ‘CMC-036’. Cacao variety ‘CMC-036’ has subsequently been asexually propagated via grafting onto open-pollinated ‘VB-1151’ rootstock.

Characteristics of this variety are provided below:
Young flush leaf color: Dark red
Axil spot on petiole: Absent
Intensity of anthocyanin on sepal: Low
Fruit shape: Elliptic
Basic constriction on fruit: Present
Shape of fruit apex: Caudate

The leaf size is medium (177.3 cm²), with 27.7 cm in length and 9.6 cm in width. The base leaf shape is obtuse, and the leaf apex is acuminate. The intensity of green in immature leaves is dark red, whereas the intensity of green in the mature leaves is medium.

The average length and width of the sepals are 8.2 mm (medium) and 2.5 mm (medium), respectively, with weak anthocyanin coloration. The ligula color is cream yellow, and the staminode has medium anthocyanin coloration, while the pedicel has moderate anthocyanin coloration.

The average length and width of the pod are 194.3 mm (medium) and 87.4 mm (medium), respectively, with the red/orange (groove) color of the mature pod. The pod surface roughness is moderately rough, with a medium depth of grooves and medium exocarp (e.g. lower and greater exocarps measuring 10.4 and 14.81 mm, respectively). The color of the pulp is light cream, and the pulp sweetness is high, with BRIX measuring 19.3. The average number of seeds per pod is 17.5.

The seed has an oblong shape in longitudinal section, with an average seed length of 29.6 mm (medium), and a width of 13.8 mm (medium). The seed thickness is 9.4 mm, and the cotyledon has dark purple color.

TABLE 35
Variety ‘CMC-036’ Harvest and Infection Data in 2020 and 2021.

| Characteristic | Value |
|---|---|
| Total reps in 2020 | 12 |
| Total pods harvested in 2020 | 140 |
| Total healthy pods harvested in 2020 | 67 |
| Pods harvested per tree in 2020 | 12 |
| Healthy pods harvested per tree in 2020 | 6 |
| Pods infected with witches' broom disease in 2020 | 14 |
| Pods infected with black pod disease in 2020 | 0 |
| Percentage of pods infected with witches' broom disease in 2020 | 10.00% |
| Percentage of pods infected with black pod disease in 2020 | 0.00% |
| Dry weight of a single bean in 2020 (g) | 0.95 |
| Total dry bean weight in 2020 (g) | 2115.03 |
| Total dry bean weight per tree in 2020 (g) | 176.25 |
| Yield in 2020 (kg/ha) | 207.097 |
| Clone yield divided by CCN 51 yield in 2020 | 1.62 |
| Total reps in 2021 | 11 |
| Total pods harvested in 2021 | 226 |
| Total healthy pods harvested in 2021 | 146 |
| Pods harvested per tree in 2021 | 21 |
| Healthy pods harvested per tree in 2021 | 13 |
| Pods infected with witches' broom disease in 2021 | 4 |
| Pods infected with black pod disease in 2021 | 0 |
| Percentage of pods infected with witches' broom disease in 2021 | 2% |
| Percentage of pods infected with black pod disease in 2021 | 0% |
| Dry weight of a single bean in 2021 (g) | 1.41 |
| Total dry bean weight in 2021 (g) | 4929.3 |
| Total dry bean weight per tree in 2021 (g) | 448.1 |
| Yield in 2021 (kg/ha) | 526.5 |
| Clone yield divided by CCN 51 yield in 2021 | 1.5 |
| Accumulated yield (kg/ha) | 733.64 |
| Number of dead plants infected with Ceratocystis cacaofunesta | 0 |
| Percentage of seedlings dead after infection with Ceratocystis cacaofunesta | 0% |
| Minimum lesion (mm) | 0 |
| Maximum lesion (mm) | 0 |
| Average lesion (mm) | 0 |
| Fat content (%) | NA |

### Variety ‘CMC-022’

In certain aspects, the present invention relates to a new and distinctive cacao plant designated as ‘CMC-022’.

The cacao variety ‘CMC-022’ originated from a controlled cross between varieties ‘MOQ-4.25’ and ‘MC-08060’ (‘CMC-029’). The breeder's reference is ‘PT-08-042-3-166-15’, and the variety designation is ‘CMC-022’. Cacao variety ‘CMC-022’ has subsequently been asexually propagated via grafting onto open-pollinated ‘VB-1151’ rootstock.

Characteristics of this variety are provided below:
Young flush leaf color: Medium red
Axil spot on petiole: Absent
Intensity of anthocyanin on sepal: Absent or very low
Fruit shape: Elliptic
Basic constriction on fruit: Present
Shape of fruit apex: Obtuse

The leaf size is medium (186.5 cm²), with 28.2 cm in length and 10.0 cm in width. The base leaf shape is obtuse, and the leaf apex is apiculate. The intensity of green in immature leaves is medium red, whereas the intensity of green in the mature leaves is medium.

The average length and width of the sepals are 7.3 mm (medium) and 2.5 mm (medium), respectively, with absent anthocyanin coloration. The ligula color is yellow, and the staminode has medium anthocyanin coloration, while pedicel has weak anthocyanin coloration.

The average length and width of the pod are 161.9 mm (medium) and 79.2 mm (medium), respectively, with the yellow color of the mature pod. The pod surface roughness is moderately rough, with a medium depth of grooves and thick exocarp (e.g. lower and greater exocarps measuring 10.0 and 16.8 mm, respectively). The color of the pulp is light cream, and the pulp sweetness is medium, with BRIX measuring 16.8. The average number of seeds per pod is 13.8.

The seed has an elliptic shape in longitudinal section, with an average seed length of 24.9 mm (medium), and a width of 14.5 mm (medium). The seed thickness is 9.6 mm, and the cotyledon has pink color.

TABLE 36
Variety ‘CMC-022’ Harvest and Infection Data in 2020 and 2021.

| Characteristic | Value |
|---|---|
| Total reps in 2020 | 12 |
| Total pods harvested in 2020 | 303 |
| Total healthy pods harvested in 2020 | 253 |
| Pods harvested per tree in 2020 | 25 |
| Healthy pods harvested per tree in 2020 | 21 |
| Pods infected with witches' broom disease in 2020 | 17 |
| Pods infected with black pod disease in 2020 | 0 |
| Percentage of pods infected with witches' broom disease in 2020 | 5.61% |
| Percentage of pods infected with black pod disease in 2020 | 0.00% |
| Dry weight of a single bean in 2020 (g) | 0.82 |
| Total dry bean weight in 2020 (g) | 4825.962 |
| Total dry bean weight per tree in 2020 (g) | 402.16 |
| Yield in 2020 (kg/ha) | 472.5421 |
| Clone yield divided by CCN 51 yield in 2020 | 3.70 |
| Total reps in 2021 | 12 |
| Total pods harvested in 2021 | 224 |
| Total healthy pods harvested in 2021 | 194 |
| Pods harvested per tree in 2021 | 19 |
| Healthy pods harvested per tree in 2021 | 16 |
| Pods infected with witches' broom disease in 2021 | 6 |
| Pods infected with black pod disease in 2021 | 0 |
| Percentage of pods infected with witches' broom disease in 2021 | 3% |
| Percentage of pods infected with black pod disease in 2021 | 0% |
| Dry weight of a single bean in 2021 (g) | 2.05 |
| Total dry bean weight in 2021 (g) | 5782.5 |
| Total dry bean weight per tree in 2021 (g) | 481.9 |
| Yield in 2021 (kg/ha) | 566.2 |
| Clone yield divided by CCN 51 yield in 2021 | 1.6 |
| Accumulated yield (kg/ha) | 1,038.75 |
| Number of dead plants infected with Ceratocystis cacaofunesta | 5 |
| Percentage of seedlings dead after infection with Ceratocystis cacaofunesta | 100% |
| Minimum lesion (mm) | 85.8 |
| Maximum lesion (mm) | 182.2 |
| Average lesion (mm) | 143.2 |
| Fat content (%) | 43.85667 |

### CT-12 Trial Data

Provided below are additional data for the varieties disclosed herein from a multi-year trial from a plot designated as “CT-12.” The “CT-12” trial is located in Bahia, Brazil.

TABLE 37
Annual yield (t/ha) of various varieties at “CT-12” trial over multiple years and
comparison to ‘CCN 51’ control variety. Yield refers to the amount of useable pods
(taking into account pod loss from disease and other pod loss sources).

| | | Yield | Yield | Yield | Yield | Yield | Cumulative | Ranking |
|---|---|---|---|---|---|---|---|---|
| | | (t/ha) | (t/ha) | (t/ha) | (t/ha) | (t/ha) | Yield | vs |
| Genotype name | Variety | 2020 | 2021 | 2022 | 2023 | 2024 | (t/ha) | CCN 51 |
| PT-08-020-1-145-02 | CMC-001 | 0.27 | 2.14 | 3.39 | 2.32 | 1.75 | 9.85 | 1.87 |
| PT-08-041-2-052-18 | CMC-008 | 0.41 | 1.29 | 2.83 | 2.67 | 1.48 | 8.68 | 1.65 |
| PT-08-041-1-037-17 | CMC-015 | 0.33 | 1.6 | 2.71 | 2.1 | 1.47 | 8.22 | 1.56 |
| PT-08-020-1-145-15 | CMC-007 | 0.38 | 1.67 | 2.33 | 2.22 | 1.34 | 7.95 | 1.51 |
| PT-08-020-1-145-12 | CMC-002 | 0.59 | 1.58 | 2.28 | 2.25 | 1.18 | 7.88 | 1.50 |
| PT-08-018-1-034-18 | CMC-009 | 0.46 | 1.25 | 2.3 | 2 | 1.59 | 7.6 | 1.44 |
| PT-08-020-2-057-12 | CMC-019 | 0.15 | 1.63 | 2.62 | 1.64 | 1.49 | 7.54 | 1.43 |
| PT-08-038-3-096-10 | CMC-006 | 0.26 | 1.84 | 2.39 | 1.86 | 1.09 | 7.44 | 1.41 |
| PT-08-034-3-154-07 | CMC-016 | 0.42 | 1.37 | 1.97 | 2.42 | 1.19 | 7.37 | 1.40 |
| PT-08-020-2-057-16 | CMC-024 | 0.06 | 1.89 | 2.32 | 1.86 | 0.94 | 7.07 | 1.34 |
| PT-08-018-2-084-18 | CMC-003 | 0.58 | 1.65 | 2.52 | 1.45 | 0.84 | 7.04 | 1.34 |
| PT-08-064-1-107-18 | CMC-027 | 0.35 | 1.22 | 1.84 | 2.32 | 1.2 | 6.93 | 1.31 |
| MCCS_MP01-192 | CMC-034 | 0.05 | 1.15 | 2.4 | 2.42 | 0.9 | 6.92 | 1.31 |
| PT-08-038-1-113-03 | CMC-010 | 0.42 | 1.55 | 2.45 | 1.74 | 0.74 | 6.9 | 1.31 |
| PT-08-057-2-173-09 | CMC-018 | 0.41 | 1.29 | 1.89 | 1.85 | 1.18 | 6.61 | 1.25 |
| PT-08-038-1-113-09 | CMC-004 | 0.86 | 1.43 | 2.11 | 1.48 | 0.72 | 6.6 | 1.25 |
| PT-08-019-3-164-18 | CMC-021 | 0.42 | 1.28 | 2.15 | 1.84 | 0.91 | 6.59 | 1.25 |
| MC-08006 | CMC-014 | 0.33 | 1.4 | 2.09 | 1.87 | 0.88 | 6.57 | 1.25 |
| PT-08-003-3-155-18 | CMC-013 | 0.58 | 1.2 | 2.02 | 1.66 | 0.89 | 6.35 | 1.20 |
| MC-13-202 | CMC-012 | 0.1 | 1.64 | 1.81 | 1.94 | 0.84 | 6.32 | 1.20 |
| PT-08-038-2-050-11 | CMC-023 | 0.17 | 1.4 | 2.39 | 1.66 | 0.66 | 6.27 | 1.19 |
| PT-08-038-3-096-02 | CMC-025 | 0.17 | 1.42 | 1.84 | 1.62 | 1.21 | 6.26 | 1.19 |
| PT-08-018-2-084-12 | CMC-005 | 0.8 | 1.04 | 1.78 | 1.55 | 1.06 | 6.22 | 1.18 |
| SJ-02 | Control | 0.02 | 1 | 1.91 | 1.96 | 1.2 | 6.09 | 1.16 |
| PT-08-045-2-172-10 | CMC-020 | 0.31 | 1.38 | 1.93 | 1.66 | 0.57 | 5.85 | 1.11 |
| FCOM-150 | Control | 0.22 | 1.24 | 1.79 | 1.49 | 1.04 | 5.79 | 1.10 |
| MCCS_MP01-718 | CMC-033 | 0.31 | 1.15 | 2.16 | 1.51 | 0.53 | 5.66 | 1.07 |
| PT-08-038-1-113-18 | CMC-035 | 0.35 | 1 | 1.79 | 1.87 | 0.62 | 5.64 | 1.07 |
| PT-08-003-1-009-05 | CMC-036 | 0.21 | 1.14 | 1.81 | 1.8 | 0.59 | 5.54 | 1.05 |
| CCN-51 | Control | 0.14 | 0.89 | 1.68 | 1.83 | 0.73 | 5.27 | 1.00 |
| PT-08-018-2-084-05 | CMC-030 | 0.3 | 1.16 | 1.71 | 1.4 | 0.67 | 5.25 | 1.00 |
| PT-08-018-1-034-01 | CMC-028 | 0.44 | 1.18 | 1.47 | 1.23 | 0.9 | 5.21 | 0.99 |
| PT-08-018-2-084-08 | CMC-026 | 0.23 | 1.09 | 1.62 | 1.42 | 0.84 | 5.19 | 0.98 |
| PT-08-038-3-096-06 | CMC-017 | 0.68 | 1.07 | 1.42 | 1.28 | 0.67 | 5.12 | 0.97 |
| PT-08-018-1-034-12 | CMC-031 | 0.44 | 0.99 | 1.58 | 1.48 | 0.6 | 5.08 | 0.96 |
| PT-08-042-3-166-15 | CMC-022 | 0.46 | 1 | 1.62 | 1.47 | 0.44 | 4.99 | 0.95 |
| PS-1319 | Control | 0.1 | 0.6 | 1.49 | 1.91 | 0.74 | 4.85 | 0.92 |
| FB-206 (BP-S) | Control | 0.3 | 0.84 | 1.36 | 1.14 | 0.69 | 4.34 | 0.82 |
| MC-08060 | CMC-029 | 0.44 | 0.81 | 1.34 | 1.25 | 0.49 | 4.32 | 0.82 |
| CP-49 | Control | 0.34 | 0.81 | 1.13 | 1.19 | 0.68 | 4.15 | 0.79 |
| MC-08092 | CMC-032 | 0.33 | 0.89 | 1.17 | 1.21 | 0.48 | 4.08 | 0.77 |
| PT-08-020-3-092-11 | CMC-011 | 0.36 | 1.1 | 1.18 | 0.8 | 0.63 | 4.06 | 0.77 |
| CEPEC-2002 (VB-1151) | Control | 0.25 | 0.98 | 1.21 | 0.95 | 0.47 | 3.86 | 0.73 |
| SIAL-70 (WB-S) | Control | 0.28 | 0.62 | 1.15 | 1.09 | 0.63 | 3.77 | 0.72 |
| PH-15 | Control | 0.05 | 0.77 | 1.43 | 0.84 | 0.31 | 3.39 | 0.64 |
| CEPEC-2004 (CEPEC-46) | Control | 0.11 | 0.54 | 1.07 | 1.38 | 0.26 | 3.36 | 0.64 |
| PH-16 | Control | 0.13 | 0.72 | 1.03 | 0.96 | 0.31 | 3.15 | 0.60 |
| Pound-7 (BP-R) | Control | 0.13 | 0.77 | 1 | 0.77 | 0.32 | 3 | 0.57 |
| IPIRANGA-1 | Control | 0.12 | 0.54 | 0.92 | 0.85 | 0.27 | 2.69 | 0.51 |
| CCN-10 | Control | 0.01 | 0.29 | 0.85 | 1.06 | 0.15 | 2.35 | 0.45 |
| CATIE-R1 | Control | 0.2 | 0.48 | 0.72 | 0.55 | 0.26 | 2.22 | 0.42 |
| IAC-1 | Control | 0.04 | 0.45 | 0.54 | 0.6 | 0.5 | 2.12 | 0.40 |
| CATIE-R6 | Control | 0.04 | 0.24 | 0.54 | 0.91 | 0.36 | 2.09 | 0.40 |
| NA-33 | Control | 0.28 | 0.3 | 0.6 | 0.5 | 0.2 | 1.87 | 0.35 |
| CATIE-R4 | Control | 0.07 | 0.15 | 0.49 | 0.67 | 0.2 | 1.58 | 0.30 |
| VB-514 | Control | 0.05 | 0.23 | 0.55 | 0.46 | 0.18 | 1.47 | 0.28 |
| PMCT-58 | Control | 0 | 0.21 | 0.39 | 0.46 | 0.24 | 1.3 | 0.25 |
| GU-144C T1 | Control | 0.11 | 0.32 | 0.33 | 0.29 | 0.17 | 1.23 | 0.23 |
| TSH-1188 (BP-R) | Control | 0 | 0.13 | 0.24 | 0.15 | 0.1 | 0.63 | 0.12 |
| SCA-6 (WB-T) | Control | 0.02 | 0.09 | 0.12 | 0.14 | 0.08 | 0.44 | 0.08 |
| GU-296H | Control | 0.02 | 0.09 | 0.14 | 0.04 | 0.03 | 0.33 | 0.06 |
| GU-171C | Control | 0.06 | 0.04 | 0.03 | 0.04 | 0.15 | 0.31 | 0.06 |

TABLE 38
Annual prevalence of WBD (Witches broom disease) for various varieties at “CT-12”
trial over multiple years and comparison to ‘CCN 51’ control variety.

| | | WBD | WBD | WBD | WBD | WBD | Total | Ranking |
|---|---|---|---|---|---|---|---|---|
| | | (%) | (%) | (%) | (%) | (%) | WBD | vs |
| Genotype name | Variety | 2020 | 2021 | 2022 | 2023 | 2024 | (%) | CCN 51 |
| PT-08-038-3-096-10 | CMC-006 | 6% | 1% | 1% | 2% | 1% | 1.3% | −64% |
| PT-08-020-3-092-11 | CMC-011 | 3% | 0% | 1% | 2% | 2% | 1.4% | −63% |
| PT-08-038-3-096-02 | CMC-025 | 3% | 2% | 1% | 1% | 2% | 1.4% | −61% |
| PT-08-020-2-057-12 | CMC-019 | 5% | 1% | 1% | 2% | 1% | 1.5% | −61% |
| PT-08-018-1-034-12 | CMC-031 | 6% | 1% | 1% | 2% | 0% | 1.6% | −57% |
| PT-08-038-3-096-06 | CMC-017 | 3% | 1% | 1% | 3% | 1% | 1.6% | −57% |
| GU-144C T1 | Control | 2% | 1% | 0% | 2% | 4% | 1.6% | −56% |
| PT-08-020-1-145-02 | CMC-001 | 11% | 1% | 0% | 2% | 3% | 1.7% | −54% |
| GU-296H | Control | 8% | 2% | 1% | 0% | 3% | 2.0% | −46% |
| PT-08-018-2-084-18 | CMC-003 | 4% | 0% | 1% | 3% | 4% | 2.0% | −46% |
| PT-08-020-2-057-16 | CMC-024 | 11% | 0% | 2% | 3% | 2% | 2.0% | −46% |
| PT-08-018-2-084-08 | CMC-026 | 9% | 1% | 1% | 2% | 2% | 2.0% | −45% |
| PT-08-020-1-145-12 | CMC-002 | 4% | 1% | 1% | 4% | 2% | 2.1% | −42% |
| PT-08-038-1-113-09 | CMC-004 | 4% | 0% | 0% | 4% | 3% | 2.2% | −42% |
| PT-08-038-2-050-11 | CMC-023 | 10% | 2% | 1% | 3% | 3% | 2.4% | −36% |
| PT-08-041-1-037-17 | CMC-015 | 9% | 1% | 2% | 3% | 1% | 2.4% | −36% |
| PT-08-018-1-034-01 | CMC-028 | 8% | 0% | 0% | 5% | 2% | 2.4% | −35% |
| PT-08-020-1-145-15 | CMC-007 | 8% | 1% | 1% | 4% | 2% | 2.6% | −31% |
| PT-08-018-1-034-18 | CMC-009 | 9% | 1% | 1% | 4% | 2% | 2.7% | −28% |
| PT-08-018-2-084-12 | CMC-005 | 7% | 1% | 0% | 4% | 2% | 2.7% | −27% |
| PT-08-018-2-084-05 | CMC-030 | 6% | 2% | 2% | 4% | 1% | 2.7% | −26% |
| MCCS_MP01-718 | CMC-033 | 3% | 1% | 2% | 5% | 3% | 2.8% | −25% |
| PT-08-019-3-164-18 | CMC-021 | 8% | 1% | 1% | 4% | 2% | 2.9% | −23% |
| PT-08-038-1-113-18 | CMC-035 | 12% | 1% | 1% | 3% | 4% | 3.0% | −21% |
| PT-08-003-1-009-05 | CMC-036 | 10% | 2% | 1% | 3% | 7% | 3.1% | −17% |
| PT-08-038-1-113-03 | CMC-010 | 4% | 3% | 2% | 3% | 8% | 3.2% | −15% |
| PT-08-064-1-107-18 | CMC-027 | 8% | 1% | 2% | 5% | 3% | 3.2% | −14% |
| MC-08092 | CMC-032 | 6% | 1% | 0% | 6% | 4% | 3.2% | −13% |
| MCCS_MP01-192 | CMC-034 | 3% | 2% | 3% | 5% | 2% | 3.3% | −12% |
| SIAL-70 (WB-S) | Control | 14% | 0% | 3% | 4% | 2% | 3.3% | −11% |
| PT-08-041-2-052-18 | CMC-008 | 7% | 2% | 2% | 5% | 3% | 3.3% | −11% |
| GU-171C | Control | 10% | 0% | 0% | 5% | 1% | 3.4% | −10% |
| CCN-51 | Control | 8% | 1% | 2% | 6% | 3% | 3.7% | 0% |
| PT-08-042-3-166-15 | CMC-022 | 6% | 2% | 2% | 6% | 3% | 3.7% | 0% |
| PT-08-034-3-154-07 | CMC-016 | 8% | 1% | 3% | 6% | 2% | 3.8% | 1% |
| PT-08-045-2-172-10 | CMC-020 | 21% | 2% | 2% | 4% | 2% | 4.1% | 11% |
| CEPEC-2004 | Control | 7% | 1% | 3% | 5% | 6% | 4.1% | 11% |
| (CEPEC-46) | | | | | | | | |
| PT-08-057-2-173-09 | CMC-018 | 7% | 2% | 2% | 8% | 3% | 4.2% | 13% |
| SCA-6 (WB-T) | Control | 14% | 3% | 5% | 6% | 1% | 4.4% | 18% |
| VB-514 | Control | 23% | 3% | 5% | 4% | 1% | 4.5% | 21% |
| MC-08060 | CMC-029 | 12% | 2% | 3% | 6% | 2% | 4.7% | 27% |
| TSH-1188 (BP-R) | Control | 0% | 0% | 7% | 6% | 6% | 4.8% | 30% |
| MC-08006 | CMC-014 | 17% | 2% | 3% | 6% | 4% | 5.0% | 34% |
| CATIE-R6 | Control | 20% | 1% | 3% | 8% | 2% | 5.2% | 39% |
| SJ-02 | Control | 26% | 2% | 7% | 8% | 3% | 6.1% | 63% |
| PT-08-003-3-155-18 | CMC-013 | 13% | 2% | 4% | 9% | 5% | 6.1% | 63% |
| FCOM-150 | Control | 9% | 1% | 3% | 9% | 9% | 6.1% | 63% |
| PS-1319 | Control | 18% | 3% | 4% | 7% | 10% | 6.6% | 78% |
| MC-13-202 | CMC-012 | 17% | 2% | 4% | 12% | 9% | 6.6% | 78% |
| IPIRANGA-1 | Control | 12% | 3% | 4% | 10% | 9% | 7.0% | 89% |
| CCN-10 | Control | 6% | 1% | 5% | 12% | 7% | 7.5% | 103% |
| FB-206 (BP-S) | Control | 22% | 3% | 5% | 7% | 3% | 8.0% | 115% |
| PH-15 | Control | 36% | 4% | 5% | 13% | 8% | 8.0% | 115% |
| CP-49 | Control | 25% | 1% | 5% | 8% | 6% | 8.1% | 118% |
| PH-16 | Control | 29% | 4% | 9% | 14% | 8% | 10.3% | 175% |
| CATIE-R4 | Control | 17% | 3% | 6% | 15% | 7% | 10.4% | 180% |
| IAC-1 | Control | 13% | 6% | 9% | 21% | 3% | 10.6% | 184% |
| NA-33 | Control | 24% | 4% | 8% | 10% | 3% | 10.9% | 193% |
| PMCT-58 | Control | 64% | 8% | 11% | 19% | 4% | 13.2% | 256% |
| CEPEC-2002 | Control | 27% | 7% | 13% | 21% | 7% | 14.4% | 288% |
| (VB-1151) | | | | | | | | |
| CATIE-R1 | Control | 35% | 6% | 8% | 24% | 16% | 17.2% | 363% |
| Pound-7 (BP-R) | Control | 37% | 6% | 11% | 26% | 22% | 17.3% | 365% |

TABLE 39
Annual prevalence of BPD (Black pod disease) for various varieties at “CT-12” trial
over multiple years and comparison to ‘CCN 51’ control variety.

| | | BPD | BPD | BPD | BPD | BPD | Cumulative | Ranking |
|---|---|---|---|---|---|---|---|---|
| | | (%) | (%) | (%) | (%) | (%) | BPD | vs |
| Genotype name | Variety | 2020 | 2021 | 2022 | 2023 | 2024 | (%) | CCN 51 |
| GU-144C T1 | Control | 0% | 0% | 0% | 0% | 0% | 0.11% | −96% |
| GU-296H | Control | 0% | 0% | 0% | 0% | 3% | 0.28% | −91% |
| CATIE-R6 | Control | 0% | 0% | 0% | 1% | 0% | 0.36% | −88% |
| PT-08-018-2-084-08 | CMC-026 | 0% | 0% | 0% | 0% | 1% | 0.41% | −87% |
| PT-08-057-2-173-09 | CMC-018 | 1% | 0% | 1% | 0% | 0% | 0.44% | −86% |
| NA-33 | Control | 1% | 1% | 0% | 0% | 0% | 0.47% | −85% |
| PT-08-038-3-096-06 | CMC-017 | 0% | 0% | 0% | 1% | 1% | 0.54% | −83% |
| PT-08-064-1-107-18 | CMC-027 | 0% | 0% | 1% | 1% | 0% | 0.58% | −81% |
| SJ-02 | Control | 5% | 1% | 0% | 1% | 1% | 0.59% | −81% |
| PT-08-020-1-145-02 | CMC-001 | 2% | 1% | 1% | 0% | 0% | 0.61% | −80.3% |
| PT-08-018-2-084-18 | CMC-003 | 3% | 0% | 1% | 0% | 1% | 0.66% | −79% |
| SIAL-70 (WB-S) | Control | 1% | 0% | 1% | 0% | 1% | 0.72% | −77% |
| PT-08-018-1-034-01 | CMC-028 | 0% | 1% | 0% | 1% | 1% | 0.77% | −75% |
| GU-171C | Control | 0% | 0% | 0% | 0% | 2% | 0.79% | −75% |
| PT-08-045-2-172-10 | CMC-020 | 1% | 0% | 1% | 1% | 0% | 0.80% | −74% |
| PT-08-003-1-009-05 | CMC-036 | 0% | 0% | 2% | 1% | 0% | 0.86% | −72% |
| PT-08-038-2-050-11 | CMC-023 | 1% | 1% | 1% | 1% | 1% | 0.87% | −72% |
| PT-08-042-3-166-15 | CMC-022 | 0% | 1% | 2% | 0% | 1% | 0.89% | −71% |
| PT-08-020-3-092-11 | CMC-011 | 3% | 0% | 2% | 0% | 0% | 0.93% | −70% |
| PT-08-018-1-034-18 | CMC-009 | 2% | 1% | 1% | 0% | 2% | 0.98% | −69% |
| PT-08-041-2-052-18 | CMC-008 | 3% | 0% | 1% | 1% | 1% | 1.00% | −68% |
| CP-49 | Control | 1% | 1% | 2% | 1% | 0% | 1.04% | −66% |
| PT-08-020-1-145-15 | CMC-007 | 1% | 1% | 2% | 1% | 0% | 1.06% | −66% |
| PT-08-038-3-096-02 | CMC-025 | 2% | 0% | 2% | 1% | 0% | 1.08% | −65% |
| CEPEC-2002 (VB-1151) | Control | 1% | 0% | 1% | 2% | 2% | 1.20% | −61% |
| CATIE-R4 | Control | 0% | 0% | 1% | 2% | 1% | 1.21% | −61% |
| PT-08-041-1-037-17 | CMC-015 | 0% | 0% | 1% | 4% | 1% | 1.28% | −59% |
| MC-08006 | CMC-014 | 1% | 1% | 3% | 1% | 1% | 1.37% | −56% |
| PH-15 | Control | 5% | 0% | 1% | 2% | 1% | 1.37% | −56% |
| PT-08-038-1-113-09 | CMC-004 | 1% | 1% | 2% | 1% | 2% | 1.40% | −55% |
| PT-08-038-1-113-03 | CMC-010 | 0% | 1% | 3% | 2% | 0% | 1.56% | −50% |
| PT-08-020-1-145-12 | CMC-002 | 1% | 1% | 3% | 2% | 0% | 1.58% | −49% |
| MC-08060 | CMC-029 | 3% | 1% | 2% | 1% | 0% | 1.70% | −45% |
| Pound-7 (BP-R) | Control | 0% | 0% | 3% | 2% | 1% | 1.71% | −45% |
| MC-13-202 | CMC-012 | 2% | 2% | 3% | 2% | 1% | 1.74% | −44% |
| MCCS_MP01-192 | CMC-034 | 7% | 1% | 4% | 1% | 1% | 1.75% | −44% |
| VB-514 | Control | 12% | 1% | 3% | 1% | 2% | 1.98% | −36% |
| PT-08-003-3-155-18 | CMC-013 | 3% | 2% | 2% | 1% | 3% | 2.06% | −33% |
| PT-08-034-3-154-07 | CMC-016 | 0% | 1% | 5% | 1% | 2% | 2.07% | −33% |
| PS-1319 | Control | 0% | 1% | 4% | 2% | 2% | 2.21% | −29% |
| PT-08-019-3-164-18 | CMC-021 | 6% | 1% | 3% | 2% | 1% | 2.23% | −28% |
| MCCS_MP01-718 | CMC-033 | 4% | 1% | 3% | 2% | 1% | 2.25% | −27% |
| MC-08092 | CMC-032 | 6% | 3% | 2% | 0% | 0% | 2.33% | −25% |
| PT-08-020-2-057-16 | CMC-024 | 15% | 3% | 2% | 2% | 2% | 2.45% | −21% |
| PT-08-018-1-034-12 | CMC-031 | 10% | 2% | 3% | 1% | 0% | 2.47% | −20% |
| PT-08-038-1-113-18 | CMC-035 | 3% | 1% | 5% | 1% | 1% | 2.48% | −20% |
| PMCT-58 | Control | 0% | 1% | 3% | 4% | 1% | 2.49% | −20% |
| CATIE-R1 | Control | 2% | 1% | 4% | 3% | 3% | 2.62% | −16% |
| FCOM-150 | Control | 9% | 1% | 5% | 1% | 1% | 2.69% | −13% |
| PT-08-020-2-057-12 | CMC-019 | 2% | 3% | 5% | 1% | 1% | 2.72% | −12% |
| TSH-1188 (BP-R) | Control | 0% | 3% | 7% | 0% | 0% | 2.76% | −11% |
| PT-08-018-2-084-05 | CMC-030 | 10% | 1% | 4% | 1% | 1% | 2.79% | −10% |
| IPIRANGA-1 | Control | 0% | 1% | 7% | 3% | 1% | 2.85% | −8% |
| CCN-10 | Control | 13% | 2% | 5% | 1% | 2% | 2.95% | −5% |
| PT-08-038-3-096-10 | CMC-006 | 11% | 1% | 7% | 2% | 0% | 3.05% | −2% |
| CCN-51 | Control | 4% | 3% | 6% | 1% | 2% | 3.10% | 0% |
| SCA-6 (WB-T) | Control | 0% | 1% | 10% | 1% | 0% | 3.10% | 0% |
| PT-08-018-2-084-12 | CMC-005 | 10% | 2% | 2% | 1% | 4% | 3.27% | 6% |
| CEPEC-2004 (CEPEC-46) | Control | 4% | 3% | 7% | 1% | 2% | 3.33% | 7% |
| PH-16 | Control | 15% | 2% | 6% | 1% | 2% | 3.75% | 21% |
| IAC-1 | Control | 0% | 3% | 14% | 4% | 3% | 5.34% | 72% |
| FB-206(BP-S) | Control | 15% | 4% | 4% | 3% | 2% | 5.36% | 73% |

TABLE 40
Number of pods harvested per tree (count) for various varieties at “CT-12” trial over multiple years.

| | | Number of pod harvested per tree (count) | | | | |
|---|---|---|---|---|---|---|
| Genotype name | Variety | 2020 | 2021 | 2022 | 2023 | 2024 |
| PT-08-020-1-145-02 | CMC-001 | 16 | 96 | 110 | 82 | 63 |
| PT-08-020-1-145-12 | CMC-002 | 24 | 60 | 67 | 71 | 43 |
| PT-08-018-2-084-18 | CMC-003 | 31 | 68 | 96 | 68 | 42 |
| PT-08-038-1-113-09 | CMC-004 | 68 | 112 | 122 | 105 | 54 |
| PT-08-018-2-084-12 | CMC-005 | 37 | 45 | 69 | 68 | 47 |
| PT-08-038-3-096-10 | CMC-006 | 13 | 95 | 97 | 79 | 55 |
| PT-08-020-1-145-15 | CMC-007 | 16 | 45 | 54 | 56 | 31 |
| PT-08-041-2-052-18 | CMC-008 | 20 | 44 | 71 | 88 | 56 |
| PT-08-018-1-034-18 | CMC-009 | 22 | 37 | 55 | 56 | 45 |
| PT-08-038-1-113-03 | CMC-010 | 16 | 54 | 71 | 59 | 30 |
| PT-08-020-3-092-11 | CMC-011 | 30 | 55 | 49 | 38 | 59 |
| MC-13-202 | CMC-012 | 4 | 48 | 45 | 50 | 25 |
| PT-08-003-3-155-18 | CMC-013 | 38 | 63 | 81 | 68 | 43 |
| MC-08006 | CMC-014 | 16 | 42 | 48 | 55 | 30 |
| PT-08-041-1-037-17 | CMC-015 | 20 | 63 | 90 | 76 | 53 |
| PT-08-034-3-154-07 | CMC-016 | 25 | 62 | 66 | 95 | 68 |
| PT-08-038-3-096-06 | CMC-017 | 60 | 77 | 68 | 73 | 34 |
| PT-08-057-2-173-09 | CMC-018 | 33 | 65 | 68 | 78 | 47 |
| PT-08-020-2-057-12 | CMC-019 | 10 | 53 | 79 | 64 | 60 |
| PT-08-045-2-172-10 | CMC-020 | 19 | 60 | 72 | 70 | 19 |
| PT-08-019-3-164-18 | CMC-021 | 28 | 52 | 63 | 66 | 30 |
| PT-08-042-3-166-15 | CMC-022 | 25 | 31 | 45 | 45 | 14 |
| PT-08-038-2-050-11 | CMC-023 | 8 | 60 | 86 | 67 | 30 |
| PT-08-020-2-057-16 | CMC-024 | 4 | 65 | 61 | 62 | 35 |
| PT-08-038-3-096-02 | CMC-025 | 13 | 54 | 58 | 56 | 42 |
| PT-08-018-2-084-08 | CMC-026 | 14 | 42 | 49 | 45 | 29 |
| PT-08-064-1-107-18 | CMC-027 | 12 | 35 | 45 | 64 | 30 |
| PT-08-018-1-034-01 | CMC-028 | 18 | 47 | 46 | 45 | 35 |
| MC-08060 | CMC-029 | 32 | 45 | 52 | 51 | 23 |
| PT-08-018-2-084-05 | CMC-030 | 16 | 41 | 49 | 47 | 23 |
| PT-08-018-1-034-12 | CMC-031 | 29 | 54 | 65 | 71 | 28 |
| MC-08092 | CMC-032 | 22 | 36 | 35 | 50 | 23 |
| MCCS_MP01-718 | CMC-033 | 13 | 28 | 43 | 38 | 15 |
| MCCS_MP01-192 | CMC-034 | 2 | 24 | 39 | 45 | 20 |
| PT-08-038-1-113-18 | CMC-035 | 16 | 41 | 62 | 76 | 28 |
| PT-08-003-1-009-05 | CMC-036 | 12 | 33 | 34 | 35 | 15 |
| CATIE-R1 | Control | 19 | 23 | 29 | 29 | 14 |
| CATIE-R4 | Control | 3 | 9 | 20 | 38 | 13 |
| CATIE-R6 | Control | 4 | 14 | 26 | 60 | 25 |
| CCN-10 | Control | 1 | 8 | 16 | 22 | 5 |
| CCN-51 | Control | 4 | 21 | 27 | 39 | 17 |
| CEPEC-2002 (VB-1151) | Control | 21 | 45 | 48 | 45 | 22 |
| CEPEC-2004 (CEPEC-46) | Control | 9 | 20 | 38 | 62 | 18 |
| CP-49 | Control | 20 | 32 | 30 | 43 | 25 |
| FB-206 (BP-S) | Control | 33 | 33 | 46 | 48 | 27 |
| FCOM-150 | Control | 13 | 39 | 58 | 66 | 41 |
| GU-144C T1 | Control | 9 | 21 | 17 | 20 | 11 |
| GU-171C | Control | 11 | 5 | 3 | 4 | 19 |
| GU-296H | Control | 3 | 10 | 10 | 4 | 3 |
| IAC-1 | Control | 2 | 25 | 27 | 41 | 39 |
| IPIRANGA-1 | Control | 7 | 16 | 20 | 23 | 11 |
| NA-33 | Control | 24 | 13 | 31 | 29 | 11 |
| PH-15 | Control | 3 | 25 | 42 | 30 | 11 |
| PH-16 | Control | 7 | 27 | 29 | 28 | 10 |
| PMCT-58 | Control | 1 | 11 | 14 | 22 | 11 |
| Pound-7 (BP-R) | Control | 8 | 24 | 32 | 28 | 12 |
| PS-1319 | Control | 5 | 16 | 31 | 48 | 21 |
| SCA-6 (WB-T) | Control | 5 | 23 | 49 | 69 | 50 |
| SIAL-70 (WB-S) | Control | 12 | 22 | 37 | 42 | 25 |
| SJ-02 | Control | 2 | 28 | 49 | 58 | 35 |
| TSH-1188 (BP-R) | Control | 0 | 3 | 4 | 3 | 3 |
| VB-514 | Control | 6 | 30 | 53 | 51 | 28 |

TABLE 41
Number of healthy pods harvested per tree (count) for various varieties at “CT-12” trial over multiple years.

| | | Number of healthy pod harvested per tree (count) | | | | |
|---|---|---|---|---|---|---|
| Genotype name | Variety | 2020 | 2021 | 2022 | 2023 | 2024 |
| PT-08-020-1-145-02 | CMC-001 | 11 | 89 | 106 | 75 | 57 |
| PT-08-020-1-145-12 | CMC-002 | 20 | 54 | 63 | 62 | 34 |
| PT-08-018-2-084-18 | CMC-003 | 25 | 63 | 93 | 59 | 37 |
| PT-08-038-1-113-09 | CMC-004 | 56 | 97 | 112 | 90 | 45 |
| PT-08-018-2-084-12 | CMC-005 | 26 | 40 | 65 | 60 | 40 |
| PT-08-038-3-096-10 | CMC-006 | 9 | 73 | 79 | 67 | 46 |
| PT-08-020-1-145-15 | CMC-007 | 13 | 42 | 50 | 52 | 28 |
| PT-08-041-2-052-18 | CMC-008 | 14 | 36 | 65 | 76 | 46 |
| PT-08-018-1-034-18 | CMC-009 | 17 | 32 | 52 | 50 | 40 |
| PT-08-038-1-113-03 | CMC-010 | 13 | 44 | 62 | 48 | 20 |
| PT-08-020-3-092-11 | CMC-011 | 22 | 54 | 46 | 35 | 44 |
| MC-13-202 | CMC-012 | 2 | 41 | 41 | 40 | 19 |
| PT-08-003-3-155-18 | CMC-013 | 22 | 42 | 66 | 53 | 34 |
| MC-08006 | CMC-014 | 12 | 36 | 42 | 48 | 25 |
| PT-08-041-1-037-17 | CMC-015 | 13 | 57 | 81 | 66 | 50 |
| PT-08-034-3-154-07 | CMC-016 | 17 | 49 | 51 | 83 | 52 |
| PT-08-038-3-096-06 | CMC-017 | 48 | 68 | 66 | 65 | 31 |
| PT-08-057-2-173-09 | CMC-018 | 28 | 57 | 64 | 68 | 43 |
| PT-08-020-2-057-12 | CMC-019 | 6 | 48 | 72 | 56 | 52 |
| PT-08-045-2-172-10 | CMC-020 | 14 | 55 | 65 | 63 | 17 |
| PT-08-019-3-164-18 | CMC-021 | 20 | 43 | 56 | 57 | 27 |
| PT-08-042-3-166-15 | CMC-022 | 21 | 28 | 42 | 39 | 12 |
| PT-08-038-2-050-11 | CMC-023 | 5 | 50 | 79 | 61 | 25 |
| PT-08-020-2-057-16 | CMC-024 | 2 | 56 | 56 | 55 | 30 |
| PT-08-038-3-096-02 | CMC-025 | 10 | 44 | 55 | 50 | 38 |
| PT-08-018-2-084-08 | CMC-026 | 9 | 39 | 47 | 43 | 26 |
| PT-08-064-1-107-18 | CMC-027 | 8 | 31 | 41 | 58 | 27 |
| PT-08-018-1-034-01 | CMC-028 | 15 | 44 | 45 | 39 | 30 |
| MC-08060 | CMC-029 | 25 | 38 | 48 | 46 | 18 |
| PT-08-018-2-084-05 | CMC-030 | 11 | 31 | 42 | 41 | 20 |
| PT-08-018-1-034-12 | CMC-031 | 21 | 49 | 61 | 67 | 26 |
| MC-08092 | CMC-032 | 17 | 33 | 34 | 44 | 20 |
| MCCS_MP01-718 | CMC-033 | 9 | 22 | 36 | 32 | 12 |
| MCCS_MP01-192 | CMC-034 | 1 | 19 | 31 | 38 | 15 |
| PT-08-038-1-113-18 | CMC-035 | 11 | 35 | 54 | 68 | 22 |
| PT-08-003-1-009-05 | CMC-036 | 6 | 24 | 29 | 29 | 11 |
| CATIE-R1 | Control | 11 | 19 | 24 | 20 | 11 |
| CATIE-R4 | Control | 2 | 8 | 17 | 30 | 8 |
| CATIE-R6 | Control | 2 | 13 | 23 | 52 | 19 |
| CCN-10 | Control | 0 | 6 | 11 | 17 | 3 |
| CCN-51 | Control | 3 | 15 | 19 | 33 | 14 |
| CEPEC-2002 (VB-1151) | Control | 14 | 39 | 40 | 33 | 17 |
| CEPEC-2004 (CEPEC-46) | Control | 6 | 17 | 31 | 53 | 13 |
| CP-49 | Control | 14 | 28 | 26 | 37 | 23 |
| FB-206 (BP-S) | Control | 15 | 27 | 41 | 42 | 24 |
| FCOM-150 | Control | 9 | 36 | 52 | 57 | 36 |
| GU-144C T1 | Control | 7 | 19 | 17 | 19 | 9 |
| GU-171C | Control | 8 | 5 | 2 | 3 | 17 |
| GU-296H | Control | 2 | 9 | 9 | 3 | 2 |
| IAC-1 | Control | 1 | 22 | 19 | 25 | 24 |
| IPIRANGA-1 | Control | 4 | 13 | 14 | 18 | 7 |
| NA-33 | Control | 17 | 12 | 28 | 25 | 9 |
| PH-15 | Control | 1 | 21 | 37 | 23 | 9 |
| PH-16 | Control | 3 | 18 | 21 | 21 | 8 |
| PMCT-58 | Control | 0 | 9 | 11 | 15 | 8 |
| Pound-7 (BP-R) | Control | 4 | 21 | 26 | 18 | 8 |
| PS-1319 | Control | 3 | 15 | 27 | 41 | 16 |
| SCA-6 (WB-T) | Control | 3 | 21 | 18 | 22 | 21 |
| SIAL-70 (WB-S) | Control | 8 | 21 | 35 | 38 | 23 |
| SJ-02 | Control | 1 | 24 | 41 | 49 | 28 |
| TSH-1188 (BP-R) | Control | 0 | 3 | 3 | 3 | 2 |
| VB-514 | Control | 4 | 23 | 44 | 39 | 17 |

TABLE 42
Pod fresh weight (g) for various varieties at “CT-12” trial over multiple years.

| | | Pod Fresh Weight (g) | | | | |
|---|---|---|---|---|---|---|
| Genotype name | Variety | 2020 | 2021 | 2022 | 2023 | 2024 |
| PT-08-020-1-145-02 | CMC-001 | 318.22 | 223.97 | 323.96 | 365.23 | 282.25 |
| PT-08-020-1-145-12 | CMC-002 | 595.82 | 254.27 | 374.66 | 409.03 | 312.28 |
| PT-08-018-2-084-18 | CMC-003 | 414.41 | 205.72 | 276.46 | 226.90 | 223.30 |
| PT-08-038-1-113-09 | CMC-004 | 680.03 | 215.49 | 183.53 | 104.09 | 188.12 |
| PT-08-018-2-084-12 | CMC-005 | 655.49 | 257.00 | 273.51 | 225.12 | 272.70 |
| PT-08-038-3-096-10 | CMC-006 | 358.49 | 224.54 | 305.21 | 269.37 | 234.88 |
| PT-08-020-1-145-15 | CMC-007 | 495.48 | 315.78 | 434.89 | 398.04 | 411.20 |
| PT-08-041-2-052-18 | CMC-008 | 455.54 | 363.81 | 459.48 | 390.68 | 246.21 |
| PT-08-018-1-034-18 | CMC-009 | 638.82 | 378.05 | 395.59 | 391.97 | 253.78 |
| PT-08-038-1-113-03 | CMC-010 | 402.78 | 219.29 | 351.61 | 364.31 | 193.58 |
| PT-08-020-3-092-11 | CMC-011 | 433.06 | 169.02 | 201.09 | 115.70 | 76.73 |
| MC-13-202 | CMC-012 | 123.41 | 270.80 | 295.76 | 290.34 | 227.04 |
| PT-08-003-3-155-18 | CMC-013 | 585.58 | 192.01 | 243.97 | 231.12 | 228.37 |
| MC-08006 | CMC-014 | 403.35 | 283.89 | 324.03 | 220.25 | 206.06 |
| PT-08-041-1-037-17 | CMC-015 | 429.92 | 304.18 | 377.88 | 389.88 | 318.81 |
| PT-08-034-3-154-07 | CMC-016 | 426.29 | 187.32 | 256.36 | 230.42 | 195.70 |
| PT-08-038-3-096-06 | CMC-017 | 631.26 | 197.60 | 229.10 | 231.63 | 211.51 |
| PT-08-057-2-173-09 | CMC-018 | 418.86 | 223.46 | 204.93 | 139.76 | 97.73 |
| PT-08-020-2-057-12 | CMC-019 | 250.87 | 280.41 | 354.24 | 262.96 | 262.22 |
| PT-08-045-2-172-10 | CMC-020 | 270.06 | 199.32 | 280.88 | 209.57 | 160.24 |
| PT-08-019-3-164-18 | CMC-021 | 542.20 | 246.43 | 327.38 | 299.79 | 342.76 |
| PT-08-042-3-166-15 | CMC-022 | 564.26 | 355.74 | 422.53 | 433.01 | 162.57 |
| PT-08-038-2-050-11 | CMC-023 | 266.01 | 293.29 | 345.73 | 327.98 | 202.86 |
| PT-08-020-2-057-16 | CMC-024 | 114.02 | 286.76 | 340.49 | 361.01 | 207.63 |
| PT-08-038-3-096-02 | CMC-025 | 314.56 | 285.70 | 415.54 | 266.85 | 312.86 |
| PT-08-018-2-084-08 | CMC-026 | 461.45 | 310.20 | 358.66 | 410.98 | 344.67 |
| PT-08-064-1-107-18 | CMC-027 | 296.50 | 294.40 | 357.76 | 363.97 | 298.76 |
| PT-08-018-1-034-01 | CMC-028 | 448.41 | 314.56 | 356.10 | 327.10 | 349.88 |
| MC-08060 | CMC-029 | 573.43 | 274.72 | 327.07 | 237.65 | 193.15 |
| PT-08-018-2-084-05 | CMC-030 | 487.19 | 290.05 | 282.57 | 132.63 | 196.67 |
| PT-08-018-1-034-12 | CMC-031 | 490.97 | 168.16 | 323.14 | 303.95 | 252.04 |
| MC-08092 | CMC-032 | 503.61 | 177.06 | 192.89 | 173.05 | 85.36 |
| MCCS_MP01-718 | CMC-033 | 399.17 | 347.98 | 497.56 | 417.47 | 329.16 |
| MCCS_MP01-192 | CMC-034 | 79.37 | 318.94 | 597.71 | 556.21 | 487.70 |
| PT-08-038-1-113-18 | CMC-035 | 371.75 | 217.70 | 301.67 | 266.73 | 166.61 |
| PT-08-003-1-009-05 | CMC-036 | 176.05 | 299.72 | 371.66 | 244.44 | 227.50 |
| CATIE-R1 | Control | 390.22 | 138.86 | 306.65 | 237.35 | 187.91 |
| CATIE-R4 | Control | 100.16 | 127.01 | 288.24 | 257.75 | 91.18 |
| CATIE-R6 | Control | 99.92 | 159.94 | 315.09 | 311.84 | 170.13 |
| CCN-10 | Control | 21.71 | 186.93 | 204.17 | 303.35 | 61.79 |
| CCN-51 | Control | 216.44 | 309.98 | 416.81 | 291.00 | 258.19 |
| CEPEC-2002 (VB-1151) | Control | 397.44 | 212.82 | 297.18 | 271.04 | 206.11 |
| CEPEC-2004 (CEPEC-46) | Control | 230.52 | 181.84 | 273.72 | 301.65 | 162.35 |
| CP-49 | Control | 425.71 | 254.08 | 348.09 | 175.69 | 149.31 |
| FB-206 (BP-S) | Control | 375.69 | 259.30 | 322.88 | 243.19 | 248.65 |
| FCOM-150 | Control | 287.04 | 332.33 | 283.56 | 276.71 | 299.09 |
| GU-144C T1 | Control | 231.48 | 174.22 | 247.43 | 179.56 | 181.59 |
| GU-171C | Control | 203.81 | 42.99 | 46.05 | 41.78 | 178.15 |
| GU-296H | Control | 70.83 | 116.15 | 140.72 | 36.11 | 38.33 |
| IAC-1 | Control | 97.23 | 252.20 | 312.41 | 394.88 | 234.66 |
| IPIRANGA-1 | Control | 185.44 | 360.03 | 301.85 | 199.67 | 275.96 |
| NA-33 | Control | 348.79 | 136.82 | 289.78 | 249.07 | 120.58 |
| PH-15 | Control | 74.97 | 196.68 | 337.95 | 247.31 | 100.68 |
| PH-16 | Control | 268.50 | 252.74 | 384.11 | 306.79 | 164.84 |
| PMCT-58 | Control | 12.14 | 207.94 | 312.02 | 328.79 | 54.00 |
| Pound-7 (BP-R) | Control | 196.39 | 221.76 | 392.89 | 264.19 | 120.00 |
| PS-1319 | Control | 143.36 | 190.43 | 408.75 | 308.46 | 185.03 |
| SCA-6 (WB-T) | Control | 54.32 | 90.75 | 135.29 | 150.13 | 115.43 |
| SIAL-70 (WB-S) | Control | 310.32 | 210.40 | 288.36 | 285.90 | 191.39 |
| SJ-02 | Control | 64.77 | 337.02 | 411.55 | 410.03 | 296.03 |
| TSH-1188 (BP-R) | Control | 7.75 | 52.17 | 314.31 | 105.41 | 15.91 |
| VB-514 | Control | 150.43 | 193.24 | 298.61 | 227.12 | 99.23 |

TABLE 43
Average dry bean weight per pod (g) for various varieties at “CT-12” trial over multiple years.

| | | Average dry bean weight per pod (g) | | | | |
|---|---|---|---|---|---|---|
| Genotype name | Variety | 2020 | 2021 | 2022 | 2023 | 2024 |
| PT-08-020-1-145-02 | CMC-001 | 22.58 | 22.04 | 29.64 | 27.62 | 28.78 |
| PT-08-020-1-145-12 | CMC-002 | 31.91 | 29.01 | 36.47 | 31.31 | 34.51 |
| PT-08-018-2-084-18 | CMC-003 | 23.23 | 23.07 | 23.03 | 20.11 | 22.44 |
| PT-08-038-1-113-09 | CMC-004 | 15.25 | 14.30 | 17.83 | 14.68 | 15.32 |
| PT-08-018-2-084-12 | CMC-005 | 25.91 | 24.31 | 24.34 | 22.32 | 26.42 |
| PT-08-038-3-096-10 | CMC-006 | 31.65 | 19.74 | 23.36 | 21.22 | 21.97 |
| PT-08-020-1-145-15 | CMC-007 | 37.37 | 37.54 | 43.86 | 38.72 | 43.97 |
| PT-08-041-2-052-18 | CMC-008 | 27.17 | 28.82 | 34.63 | 28.37 | 29.71 |
| PT-08-018-1-034-18 | CMC-009 | 33.13 | 33.78 | 37.65 | 34.71 | 37.03 |
| PT-08-038-1-113-03 | CMC-010 | 30.26 | 25.10 | 30.09 | 31.22 | 34.22 |
| PT-08-020-3-092-11 | CMC-011 | 22.17 | 19.92 | 22.65 | 19.76 | 15.40 |
| MC-13-202 | CMC-012 | 36.47 | 38.09 | 38.36 | 37.41 | 43.24 |
| PT-08-003-3-155-18 | CMC-013 | 25.08 | 22.93 | 24.84 | 22.94 | 26.16 |
| MC-08006 | CMC-014 | 30.40 | 31.01 | 35.94 | 30.53 | 33.79 |
| PT-08-041-1-037-17 | CMC-015 | 23.87 | 25.53 | 28.13 | 25.60 | 29.58 |
| PT-08-034-3-154-07 | CMC-016 | 27.15 | 23.36 | 28.16 | 24.83 | 23.27 |
| PT-08-038-3-096-06 | CMC-017 | 16.23 | 17.24 | 19.23 | 17.45 | 19.98 |
| PT-08-057-2-173-09 | CMC-018 | 23.17 | 21.46 | 27.02 | 22.26 | 22.15 |
| PT-08-020-2-057-12 | CMC-019 | 30.09 | 28.07 | 34.01 | 27.95 | 29.11 |
| PT-08-045-2-172-10 | CMC-020 | 25.87 | 24.53 | 26.40 | 22.88 | 29.18 |
| PT-08-019-3-164-18 | CMC-021 | 27.46 | 29.37 | 30.14 | 26.57 | 33.35 |
| PT-08-042-3-166-15 | CMC-022 | 25.88 | 33.39 | 37.66 | 31.54 | 35.19 |
| PT-08-038-2-050-11 | CMC-023 | 26.38 | 22.87 | 26.29 | 21.31 | 22.99 |
| PT-08-020-2-057-16 | CMC-024 | 32.83 | 29.35 | 32.59 | 29.61 | 29.69 |
| PT-08-038-3-096-02 | CMC-025 | 26.70 | 28.95 | 27.53 | 26.63 | 27.58 |
| PT-08-018-2-084-08 | CMC-026 | 27.15 | 23.90 | 31.09 | 26.59 | 29.81 |
| PT-08-064-1-107-18 | CMC-027 | 32.87 | 34.12 | 39.09 | 34.23 | 40.14 |
| PT-08-018-1-034-01 | CMC-028 | 27.37 | 22.02 | 28.60 | 26.71 | 28.38 |
| MC-08060 | CMC-029 | 19.18 | 23.03 | 22.84 | 21.47 | 22.81 |
| PT-08-018-2-084-05 | CMC-030 | 29.98 | 28.23 | 27.91 | 25.82 | 36.44 |
| PT-08-018-1-034-12 | CMC-031 | 23.03 | 17.91 | 22.72 | 20.16 | 21.67 |
| MC-08092 | CMC-032 | 25.21 | 24.08 | 27.70 | 25.75 | 20.71 |
| MCCS_MP01-718 | CMC-033 | 39.00 | 37.26 | 42.79 | 37.09 | 37.75 |
| MCCS_MP01-192 | CMC-034 | 50.41 | 45.39 | 49.04 | 48.82 | 52.81 |
| PT-08-038-1-113-18 | CMC-035 | 25.20 | 22.75 | 24.52 | 23.40 | 24.23 |
| PT-08-003-1-009-05 | CMC-036 | 43.44 | 42.14 | 44.45 | 41.85 | 48.50 |
| CATIE-R1 | Control | 19.93 | 20.87 | 21.82 | 21.32 | 23.82 |
| CATIE-R4 | Control | 20.79 | 16.12 | 19.60 | 17.62 | 21.22 |
| CATIE-R6 | Control | 17.86 | 15.13 | 15.95 | 14.68 | 16.75 |
| CCN-10 | Control | 40.09 | 42.93 | 39.39 | 42.34 | 46.89 |
| CCN-51 | Control | 45.68 | 44.96 | 48.60 | 46.18 | 46.32 |
| CEPEC-2002 (VB-1151) | Control | 19.55 | 22.56 | 26.98 | 22.22 | 26.72 |
| CEPEC-2004 (CEPEC-46) | Control | 19.49 | 20.69 | 23.10 | 19.46 | 21.46 |
| CP-49 | Control | 25.90 | 24.80 | 28.84 | 24.46 | 26.13 |
| FB-206 (BP-S) | Control | 23.34 | 27.62 | 26.35 | 23.10 | 28.14 |
| FCOM-150 | Control | 29.10 | 29.44 | 31.50 | 23.24 | 28.37 |
| GU-144C T1 | Control | 17.48 | 13.58 | 17.11 | 16.09 | 17.45 |
| GU-171C | Control | 6.20 | 5.62 | 8.17 | 10.86 | 8.21 |
| GU-296H | Control | 10.11 | 9.43 | 13.70 | 11.01 | 18.39 |
| IAC-1 | Control | 28.18 | 18.65 | 16.21 | 16.09 | 20.32 |
| IPIRANGA-1 | Control | 30.80 | 32.54 | 35.67 | 33.96 | 33.97 |
| NA-33 | Control | 16.33 | 19.74 | 19.71 | 17.98 | 19.21 |
| PH-15 | Control | 28.86 | 31.16 | 33.49 | 29.84 | 29.85 |
| PH-16 | Control | 36.71 | 37.88 | 35.81 | 34.10 | 36.71 |
| PMCT-58 | Control | 15.40 | 20.20 | 23.82 | 22.68 | 24.72 |
| Pound-7 (BP-R) | Control | 35.89 | 33.75 | 31.19 | 28.38 | 33.78 |
| PS-1319 | Control | 31.43 | 30.36 | 41.84 | 34.63 | 40.08 |
| SCA-6 (WB-T) | Control | 12.93 | 3.96 | 4.62 | 5.27 | 4.58 |
| SIAL-70 (WB-S) | Control | 31.82 | 24.81 | 28.28 | 25.29 | 25.67 |
| SJ-02 | Control | 36.98 | 33.94 | 37.46 | 33.32 | 37.90 |
| TSH-1188 (BP-R) | Control | 2.90 | 34.60 | 57.38 | 41.58 | 44.52 |
| VB-514 | Control | 12.11 | 8.26 | 9.87 | 8.76 | 9.43 |

TABLE 44
Average bean size (g) for various varieties at “CT-12” trial over multiple years.

| | | Average bean size (g) | | | | |
|---|---|---|---|---|---|---|
| Genotype name | Variety | 2020 | 2021 | 2022 | 2023 | 2024 |
| PT-08-020-1-145-02 | CMC-001 | 0.88 | 0.91 | 1.07 | 0.94 | 0.89 |
| PT-08-020-1-145-12 | CMC-002 | 1.04 | 1.11 | 1.34 | 1.18 | 0.96 |
| PT-08-018-2-084-18 | CMC-003 | 0.90 | 0.96 | 0.98 | 0.87 | 0.78 |
| PT-08-038-1-113-09 | CMC-004 | 0.58 | 0.54 | 0.64 | 0.63 | 0.63 |
| PT-08-018-2-084-12 | CMC-005 | 1.05 | 1.08 | 1.11 | 1.02 | 0.99 |
| PT-08-038-3-096-10 | CMC-006 | 0.93 | 0.85 | 0.85 | 0.82 | 0.79 |
| PT-08-020-1-145-15 | CMC-007 | 1.20 | 1.30 | 1.38 | 1.37 | 1.15 |
| PT-08-041-2-052-18 | CMC-008 | 1.04 | 1.35 | 1.36 | 1.20 | 1.01 |
| PT-08-018-1-034-18 | CMC-009 | 1.27 | 1.34 | 1.44 | 1.24 | 1.26 |
| PT-08-038-1-113-03 | CMC-010 | 1.07 | 1.17 | 1.19 | 1.13 | 1.02 |
| PT-08-020-3-092-11 | CMC-011 | 0.80 | 0.85 | 0.96 | 0.76 | 0.57 |
| MC-13-202 | CMC-012 | 1.28 | 1.43 | 1.43 | 1.40 | 1.33 |
| PT-08-003-3-155-18 | CMC-013 | 0.96 | 1.00 | 0.94 | 0.86 | 0.86 |
| MC-08006 | CMC-014 | 1.06 | 1.23 | 1.25 | 1.22 | 1.11 |
| PT-08-041-1-037-17 | CMC-015 | 1.10 | 1.17 | 1.31 | 1.25 | 1.06 |
| PT-08-034-3-154-07 | CMC-016 | 0.95 | 0.92 | 1.09 | 0.97 | 0.89 |
| PT-08-038-3-096-06 | CMC-017 | 0.74 | 0.78 | 0.73 | 0.78 | 0.85 |
| PT-08-057-2-173-09 | CMC-018 | 1.09 | 1.27 | 1.31 | 1.07 | 0.96 |
| PT-08-020-2-057-12 | CMC-019 | 0.97 | 1.09 | 1.16 | 1.06 | 0.92 |
| PT-08-045-2-172-10 | CMC-020 | 1.04 | 1.20 | 1.23 | 1.07 | 1.12 |
| PT-08-019-3-164-18 | CMC-021 | 0.98 | 1.14 | 1.14 | 1.02 | 1.04 |
| PT-08-042-3-166-15 | CMC-022 | 1.13 | 1.22 | 1.39 | 1.28 | 0.98 |
| PT-08-038-2-050-11 | CMC-023 | 0.87 | 0.82 | 0.81 | 0.74 | 0.73 |
| PT-08-020-2-057-16 | CMC-024 | 1.13 | 1.26 | 1.28 | 1.15 | 1.04 |
| PT-08-038-3-096-02 | CMC-025 | 0.93 | 1.03 | 0.99 | 0.92 | 0.95 |
| PT-08-018-2-084-08 | CMC-026 | 1.24 | 1.22 | 1.26 | 1.20 | 1.10 |
| PT-08-064-1-107-18 | CMC-027 | 1.13 | 1.26 | 1.29 | 1.17 | 1.12 |
| PT-08-018-1-034-01 | CMC-028 | 0.98 | 0.99 | 1.09 | 1.05 | 0.89 |
| MC-08060 | CMC-029 | 0.69 | 0.89 | 0.77 | 0.71 | 0.78 |
| PT-08-018-2-084-05 | CMC-030 | 1.01 | 1.02 | 0.95 | 0.86 | 0.98 |
| PT-08-018-1-034-12 | CMC-031 | 0.88 | 0.80 | 0.98 | 0.87 | 0.88 |
| MC-08092 | CMC-032 | 1.00 | 1.19 | 1.08 | 1.05 | 0.92 |
| MCCS_MP01-718 | CMC-033 | 1.03 | 1.16 | 1.24 | 1.03 | 1.07 |
| MCCS_MP01-192 | CMC-034 | 1.32 | 1.26 | 1.39 | 1.32 | 1.28 |
| PT-08-038-1-113-18 | CMC-035 | 0.89 | 0.89 | 0.92 | 0.89 | 1.00 |
| PT-08-003-1-009-05 | CMC-036 | 1.17 | 1.37 | 1.19 | 1.23 | 1.21 |
| CATIE-R1 | Control | 1.11 | 1.29 | 1.16 | 1.17 | 1.12 |
| CATIE-R4 | Control | 1.18 | 1.14 | 1.27 | 1.23 | 1.17 |
| CATIE-R6 | Control | 1.23 | 1.10 | 1.28 | 1.28 | 1.35 |
| CCN-10 | Control | 1.49 | 1.97 | 1.89 | 1.85 | 1.82 |
| CCN-51 | Control | 1.38 | 1.41 | 1.29 | 1.27 | 1.33 |
| CEPEC-2002 (VB-1151) | Control | 0.82 | 0.84 | 0.85 | 0.79 | 0.77 |
| CEPEC-2004 (CEPEC-46) | Control | 0.90 | 1.05 | 1.12 | 0.92 | 0.93 |
| CP-49 | Control | 0.92 | 1.01 | 0.96 | 0.86 | 0.92 |
| FB-206 (BP-S) | Control | 0.77 | 1.03 | 0.89 | 0.80 | 0.85 |
| FCOM-150 | Control | 0.86 | 0.93 | 1.00 | 0.84 | 0.88 |
| GU-144C T1 | Control | 0.81 | 0.80 | 0.93 | 0.92 | 0.89 |
| GU-171C | Control | 0.53 | 0.39 | 0.49 | 0.71 | 0.57 |
| GU-296H | Control | 0.70 | 0.73 | 0.84 | 0.81 | 0.78 |
| IAC-1 | Control | 1.18 | 1.26 | 1.36 | 1.22 | 1.17 |
| IPIRANGA-1 | Control | 0.87 | 1.06 | 1.03 | 0.93 | 0.92 |
| NA-33 | Control | 0.86 | 0.97 | 1.04 | 0.97 | 1.01 |
| PH-15 | Control | 0.87 | 1.12 | 1.02 | 1.01 | 0.90 |
| PH-16 | Control | 1.17 | 1.26 | 1.10 | 1.03 | 1.01 |
| PMCT-58 | Control | 0.93 | 1.03 | 1.16 | 1.19 | 1.01 |
| Pound-7 (BP-R) | Control | 1.10 | 1.09 | 1.12 | 0.99 | 1.07 |
| PS-1319 | Control | 0.98 | 1.07 | 1.26 | 1.08 | 1.03 |
| SCA-6 (WB-T) | Control | 0.61 | 0.61 | 0.79 | 0.71 | 0.63 |
| SIAL-70 (WB-S) | Control | 0.95 | 0.98 | 1.06 | 0.92 | 0.81 |
| SJ-02 | Control | 1.37 | 1.15 | 1.24 | 1.09 | 1.13 |
| TSH-1188 (BP-R) | Control | 0.58 | 1.29 | 1.57 | 1.46 | 1.17 |
| VB-514 | Control | 0.73 | 0.63 | 0.83 | 0.72 | 0.78 |

TABLE 45
Average seeds per pod (count) for various varieties at “CT-12” trial over multiple years.

| | | Average seeds per pod (count) | | | | |
|---|---|---|---|---|---|---|
| Genotype name | Variety | 2020 | 2021 | 2022 | 2023 | 2024 |
| PT-08-020-1-145-02 | CMC-001 | 27 | 34 | 34 | 33 | 31 |
| PT-08-020-1-145-12 | CMC-002 | 32 | 34 | 34 | 34 | 35 |
| PT-08-018-2-084-18 | CMC-003 | 27 | 30 | 30 | 26 | 29 |
| PT-08-038-1-113-09 | CMC-004 | 27 | 29 | 33 | 34 | 26 |
| PT-08-018-2-084-12 | CMC-005 | 26 | 29 | 30 | 26 | 27 |
| PT-08-038-3-096-10 | CMC-006 | 34 | 27 | 29 | 27 | 29 |
| PT-08-020-1-145-15 | CMC-007 | 32 | 35 | 34 | 33 | 35 |
| PT-08-041-2-052-18 | CMC-008 | 26 | 30 | 30 | 29 | 31 |
| PT-08-018-1-034-18 | CMC-009 | 27 | 30 | 30 | 30 | 29 |
| PT-08-038-1-113-03 | CMC-010 | 28 | 33 | 33 | 31 | 31 |
| PT-08-020-3-092-11 | CMC-011 | 29 | 29 | 28 | 28 | 28 |
| MC-13-202 | CMC-012 | 29 | 31 | 28 | 31 | 32 |
| PT-08-003-3-155-18 | CMC-013 | 27 | 32 | 31 | 29 | 31 |
| MC-08006 | CMC-014 | 29 | 30 | 34 | 29 | 27 |
| PT-08-041-1-037-17 | CMC-015 | 22 | 29 | 30 | 28 | 27 |
| PT-08-034-3-154-07 | CMC-016 | 28 | 34 | 31 | 33 | 29 |
| PT-08-038-3-096-06 | CMC-017 | 23 | 29 | 29 | 26 | 26 |
| PT-08-057-2-173-09 | CMC-018 | 22 | 24 | 23 | 23 | 23 |
| PT-08-020-2-057-12 | CMC-019 | 30 | 32 | 33 | 33 | 30 |
| PT-08-045-2-172-10 | CMC-020 | 27 | 30 | 29 | 32 | 26 |
| PT-08-019-3-164-18 | CMC-021 | 29 | 35 | 32 | 29 | 36 |
| PT-08-042-3-166-15 | CMC-022 | 23 | 30 | 32 | 29 | 27 |
| PT-08-038-2-050-11 | CMC-023 | 30 | 33 | 34 | 31 | 30 |
| PT-08-020-2-057-16 | CMC-024 | 27 | 33 | 32 | 29 | 27 |
| PT-08-038-3-096-02 | CMC-025 | 29 | 28 | 31 | 28 | 30 |
| PT-08-018-2-084-08 | CMC-026 | 22 | 24 | 27 | 24 | 24 |
| PT-08-064-1-107-18 | CMC-027 | 30 | 30 | 29 | 34 | 36 |
| PT-08-018-1-034-01 | CMC-028 | 30 | 31 | 31 | 30 | 28 |
| MC-08060 | CMC-029 | 28 | 28 | 37 | 29 | 28 |
| PT-08-018-2-084-05 | CMC-030 | 30 | 30 | 31 | 31 | 36 |
| PT-08-018-1-034-12 | CMC-031 | 29 | 29 | 28 | 26 | 25 |
| MC-08092 | CMC-032 | 26 | 28 | 26 | 27 | 22 |
| MCCS_MP01-718 | CMC-033 | 36 | 38 | 37 | 34 | 34 |
| MCCS_MP01-192 | CMC-034 | 38 | 42 | 38 | 40 | 37 |
| PT-08-038-1-113-18 | CMC-035 | 28 | 28 | 31 | 28 | 23 |
| PT-08-003-1-009-05 | CMC-036 | 37 | 35 | 41 | 34 | 35 |
| CATIE-R1 | Control | 18 | 21 | 19 | 17 | 18 |
| CATIE-R4 | Control | 18 | 19 | 18 | 15 | 18 |
| CATIE-R6 | Control | 15 | 19 | 16 | 12 | 11 |
| CCN-10 | Control | 27 | 25 | 23 | 23 | 28 |
| CCN-51 | Control | 34 | 38 | 37 | 38 | 33 |
| CEPEC-2002 (VB-1151) | Control | 25 | 36 | 32 | 32 | 34 |
| CEPEC-2004 (CEPEC-46) | Control | 21 | 21 | 22 | 21 | 20 |
| CP-49 | Control | 30 | 33 | 29 | 31 | 30 |
| FB-206 (BP-S) | Control | 32 | 36 | 35 | 33 | 33 |
| FCOM-150 | Control | 34 | 37 | 36 | 31 | 30 |
| GU-144C T1 | Control | 21 | 17 | 21 | 18 | 18 |
| GU-171C | Control | 12 | 12 | 12 | 15 | 10 |
| GU-296H | Control | 14 | 11 | 16 | 15 | 13 |
| IAC-1 | Control | 22 | 21 | 16 | 15 | 17 |
| IPIRANGA-1 | Control | 34 | 36 | 38 | 35 | 38 |
| NA-33 | Control | 20 | 15 | 27 | 18 | 20 |
| PH-15 | Control | 32 | 34 | 36 | 32 | 31 |
| PH-16 | Control | 31 | 35 | 33 | 33 | 31 |
| PMCT-58 | Control | 16 | 25 | 21 | 19 | 18 |
| Pound-7 (BP-R) | Control | 33 | 31 | 31 | 26 | 30 |
| PS-1319 | Control | 33 | 32 | 36 | 31 | 34 |
| SCA-6 (WB-T) | Control | 19 | 8 | 7 | 7 | 5 |
| SIAL-70 (WB-S) | Control | 34 | 31 | 30 | 29 | 30 |
| SJ-02 | Control | 32 | 34 | 31 | 33 | 39 |
| TSH-1188 (BP-R) | Control | 5 | 31 | 38 | 25 | 33 |
| VB-514 | Control | 17 | 14 | 14 | 14 | 10 |

TABLE 46
Average aborted beans per pod (count) for various varieties at “CT-12” trial over multiple years.

| | | Average aborted beans per pod (count) | | | | |
|---|---|---|---|---|---|---|
| Genotype name | Variety | 2020 | 2021 | 2022 | 2023 | 2024 |
| PT-08-020-1-145-02 | CMC-001 | 2 | 1 | 1 | 1 | 1 |
| PT-08-020-1-145-12 | CMC-002 | 2 | 1 | 0 | 1 | 1 |
| PT-08-018-2-084-18 | CMC-003 | 1 | 0 | 1 | 1 | 1 |
| PT-08-038-1-113-09 | CMC-004 | 3 | 4 | 1 | 1 | 5 |
| PT-08-018-2-084-12 | CMC-005 | 2 | 1 | 1 | 1 | 1 |
| PT-08-038-3-096-10 | CMC-006 | 2 | 1 | 1 | 1 | 2 |
| PT-08-020-1-145-15 | CMC-007 | 1 | 0 | 1 | 1 | 1 |
| PT-08-041-2-052-18 | CMC-008 | 6 | 2 | 3 | 3 | 2 |
| PT-08-018-1-034-18 | CMC-009 | 2 | 1 | 1 | 1 | 2 |
| PT-08-038-1-113-03 | CMC-010 | 3 | 3 | 1 | 2 | 2 |
| PT-08-020-3-092-11 | CMC-011 | 2 | 3 | 3 | 3 | 4 |
| MC-13-202 | CMC-012 | 2 | 1 | 1 | 1 | 2 |
| PT-08-003-3-155-18 | CMC-013 | 4 | 2 | 1 | 2 | 5 |
| MC-08006 | CMC-014 | 1 | 1 | 1 | 1 | 3 |
| PT-08-041-1-037-17 | CMC-015 | 3 | 1 | 0 | 1 | 2 |
| PT-08-034-3-154-07 | CMC-016 | 5 | 3 | 2 | 1 | 5 |
| PT-08-038-3-096-06 | CMC-017 | 2 | 1 | 1 | 2 | 3 |
| PT-08-057-2-173-09 | CMC-018 | 2 | 2 | 1 | 1 | 3 |
| PT-08-020-2-057-12 | CMC-019 | 2 | 1 | 1 | 1 | 2 |
| PT-08-045-2-172-10 | CMC-020 | 3 | 2 | 1 | 2 | 2 |
| PT-08-019-3-164-18 | CMC-021 | 4 | 2 | 1 | 1 | 1 |
| PT-08-042-3-166-15 | CMC-022 | 8 | 6 | 4 | 3 | 8 |
| PT-08-038-2-050-11 | CMC-023 | 1 | 1 | 1 | 2 | 3 |
| PT-08-020-2-057-16 | CMC-024 | 1 | 1 | 1 | 1 | 1 |
| PT-08-038-3-096-02 | CMC-025 | 2 | 1 | 2 | 2 | 3 |
| PT-08-018-2-084-08 | CMC-026 | 2 | 1 | 1 | 1 | 1 |
| PT-08-064-1-107-18 | CMC-027 | 4 | 3 | 2 | 2 | 2 |
| PT-08-018-1-034-01 | CMC-028 | 2 | 1 | 1 | 2 | 2 |
| MC-08060 | CMC-029 | 3 | 2 | 2 | 2 | 5 |
| PT-08-018-2-084-05 | CMC-030 | 2 | 1 | 1 | 1 | 1 |
| PT-08-018-1-034-12 | CMC-031 | 1 | 2 | 1 | 1 | 3 |
| MC-08092 | CMC-032 | 1 | 1 | 1 | 1 | 4 |
| MCCS_MP01-718 | CMC-033 | 4 | 3 | 1 | 2 | 4 |
| MCCS_MP01-192 | CMC-034 | 2 | 3 | 1 | 1 | 2 |
| PT-08-038-1-113-18 | CMC-035 | 3 | 2 | 2 | 1 | 3 |
| PT-08-003-1-009-05 | CMC-036 | 4 | 1 | 1 | 1 | 2 |
| CATIE-R1 | Control | 6 | 7 | 6 | 5 | 6 |
| CATIE-R4 | Control | 5 | 4 | 4 | 4 | 5 |
| CATIE-R6 | Control | 3 | 3 | 2 | 2 | 5 |
| CCN-10 | Control | 8 | 8 | 5 | 4 | 5 |
| CCN-51 | Control | 3 | 1 | 1 | 1 | 2 |
| CEPEC-2002 (VB-1151) | Control | 2 | 1 | 1 | 1 | 1 |
| CEPEC-2004 (CEPEC-46) | Control | 3 | 1 | 1 | 1 | 2 |
| CP-49 | Control | 3 | 4 | 2 | 3 | 5 |
| FB-206 (BP-S) | Control | 2 | 1 | 1 | 4 | 2 |
| FCOM-150 | Control | 3 | 1 | 1 | 1 | 4 |
| GU-144C T1 | Control | 2 | 3 | 2 | 2 | 2 |
| GU-171C | Control | 5 | 5 | 4 | 3 | 4 |
| GU-296H | Control | 3 | 3 | 3 | 3 | 1 |
| IAC-1 | Control | 1 | 1 | 1 | 1 | 1 |
| IPIRANGA-1 | Control | 4 | 2 | 1 | 1 | 3 |
| NA-33 | Control | 1 | 0 | 1 | 1 | 1 |
| PH-15 | Control | 4 | 1 | 2 | 2 | 5 |
| PH-16 | Control | 2 | 1 | 2 | 1 | 3 |
| PMCT-58 | Control | 1 | 1 | 1 | 1 | 2 |
| Pound-7 (BP-R) | Control | 2 | 1 | 2 | 1 | 2 |
| PS-1319 | Control | 4 | 3 | 2 | 4 | 2 |
| SCA-6 (WB-T) | Control | 1 | 1 | 1 | 1 | 1 |
| SIAL-70 (WB-S) | Control | 2 | 3 | 1 | 2 | 3 |
| SJ-02 | Control | 6 | 1 | 1 | 1 | 1 |
| TSH-1188 (BP-R) | Control | 31 | 2 | 2 | 2 | NA |
| VB-514 | Control | 4 | 3 | 1 | 2 | 3 |

### Cumulative CT-12 Trial Data

Provided below are cumulative data for the varieties disclosed herein from a multi-year trial from a plot designated as “CT-12.” The “CT-12” trial is located in Bahia, Brazil.

TABLE 47
Variety and data information for this cumulative CT-12 trial data

| gen | cmc_name | biome | total_num_trees | count |
|---|---|---|---|---|
| PT-08-020-1-145-02 | CMC-001 | MCCS_CT12_Full_Sun | 12 | 575 |
| PT-08-041-2-052-18 | CMC-008 | MCCS_CT12_Full_Sun | 15 | 720 |
| MCCS_MP01-192 | CMC-034 | MCCS_CT12_Full_Sun | 15 | 710 |
| PT-08-020-1-145-15 | CMC-007 | MCCS_CT12_Full_Sun | 15 | 719 |
| PT-08-041-1-037-17 | CMC-015 | MCCS_CT12_Full_Sun | 15 | 717 |
| PT-08-038-3-096-10 | CMC-006 | MCCS_CT12_Full_Sun | 12 | 573 |
| PT-08-020-1-145-13 | PT-08-020-1- | MCCS_CT12_Full_Sun | 12 | 589 |
| | 145-13 | | | |
| PT-08-020-1-145-12 | CMC-002 | MCCS_CT12_Full_Sun | 12 | 580 |
| PT-08-038-1-113-03 | CMC-010 | MCCS_CT12_Full_Sun | 15 | 725 |
| PT-08-064-2-122-06 | PT-08-064-2- | MCCS_CT12_Full_Sun | 12 | 572 |
| | 122-06 | | | |
| PT-08-018-1-034-18 | CMC-009 | MCCS_CT12_Full_Sun | 12 | 584 |
| PT-08-020-2-057-16 | CMC-024 | MCCS_CT12_Full_Sun | 12 | 577 |
| PT-08-020-2-057-12 | CMC-019 | MCCS_CT12_Full_Sun | 12 | 582 |
| PT-08-018-2-084-18 | CMC-003 | MCCS_CT12_Full_Sun | 15 | 721 |
| MC-08086 | MC-08086 | MCCS_CT12_Full_Sun | 15 | 711 |
| PT-08-064-1-107-18 | CMC-027 | MCCS_CT12_Full_Sun | 12 | 572 |
| PT-08-019-3-164-18 | CMC-021 | MCCS_CT12_Full_Sun | 12 | 586 |
| PT-08-034-3-154-07 | CMC-016 | MCCS_CT12_Full_Sun | 12 | 585 |
| SJ-02 | SJ-02 | MCCS_CT12_Full_Sun | 12 | 573 |
| PT-08-038-1-113-18 | CMC-035 | MCCS_CT12_Full_Sun | 15 | 713 |
| MCCS_MP01-718 | CMC-033 | MCCS_CT12_Full_Sun | 12 | 575 |
| PT-08-003-3-155-18 | CMC-013 | MCCS_CT12_Full_Sun | 15 | 730 |
| MC-13-050 | MC-13-050 | MCCS_CT12_Full_Sun | 15 | 711 |
| MCCS_MP01-427 | MCCS_MP01- | MCCS_CT12_Full_Sun | 12 | 569 |
| | 427 | | | |
| PT-08-038-3-096-02 | CMC-025 | MCCS_CT12_Full_Sun | 15 | 725 |
| PT-08-038-1-113-09 | CMC-004 | MCCS_CT12_Full_Sun | 12 | 587 |
| MP01-492 | MP01-492 | MCCS_CT12_Full_Sun | 15 | 719 |
| MP01-619 (WB-R) | MP01-619 | MCCS_CT12_Full_Sun | 15 | 713 |
| | (WB-R) | | | |
| PT-08-045-2-172-10 | CMC-020 | MCCS_CT12_Full_Sun | 15 | 721 |
| CCN-51 | CCN-51 | MCCS_CT12_Full_Sun | 12 | 575 |
| MC-13-259 | MC-13-259 | MCCS_CT12_Full_Sun | 15 | 714 |
| PT-08-061-3-163-12 | PT-08-061-3- | MCCS_CT12_Full_Sun | 12 | 581 |
| | 163-12 | | | |
| PT-08-038-2-050-11 | CMC-023 | MCCS_CT12_Full_Sun | 12 | 571 |
| PT-08-063-1-065-12 | PT-08-063-1- | MCCS_CT12_Full_Sun | 12 | 576 |
| | 065-12 | | | |
| PT-08-020-3-092-05 | PT-08-020-3- | MCCS_CT12_Full_Sun | 15 | 716 |
| | 092-05 | | | |
| PT-08-063-1-065-14 | PT-08-063-1- | MCCS_CT12_Full_Sun | 12 | 571 |
| | 065-14 | | | |
| PT-08-018-1-034-01 | CMC-028 | MCCS_CT12_Full_Sun | 15 | 720 |
| MC-08006 | CMC-014 | MCCS_CT12_Full_Sun | 15 | 728 |
| PT-08-003-1-009-05 | CMC-036 | MCCS_CT12_Full_Sun | 12 | 574 |
| PT-08-020-2-057-01 | PT-08-020-2- | MCCS_CT12_Full_Sun | 12 | 578 |
| | 057-01 | | | |
| FCOM-150 | FCOM-150 | MCCS_CT12_Full_Sun | 12 | 576 |
| MCCS_MP01-522 | MCCS_MP01- | MCCS_CT12_Full_Sun | 15 | 712 |
| | 522 | | | |
| PT-08-018-2-084-12 | CMC-005 | MCCS_CT12_Full_Sun | 12 | 575 |
| PS-1319 | PS-1319 | MCCS_CT12_Full_Sun | 12 | 568 |
| PT-08-069-2-083-10 | PT-08-069-2- | MCCS_CT12_Full_Sun | 12 | 568 |
| | 083-10 | | | |
| PT-08-042-3-166-15 | CMC-022 | MCCS_CT12_Full_Sun | 12 | 589 |
| PT-08-063-3-157-09 | PT-08-063-3- | MCCS_CT12_Full_Sun | 15 | 715 |
| | 157-09 | | | |
| PT-08-038-3-096-06 | CMC-017 | MCCS_CT12_Full_Sun | 12 | 605 |
| MC-13-202 | CMC-012 | MCCS_CT12_Full_Sun | 15 | 719 |
| PT-08-041-3-093-04 | PT-08-041-3- | MCCS_CT12_Full_Sun | 12 | 581 |
| | 093-04 | | | |
| PT-08-063-3-157-11 | PT-08-063-3- | MCCS_CT12_Full_Sun | 12 | 580 |
| | 157-11 | | | |
| PT-08-063-3-157-01 | PT-08-063-3- | MCCS_CT12_Full_Sun | 12 | 577 |
| | 157-01 | | | |
| PT-08-038-3-096-01 | PT-08-038-3- | MCCS_CT12_Full_Sun | 12 | 573 |
| | 096-01 | | | |
| PT-08-018-2-084-17 | PT-08-018-2- | MCCS_CT12_Full_Sun | 15 | 725 |
| | 084-17 | | | |
| PT-08-020-3-092-10 | PT-08-020-3- | MCCS_CT12_Full_Sun | 12 | 579 |
| | 092-10 | | | |
| PT-08-018-2-084-08 | CMC-026 | MCCS_CT12_Full_Sun | 15 | 727 |
| PT-08-020-1-145-18 | PT-08-020-1- | MCCS_CT12_Full_Sun | 12 | 570 |
| | 145-18 | | | |
| PT-08-018-2-084-05 | CMC-030 | MCCS_CT12_Full_Sun | 12 | 584 |
| PT-08-018-1-034-12 | CMC-031 | MCCS_CT12_Full_Sun | 12 | 572 |
| PT-08-063-1-065-11 | PT-08-063-1- | MCCS_CT12_Full_Sun | 12 | 583 |
| | 065-11 | | | |
| MC-08103 | MC-08103 | MCCS_CT12_Full_Sun | 15 | 723 |
| MCCS_MP01-734 | MCCS_MP01- | MCCS_CT12_Full_Sun | 15 | 710 |
| | 734 | | | |
| PT-08-063-1-065-17 | PT-08-063-1- | MCCS_CT12_Full_Sun | 12 | 573 |
| | 065-17 | | | |
| PT-08-057-2-173-09 | CMC-018 | MCCS_CT12_Full_Sun | 15 | 734 |
| MCCS_MP01-414 | MCCS_MP01- | MCCS_CT12_Full_Sun | 12 | 569 |
| | 414 | | | |
| PT-08-020-3-092-01 | PT-08-020-3- | MCCS_CT12_Full_Sun | 12 | 580 |
| | 092-01 | | | |
| MC-08061 | MC-08061 | MCCS_CT12_Full_Sun | 15 | 729 |
| PT-08-059-2-086-18 | PT-08-059-2- | MCCS_CT12_Full_Sun | 12 | 570 |
| | 086-18 | | | |
| PT-08-020-2-057-10 | PT-08-020-2- | MCCS_CT12_Full_Sun | 12 | 577 |
| | 057-10 | | | |
| PT-08-018-1-034-02 | PT-08-018-1- | MCCS_CT12_Full_Sun | 15 | 734 |
| | 034-02 | | | |
| PT-08-038-2-050-01 | PT-08-038-2- | MCCS_CT12_Full_Sun | 12 | 573 |
| | 050-01 | | | |
| MC-13-083 | MC-13-083 | MCCS_CT12_Full_Sun | 15 | 729 |
| PT-08-038-3-096-07 | PT-08-038-3- | MCCS_CT12_Full_Sun | 11 | 520 |
| | 096-07 | | | |
| MC-08060 | CMC-029 | MCCS_CT12_Full_Sun | 12 | 592 |
| MC-08091 | MC-08091 | MCCS_CT12_Full_Sun | 12 | 570 |
| FB-206 (BP-S) | FB-206 (BP-S) | MCCS_CT12_Full_Sun | 12 | 579 |
| PT-08-038-2-050-08 | PT-08-038-2- | MCCS_CT12_Full_Sun | 12 | 581 |
| | 050-08 | | | |
| PT-08-061-2-048-10 | PT-08-061-2- | MCCS_CT12_Full_Sun | 15 | 726 |
| | 048-10 | | | |
| PT-08-063-2-117-11 | PT-08-063-2- | MCCS_CT12_Full_Sun | 12 | 575 |
| | 117-11 | | | |
| PT-08-018-3-178-09 | PT-08-018-3- | MCCS_CT12_Full_Sun | 12 | 574 |
| | 178-09 | | | |
| SIAL-70 (WB-S) | SIAL-70 (WB-S) | MCCS_CT12_Full_Sun | 12 | 568 |
| PT-08-069-2-083-18 | PT-08-069-2- | MCCS_CT12_Full_Sun | 12 | 571 |
| | 083-18 | | | |
| CEPEC-2004 | CEPEC-2004 | MCCS_CT12_Full_Sun | 12 | 575 |
| (CEPEC-46) | (CEPEC-46) | | | |
| PT-08-038-1-113-05 | PT-08-038-1- | MCCS_CT12_Full_Sun | 13 | 640 |
| | 113-05 | | | |
| CEPEC-2002 | CEPEC-2002 | MCCS_CT12_Full_Sun | 12 | 581 |
| (VB-1151) | (VB-1151) | | | |
| PT-08-020-2-057-18 | PT-08-020-2- | MCCS_CT12_Full_Sun | 12 | 569 |
| | 057-18 | | | |
| PT-08-003-1-009-12 | PT-08-003-1- | MCCS_CT12_Full_Sun | 12 | 574 |
| | 009-12 | | | |
| MC-08005 | MC-08005 | MCCS_CT12_Full_Sun | 12 | 578 |
| MC-08093 | MC-08093 | MCCS_CT12_Full_Sun | 15 | 719 |
| PH-15 | PH-15 | MCCS_CT12_Full_Sun | 12 | 568 |
| PT-08-020-1-145-07 | PT-08-020-1- | MCCS_CT12_Full_Sun | 12 | 576 |
| | 145-07 | | | |
| PH-16 | PH-16 | MCCS_CT12_Full_Sun | 12 | 570 |
| Pound-7 (BP-R) | Pound-7 (BP-R) | MCCS_CT12_Full_Sun | 12 | 573 |
| PT-08-020-3-092-07 | PT-08-020-3- | MCCS_CT12_Full_Sun | 12 | 574 |
| | 092-07 | | | |
| PT-08-069-3-156-10 | PT-08-069-3- | MCCS_CT12_Full_Sun | 12 | 571 |
| | 156-10 | | | |
| CP-49 | CP-49 | MCCS_CT12_Full_Sun | 12 | 577 |
| MC-08017 | MC-08017 | MCCS_CT12_Full_Sun | 15 | 720 |
| PT-08-020-3-092-11 | CMC-011 | MCCS_CT12_Full_Sun | 12 | 581 |
| MC-08171 | MC-08171 | MCCS_CT12_Full_Sun | 12 | 572 |
| PT-08-018-2-084-07 | PT-08-018-2- | MCCS_CT12_Full_Sun | 12 | 598 |
| | 084-07 | | | |
| PT-08-006-1-039-15 | PT-08-006-1- | MCCS_CT12_Full_Sun | 15 | 740 |
| | 039-15 | | | |
| CCN-10 | CCN-10 | MCCS_CT12_Full_Sun | 12 | 569 |
| MC-08059 | MC-08059 | MCCS_CT12_Full_Sun | 15 | 727 |
| MC-08092 | CMC-032 | MCCS_CT12_Full_Sun | 15 | 732 |
| IPIRANGA-1 | IPIRANGA-1 | MCCS_CT12_Full_Sun | 12 | 581 |
| CATIE-R6 | CATIE-R6 | MCCS_CT12_Full_Sun | 15 | 714 |
| IAC-1 | IAC-1 | MCCS_CT12_Full_Sun | 12 | 570 |
| CATIE-R1 | CATIE-R1 | MCCS_CT12_Full_Sun | 15 | 720 |
| PT-08-020-3-092-14 | PT-08-020-3- | MCCS_CT12_Full_Sun | 12 | 574 |
| | 092-14 | | | |
| CATIE-R4 | CATIE-R4 | MCCS_CT12_Full_Sun | 15 | 710 |
| NA-33 | NA-33 | MCCS_CT12_Full_Sun | 12 | 577 |
| VB-514 | VB-514 | MCCS_CT12_Full_Sun | 12 | 570 |
| PMCT-58 | PMCT-58 | MCCS_CT12_Full_Sun | 15 | 711 |
| MC-08114 | MC-08114 | MCCS_CT12_Full_Sun | 12 | 580 |
| GU-144C T1 | GU-144C T1 | MCCS_CT12_Full_Sun | 12 | 582 |
| PT-08-018-2-084-10 | PT-08-018-2- | MCCS_CT12_Full_Sun | 12 | 569 |
| | 084-10 | | | |
| TSH-1188 (BP-R) | TSH-1188 (BP-R) | MCCS_CT12_Full_Sun | 12 | 568 |
| MC-08024 | MC-08024 | MCCS_CT12_Full_Sun | 12 | 568 |
| SCA-6 (WB-T) | SCA-6 (WB-T) | MCCS_CT12_Full_Sun | 12 | 578 |
| GU-171C | GU-171C | MCCS_CT12_Full_Sun | 12 | 582 |
| GU-296H | GU-296H | MCCS_CT12_Full_Sun | 12 | 576 |

TABLE 48
TDBW data

| | mean_ | | TDBW | TDBW/ |
|---|---|---|---|---|
| | tdbw_ | % of | @ 1175 | year |
| variety | ct12_pt | CCN-51 | trees/ha | (T/ha) |
| CMC-001 | 9449.33 | 158 | 11.10 | 2.22 |
| CMC-008 | 9023.58 | 151 | 10.60 | 2.12 |
| CMC-034 | 8120.42 | 136 | 9.54 | 1.91 |
| CMC-007 | 7936 | 133 | 9.32 | 1.86 |
| CMC-015 | 7925.79 | 133 | 9.31 | 1.86 |
| CMC-006 | 7649 | 128 | 8.99 | 1.80 |
| PT-08-020-1-145-13 | 7423.48 | 124 | 8.72 | 1.74 |
| CMC-002 | 7343.11 | 123 | 8.63 | 1.73 |
| CMC-010 | 7302.27 | 122 | 8.58 | 1.72 |
| PT-08-064-2-122-06 | 6819.44 | 114 | 8.01 | 1.60 |
| CMC-009 | 6815.33 | 114 | 8.01 | 1.60 |
| CMC-024 | 6761.85 | 113 | 7.95 | 1.59 |
| CMC-019 | 6754.79 | 113 | 7.94 | 1.59 |
| CMC-003 | 6719.87 | 112 | 7.90 | 1.58 |
| MC-08086 | 6552.27 | 110 | 7.70 | 1.54 |
| CMC-027 | 6513.88 | 109 | 7.65 | 1.53 |
| CMC-021 | 6357.97 | 106 | 7.47 | 1.49 |
| CMC-016 | 6290.12 | 105 | 7.39 | 1.48 |
| SJ-02 | 6288.22 | 105 | 7.39 | 1.48 |
| CMC-035 | 6253.79 | 105 | 7.35 | 1.47 |
| CMC-033 | 6218.79 | 104 | 7.31 | 1.46 |
| CMC-013 | 6218.77 | 104 | 7.31 | 1.46 |
| MC-13-050 | 6208.6 | 104 | 7.30 | 1.46 |
| MCCS_MP01-427 | 6202.59 | 104 | 7.29 | 1.46 |
| CMC-025 | 6109.4 | 102 | 7.18 | 1.44 |
| CMC-004 | 6067.25 | 102 | 7.13 | 1.43 |
| MP01-492 | 6033.82 | 101 | 7.09 | 1.42 |
| MP01-619 (WB-R) | 6024.43 | 101 | 7.08 | 1.42 |
| CMC-020 | 6021.46 | 101 | 7.08 | 1.42 |
| CCN-51 | 5976.58 | 100 | 7.02 | 1.40 |
| MC-13-259 | 5973.72 | 100 | 7.02 | 1.40 |
| PT-08-061-3-163-12 | 5957.39 | 100 | 7.00 | 1.40 |
| CMC-023 | 5929.16 | 99 | 6.97 | 1.39 |
| PT-08-063-1-065-12 | 5856.24 | 98 | 6.88 | 1.38 |
| PT-08-020-3-092-05 | 5797.37 | 97 | 6.81 | 1.36 |
| PT-08-063-1-065-14 | 5750.34 | 96 | 6.76 | 1.35 |
| CMC-028 | 5725.11 | 96 | 6.73 | 1.35 |
| CMC-014 | 5662.81 | 95 | 6.65 | 1.33 |
| CMC-036 | 5648.12 | 95 | 6.64 | 1.33 |
| PT-08-020-2-057-01 | 5645.01 | 94 | 6.63 | 1.33 |
| FCOM-150 | 5508.2 | 92 | 6.47 | 1.29 |
| MCCS_MP01-522 | 5475.14 | 92 | 6.43 | 1.29 |
| CMC-005 | 5401.43 | 90 | 6.35 | 1.27 |
| PS-1319 | 5355.1 | 90 | 6.29 | 1.26 |
| PT-08-069-2-083-10 | 5334.27 | 89 | 6.27 | 1.25 |
| CMC-022 | 5326.18 | 89 | 6.26 | 1.25 |
| PT-08-063-3-157-09 | 5264.46 | 88 | 6.19 | 1.24 |
| CMC-017 | 5240.82 | 88 | 6.16 | 1.23 |
| CMC-012 | 5153.37 | 86 | 6.06 | 1.21 |
| PT-08-041-3-093-04 | 5121.82 | 86 | 6.02 | 1.20 |
| PT-08-063-3-157-11 | 5076.06 | 85 | 5.96 | 1.19 |
| PT-08-063-3-157-01 | 5032.55 | 84 | 5.91 | 1.18 |
| PT-08-038-3-096-01 | 5009.01 | 84 | 5.89 | 1.18 |
| PT-08-018-2-084-17 | 4964.71 | 83 | 5.83 | 1.17 |
| PT-08-020-3-092-10 | 4954.2 | 83 | 5.82 | 1.16 |
| CMC-026 | 4882.64 | 82 | 5.74 | 1.15 |
| PT-08-020-1-145-18 | 4836.83 | 81 | 5.68 | 1.14 |
| CMC-030 | 4822.16 | 81 | 5.67 | 1.13 |
| CMC-031 | 4798 | 80 | 5.64 | 1.13 |
| PT-08-063-1-065-11 | 4733.6 | 79 | 5.56 | 1.11 |
| MC-08103 | 4718.76 | 79 | 5.54 | 1.11 |
| MCCS_MP01-734 | 4651.64 | 78 | 5.47 | 1.09 |
| PT-08-063-1-065-17 | 4609.5 | 77 | 5.42 | 1.08 |
| CMC-018 | 4592.21 | 77 | 5.40 | 1.08 |
| MCCS_MP01-414 | 4547.99 | 76 | 5.34 | 1.07 |
| PT-08-020-3-092-01 | 4482.82 | 75 | 5.27 | 1.05 |
| MC-08061 | 4450.74 | 74 | 5.23 | 1.05 |
| PT-08-059-2-086-18 | 4445.27 | 74 | 5.22 | 1.04 |
| PT-08-020-2-057-10 | 4367.4 | 73 | 5.13 | 1.03 |
| PT-08-018-1-034-02 | 4281.5 | 72 | 5.03 | 1.01 |
| PT-08-038-2-050-01 | 4247.51 | 71 | 4.99 | 1.00 |
| MC-13-083 | 4161.34 | 70 | 4.89 | 0.98 |
| PT-08-038-3-096-07 | 4134.95 | 69 | 4.86 | 0.97 |
| CMC-029 | 4018.07 | 67 | 4.72 | 0.94 |
| MC-08091 | 3992.73 | 67 | 4.69 | 0.94 |
| FB-206 (BP-S) | 3859.15 | 65 | 4.53 | 0.91 |
| PT-08-038-2-050-08 | 3858.57 | 65 | 4.53 | 0.91 |
| PT-08-061-2-048-10 | 3853.88 | 64 | 4.53 | 0.91 |
| PT-08-063-2-117-11 | 3828.87 | 64 | 4.50 | 0.90 |
| PT-08-018-3-178-09 | 3756.54 | 63 | 4.41 | 0.88 |
| SIAL-70 (WB-S) | 3742.79 | 63 | 4.40 | 0.88 |
| PT-08-069-2-083-18 | 3718.77 | 62 | 4.37 | 0.87 |
| CEPEC-2004 (CEPEC-46) | 3709.66 | 62 | 4.36 | 0.87 |
| PT-08-038-1-113-05 | 3680.73 | 62 | 4.32 | 0.86 |
| CEPEC-2002 (VB-1151) | 3653.04 | 61 | 4.29 | 0.86 |
| PT-08-020-2-057-18 | 3538.52 | 59 | 4.16 | 0.83 |
| PT-08-003-1-009-12 | 3531.05 | 59 | 4.15 | 0.83 |
| MC-08005 | 3486.03 | 58 | 4.10 | 0.82 |
| MC-08093 | 3474.37 | 58 | 4.08 | 0.82 |
| PH-15 | 3390.2 | 57 | 3.98 | 0.80 |
| PT-08-020-1-145-07 | 3310.46 | 55 | 3.89 | 0.78 |
| PH-16 | 3289.68 | 55 | 3.87 | 0.77 |
| Pound-7 (BP-R) | 3190.15 | 53 | 3.75 | 0.75 |
| PT-08-020-3-092-07 | 3131.42 | 52 | 3.68 | 0.74 |
| PT-08-069-3-156-10 | 3103.16 | 52 | 3.65 | 0.73 |
| CP-49 | 3086.28 | 52 | 3.63 | 0.73 |
| MC-08017 | 3078.47 | 52 | 3.62 | 0.72 |
| CMC-011 | 3064.49 | 51 | 3.60 | 0.72 |
| MC-08171 | 3039.17 | 51 | 3.57 | 0.71 |
| PT-08-018-2-084-07 | 2972.24 | 50 | 3.49 | 0.70 |
| PT-08-006-1-039-15 | 2897.4 | 48 | 3.40 | 0.68 |
| CCN-10 | 2765.49 | 46 | 3.25 | 0.65 |
| MC-08059 | 2760.84 | 46 | 3.24 | 0.65 |
| CMC-032 | 2726.47 | 46 | 3.20 | 0.64 |
| IPIRANGA-1 | 2593.37 | 43 | 3.05 | 0.61 |
| CATIE-R6 | 2580.07 | 43 | 3.03 | 0.61 |
| IAC-1 | 2214.97 | 37 | 2.60 | 0.52 |
| CATIE-R1 | 2168.54 | 36 | 2.55 | 0.51 |
| PT-08-020-3-092-14 | 2149.56 | 36 | 2.53 | 0.51 |
| CATIE-R4 | 1980.05 | 33 | 2.33 | 0.47 |
| NA-33 | 1898.12 | 32 | 2.23 | 0.45 |
| VB-514 | 1857.93 | 31 | 2.18 | 0.44 |
| PMCT-58 | 1614.31 | 27 | 1.90 | 0.38 |
| MC-08114 | 1608.46 | 27 | 1.89 | 0.38 |
| GU-144C T1 | 1303.46 | 22 | 1.53 | 0.31 |
| PT-08-018-2-084-10 | 941.88 | 16 | 1.11 | 0.22 |
| TSH-1188 (BP-R) | 927.18 | 16 | 1.09 | 0.22 |
| MC-08024 | 634.57 | 11 | 0.75 | 0.15 |
| SCA-6 (WB-T) | 444.68 | 7 | 0.52 | 0.10 |
| GU-171C | 371.81 | 6 | 0.44 | 0.09 |
| GU-296H | 348.88 | 6 | 0.41 | 0.08 |

TABLE 49
Additional TDBW data

| | TDBW | TDBW/ | |
|---|---|---|---|
| | @1500 | year | |
| variety | trees/ha | (T/ha) | mean_bean_size_ct12_pt |
| CMC-001 | 14.17 | 2.83 | 0.96 |
| CMC-008 | 13.54 | 2.71 | 1.19 |
| CMC-034 | 12.18 | 2.44 | 1.34 |
| CMC-007 | 11.90 | 2.38 | 1.3 |
| CMC-015 | 11.89 | 2.38 | 1.19 |
| CMC-006 | 11.47 | 2.29 | 0.85 |
| PT-08-020-1-145-13 | 11.14 | 2.23 | 1.14 |
| CMC-002 | 11.01 | 2.20 | 1.18 |
| CMC-010 | 10.95 | 2.19 | 1.12 |
| PT-08-064-2-122-06 | 10.23 | 2.05 | 1.36 |
| CMC-009 | 10.22 | 2.04 | 1.32 |
| CMC-024 | 10.14 | 2.03 | 1.2 |
| CMC-019 | 10.13 | 2.03 | 1.07 |
| CMC-003 | 10.08 | 2.02 | 0.91 |
| MC-08086 | 9.83 | 1.97 | 1.14 |
| CMC-027 | 9.77 | 1.95 | 1.22 |
| CMC-021 | 9.54 | 1.91 | 1.06 |
| CMC-016 | 9.44 | 1.89 | 0.99 |
| SJ-02 | 9.43 | 1.89 | 1.16 |
| CMC-035 | 9.38 | 1.88 | 0.91 |
| CMC-033 | 9.33 | 1.87 | 1.14 |
| CMC-013 | 9.33 | 1.87 | 0.93 |
| MC-13-050 | 9.31 | 1.86 | 1.16 |
| MCCS_MP01-427 | 9.30 | 1.86 | 1.4 |
| CMC-025 | 9.16 | 1.83 | 0.96 |
| CMC-004 | 9.10 | 1.82 | 0.61 |
| MP01-492 | 9.05 | 1.81 | 1.27 |
| MP01-619 (WB-R) | 9.04 | 1.81 | 1.07 |
| CMC-020 | 9.03 | 1.81 | 1.16 |
| CCN-51 | 8.96 | 1.79 | 1.33 |
| MC-13-259 | 8.96 | 1.79 | 1.46 |
| PT-08-061-3-163-12 | 8.94 | 1.79 | 0.86 |
| CMC-023 | 8.89 | 1.78 | 0.79 |
| PT-08-063-1-065-12 | 8.78 | 1.76 | 1.03 |
| PT-08-020-3-092-05 | 8.70 | 1.74 | 0.84 |
| PT-08-063-1-065-14 | 8.63 | 1.73 | 1.12 |
| CMC-028 | 8.59 | 1.72 | 1.02 |
| CMC-014 | 8.49 | 1.70 | 1.22 |
| CMC-036 | 8.47 | 1.69 | 1.24 |
| PT-08-020-2-057-01 | 8.47 | 1.69 | 0.82 |
| FCOM-150 | 8.26 | 1.65 | 0.91 |
| MCCS_MP01-522 | 8.21 | 1.64 | 1.28 |
| CMC-005 | 8.10 | 1.62 | 1.06 |
| PS-1319 | 8.03 | 1.61 | 1.12 |
| PT-08-069-2-083-10 | 8.00 | 1.60 | 1.46 |
| CMC-022 | 7.99 | 1.60 | 1.24 |
| PT-08-063-3-157-09 | 7.90 | 1.58 | 1.55 |
| CMC-017 | 7.86 | 1.57 | 0.78 |
| CMC-012 | 7.73 | 1.55 | 1.39 |
| PT-08-041-3-093-04 | 7.68 | 1.54 | 1.34 |
| PT-08-063-3-157-11 | 7.61 | 1.52 | 1.17 |
| PT-08-063-3-157-01 | 7.55 | 1.51 | 1.3 |
| PT-08-038-3-096-01 | 7.51 | 1.50 | 1.01 |
| PT-08-018-2-084-17 | 7.45 | 1.49 | 1.19 |
| PT-08-020-3-092-10 | 7.43 | 1.49 | 1.09 |
| CMC-026 | 7.32 | 1.46 | 1.23 |
| PT-08-020-1-145-18 | 7.26 | 1.45 | 1.03 |
| CMC-030 | 7.23 | 1.45 | 0.98 |
| CMC-031 | 7.20 | 1.44 | 0.9 |
| PT-08-063-1-065-11 | 7.10 | 1.42 | 1.32 |
| MC-08103 | 7.08 | 1.42 | 0.96 |
| MCCS_MP01-734 | 6.98 | 1.40 | 1.45 |
| PT-08-063-1-065-17 | 6.91 | 1.38 | 1.11 |
| CMC-018 | 6.89 | 1.38 | 1.17 |
| MCCS_MP01-414 | 6.82 | 1.36 | 1.18 |
| PT-08-020-3-092-01 | 6.72 | 1.34 | 0.86 |
| MC-08061 | 6.68 | 1.34 | 0.78 |
| PT-08-059-2-086-18 | 6.67 | 1.33 | 1.38 |
| PT-08-020-2-057-10 | 6.55 | 1.31 | 0.96 |
| PT-08-018-1-034-02 | 6.42 | 1.28 | 1.22 |
| PT-08-038-2-050-01 | 6.37 | 1.27 | 0.87 |
| MC-13-083 | 6.24 | 1.25 | 1.22 |
| PT-08-038-3-096-07 | 6.20 | 1.24 | 0.92 |
| CMC-029 | 6.03 | 1.21 | 0.75 |
| MC-08091 | 5.99 | 1.20 | 1.08 |
| FB-206 (BP-S) | 5.79 | 1.16 | 0.87 |
| PT-08-038-2-050-08 | 5.79 | 1.16 | 0.7 |
| PT-08-061-2-048-10 | 5.78 | 1.16 | 1.17 |
| PT-08-063-2-117-11 | 5.74 | 1.15 | 1.22 |
| PT-08-018-3-178-09 | 5.63 | 1.13 | 1.12 |
| SIAL-70 (WB-S) | 5.61 | 1.12 | 0.95 |
| PT-08-069-2-083-18 | 5.58 | 1.12 | 0.99 |
| CEPEC-2004 | 5.56 | 1.11 | 0.98 |
| (CEPEC-46) | | | |
| PT-08-038-1-113-05 | 5.52 | 1.10 | 0.83 |
| CEPEC-2002 | 5.48 | 1.10 | 0.84 |
| (VB-1151) | | | |
| PT-08-020-2-057-18 | 5.31 | 1.06 | 1.17 |
| PT-08-003-1-009-12 | 5.30 | 1.06 | 1.08 |
| MC-08005 | 5.23 | 1.05 | 0.86 |
| MC-08093 | 5.21 | 1.04 | 1.12 |
| PH-15 | 5.09 | 1.02 | 1 |
| PT-08-020-1-145-07 | 4.97 | 0.99 | 1.11 |
| PH-16 | 4.93 | 0.99 | 1.11 |
| Pound-7 (BP-R) | 4.79 | 0.96 | 1.05 |
| PT-08-020-3-092-07 | 4.70 | 0.94 | 1.14 |
| PT-08-069-3-156-10 | 4.65 | 0.93 | 0.93 |
| CP-49 | 4.63 | 0.93 | 0.95 |
| MC-08017 | 4.62 | 0.92 | 1.04 |
| CMC-011 | 4.60 | 0.92 | 0.85 |
| MC-08171 | 4.56 | 0.91 | 1.05 |
| PT-08-018-2-084-07 | 4.46 | 0.89 | 0.98 |
| PT-08-006-1-039-15 | 4.35 | 0.87 | 0.81 |
| CCN-10 | 4.15 | 0.83 | 1.91 |
| MC-08059 | 4.14 | 0.83 | 0.69 |
| CMC-032 | 4.09 | 0.82 | 1.07 |
| IPIRANGA-1 | 3.89 | 0.78 | 0.99 |
| CATIE-R6 | 3.87 | 0.77 | 1.26 |
| IAC-1 | 3.32 | 0.66 | 1.25 |
| CATIE-R1 | 3.25 | 0.65 | 1.18 |
| PT-08-020-3-092-14 | 3.22 | 0.64 | 1.06 |
| CATIE-R4 | 2.97 | 0.59 | 1.23 |
| NA-33 | 2.85 | 0.57 | 0.97 |
| VB-514 | 2.79 | 0.56 | 0.74 |
| PMCT-58 | 2.42 | 0.48 | 1.15 |
| MC-08114 | 2.41 | 0.48 | 1 |
| GU-144C T1 | 1.96 | 0.39 | 0.93 |
| PT-08-018-2-084-10 | 1.41 | 0.28 | 1.4 |
| TSH-1188 (BP-R) | 1.39 | 0.28 | 1.5 |
| MC-08024 | 0.95 | 0.19 | 1.43 |
| SCA-6 (WB-T) | 0.67 | 0.13 | 0.71 |
| GU-171C | 0.56 | 0.11 | 0.58 |
| GU-296H | 0.52 | 0.10 | 0.8 |

TABLE 50
Variety data

| | | | | | | % | | % | | | | |
|---|---|---|---|---|---|---|---|---|---|---|---|---|
| variety | tdbw_ct12_pt | nph | nhph | nps | wbd | WBD | bpd | BPD | ol | paboa | npg | tpl |
| CMC-001 | 113391.95 | 4845 | 4466 | 1456 | 86 | 1.78 | 29 | 0.60 | 83 | 2 | 72 | 371 |
| CMC-008 | 135353.68 | 4887 | 4189 | 1564 | 180 | 3.68 | 42 | 0.86 | 172 | 0 | 43 | 699 |
| CMC-034 | 121806.30 | 2532 | 2071 | 759 | 89 | 3.52 | 37 | 1.46 | 68 | 0 | 8 | 469 |
| CMC-007 | 119040.00 | 3338 | 3062 | 1120 | 85 | 2.55 | 36 | 1.08 | 47 | 0 | 36 | 274 |
| CMC-015 | 118886.85 | 4890 | 4340 | 1552 | 129 | 2.64 | 62 | 1.27 | 159 | 0 | 22 | 545 |
| CMC-006 | 91788.04 | 4639 | 3792 | 1605 | 79 | 1.70 | 129 | 2.78 | 74 | 0 | 66 | 854 |
| PT-08-020-1- | 89081.80 | 2851 | 2525 | 1025 | 91 | 3.19 | 51 | 1.79 | 41 | 0 | 15 | 324 |
| 145-13 | | | | | | | | | | | | |
| CMC-002 | 88117.33 | 3337 | 2947 | 972 | 79 | 2.37 | 51 | 1.53 | 67 | 0 | 106 | 386 |
| CMC-010 | 109534.02 | 4098 | 3394 | 979 | 125 | 3.05 | 54 | 1.32 | 100 | 0 | 122 | 702 |
| PT-08-064-2- | 81833.27 | 2098 | 1696 | 687 | 107 | 5.10 | 66 | 3.15 | 84 | 0 | 12 | 402 |
| 122-06 | | | | | | | | | | | | |
| CMC-009 | 81783.93 | 2565 | 2314 | 1029 | 69 | 2.69 | 23 | 0.90 | 42 | 0 | 26 | 256 |
| CMC-024 | 81142.15 | 2915 | 2559 | 986 | 68 | 2.33 | 71 | 2.44 | 55 | 0 | 34 | 344 |
| CMC-019 | 81057.47 | 3169 | 2793 | 1218 | 51 | 1.61 | 82 | 2.59 | 87 | 1 | 99 | 380 |
| CMC-003 | 100798.10 | 4991 | 4555 | 1937 | 97 | 1.94 | 30 | 0.60 | 113 | 0 | 73 | 436 |
| MC-08086 | 98284.09 | 2483 | 2125 | 734 | 88 | 3.54 | 49 | 1.97 | 79 | 0 | 11 | 361 |
| CMC-027 | 78166.56 | 2387 | 2123 | 866 | 87 | 3.64 | 13 | 0.54 | 55 | 0 | 7 | 265 |
| CMC-021 | 76295.65 | 3026 | 2587 | 1151 | 89 | 2.94 | 64 | 2.12 | 54 | 0 | 47 | 435 |
| CMC-016 | 75481.40 | 3512 | 2804 | 1051 | 145 | 4.13 | 71 | 2.02 | 109 | 0 | 17 | 701 |
| SJ-02 | 75458.60 | 2395 | 2025 | 803 | 154 | 6.43 | 12 | 0.50 | 48 | 0 | 15 | 373 |
| CMC-035 | 93806.92 | 4166 | 3638 | 1324 | 123 | 2.95 | 84 | 2.02 | 82 | 0 | 20 | 522 |
| CMC-033 | 74625.46 | 2003 | 1637 | 757 | 62 | 3.10 | 39 | 1.95 | 56 | 0 | 5 | 369 |
| CMC-013 | 93281.50 | 4777 | 3544 | 1505 | 317 | 6.64 | 96 | 2.01 | 188 | 3 | 96 | 1231 |
| MC-13-050 | 93128.93 | 3244 | 2771 | 965 | 138 | 4.25 | 49 | 1.51 | 96 | 1 | 5 | 471 |
| MCCS_MP01- | 74431.10 | 1705 | 1299 | 533 | 135 | 7.92 | 16 | 0.94 | 51 | 0 | 3 | 406 |
| 427 | | | | | | | | | | | | |
| CMC-025 | 91641.06 | 3629 | 3219 | 1539 | 58 | 1.60 | 37 | 1.02 | 64 | 0 | 38 | 404 |
| CMC-004 | 72806.96 | 5627 | 4892 | 2496 | 125 | 2.22 | 73 | 1.30 | 243 | 1 | 143 | 717 |
| MP01-492 | 90507.34 | 2646 | 2122 | 732 | 135 | 5.10 | 22 | 0.83 | 78 | 2 | 8 | 529 |
| MP01-619 | 90366.47 | 2812 | 2386 | 962 | 69 | 2.45 | 100 | 3.56 | 53 | 0 | 9 | 433 |
| (WB-R) | | | | | | | | | | | | |
| CMC-020 | 90321.97 | 4079 | 3669 | 1191 | 171 | 4.19 | 31 | 0.76 | 68 | 0 | 34 | 417 |
| CCN-51 | 71719.01 | 1616 | 1291 | 501 | 60 | 3.71 | 43 | 2.66 | 47 | 0 | 10 | 327 |
| MC-13-259 | 89605.85 | 2609 | 1973 | 787 | 223 | 8.55 | 72 | 2.76 | 114 | 0 | 12 | 616 |
| PT-08-061-3- | 71488.64 | 3963 | 3143 | 1097 | 405 | 10.22 | 54 | 1.36 | 113 | 0 | 64 | 810 |
| 163-12 | | | | | | | | | | | | |
| CMC-023 | 71149.90 | 3274 | 2873 | 1075 | 86 | 2.63 | 27 | 0.82 | 85 | 0 | 10 | 400 |
| PT-08-063-1- | 70274.93 | 2632 | 2327 | 887 | 139 | 5.28 | 23 | 0.87 | 61 | 2 | 10 | 307 |
| 065-12 | | | | | | | | | | | | |
| PT-08-020-3- | 86960.60 | 4403 | 4021 | 1415 | 71 | 1.61 | 28 | 0.64 | 125 | 0 | 61 | 342 |
| 092-05 | | | | | | | | | | | | |
| PT-08-063-1- | 69004.11 | 2311 | 1750 | 742 | 164 | 7.10 | 89 | 3.85 | 77 | 0 | 20 | 560 |
| 065-14 | | | | | | | | | | | | |
| CMC-028 | 85876.71 | 3452 | 3134 | 1114 | 99 | 2.87 | 22 | 0.64 | 114 | 0 | 12 | 305 |
| CMC-014 | 84942.09 | 2776 | 2357 | 900 | 155 | 5.58 | 39 | 1.40 | 38 | 0 | 8 | 423 |
| CMC-036 | 67777.45 | 1695 | 1338 | 579 | 55 | 3.24 | 13 | 0.77 | 97 | 0 | 3 | 356 |
| PT-08-020-2- | 67740.13 | 4343 | 3671 | 1770 | 51 | 1.17 | 27 | 0.62 | 122 | 2 | 391 | 665 |
| 057-01 | | | | | | | | | | | | |
| FCOM-150 | 66098.46 | 2824 | 2460 | 996 | 181 | 6.41 | 78 | 2.76 | 36 | 0 | 14 | 366 |
| MCCS_MP01- | 82127.04 | 2041 | 1374 | 583 | 99 | 4.85 | 68 | 3.33 | 44 | 1 | 28 | 669 |
| 522 | | | | | | | | | | | | |
| CMC-005 | 64817.20 | 3131 | 2739 | 1189 | 87 | 2.78 | 97 | 3.10 | 46 | 0 | 27 | 380 |
| PS-1319 | 64261.15 | 1822 | 1542 | 589 | 145 | 7.96 | 32 | 1.76 | 43 | 0 | 10 | 284 |
| PT-08-069-2- | 64011.20 | 2186 | 1621 | 603 | 230 | 10.52 | 87 | 3.98 | 53 | 0 | 8 | 570 |
| 083-10 | | | | | | | | | | | | |
| CMC-022 | 63914.15 | 2193 | 1956 | 869 | 84 | 3.83 | 17 | 0.78 | 73 | 0 | 4 | 235 |
| PT-08-063-3- | 78966.97 | 1881 | 1468 | 687 | 196 | 10.42 | 39 | 2.07 | 39 | 0 | 2 | 417 |
| 157-09 | | | | | | | | | | | | |
| CMC-017 | 62889.85 | 4139 | 3705 | 1788 | 74 | 1.79 | 20 | 0.48 | 56 | 1 | 83 | 425 |
| CMC-012 | 77300.58 | 2370 | 1995 | 875 | 165 | 6.96 | 41 | 1.73 | 55 | 0 | 56 | 376 |
| PT-08-041-3- | 61461.85 | 2134 | 1796 | 819 | 96 | 4.50 | 34 | 1.59 | 43 | 0 | 14 | 337 |
| 093-04 | | | | | | | | | | | | |
| PT-08-063-3- | 60912.68 | 2150 | 1702 | 820 | 206 | 9.58 | 29 | 1.35 | 44 | 0 | 11 | 446 |
| 157-11 | | | | | | | | | | | | |
| PT-08-063-3- | 60390.63 | 1969 | 1496 | 663 | 137 | 6.96 | 45 | 2.29 | 95 | 0 | 6 | 467 |
| 157-01 | | | | | | | | | | | | |
| PT-08-038-3- | 60108.15 | 2503 | 1910 | 719 | 93 | 3.72 | 24 | 0.96 | 72 | 0 | 12 | 583 |
| 096-01 | | | | | | | | | | | | |
| PT-08-018-2- | 74470.63 | 2757 | 2423 | 1161 | 68 | 2.47 | 38 | 1.38 | 66 | 0 | 33 | 336 |
| 084-17 | | | | | | | | | | | | |
| PT-08-020-3- | 59450.46 | 3101 | 2666 | 921 | 123 | 3.97 | 46 | 1.48 | 89 | 1 | 109 | 425 |
| 092-10 | | | | | | | | | | | | |
| CMC-026 | 73239.55 | 2826 | 2596 | 1204 | 59 | 2.09 | 11 | 0.39 | 44 | 0 | 29 | 229 |
| PT-08-020-1- | 58041.90 | 2636 | 2340 | 1064 | 123 | 4.67 | 26 | 0.99 | 42 | 0) | 71 | 292 |
| 145-18 | | | | | | | | | | | | |
| CMC-030 | 57865.97 | 2166 | 1791 | 962 | 69 | 3.19 | 57 | 2.63 | 37 | 0 | 13 | 369 |
| CMC-031 | 57576.03 | 3188 | 2910 | 1189 | 52 | 1.63 | 76 | 2.38 | 57 | 0 | 18 | 278 |
| PT-08-063-1- | 56803.22 | 1981 | 1508 | 733 | 207 | 10.45 | 21 | 1.06 | 65 | 15 | 19 | 470 |
| 065-11 | | | | | | | | | | | | |
| MC-08103 | 70781.44 | 2816 | 2234 | 939 | 271 | 9.62 | 44 | 1.56 | 48 | 0 | 22 | 579 |
| MCCS_MP01- | 69774.60 | 1268 | 903 | 469 | 61 | 4.81 | 47 | 3.71 | 22 | 1 | 3 | 367 |
| 734 | | | | | | | | | | | | |
| PT-08-063-1- | 55314.04 | 2116 | 1678 | 661 | 241 | 11.39 | 37 | 1.75 | 90 | 0 | 11 | 452 |
| 065-17 | | | | | | | | | | | | |
| CMC-018 | 68883.17 | 3460 | 3074 | 1249 | 144 | 4.16 | 18 | 0.52 | 97 | 0 | 15 | 391 |
| MCCS_MP01- | 54575.90 | 1082 | 845 | 347 | 36 | 3.33 | 58 | 5.36 | 12 | 0 | 2 | 239 |
| 414 | | | | | | | | | | | | |
| PT-08-020-3- | 53793.89 | 2962 | 2642 | 1057 | 71 | 2.40 | 18 | 0.61 | 86 | 0 | 103 | 324 |
| 092-01 | | | | | | | | | | | | |
| MC-08061 | 66761.09 | 3206 | 2801 | 962 | 100 | 3.12 | 73 | 2.28 | 55 | 0 | 22 | 405 |
| PT-08-059-2- | 53343.20 | 1216 | 897 | 526 | 83 | 6.83 | 50 | 4.11 | 29 | 0 | 3 | 272 |
| 086-18 | | | | | | | | | | | | |
| PT-08-020-2- | 52408.75 | 2860 | 2576 | 937 | 65 | 2.27 | 11 | 0.38 | 34 | 0 | 110 | 286 |
| 057-10 | | | | | | | | | | | | |
| PT-08-018-1- | 64222.45 | 2349 | 2042 | 1209 | 89 | 3.79 | 18 | 0.77 | 53 | 0 | 41 | 309 |
| 034-02 | | | | | | | | | | | | |
| PT-08-038-2- | 50970.13 | 2765 | 2453 | 980 | 89 | 3.22 | 26 | 0.94 | 69 | 0 | 9 | 314 |
| 050-01 | | | | | | | | | | | | |
| MC-13-083 | 62420.04 | 2250 | 1879 | 787 | 118 | 5.24 | 28 | 1.24 | 29 | 0 | 22 | 374 |
| PT-08-038-3- | 45484.48 | 2005 | 1700 | 792 | 98 | 4.89 | 13 | 0.65 | 22 | 0 | 29 | 310 |
| 096-07 | | | | | | | | | | | | |
| CMC-029 | 48216.80 | 2455 | 2115 | 1182 | 121 | 4.93 | 40 | 1.63 | 33 | 0 | 38 | 341 |
| MC-08091 | 47912.79 | 1946 | 1539 | 497 | 97 | 4.98 | 90 | 4.62 | 75 | 0 | 112 | 403 |
| FB-206 (BP-S) | 46309.82 | 2238 | 1780 | 883 | 186 | 8.31 | 112 | 5.00 | 81 | 0 | 21 | 461 |
| PT-08-038-2- | 46302.84 | 2955 | 2586 | 1248 | 100 | 3.38 | 23 | 0.78 | 46 | 1 | 84 | 370 |
| 050-08 | | | | | | | | | | | | |
| PT-08-061-2- | 57808.20 | 1631 | 1396 | 750 | 109 | 6.68 | 30 | 1.84 | 24 | 0 | 3 | 235 |
| 048-10 | | | | | | | | | | | | |
| PT-08-063-2- | 45946.43 | 1817 | 1503 | 623 | 166 | 9.14 | 34 | 1.87 | 38 | 0 | 7 | 315 |
| 117-11 | | | | | | | | | | | | |
| PT-08-018-3- | 45078.50 | 1921 | 1725 | 724 | 30 | 1.56 | 14 | 0.73 | 30 | 0 | 16 | 186 |
| 178-09 | | | | | | | | | | | | |
| SIAL-70 | 44913.50 | 1890 | 1734 | 740 | 60 | 3.17 | 12 | 0.63 | 41 | 0 | 20 | 161 |
| (WB-S) | | | | | | | | | | | | |
| PT-08-069-2- | 44625.23 | 1397 | 1109 | 525 | 166 | 11.88 | 23 | 1.65 | 27 | 0 | 6 | 302 |
| 083-18 | | | | | | | | | | | | |
| CEPEC-2004 | 44515.95 | 2174 | 1773 | 605 | 96 | 4.42 | 61 | 2.81 | 67 | 0 | 19 | 397 |
| (CEPEC-46) | | | | | | | | | | | | |
| PT-08-038-1 | 47849.46 | 2732 | 2264 | 1012 | 73 | 2.67 | 34 | 1.24 | 74 | 0 | 44 | 461 |
| 113-05 | | | | | | | | | | | | |
| CEPEC-2002 | 43836.42 | 2314 | 1836 | 806 | 332 | 14.35 | 29 | 1.25 | 64 | 0 | 14 | 474 |
| (VB-1151) | | | | | | | | | | | | |
| PT-08-020-2- | 42462.20 | 2001 | 1694 | 682 | 72 | 3.60 | 71 | 3.55 | 39 | 0 | 86 | 306 |
| 057-18 | | | | | | | | | | | | |
| PT-08-003-1- | 42372.59 | 1216 | 835 | 456 | 55 | 4.52 | 42 | 3.45 | 47 | 0 | 15 | 378 |
| 009-12 | | | | | | | | | | | | |
| MC-08005 | 41832.38 | 1625 | 1448 | 509 | 29 | 1.78 | 34 | 2.09 | 26 | 0 | 13 | 178 |
| MC-08093 | 52115.49 | 1778 | 1262 | 617 | 107 | 6.02 | 97 | 5.46 | 113 | 0 | 22 | 509 |
| PH-15 | 40682.39 | 1524 | 1256 | 608 | 136 | 8.92 | 18 | 1.18 | 46 | 0 | 17 | 272 |
| PT-08-020-1- | 39725.52 | 2066 | 1836 | 750 | 66 | 3.19 | 19 | 0.92 | 39 | 0 | 47 | 223 |
| 145-07 | | | | | | | | | | | | |
| PH-16 | 39476.14 | 1366 | 986 | 538 | 145 | 10.61 | 46 | 3.37 | 35 | 0 | 6 | 384 |
| Pound-7 | 38281.83 | 1494 | 1127 | 523 | 253 | 16.93 | 24 | 1.61 | 38 | 0 | 6 | 371 |
| (BP-R) | | | | | | | | | | | | |
| PT-08-020-3- | 37577.04 | 2555 | 2121 | 836 | 86 | 3.37 | 94 | 3.68 | 62 | 0 | 155 | 439 |
| 092-07 | | | | | | | | | | | | |
| PT-08-069-3- | 37237.89 | 1645 | 1312 | 571 | 148 | 9.00 | 68 | 4.13 | 57 | 0 | 27 | 336 |
| 156-10 | | | | | | | | | | | | |
| CP-49 | 37035.31 | 1523 | 1291 | 564 | 126 | 8.27 | 15 | 0.98 | 20 | 0 | 5 | 249 |
| MC-08017 | 46177.04 | 1837 | 1620 | 678 | 62 | 3.38 | 44 | 2.40 | 69 | 0 | 10 | 248 |
| CMC-011 | 36773.88 | 2302 | 2038 | 850 | 31 | 1.35 | 21 | 0.91 | 113 | 0 | 72 | 263 |
| MC-08171 | 36470.04 | 1668 | 1233 | 442 | 173 | 10.37 | 21 | 1.26 | 165 | 0 | 7 | 436 |
| PT-08-018-2- | 35666.86 | 1752 | 1511 | 767 | 53 | 3.03 | 20 | 1.14 | 75 | 0 | 5 | 231 |
| 084-07 | | | | | | | | | | | | |
| PT-08-006-1- | 43461.07 | 1936 | 1650 | 872 | 58 | 3.00 | 24 | 1.24 | 98 | 0 | 7 | 290 |
| 039-15 | | | | | | | | | | | | |
| CCN-10 | 33185.91 | 824 | 594 | 321 | 80 | 9.71 | 19 | 2.31 | 34 | 0 | 8 | 235 |
| MC-08059 | 41412.65 | 2448 | 2091 | 963 | 95 | 3.88 | 36 | 1.47 | 58 | 0 | 25 | 365 |
| CMC-032 | 40897.10 | 1862 | 1658 | 727 | 62 | 3.33 | 41 | 2.20 | 69 | 0 | 5 | 210 |
| IPIRANGA-1 | 31120.48 | 977 | 722 | 441 | 73 | 7.47 | 26 | 2.66 | 33 | 0 | 5 | 252 |
| CATIE-R6 | 38701.08 | 2615 | 2212 | 1054 | 181 | 6.92 | 9 | 0.34 | 45 | 0 | 14 | 403 |
| IAC-1 | 26579.59 | 1888 | 1287 | 635 | 247 | 13.08 | 95 | 5.03 | 43 | 0 | 154 | 600 |
| CATIE-R1 | 32528.15 | 1898 | 1438 | 684 | 320 | 16.86 | 47 | 2.48 | 52 | 0 | 16 | 469 |
| PT-08-020-3- | 25794.76 | 1669 | 1385 | 619 | 79 | 4.73 | 24 | 1.44 | 30 | 0 | 136 | 284 |
| 092-14 | | | | | | | | | | | | |
| CATIE-R4 | 29700.77 | 1719 | 1324 | 707 | 247 | 14.37 | 18 | 1.05 | 23 | 0 | 42 | 397 |
| NA-33 | 22777.45 | 1466 | 1236 | 645 | 161 | 10.98 | 7 | 0.48 | 23 | 0 | 23 | 236 |
| VB-514 | 22295.15 | 2855 | 2211 | 845 | 183 | 6.41 | 56 | 1.96 | 219 | 0 | 75 | 645 |
| PMCT-58 | 24214.63 | 1272 | 908 | 544 | 229 | 18.00 | 38 | 2.99 | 27 | 0 | 26 | 368 |
| MC-08114 | 19301.48 | 1288 | 1027 | 572 | 124 | 9.63 | 13 | 1.01 | 35 | 0 | 38 | 261 |
| GU-144C T1 | 15641.57 | 1064 | 983 | 524 | 22 | 2.07 | 1 | 0.09 | 10 | 0 | 26 | 85 |
| PT-08-018-2- | 11302.50 | 412 | 349 | 232 | 24 | 5.83 | 4 | 0.97 | 21 | 0 | 2 | 63 |
| 084-10 | | | | | | | | | | | | |
| TSH-1188 | 11126.10 | 240 | 194 | 118 | 22 | 9.17 | 4 | 1.67 | 3 | 0 | 2 | 49 |
| (BP-R) | | | | | | | | | | | | |
| MC-08024 | 7614.80 | 188 | 126 | 92 | 23 | 12.23 | 7 | 3.72 | 5 | 0 | 1 | 64 |
| SCA-6 | 5336.15 | 2538 | 1102 | 629 | 141 | 5.56 | 74 | 2.92 | 1092 | 1 | 96 | 1439 |
| (WB-T) | | | | | | | | | | | | |
| GU-171C | 4461.71 | 615 | 528 | 287 | 19 | 3.09 | 4 | 0.65 | 28 | 0 | 22 | 81 |
| GU-296H | 4186.58 | 401 | 333 | 239 | 11 | 2.74 | 1 | 0.25 | 15 | 2 | 20 | 63 |

TABLE 51
Mean variety data

| | mean | mean | mean | mean | mean | mean | mean | mean | mean |
|---|---|---|---|---|---|---|---|---|---|
| variety | nph | nhph | nps | wbd | bpd | ol | paboa | npg | tpl |
| CMC-001 | 403.75 | 372.17 | 121.33 | 7.17 | 2.42 | 6.92 | 0.17 | 6.00 | 30.92 |
| CMC-008 | 325.80 | 279.27 | 104.27 | 12.00 | 2.80 | 11.47 | 0.00 | 2.87 | 46.60 |
| CMC-034 | 168.80 | 138.07 | 50.60 | 5.93 | 2.47 | 4.53 | 0.00 | 0.53 | 31.27 |
| CMC-007 | 222.53 | 204.13 | 74.67 | 5.67 | 2.40 | 3.13 | 0.00 | 2.40 | 18.27 |
| CMC-015 | 326.00 | 289.33 | 103.47 | 8.60 | 4.13 | 10.60 | 0.00 | 1.47 | 36.33 |
| CMC-006 | 386.58 | 316.00 | 133.75 | 6.58 | 10.75 | 6.17 | 0.00 | 5.50 | 71.17 |
| PT-08-020-1-145-13 | 237.58 | 210.42 | 85.42 | 7.58 | 4.25 | 3.42 | 0.00 | 1.25 | 27.00 |
| CMC-002 | 278.08 | 245.58 | 81.00 | 6.58 | 4.25 | 5.58 | 0.00 | 8.83 | 32.17 |
| CMC-010 | 273.20 | 226.27 | 65.27 | 8.33 | 3.60 | 6.67 | 0.00 | 8.13 | 46.80 |
| PT-08-064-2-122-06 | 174.83 | 141.33 | 57.25 | 8.92 | 5.50 | 7.00 | 0.00 | 1.00 | 33.50 |
| CMC-009 | 213.75 | 192.83 | 85.75 | 5.75 | 1.92 | 3.50 | 0.00 | 2.17 | 21.33 |
| CMC-024 | 242.92 | 213.25 | 82.17 | 5.67 | 5.92 | 4.58 | 0.00 | 2.83 | 28.67 |
| CMC-019 | 264.08 | 232.75 | 101.50 | 4.25 | 6.83 | 7.25 | 0.08 | 8.25 | 31.67 |
| CMC-003 | 332.73 | 303.67 | 129.13 | 6.47 | 2.00 | 7.53 | 0.00 | 4.87 | 29.07 |
| MC-08086 | 165.53 | 141.67 | 48.93 | 5.87 | 3.27 | 5.27 | 0.00 | 0.73 | 24.07 |
| CMC-027 | 198.92 | 176.92 | 72.17 | 7.25 | 1.08 | 4.58 | 0.00 | 0.58 | 22.08 |
| CMC-021 | 252.17 | 215.58 | 95.92 | 7.42 | 5.33 | 4.50 | 0.00 | 3.92 | 36.25 |
| CMC-016 | 292.67 | 233.67 | 87.58 | 12.08 | 5.92 | 9.08 | 0.00 | 1.42 | 58.42 |
| SJ-02 | 199.58 | 168.75 | 66.92 | 12.83 | 1.00 | 4.00 | 0.00 | 1.25 | 31.08 |
| CMC-035 | 277.73 | 242.53 | 88.27 | 8.20 | 5.60 | 5.47 | 0.00 | 1.33 | 34.80 |
| CMC-033 | 166.92 | 136.42 | 63.08 | 5.17 | 3.25 | 4.67 | 0.00 | 0.42 | 30.75 |
| CMC-013 | 318.47 | 236.27 | 100.33 | 21.13 | 6.40 | 12.53 | 0.20 | 6.40 | 82.07 |
| MC-13-050 | 216.27 | 184.73 | 64.33 | 9.20 | 3.27 | 6.40 | 0.07 | 0.33 | 31.40 |
| MCCS_MP01-427 | 142.08 | 108.25 | 44.42 | 11.25 | 1.33 | 4.25 | 0.00 | 0.25 | 33.83 |
| CMC-025 | 241.93 | 214.60 | 102.60 | 3.87 | 2.47 | 4.27 | 0.00 | 2.53 | 26.93 |
| CMC-004 | 468.92 | 407.67 | 208.00 | 10.42 | 6.08 | 20.25 | 0.08 | 11.92 | 59.75 |
| MP01-492 | 176.40 | 141.47 | 48.80 | 9.00 | 1.47 | 5.20 | 0.13 | 0.53 | 35.27 |
| MP01-619 (WB-R) | 187.47 | 159.07 | 64.13 | 4.60 | 6.67 | 3.53 | 0.00 | 0.60 | 28.87 |
| CMC-020 | 271.93 | 244.60 | 79.40 | 11.40 | 2.07 | 4.53 | 0.00 | 2.27 | 27.80 |
| CCN-51 | 134.67 | 107.58 | 41.75 | 5.00 | 3.58 | 3.92 | 0.00 | 0.83 | 27.25 |
| MC-13-259 | 173.93 | 131.53 | 52.47 | 14.87 | 4.80 | 7.60 | 0.00 | 0.80 | 41.07 |
| PT-08-061-3-163-12 | 330.25 | 261.92 | 91.42 | 33.75 | 4.50 | 9.42 | 0.00 | 5.33 | 67.50 |
| CMC-023 | 272.83 | 239.42 | 89.58 | 7.17 | 2.25 | 7.08 | 0.00 | 0.83 | 33.33 |
| PT-08-063-1-065-12 | 219.33 | 193.92 | 73.92 | 11.58 | 1.92 | 5.08 | 0.17 | 0.83 | 25.58 |
| PT-08-020-3-092-05 | 293.53 | 268.07 | 94.33 | 4.73 | 1.87 | 8.33 | 0.00 | 4.07 | 22.80 |
| PT-08-063-1-065-14 | 192.58 | 145.83 | 61.83 | 13.67 | 7.42 | 6.42 | 0.00 | 1.67 | 46.67 |
| CMC-028 | 230.13 | 208.93 | 74.27 | 6.60 | 1.47 | 7.60 | 0.00 | 0.80 | 20.33 |
| CMC-014 | 185.07 | 157.13 | 60.00 | 10.33 | 2.60 | 2.53 | 0.00 | 0.53 | 28.20 |
| CMC-036 | 141.25 | 111.50 | 48.25 | 4.58 | 1.08 | 8.08 | 0.00 | 0.25 | 29.67 |
| PT-08-020-2-057-01 | 361.92 | 305.92 | 147.50 | 4.25 | 2.25 | 10.17 | 0.17 | 32.58 | 55.42 |
| FCOM-150 | 235.33 | 205.00 | 83.00 | 15.08 | 6.50 | 3.00 | 0.00 | 1.17 | 30.50 |
| MCCS_MP01-522 | 136.07 | 91.60 | 38.87 | 6.60 | 4.53 | 2.93 | 0.07 | 1.87 | 44.60 |
| CMC-005 | 260.92 | 228.25 | 99.08 | 7.25 | 8.08 | 3.83 | 0.00 | 2.25 | 31.67 |
| PS-1319 | 151.83 | 128.50 | 49.08 | 12.08 | 2.67 | 3.58 | 0.00 | 0.83 | 23.67 |
| PT-08-069-2-083-10 | 182.17 | 135.08 | 50.25 | 19.17 | 7.25 | 4.42 | 0.00 | 0.67 | 47.50 |
| CMC-022 | 182.75 | 163.00 | 72.42 | 7.00 | 1.42 | 6.08 | 0.00 | 0.33 | 19.58 |
| PT-08-063-3-157-09 | 125.40 | 97.87 | 45.80 | 13.07 | 2.60 | 2.60 | 0.00 | 0.13 | 27.80 |
| CMC-017 | 344.92 | 308.75 | 149.00 | 6.17 | 1.67 | 4.67 | 0.08 | 6.92 | 35.42 |
| CMC-012 | 158.00 | 133.00 | 58.33 | 11.00 | 2.73 | 3.67 | 0.00 | 3.73 | 25.07 |
| PT-08-041-3-093-04 | 177.83 | 149.67 | 68.25 | 8.00 | 2.83 | 3.58 | 0.00 | 1.17 | 28.08 |
| PT-08-063-3-157-11 | 179.17 | 141.83 | 68.33 | 17.17 | 2.42 | 3.67 | 0.00 | 0.92 | 37.17 |
| PT-08-063-3-157-01 | 164.08 | 124.67 | 55.25 | 11.42 | 3.75 | 7.92 | 0.00 | 0.50 | 38.92 |
| PT-08-038-3-096-01 | 208.58 | 159.17 | 59.92 | 7.75 | 2.00 | 6.00 | 0.00 | 1.00 | 48.58 |
| PT-08-018-2-084-17 | 183.80 | 161.53 | 77.40 | 4.53 | 2.53 | 4.40 | 0.00 | 2.20 | 22.40 |
| PT-08-020-3-092-10 | 258.42 | 222.17 | 76.75 | 10.25 | 3.83 | 7.42 | 0.08 | 9.08 | 35.42 |
| CMC-026 | 188.40 | 173.07 | 80.27 | 3.93 | 0.73 | 2.93 | 0.00 | 1.93 | 15.27 |
| PT-08-020-1-145-18 | 219.67 | 195.00 | 88.67 | 10.25 | 2.17 | 3.50 | 0.00 | 5.92 | 24.33 |
| CMC-030 | 180.50 | 149.25 | 80.17 | 5.75 | 4.75 | 3.08 | 0.00 | 1.08 | 30.75 |
| CMC-031 | 265.67 | 242.50 | 99.08 | 4.33 | 6.33 | 4.75 | 0.00 | 1.50 | 23.17 |
| PT-08-063-1-065-11 | 165.08 | 125.67 | 61.08 | 17.25 | 1.75 | 5.42 | 1.25 | 1.58 | 39.17 |
| MC-08103 | 187.73 | 148.93 | 62.60 | 18.07 | 2.93 | 3.20 | 0.00 | 1.47 | 38.60 |
| MCCS_MP01-734 | 84.53 | 60.20 | 31.27 | 4.07 | 3.13 | 1.47 | 0.07 | 0.20 | 24.47 |
| PT-08-063-1-065-17 | 176.33 | 139.83 | 55.08 | 20.08 | 3.08 | 7.50 | 0.00 | 0.92 | 37.67 |
| CMC-018 | 230.67 | 204.93 | 83.27 | 9.60 | 1.20 | 6.47 | 0.00 | 1.00 | 26.07 |
| MCCS_MP01-414 | 90.17 | 70.42 | 28.92 | 3.00 | 4.83 | 1.00 | 0.00 | 0.17 | 19.92 |
| PT-08-020-3-092-01 | 246.83 | 220.17 | 88.08 | 5.92 | 1.50 | 7.17 | 0.00 | 8.58 | 27.00 |
| MC-08061 | 213.73 | 186.73 | 64.13 | 6.67 | 4.87 | 4.33 | 0.00 | 1.47 | 27.00 |
| PT-08-059-2-086-18 | 101.33 | 74.75 | 43.83 | 6.92 | 4.17 | 2.42 | 0.00 | 0.25 | 22.67 |
| PT-08-020-2-057-10 | 238.33 | 214.67 | 78.08 | 5.42 | 0.92 | 2.83 | 0.00 | 9.17 | 23.83 |
| PT-08-018-1-034-02 | 156.60 | 136.13 | 80.60 | 5.93 | 1.20 | 3.53 | 0.00 | 2.73 | 20.60 |
| PT-08-038-2-050-01 | 230.42 | 204.42 | 81.67 | 7.42 | 2.17 | 5.75 | 0.00 | 0.75 | 26.17 |
| MC-13-083 | 150.00 | 125.27 | 52.47 | 7.87 | 1.87 | 1.93 | 0.00 | 1.47 | 24.93 |
| PT-08-038-3-096-07 | 182.27 | 154.55 | 72.00 | 8.91 | 1.18 | 2.00 | 0.00 | 2.64 | 28.18 |
| CMC-029 | 204.58 | 176.25 | 98.50 | 10.08 | 3.33 | 2.75 | 0.00 | 3.17 | 28.42 |
| MC-08091 | 162.17 | 128.25 | 41.42 | 8.08 | 7.50 | 6.25 | 0.00 | 9.33 | 33.58 |
| FB-206 (BP-S) | 186.50 | 148.33 | 73.58 | 15.50 | 9.33 | 6.75 | 0.00 | 1.75 | 38.42 |
| PT-08-038-2-050-08 | 246.25 | 215.50 | 104.00 | 8.33 | 1.92 | 3.83 | 0.08 | 7.00 | 30.83 |
| PT-08-061-2-048-10 | 108.73 | 93.07 | 50.00 | 7.27 | 2.00 | 1.60 | 0.00 | 0.20 | 15.67 |
| PT-08-063-2-117-11 | 151.42 | 125.25 | 51.92 | 13.83 | 2.83 | 3.17 | 0.00 | 0.58 | 26.25 |
| PT-08-018-3-178-09 | 160.08 | 143.75 | 60.33 | 2.50 | 1.17 | 2.50 | 0.00 | 1.33 | 15.50 |
| SIAL-70 (WB-S) | 157.50 | 144.50 | 61.67 | 5.00 | 1.00 | 3.42 | 0.00 | 1.67 | 13.42 |
| PT-08-069-2-083-18 | 116.42 | 92.42 | 43.75 | 13.83 | 1.92 | 2.25 | 0.00 | 0.50 | 25.17 |
| CEPEC-2004 (CEPEC-46) | 181.17 | 147.75 | 50.42 | 8.00 | 5.08 | 5.58 | 0.00 | 1.58 | 33.08 |
| PT-08-038-1-113-05 | 210.15 | 174.15 | 77.85 | 5.62 | 2.62 | 5.69 | 0.00 | 3.38 | 35.46 |
| CEPEC-2002 (VB-1151) | 192.83 | 153.00 | 67.17 | 27.67 | 2.42 | 5.33 | 0.00 | 1.17 | 39.50 |
| PT-08-020-2-057-18 | 166.75 | 141.17 | 56.83 | 6.00 | 5.92 | 3.25 | 0.00 | 7.17 | 25.50 |
| PT-08-003-1-009-12 | 101.33 | 69.58 | 38.00 | 4.58 | 3.50 | 3.92 | 0.00 | 1.25 | 31.50 |
| MC-08005 | 135.42 | 120.67 | 42.42 | 2.42 | 2.83 | 2.17 | 0.00 | 1.08 | 14.83 |
| MC-08093 | 118.53 | 84.13 | 41.13 | 7.13 | 6.47 | 7.53 | 0.00 | 1.47 | 33.93 |
| PH-15 | 127.00 | 104.67 | 50.67 | 11.33 | 1.50 | 3.83 | 0.00 | 1.42 | 22.67 |
| PT-08-020-1-145-07 | 172.17 | 153.00 | 62.50 | 5.50 | 1.58 | 3.25 | 0.00 | 3.92 | 18.58 |
| PH-16 | 113.83 | 82.17 | 44.83 | 12.08 | 3.83 | 2.92 | 0.00 | 0.50 | 32.00 |
| Pound-7 (BP-R) | 124.50 | 93.92 | 43.58 | 21.08 | 2.00 | 3.17 | 0.00 | 0.50 | 30.92 |
| PT-08-020-3-092-07 | 212.92 | 176.75 | 69.67 | 7.17 | 7.83 | 5.17 | 0.00 | 12.92 | 36.58 |
| PT-08-069-3-156-10 | 137.08 | 109.33 | 47.58 | 12.33 | 5.67 | 4.75 | 0.00 | 2.25 | 28.00 |
| CP-49 | 126.92 | 107.58 | 47.00 | 10.50 | 1.25 | 1.67 | 0.00 | 0.42 | 20.75 |
| MC-08017 | 122.47 | 108.00 | 45.20 | 4.13 | 2.93 | 4.60 | 0.00 | 0.67 | 16.53 |
| CMC-011 | 191.83 | 169.83 | 70.83 | 2.58 | 1.75 | 9.42 | 0.00 | 6.00 | 21.92 |
| MC-08171 | 139.00 | 102.75 | 36.83 | 14.42 | 1.75 | 13.75 | 0.00 | 0.58 | 36.33 |
| PT-08-018-2-084-07 | 146.00 | 125.92 | 63.92 | 4.42 | 1.67 | 6.25 | 0.00 | 0.42 | 19.25 |
| PT-08-006-1-039-15 | 129.07 | 110.00 | 58.13 | 3.87 | 1.60 | 6.53 | 0.00 | 0.47 | 19.33 |
| CCN-10 | 68.67 | 49.50 | 26.75 | 6.67 | 1.58 | 2.83 | 0.00 | 0.67 | 19.58 |
| MC-08059 | 163.20 | 139.40 | 64.20 | 6.33 | 2.40 | 3.87 | 0.00 | 1.67 | 24.33 |
| CMC-032 | 124.13 | 110.53 | 48.47 | 4.13 | 2.73 | 4.60 | 0.00 | 0.33 | 14.00 |
| IPIRANGA-1 | 81.42 | 60.17 | 36.75 | 6.08 | 2.17 | 2.75 | 0.00 | 0.42 | 21.00 |
| CATIE-R6 | 174.33 | 147.47 | 70.27 | 12.07 | 0.60 | 3.00 | 0.00 | 0.93 | 26.87 |
| IAC-1 | 157.33 | 107.25 | 52.92 | 20.58 | 7.92 | 3.58 | 0.00 | 12.83 | 50.00 |
| CATIE-R1 | 126.53 | 95.87 | 45.60 | 21.33 | 3.13 | 3.47 | 0.00 | 1.07 | 31.27 |
| PT-08-020-3-092-14 | 139.08 | 115.42 | 51.58 | 6.58 | 2.00 | 2.50 | 0.00 | 11.33 | 23.67 |
| CATIE-R4 | 114.60 | 88.27 | 47.13 | 16.47 | 1.20 | 1.53 | 0.00 | 2.80 | 26.47 |
| NA-33 | 122.17 | 103.00 | 53.75 | 13.42 | 0.58 | 1.92 | 0.00 | 1.92 | 19.67 |
| VB-514 | 237.92 | 184.25 | 70.42 | 15.25 | 4.67 | 18.25 | 0.00 | 6.25 | 53.75 |
| PMCT-58 | 84.80 | 60.53 | 36.27 | 15.27 | 2.53 | 1.80 | 0.00 | 1.73 | 24.53 |
| MC-08114 | 107.33 | 85.58 | 47.67 | 10.33 | 1.08 | 2.92 | 0.00 | 3.17 | 21.75 |
| GU-144C T1 | 88.67 | 81.92 | 43.67 | 1.83 | 0.08 | 0.83 | 0.00 | 2.17 | 7.08 |
| PT-08-018-2-084-10 | 34.33 | 29.08 | 19.33 | 2.00 | 0.33 | 1.75 | 0.00 | 0.17 | 5.25 |
| TSH-1188 (BP-R) | 20.00 | 16.17 | 9.83 | 1.83 | 0.33 | 0.25 | 0.00 | 0.17 | 4.08 |
| MC-08024 | 15.67 | 10.50 | 7.67 | 1.92 | 0.58 | 0.42 | 0.00 | 0.08 | 5.33 |
| SCA-6 (WB-T) | 211.50 | 91.83 | 52.42 | 11.75 | 6.17 | 91.00 | 0.08 | 8.00 | 119.92 |
| GU-171C | 51.25 | 44.00 | 23.92 | 1.58 | 0.33 | 2.33 | 0.00 | 1.83 | 6.75 |
| GU-296H | 33.42 | 27.75 | 19.92 | 0.92 | 0.08 | 1.25 | 0.17 | 1.67 | 5.25 |

What we claim is:

1. A new and distinct variety of cacao plant as shown and described herein, wherein the variety is selected from the group consisting of ‘CMC-001’, ‘CMC-002’, ‘CMC-003’, ‘CMC-004’, ‘CMC-005’, ‘CMC-006’, ‘CMC-007’, ‘CMC-008’, ‘CMC-009’, ‘CMC-010’, ‘CMC-011’, ‘CMC-012’, ‘CMC-013’, ‘CMC-014’, ‘CMC-015’, ‘CMC-016’, ‘CMC-017’, ‘CMC-018’, ‘CMC-019’, ‘CMC-020’, ‘CMC-021’, ‘CMC-023’, ‘CMC-024’, ‘CMC-025’, ‘CMC-026’, ‘CMC-027’, ‘CMC-028’, ‘CMC-029’, ‘CMC-030’, ‘CMC-031’, ‘CMC-032’, ‘CMC-033’, ‘CMC-034’, ‘CMC-035’, ‘CMC-036’, and ‘CMC-022’.
